(19) United States
(12) Patent Application Publication
(10) Pub. No.: US 2026/0097046 A1
(43) Pub. Date: Apr. 9, 2026

(54) PLASMODIUM FALCIPARUM BLOOD STAGE INHIBITORS

(71) Applicants: University of Georgia Research Foundation, Inc., Athens, GA (US); Northeastern University, Boston, MA (US)

(72) Inventors: Alicia N. WAGNER, Jamaica Plain, MA (US); Roger TROMBLEY, Boston, MA (US); Maris E. PODGURSKI, Arundel, ME (US); Jacqueline R. SMITH, Stow, MA (US); Meng CUI, Boston, MA (US); Adriana A. MARIN, Bogotá (CO); Steven P. MAHER, Bogart, GA (US); Dennis E. KYLE, Athens, GA (US); Roman MANETSCH, Boston, MA (US); Anthony A. RUBERTO, Athens, GA (US)

(21) Appl. No.: 19/417,523

(22) Filed: Dec. 12, 2025

Related U.S. Application Data
(63) Continuation-in-part of application No. PCT/US2024/033498, filed on Jun. 12, 2024.
(60) Provisional application No. 63/507,757, filed on Jun. 13, 2023.

(51) Int. Cl. A61K 31/551; A61K 31/4164; A61K 31/4192; A61K 31/4196; A61K 31/421; A61K 31/4245; A61K 31/426; A61K 31/427; A61K 31/429; A61K 31/4418; A61K 31/4439; A61K 31/454; A61K 31/455; A61K 31/496; A61K 31/497; A61K 31/498; A61K 31/501; A61K 31/506; A61K 31/536; A61K 31/5377; A61K 31/538; A61P 33/06; C07B 59/00; C07D 213/50; C07D 213/81; C07D 233/90; C07D 237/24; C07D 239/28; C07D 241/24; C07D 249/04; C07D 249/10; C07D 263/34; C07D 271/10; C07D 277/56; C07D 401/04; C07D 401/06; C07D 401/12; C07D 403/04; C07D 403/06; C07D 405/12; C07D 413/04; C07D 413/06; C07D 413/12; C07D 417/12; C07D 513/04

(52) U.S. Cl. CPC A61K 31/551; A61K 31/4164; A61K 31/4192; A61K 31/4196; A61K 31/421; A61K 31/4245; A61K 31/426; A61K 31/427; A61K 31/429; A61K 31/4418; A61K 31/4439; A61K 31/454; A61K 31/455; A61K 31/496; A61K 31/497; A61K 31/498; A61K 31/501; A61K 31/506; A61K 31/536; A61K 31/5377; A61K 31/538; A61P 33/06; C07B 59/002; C07D 213/50; C07D 213/81; C07D 233/90; C07D 237/24; C07D 239/28; C07D 241/24; C07D 249/04; C07D 249/10; C07D 263/34; C07D 271/10; C07D 277/56; C07D 401/04; C07D 401/06; C07D 401/12; C07D 403/04; C07D 403/06; C07D 405/12; C07D 413/04; C07D 413/06; C07D 413/12; C07D 417/12; C07D 513/04

(57) ABSTRACT

In one aspect, the disclosure relates to compounds that inhibit Plasmodium falciparum asexual blood stage parasites (PfABS) In one aspect, the compounds exhibit sub-millimolar potency against the intraerythrocytic stages of Plasmodium falciparum and other Plasmodium species. In another aspect, the compounds are soluble in aqueous solutions at pH 7.4, making them suitable for oral administration to patients. Also disclosed are methods of making the compounds, pharmaceutical compositions comprising the same, and methods of treating or preventing malaria using the same. This abstract is intended as a scanning tool for purposes of searching in the particular art and is not intended to be limiting of the present disclosure.

### CROSS REFERENCE TO RELATED APPLICATIONS

This application is a continuation-in-part of PCT/US2024/033498 filed on Jun. 12, 2024, which claims the benefit of U.S. Provisional Application No. 63/507,757 filed on Jun. 13, 2023, each of which is incorporated herein by reference in its entirety.

### STATEMENT REGARDING FEDERALLY SPONSORED RESEARCH OR DEVELOPMENT

This invention was made with government support under grant numbers 1R01AI144464 and R01AI153290 awarded by the National Institutes of Health. The government has certain rights in the invention.

### BACKGROUND

Malaria is the most detrimental parasitic disease today. It infected approximately 249 million people in 2023 alone, with approximately 608,000 individuals ultimately succumbing to this disease. While there have been recent developments in antimalarial treatments and the global malaria incidence has decreased by 23% since the turn of the decade, there is still much to be done to eradicate this disease. With the rise of drug resistance against current antimalarial treatment options, there is an urgent need for the development of novel, structurally unique antimalarial drugs that act upon new parasite targets.

Currently, artemisinin combination therapies (ACTs) are the frontline treatment for treating malaria, which is caused by six different species of Plasmodium. P. falciparum causes the most cases of malaria annually, while P. vivax is the most widespread globally. While monotherapies have been used to treat malaria for decades, combination therapies are now used to overcome widespread resistance to legacy antimalarials such as chloroquine and the rapid development and spread of resistance to new antimalarials such as mefloquine. Typically, combination therapies include a fast-acting component paired with a long-lasting component. For ACTs, the fast-acting component is an artemisinin analog which results in rapid reduction of blood parasitemia (10¹² parasites in an infected individual) to prevent or reverse malaria symptoms while the long-acting component completely removes all parasites from the patient, resulting in cure. Over the past decades, successful use of malaria control strategies including ACTs have resulted in a remarkable decrease in cases and deaths. However, artemisinin resistance is now widespread in Asia and present in Africa, threatening the future efficacy of ACTs. To avoid the resurgence of malaria globally, triple combination ACTs are undergoing trials, but these developments show new drugs active against new protein targets are needed for continued malaria control and eventual elimination.

Desired properties for new antimalarials include safety, rapid mechanism of action, and inexpensive production. New antimalarials should also be able to target strains of malaria resistant to other treatments. These needs and other needs are satisfied by the present disclosure.

### SUMMARY

In accordance with the purpose(s) of the present disclosure, as embodied and broadly described herein, the disclosure, in one aspect, relates to compounds that inhibit Plasmodium falciparum asexual blood stage parasites (PfABS) and other Plasmodium species. In one aspect, the compounds exhibit sub-millimolar potency against the intraerythrocytic stages of Plasmodium falciparum. In another aspect, the compounds are soluble in aqueous solutions at pH 7.4, making them suitable for oral administration to patients. Also disclosed are methods of making the compounds, pharmaceutical compositions comprising the same, and methods of treating or preventing malaria using the same.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows potency of APZ-2199 or APZ-2161 against W2 parent and resistant clones. Clones C—were generated against AP-2161, Clones D—were generated against APZ-2199. Datapoints represent a pEC₅₀ (−log EC₅₀, M) calculated from an independent experiment, bars represent SD, *p<0.05, **p<0.01 Wilcoxon test.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein are several inhibitors of Plasmodium falciparum asexual blood stage parasites (PfABS). In one aspect, when synthesized and tested, the disclosed compounds exhibited potency against the intraerythrocytic stages of Plasmodium falciparum. Also disclosed are several scaffolds and eight chemotypes displaying sub-micromolar potency against the intraerythrocytic stages of Plasmodium falciparum.

In one aspect, the disclosed compounds have Formula I.

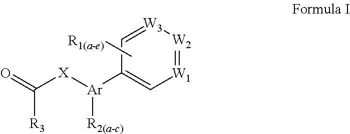

wherein each of R₁ₐ—R₁ₑ is independently selected from hydrogen, Cl, CH₃, OCH₃, CH₂CH₃, CF₃, CH(CH₃)₂, OCF₃, OCF₂H, CF₂H, NH₂, C(═O)CF₃, S(═O)₂CH₃, cyclobutyl, cyclopentyl, cyclohexyl, or any combination thereof;
wherein W₁, W₂, and W₃ are independently N or substituted or unsubstituted C;
wherein Ar is a 5- or 6-membered aromatic or heteroaromatic ring or a bridged cycloalkyl or heterocycloalkyl ring;
   wherein each of R₂ₐ—R_{2c}, if present, is hydrogen, C1-C4 linear or branched alkyl, or when
   R₄ is present, one of R₂ₐ—R_{2c} and R₄ together form a C1-C4 alkyl bridge;
wherein X is NR₈ or is absent;
   wherein R₈ is C1-C4 linear or branched alkyl, CH₂CF₃, or hydrogen;
wherein R₃ is selected from

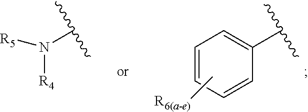

wherein R₄ is selected from deuterated or undeuterated C1-C4 linear or branched alkyl; hydrogen; or together with R₅ forms a substituted or unsubstituted cycloalkyl, heterocycloalkyl, aryl, or heteroaryl group containing from 3 to 9 ring atoms, or R₄ and one of R₂ₐ—R_{2c} together form a C1-C4 alkyl bridge;
wherein R₅ is selected from substituted or unsubstituted C3-C9 cycloalkyl, heterocycloalkyl, aryl, or heteroaryl, or together with R₄ forms a substituted or unsubstituted cycloalkyl, heterocycloalkyl, aryl, or heteroaryl group containing from 3 to 9 ring atoms; and
wherein each of R₆ₐ—R₆ₑ is independently selected from hydrogen, halogen, hydroxyl, cyano, nitro, substituted or unsubstituted C1-C4 linear or branched alkyl, alkoxy, alkylsulfonyl, substituted or unsubstituted amino, substituted or unsubstituted amide, substituted or unsubstituted ketone, or any combination thereof;
provided the compound of Formula I is not

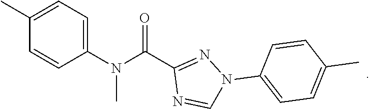

In one aspect, each of R₁ₐ—R₁ₑ is independently selected from hydrogen, Cl, CH₃, OCH₃, CH₂CH₃, CF₃, CH(CH₃)₂, OCF₃, OCF₂H, CF₂H, cyclobutyl, cyclopentyl, cyclohexyl, or any combination thereof. In another aspect, the compound has the structure

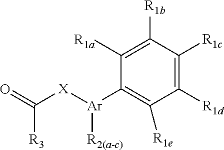

wherein R₁ₐ, R_{1b}, R_{1d}, and R₁ₑ are hydrogen and R₁ₑ is selected from hydrogen, Cl, CH₃, OCH₃, CH₂CH₃, CF₃, CH(CH₃)₂OCF₃, OCF₂H, CF₂H, cyclobutyl, cyclopentyl, or cyclohexyl. In another aspect, R₁ₐ and R_{1b} are hydrogen, R_{1c} is selected from hydrogen, Cl, CH₃, OCH₃, CH₂CH₃, CF₃, CH(CH₃)₂, OCF₃, OCF₂H, CF₂H, cyclobutyl, cyclopentyl, or cyclohexyl, and R₁ₐ and R₁ₑ are independently selected from hydrogen, Cl, and CH₃.

In one aspect, the compound has the structure

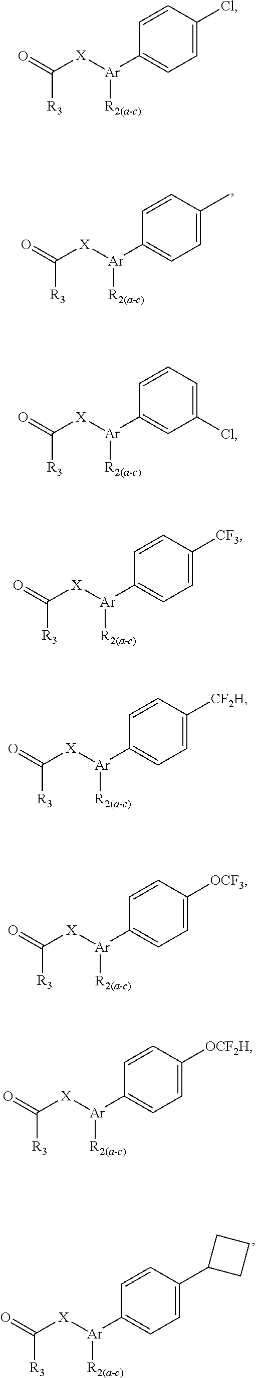

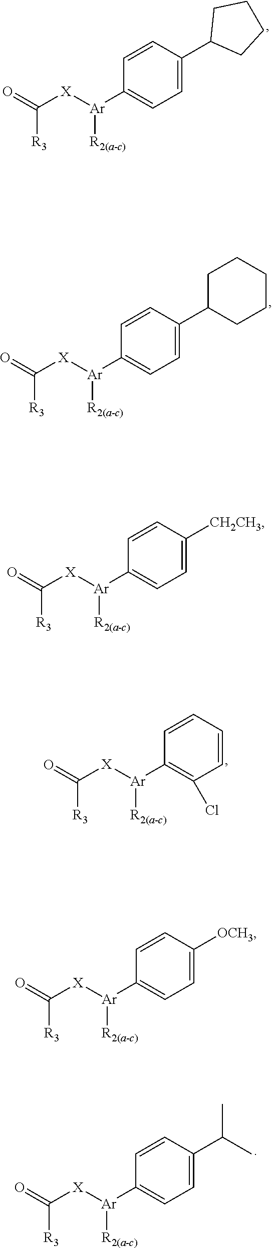

In another aspect, in the disclosed compound, each of R₂ₐ—R_{2c}, if present, is hydrogen or methyl, or when R₄ is present, one of R₂ₐ—R_{2c} and R₄ together form a C1 alkyl bridge.

In some aspects, R₃ can be

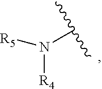

wherein R₄ is hydrogen, methyl, ethyl, isopropyl, tert-butyl, CD₃, CH₂CF₃, or together with R₅ forms a substituted or unsubstituted cycloalkyl or heterocycloalkyl, group containing from 3 to 9 ring atoms, or R₄ and one of R₂ₐ—R_{2c}, if present, together form a C1 alkyl bridge; and R₅ is selected from substituted or unsubstituted C3-C9 cycloalkyl, heterocycloalkyl, aryl, or heteroaryl, or together with R₄ forms a substituted or unsubstituted cycloalkyl or heterocycloalkyl group containing from 3 to 9 ring atoms. Further in this aspect, R₃ can be selected from

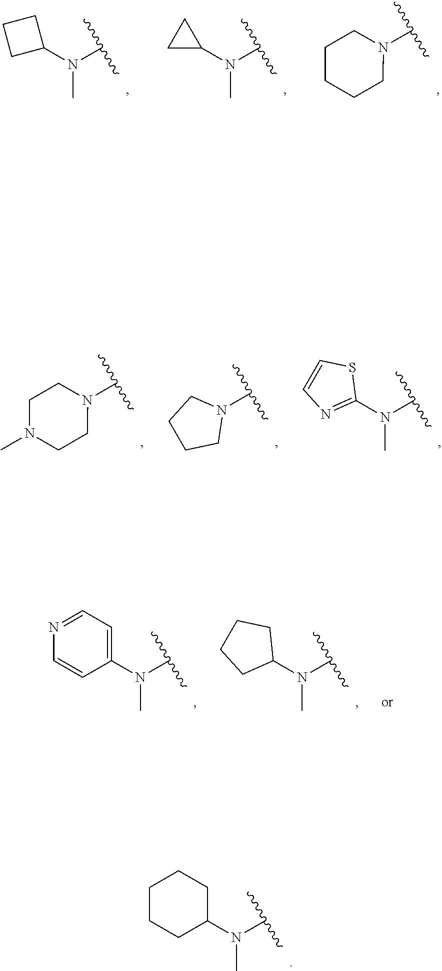

In another aspect, R₄ can be H, methyl, ethyl, isopropyl, tert-butyl, or CD₃, and R₅ is

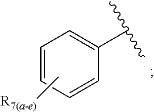

furthermore, each of R₇ₐ—R₇ₑ is independently selected from hydrogen, halogen, hydroxyl, cyano, nitro, trifluoromethoxy, substituted or unsubstituted C1-C4 linear or branched alkyl, alkoxy, alkylsulfonyl, substituted or unsubstituted amino, substituted or unsubstituted amide, or any combination thereof.

In an alternative aspect, R₃ can be

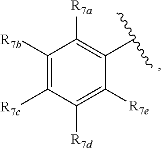

while R₇ₐ and R_{7b} are hydrogen; and R_{7c} is selected from Cl, CH₃, OCH₃, CN, H, SO₂CH₃, COCH₃, NO₂, NHCOCH₃, CF₃, OH, F, N(CH₃)₂, CH₂CH₃, CH(CH₃)₂, or C(CH₃)₃; and while R₇ₐ is selected from Cl, H, CH₃, or CF₃; and while R₇ₑ is selected from H or CH₃. Further in this aspect, R₇ₐ, R_{7b}, R_{7d}, and R₇ₑ are hydrogen and R_{7c} is selected from Cl or CH₃.

In some aspects, R₃ can be

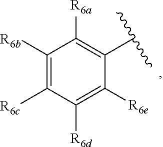

and R₆ₐ—R₆ₑ are independently selected from Cl, CH₃, OCH₃, CN, H, SO₂CH₃, COCH₃, NO₂, NHCOCH₃, CF₃, OH, F, N(CH₃)₂, CH₂CH₃, CH(CH₃)₂, C(CH₃)₃, or any combination thereof. In one aspect, R₆ₐ, R_{6b}, R_{6d}, and R₆ₑ are hydrogen, and R_{6c} is selected from Cl or methyl.

In one aspect, X is absent. In an alternative aspect, X is present and R₈ is H or methyl.

In any of these aspects, Ar can be selected from,

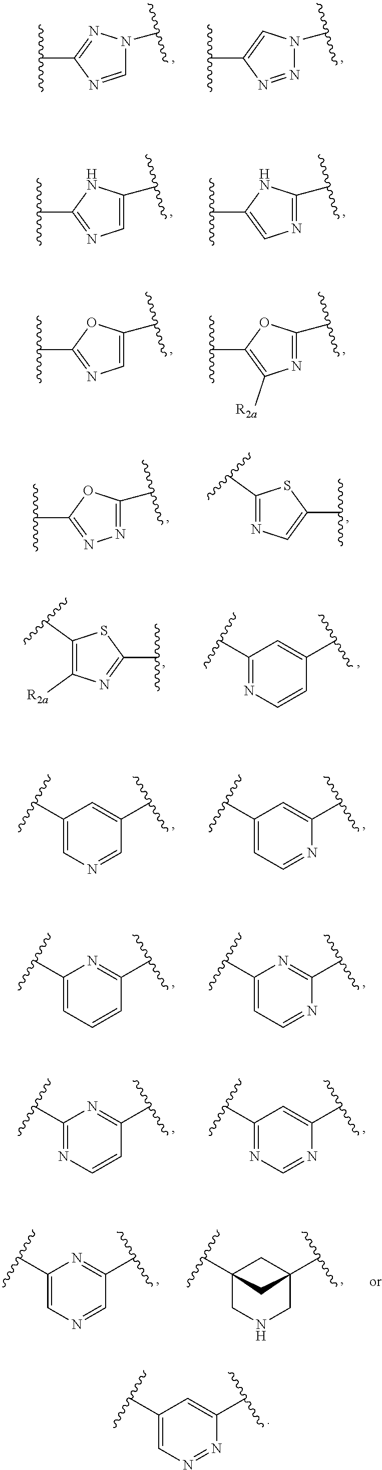

In a further aspect, the compound can have a formula

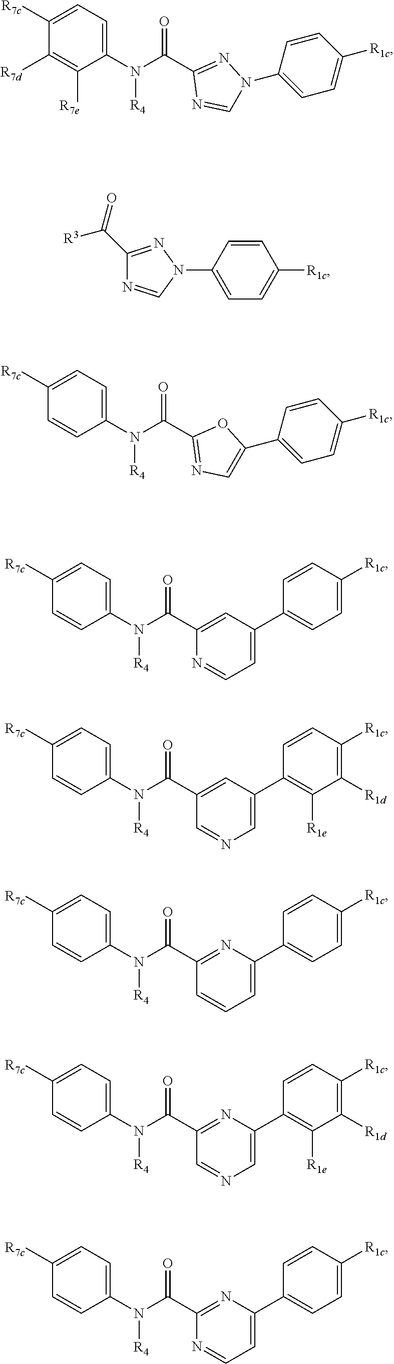

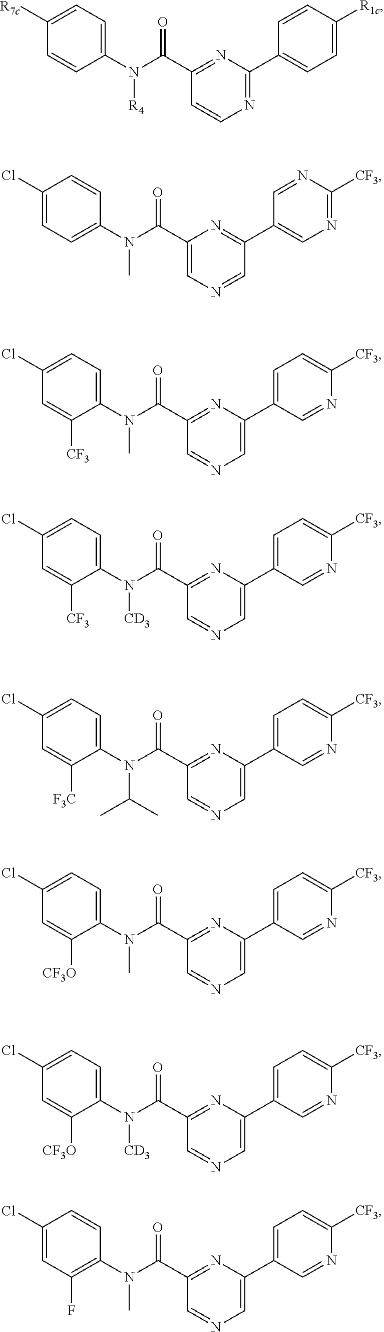

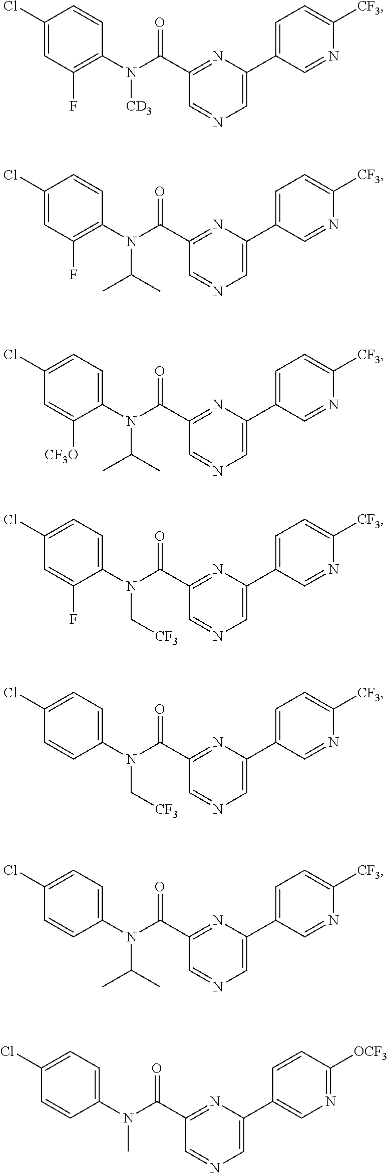

or any combination thereof.

In still another aspect, the compound can be selected from

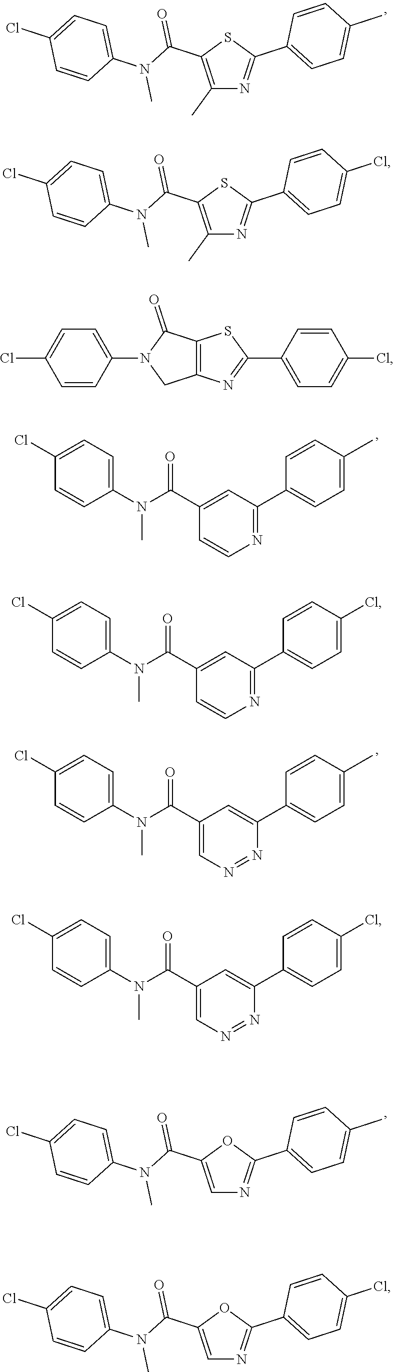

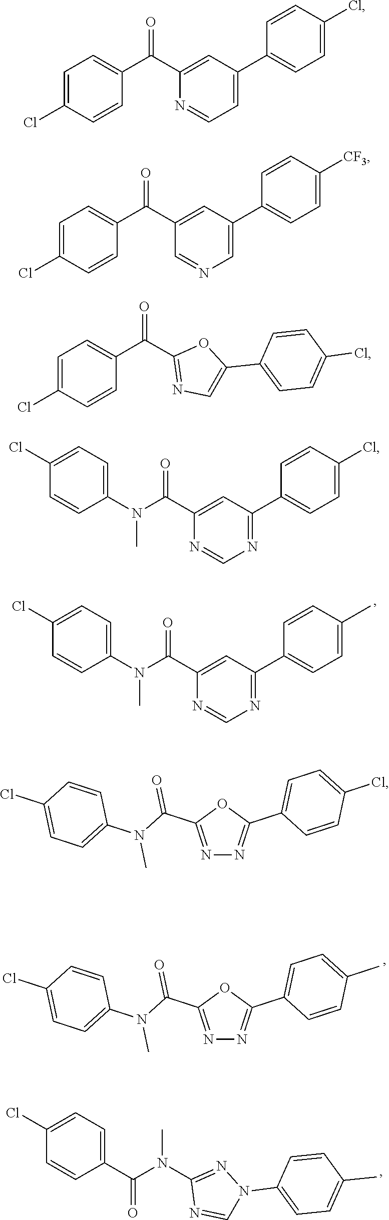

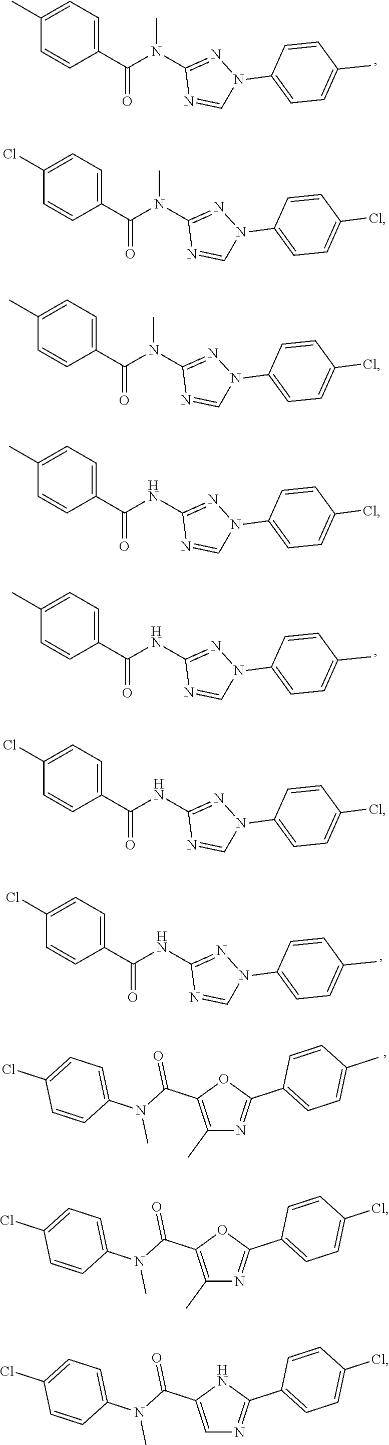

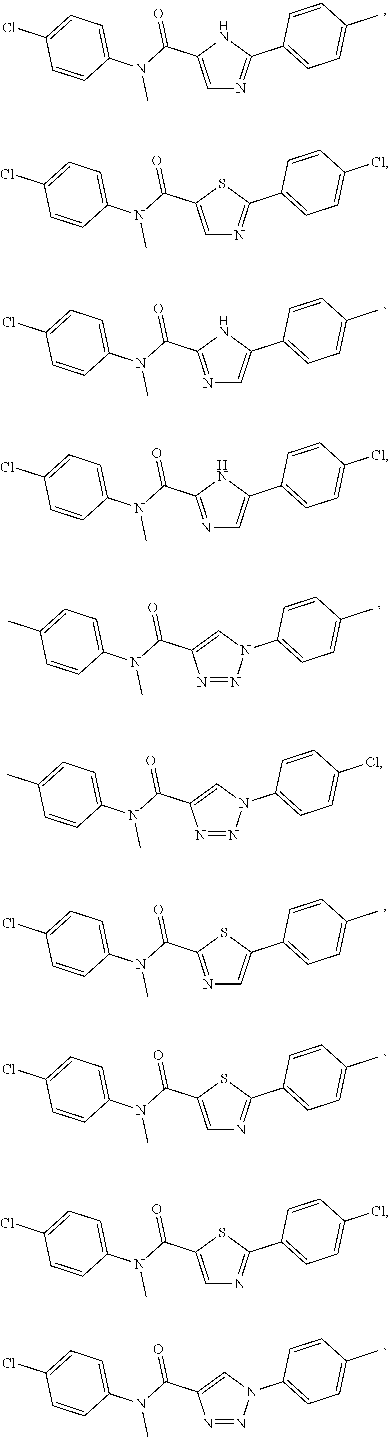

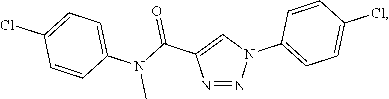 [SMILES: CN(C(=O)c1cn(-c2ccc(Cl)cc2)nn1)c1ccc(Cl)cc1]

or any combination thereof.

In yet another aspect, the compound can be selected from

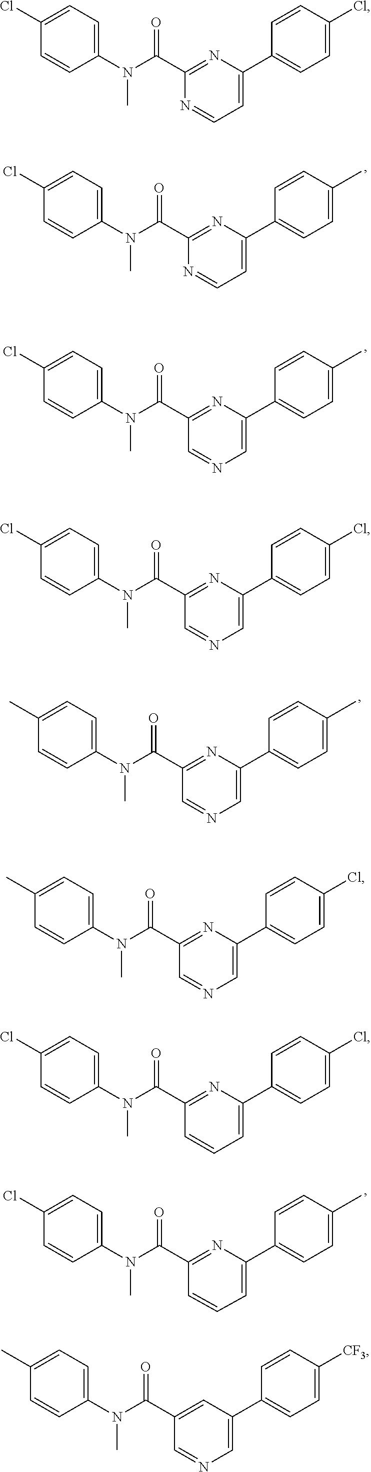

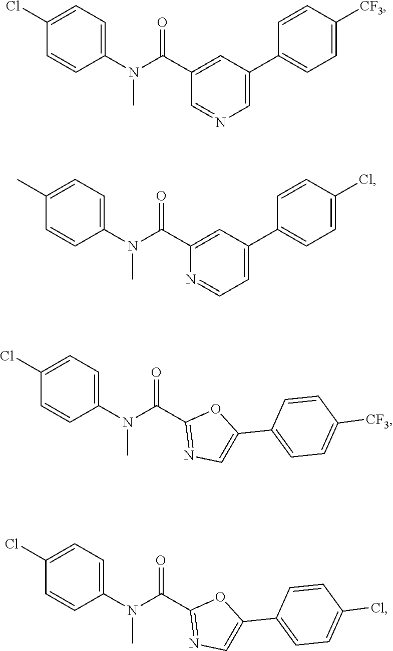 [SMILES: CN(C(=O)c1cncc(-c2ccc(C(F)(F)F)cc2)c1)c1ccc(Cl)cc1 ; Cc1ccc(N(C)C(=O)c2cc(-c3ccc(Cl)cc3)ccn2)cc1 ; CN(C(=O)c1ncc(-c2ccc(C(F)(F)F)cc2)o1)c1ccc(Cl)cc1 ; CN(C(=O)c1ncc(-c2ccc(Cl)cc2)o1)c1ccc(Cl)cc1]

or any combination thereof.

In another aspect, the compounds have Formula II:

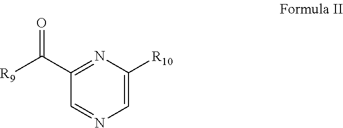

wherein R₁₀ is selected from substituted or unsubstituted C5-C6 cycloalkyl, substituted or unsubstituted C5-C6 heterocycloalkyl, substituted or unsubstituted C5-C6 cycloalkenyl, substituted or unsubstituted C5-C6 heterocycloalkenyl,

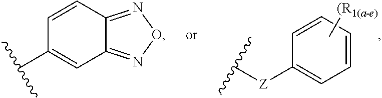

wherein Z is NH or is absent,
wherein each of R₁ₐ—R₁ₑ is independently selected from hydrogen, Cl, CH₃, OCH₃, CH₂CH₃, CF₃, CH(CH₃)₂, OCF₃, OCF₂H, CF₂H, cyclobutyl, cyclopentyl, cyclohexyl, or any combination thereof,
wherein R₉ is —Y—R₁₃;
   wherein Y is selected from OR₁₂, NR₁₁R₁₂, CHR₁₁, or C(R₁₁)₂;
   wherein R₁₁ is deuterated or undeuterated C1-C4 alkyl;
   wherein R₁₂ is a substituted or unsubstituted C5-C7 cycloalkyl, heterocycloalkyl, aryl, or heteroaryl group; or
   wherein R₁₁ and R₁₂ together form a substituted or unsubstituted C5-C10 cycloalkyl, heterocycloalkyl, aryl, or heteroaryl group.

In one aspect, R₁₀ can be

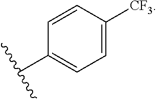 [SMILES: *c1ccc(C(F)(F)F)cc1]

In some aspects, R₁₁, when present, is methyl, ethyl, isopropyl, tert-butyl, or CD₃. In another aspect, R₁₁ and R₁₂ together form a fused bicyclic heterocycloalkyl or heteroaryl ring system.

In an aspect, the compound of Formula II can have the structure

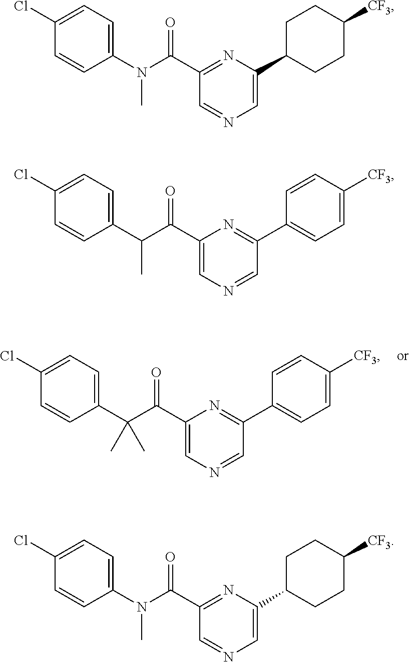 [SMILES: CN(C(=O)c1cncc([C@H]2CC[C@@H](C(F)(F)F)CC2)n1)c1ccc(Cl)cc1 ; CC(C(=O)c1cncc(-c2ccc(C(F)(F)F)cc2)n1)c1ccc(Cl)cc1 ; CC(C)(C(=O)c1cncc(-c2ccc(C(F)(F)F)cc2)n1)c1ccc(Cl)cc1 ; CN(C(=O)c1cncc([C@H]2CC[C@H](C(F)(F)F)CC2)n1)c1ccc(Cl)cc1]

In one aspect, disclosed herein are pharmaceutical compositions including a disclosed compound or a pharmaceutically acceptable salt thereof. In a further aspect, the pharmaceutical compositions can have an EC₅₀ against Plasmodium falciparum: asexual blood stages (W2) of less than or equal to 1 μM, or less than or equal to 0.1 μM.

Furthermore, disclosed herein is a method for treating or preventing an infection caused by a Plasmodium species in a subject. In one aspect, the infection can be malaria. In another aspect, the method includes administering a therapeutically effective amount of a disclosed compound or pharmaceutical composition to the subject. In some aspects, the malaria can be a drug-resistant strain of malaria.

In a further aspect, the method can include administering at least one additional anti-malarial treatment to the subject. In a further aspect, the at least one anti-malarial treatment can be atovaquone-proguanil, chloroquine, doxycycline, mefloquine, primaquine, tafenoquine, artesunate, clindamycin, hydroxychloroquine, quinidine gluconate-sulfate, quinine dihydrochloride, quinine sulfate, or any combination thereof.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,”, “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound,” “a halogen,” or “an alkyl group,” include, but are not limited to, mixtures or combinations of two or more such compounds, halogens, or alkyl groups, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a compound refers to an amount that is sufficient to achieve the desired improvement in the property modulated by the formulation component, e.g. achieving the prevention or effective treatment of a malaria infection. The specific level in terms of wt % in a composition required as an effective amount will depend upon a variety of factors including the strain of malaria, age and weight of patient, and any concurrent treatments or preventatives being administered.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### Chemical Definitions

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “alkanediyl” as used herein, refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂— (methylene), —CH₂CH₂—, —CH₂C(CH₃)₂CH₂—, and —CH₂CH₂CH₂— are non-limiting examples of alkanediyl groups.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or -OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) and —N(-alkyl)₂, where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen” or “halide,” as used herein can be used interchangeably and refer to F, Cl, Br, or I.

The terms “pseudohalide,” “pseudohalogen” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxy” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula -SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” . . . “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, =NNHC(O)R*, =NNHC(O)OR*, =NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

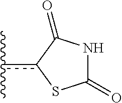

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

“Inorganic radicals,” as the term is defined and used herein, contain no carbon atoms and therefore comprise only atoms other than carbon. Inorganic radicals comprise bonded combinations of atoms selected from hydrogen, nitrogen, oxygen, silicon, phosphorus, sulfur, selenium, and halogens such as fluorine, chlorine, bromine, and iodine, which can be present individually or bonded together in their chemically stable combinations. Inorganic radicals have 10 or fewer, or preferably one to six or one to four inorganic atoms as listed above bonded together. Examples of inorganic radicals include, but not limited to, amino, hydroxy, halogens, nitro, thiol, sulfate, phosphate, and like commonly known inorganic radicals. The inorganic radicals do not have bonded therein the metallic elements of the periodic table (such as the alkali metals, alkaline earth metals, transition metals, lanthanide metals, or actinide metals), although such metal ions can sometimes serve as a pharmaceutically acceptable cation for anionic inorganic radicals such as a sulfate, phosphate, or like anionic inorganic radical. Inorganic radicals do not comprise metalloids elements such as boron, aluminum, gallium, germanium, arsenic, tin, lead, or tellurium, or the noble gas elements, unless otherwise specifically indicated elsewhere herein.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the disclosed formulas, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral carbon.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, sulfur, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds further comprise prodrugs thereof and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

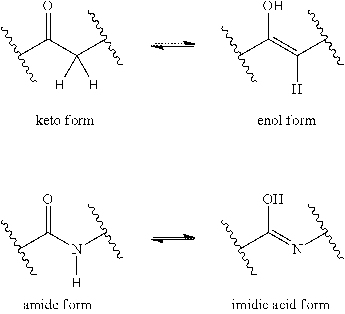 [SMILES: *CC(*)=O ; */C=C(/*)O ; *NC(*)=O ; */N=C(/*)O]

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

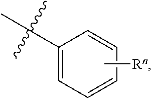

which is understood to be equivalent to a formula:

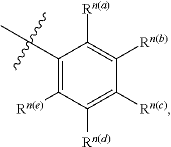

wherein n is typically an integer. That is, Rⁿ is understood to represent five independent substituents, R^{n(a)}, R^{n(b)}, R^{n(c)}, R^{n(d)}, and R^{n(e)}. By “independent substituents,” it is meant that each R substituent can be independently defined. For example, if in one instance Rₙ₍ₐ₎ is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

### Pharmaceutical Definitions

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as malaria. The effect can be therapeutic in terms of a partial or complete cure of a disease, condition, symptom or adverse effect attributed to the disease, disorder, or condition. The term “treatment” as used herein can include any treatment of malaria in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1-to-C 6 alkyl esters and C 5-to-C 7 cycloalkyl esters, although C 1-to-C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1-to-C 6 alkyl amines and secondary C 1-to-C 6 dialkyl amines. In the case of secondary amines, the amine can also be in the form of a 5- or 6-membered heterocycle containing one nitrogen atom. Amides derived from ammonia, C 1-to-C 3 alkyl primary amides and C 1-to-C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

The term “pharmaceutically acceptable co-crystal” means one that is compatible with the other ingredients of the formulation and not deleterious to the recipient thereof.

In a further aspect, the disclosed compounds can be isolated as solvates and, in particular, as hydrates of a disclosed compound, which can be obtained, for example, by crystallization from a solvent or from aqueous solution. In this connection, one, two, three or any arbitrary number of solvate or water molecules can combine with the compounds according to the invention to form solvates and hydrates.

The disclosed compounds can be used in the form of salts derived from inorganic or organic acids. Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties comprising one or more nitrogen groups with a suitable acid. In various aspects, acids which may be employed to form pharmaceutically acceptable acid addition salts include such inorganic acids as hydrochloric acid, sulfuric acid and phosphoric acid and such organic acids as oxalic acid, maleic acid, succinic acid and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1′-methylene-bis-(2-hydroxy-3-naphthoate)) salts. Also, basic nitrogen-containing groups can be quaternized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

Basic addition salts can be prepared in situ during the final isolation and purification of a disclosed compound, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral administration, although the most suitable route in any given case will depend on the particular host, and nature and severity of the conditions for which the active ingredient is being administered. In a further aspect, the disclosed pharmaceutical composition can be formulated to allow administration orally.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids that can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral. Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Identification and Synthesis of Compounds

A virtual high-throughput screen was conducted. From an initial library of over 200,000 drug-like compounds, hits were identified using virtual docking studies. Compounds were PAINS filtered and scored. An in vitro blood stage assay removed 37 compounds, leaving 2 active hits, which were used as scaffolds for structure activity relationship (SAR) studies in order to optimize solubility, in vitro activity, and other properties.

Unless otherwise noted, all reagents and solvents were purchased from commercial sources and used without further purification. Tetrahydrofuran (THF) was distilled from benzophenone and sodium metal under a positive pressure argon atmosphere immediately before use. Column chromatography was carried out using Sorbtec silica gel 60 Å (particle size 40-63 μm) and analytical thin layer chromatography was performed on 0.25 mm silica gel 60 F254 precoated plates from EMD Millipore. Microwave reactions were performed in an Anton Paar Monowave 400. Proton nuclear magnetic resonance (¹H NMR) and proton decoupled carbon nuclear magnetic resonance (¹³C NMR) spectra were recorded at ambient temperature on a Bruker 500 or 700 MHz spectrometer or a Varian 400 or 500 MHz spectrometer. All H NMR experiments are reported in δ units, parts per million (ppm) downfield of trimethyl silane (TMS) and were measured relative to the residual proton signals of chloroform (δ 7.26), methanol (δ 3.31), acetone (δ 2.05), and dimethylsulfoxide (δ 2.50). Data for ¹H NMR are reported as follows: chemical shift (δ ppm), multiplicity (bs=broad singlet, s=singlet, d=doublet, dd=doublet of doublets, dt=doublet of triplets, ddt=doublet of doublet of triplets, dtd=double of triplet of doublets, t=triplet, tt=triplet of triplets, q=quartet, p=pentet, hept=heptet, m=multiplet), coupling constant (Hz), and integration. All ¹³C NMR spectra are reported in δ units, parts per million (ppm) downfield of TMS, and were measured relative to the residual proton signals of chloroform (δ 77.1 ppm), methanol (δ 49.0 ppm), acetone (δ 29.8), and dimethylsulfoxide (δ 39.5). Data for ¹³C NMR are reported as follows: chemical shift (δ ppm), multiplicity where appropriate (d=doublet, t=triplet, q=quartet, m=multiplet), and coupling constant (Hz). NMR data was analyzed by using MestReNova Software version 12.0.1.

Abbreviations used herein: EA=ethyl acetate; Hex=hexanes; MeOH=methanol; DCM=dichloromethane; LC-MS=liquid chromatography-mass spectrometry; TLC=thin layer chromatography; HATU=1-((dimethylamino)(dimethyliminio)methyl)-1H-[1,2,3]triazolo[4,5-b]pyridine 3-oxide hexafluorophosphate(V); EDC HCl=N-Ethyl-N′-(3-dimethylaminopropyl)carbodiimide hydrochloride; Xantphos=(9,9-dimethyl-9H-xanthene-4,5-diyl)bis(diphenylphosphane).

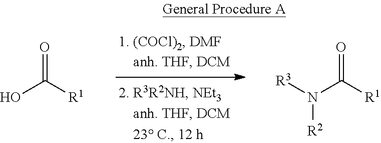

In a flame-dried round bottom flask under argon, the respective carboxylic acid (1 equiv) was dissolved in anhydrous dichloromethane (0.12 M), anhydrous tetrahydrofuran (0.91 M), and a catalytic amount of anhydrous N,N-dimethylformamide. The reaction mixture was then cooled to 0° C. and oxalyl chloride (3 equiv) was added slowly. Upon complete formation of the acid chloride intermediate, as monitored by LC-MS, the reaction mixture was concentrated in vacuo. Anhydrous dichloromethane and tetrahydrofuran were added back to the reaction mixture in their respective amounts, followed by the respective amine (3 equiv) and triethylamine (2 equiv). The reaction mixture was then stirred for 12 hours at room temperature. Upon reaction completion, the reaction mixture was diluted in dichloromethane and saturated aqueous sodium bicarbonate. The aqueous layer was extracted thrice with dichloromethane. The combined organic layers were dried over sodium sulfate and concentrated in vacuo. The crude product was purified by flash column chromatography to afford the title compound.

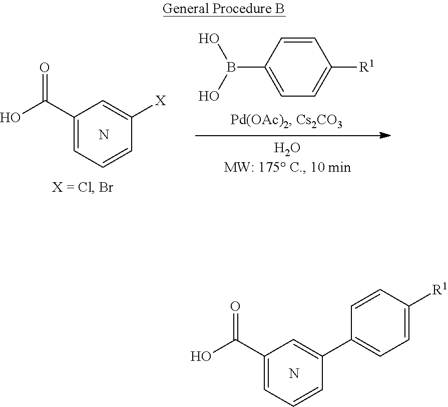

In a microwave vial, while stirring, carboxylic acid (1 equiv) and cesium carbonate (3 equiv) were dissolved in water (0.50 M). Palladium (II) acetate (1 mol %) and respective boronic acid (2 equiv) were added. Once well mixed, the reaction mixture was heated at 175° C. for 10 minutes in a microwave reactor (Anton Paar Monowave 400). Once cooled to 70° C., the reaction mixture was filtered through celite and washed with ethyl acetate and minimal water. The filtrate was transferred to a separatory funnel, basified to pH 12-13 with sodium hydroxide (3 M), and extracted thrice with diethyl ether. The aqueous layer was then acidified to pH 2-3 with hydrochloric acid (3 M). In the event of precipitate formation, the title compound was isolated via vacuum filtration. In instances where no precipitate forms, the acidified aqueous layer was extracted thrice with diethyl ether. The combined organic layers were dried over sodium sulfate and concentrated in vacuo to afford the title compound.

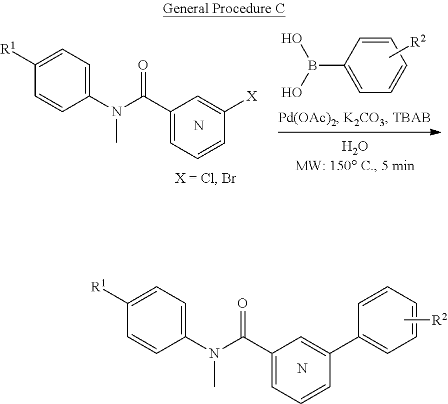

In a microwave vial, while stirring, carboxylic acid (1 equiv) and potassium carbonate (3 equiv) were dissolved in water (0.50 M). Palladium (II) acetate (0.4 mol %), respective boronic acid (1 equiv), and tetrabutylammonium bromide (1 equiv) were added. Once well mixed, the reaction mixture was heated at 150° C. for 5 minutes in a microwave reactor (Anton Paar Monowave 400). Once cooled to 70° C., the reaction mixture was filtered through celite and washed with ethyl acetate and minimal water. The filtrate was transferred to a separatory funnel and extracted thrice with diethyl ether. The combined organic layers were dried over sodium sulfate and concentrated in vacuo to afford the title compound. The crude product was purified by flash column chromatography to afford the title compound.

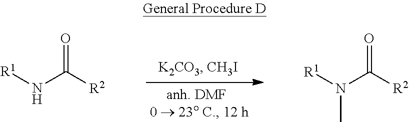

In a flame-dried round bottom flask under argon, the respective amide (1 equiv) was dissolved in anhydrous N,N-dimethylformamide (0.20 M). Potassium carbonate (2 equiv) was added and the reaction mixture was cooled to 0° C. After stirring for 15 minutes at 0° C., iodomethane (2 equiv) was added slowly. The reaction mixture was allowed to warm to room temperature and stirred for an additional 12 hours. Upon complete consumption of the amide, the reaction mixture was diluted in ethyl acetate and transferred to a separatory funnel. The organic layer was washed with brine thrice, dried over sodium sulfate, and concentrated in vacuo. The crude product was purified by flash column chromatography to afford the title compound.

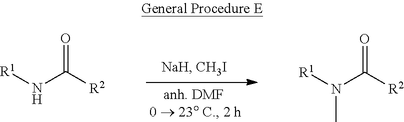

In a flame-dried round bottom flask under argon, the respective amide (1 equiv) was dissolved in anhydrous N,N-dimethylformamide (0.20 M). The flask was cooled to 0° C. and sodium hydride (60%, dispersion in paraffin liquid, 3 equiv) was added slowly. After stirring for 15 minutes at 0° C., iodomethane (2 equiv) was added slowly. The reaction mixture was stirred at 0° C. for an additional 2 hours. Upon complete consumption of the amide, the reaction mixture was diluted in ethyl acetate and transferred to a separatory funnel. The organic layer was washed with brine thrice, dried over sodium sulfate, and concentrated in vacuo. The crude product was purified by flash column chromatography to afford the title compound.

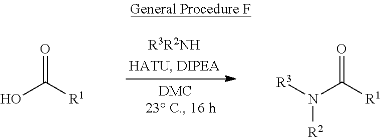

In a round bottom flask, the respective carboxylic acid (1 equiv) was dissolved in dimethyl carbonate (0.10 M). N,N-diisopropylethylamine (2 equiv), HATU (1.2 equiv), and respective amine (1.1 equiv) were added, and the reaction mixture was stirred at room temperature for 16 hours. Upon complete consumption of the acid, as monitored by LC-MS, the reaction mixture was diluted in water and extracted thrice with ethyl acetate. The combined organic layers were dried over sodium sulfate and concentrated in vacuo. The crude product was purified by flash column chromatography to afford the title compound. 1,2,4-triazole, Representative Intermediate

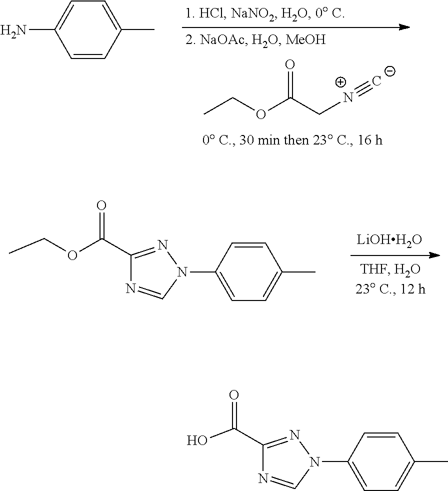

### 1-(p-tolyl)-1H-1,2,4-triazole-3-carboxylic acid

In a round bottom flask, p-toluidine (9.81 mmol, 1.11 equiv) was dissolved in water (1.40 M) and concentrated hydrochloric acid (29.5 mmol, 3.3 equiv). The reaction mixture was then cooled to 0° C. and a solution of sodium nitrite (9.81 mmol, 1.11 equiv) in water (9.4 M) was added dropwise. Sodium acetate (61.88 mmol, 7 equiv) and ethyl isocyanoacetate (8.84 mmol, 1 equiv) were then dissolved in water (0.86 M) and methanol (8.6 M). This solution was added to the reaction mixture very slowly, taking care to not allow the reaction mixture to rise above 5° C. After 30 minutes at 0° C., the reaction was warmed to room temperature and stirred for 16 hours. The orange precipitate was isolated via vacuum filtration and carried into the next step without further purification.

In a round bottom flask, the crude ester (ethyl 1-(p-tolyl)-1H-1,2,4-triazole-3-carboxylate) (8.84 mmol, 1 equiv) was dissolved in tetrahydrofuran (0.13 M) and water (0.40 M). Following addition of lithium hydroxide monohydrate (26.5 mmol, 3 equiv), the reaction mixture was stirred at room temperature for 12 hours. Upon complete consumption of ester, the reaction mixture was basified to pH 12-13 with sodium hydroxide (3 M) and extracted thrice with dichloromethane. The aqueous layer was then acidified to pH 2-3 with hydrochloric acid (3 M). The formed precipitate was isolated via vacuum filtration to afford the title compound as a light orange solid (19%, over 2 steps). ¹H NMR (400 MHz, CD₃OD) δ 9.13 (s, 1H), 7.74 (d, J=8.4 Hz, 2H), 7.39 (d, J=8.4 Hz, 2H), 2.42 (s, 3H); ¹³C NMR (125 MHz, CD₃OD) δ 160.91, 155.12, 143.11, 139.15, 134.38, 130.01, 119.96, 19.64.

### 1,2,3-triazole, Representative Intermediate

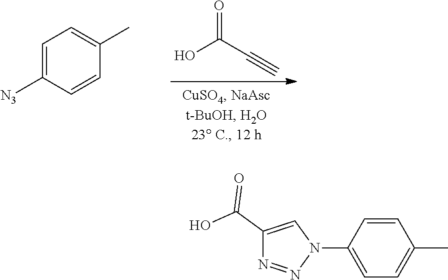

### 1-(p-tolyl)-1H-1,2,3-triazole-4-carboxylic acid

In a round bottom flask, copper (II) sulfate (0.467 mmol, 0.1 equiv) and sodium ascorbate (0.933 mmol, 0.2 equiv) were dissolved in water (1 M). 1-azido-4-methylbenzene (4.67 mmol, 1 equiv), tert-butanol (1 M), and propiolic acid (5.60 mmol, 1.2 equiv) were then added to the flask. The reaction mixture was sealed with a glass stopper and stirred at room temperature for 12 hours. The reaction was basified to pH 12-13 with sodium hydroxide (3 M) and extracted thrice with ethyl acetate. The aqueous layer was then acidified to pH 2-3 with hydrochloric acid (3 M) and extracted thrice with ethyl acetate. The combined organic layers were dried over sodium sulfate and concentrated in vacuo to afford the title compound as an orange solid (16%). ¹H NMR (500 MHz, CD₃OD) δ 8.97 (s, 1H), 7.73 (d, J=8.2 Hz, 2H), 7.38 (d, J=8.2 Hz, 2H), 2.40 (s, 3H); ¹³C NMR (125 MHz, CD₃OD) δ 163.26, 141.86, 141.06, 135.62, 131.43, 127.53, 121.71, 21.08.

### Reverse Triazole, Representative Intermediate

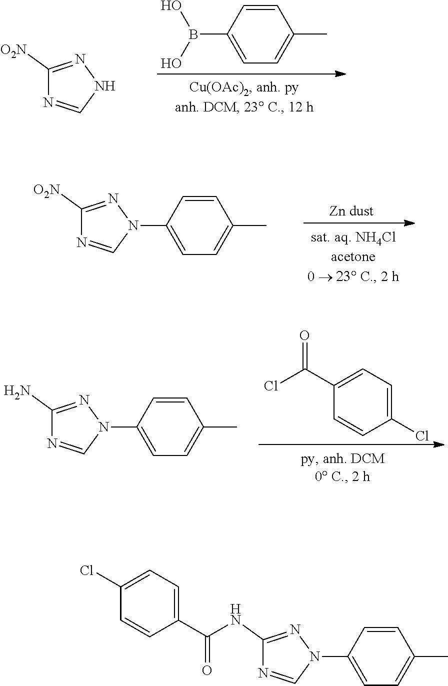

### 3-nitro-1-(p-tolyl)-1H-1,2,4-triazole

In a flame-dried round bottom flask under argon, 3-nitro-1H-1,2,4-triazole (13.2 mmol, 1 equiv) and p-tolylboronic acid (14.5 mmol, 1.1 equiv) were dissolved in anhydrous dichloromethane (0.17 M). Copper (II) acetate (19.7 mmol, 1.5 equiv) and anhydrous pyridine (26.3 mmol, 2 equiv) were then added and the reaction mixture was heated to 30° C. for 12 hours. Upon reaction completion via LC-MS, the solids were removed by vacuum filtration and washed with dichloromethane. The filtrate was washed with water thrice. Brine was added to the combined aqueous layers, which was extracted with dichloromethane. The combined organic layers were dried over sodium sulfate and concentrated in vacuo. The crude product was purified by flash column chromatography with hexanes/ethyl acetate, followed by recrystallization in hexanes to afford the title compound as a yellow solid (50%). R_{f} (30% EA/Hex)=0.31; ¹H NMR (500 MHz, CDCl₃) δ 8.57 (s, 1H), 7.62 (d, J=8.4 Hz, 2H), 7.37 (d, J=8.4 Hz, 2H), 2.45 (s, 3H); ¹³C NMR (175 MHz, CDCl₃) δ 142.19, 140.67, 133.59, 130.82, 120.59, 21.33.

### 1-(p-tolyl)-1H-1,2,4-triazol-3-amine

In a round bottom flask, 3-nitro-1-(p-tolyl)-1H-1,2,4-triazole (6.61 mmol, 1 equiv) was dissolved in saturated ammonium chloride (0.37 M) and acetone (0.09 M). The reaction mixture was cooled to 0° C. and zinc dust (33.1 mmol, 5 equiv) was added at 0° C. The reaction mixture was warmed to room temperature and stirred for 2 hours. Upon reaction completion via LC-MS, the reaction mixture was diluted in water and dichloromethane and the aqueous layer was extracted thrice with dichloromethane. The combined organic layers were dried over sodium sulfate and concentrated in vacuo to afford the title compound as a light yellow solid (56%). ¹H NMR (500 MHz, (CD₃)₂SO) δ 8.73 (s, 1H), 7.58 (d, J=8.3 Hz, 2H), 7.26 (d, J=8.3 Hz, 2H), 5.63 (s, 2H), 2.30 (s, 3H); ¹³C NMR (125 MHz, CD₃OD) δ 164.41, 140.48, 135.52, 134.85, 129.91, 117.78, 20.46.

### 4-chloro-N-(1-(p-tolyl)-1H-1,2,4-triazol-3-yl)benzamide

In a flame-dried round bottom flask under argon, 1-(p-tolyl)-1H-1,2,4-triazol-3-amine (1.15 mmol, 1 equiv) was dissolved in anhydrous dichloromethane (0.20 M) and anhydrous pyridine (3.44 mmol, 3 equiv). The flask was cooled to 0° C. and 4-chlorobenzoyl chloride (1.26 mmol, 2 equiv) was added. The reaction mixture was warmed to room temperature and stirred for 2 hours. Upon complete consumption of amine, the reaction mixture was diluted in water and dichloromethane and the aqueous layer was extracted thrice with dichloromethane. The combined organic layers were dried over sodium sulfate and concentrated in vacuo. The crude product was purified by flash column chromatography with hexanes/ethyl acetate, followed by recrystallization in hexanes to afford the title compound as a fluffy white solid (22%). R_{f}(50% EA/Hex)=0.19; ¹H NMR (500 MHz, (CD₃)₂SO) δ 11.05 (s, 1H), 9.18 (s, 1H), 8.01 (d, J=8.5 Hz, 2H), 7.73 (d, J=8.3 Hz, 2H), 7.60 (d, J=8.5 Hz, 2H), 7.37 (d, J=8.3 Hz, 2H), 2.36 (s, 3H); ¹³C NMR (125 MHz, (CD₃)₂SO) δ 164.76, 157.23, 142.15, 137.61, 137.36, 134.94, 132.84, 130.61, 130.34, 129.03, 119.37, 21.00.

### 5-imidazole-2-carboxamide, Representative Intermediate

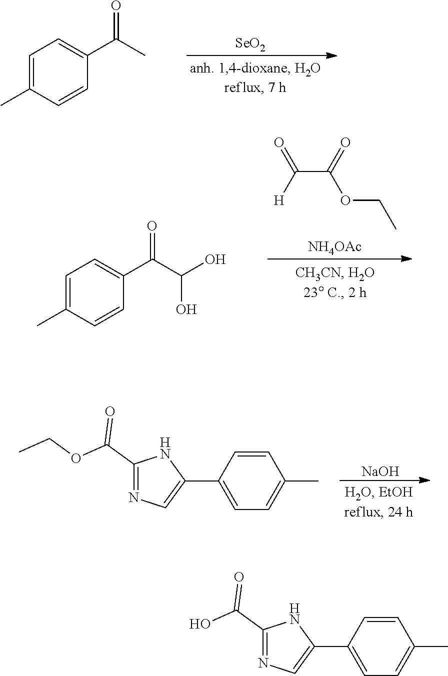

### ethyl 5-(p-tolyl)-1H-imidazole-2-carboxylate

In a round bottom flask under argon, selenium dioxide (7.45 mmol, 2 equiv) was dissolved in anhydrous 1,4-dioxane (1 M). Water (7 drops) and 1-(p-tolyl)ethan-1-one (3.73 mmol, 1 equiv) were added and the flask was heated at reflux for 7 hours. Upon complete consumption of the acetophenone, the flask was cooled to room temperature. The reaction mixture was diluted in dichloromethane (0.25 M), filtered through a pad of celite, and the filtrate was concentrated in vacuo. The resulting residue was diluted in water (0.53 M), heated at reflux for 10 minutes, then cooled to 0° C. The resulting white precipitate was isolated via vacuum filtration and immediately carried into the next step without further purification due to product instability.

Polymerized ethyl 2-oxoacetate in 47% toluene (2.05 mL, 11.2 mmol, 3 equiv) was heated to 60° C. for 15 minutes before it was added dropwise to a stirring solution of ammonium acetate (11.2 mmol, 3 equiv) in water (1.54 M) and acetonitrile (0.77 M) at 0° C. A solution of 2,2-dihydroxy-1-(p-tolyl)ethan-1-one (3.73 mmol, 1 equiv) dissolved in acetonitrile (0.77 M) was then added dropwise at 0° C. After 30 minutes at 0° C., the reaction mixture was warmed to room temperature and stirred for 2 hours. Upon complete diol consumption of the diol, the reaction mixture was concentrated in vacuo. The resulting residue was diluted in water and extracted thrice with dichloromethane. The combined organic layers were washed with brine, dried over sodium sulfate, and concentrated in vacuo. The crude product was purified by flash column chromatography with hexanes/ethyl acetate, followed by recrystallization from dichloromethane/hexanes to afford the title compound as a light yellow solid (36%). R_{f} (50% EA/Hex)=0.24; ¹H NMR (500 MHz, CDCl₃) δ 10.87 (bs, 1H), 7.87-7.40 (m, 3H), 7.26-7.13 (m, 2H), 4.47 (q, J=7.2 Hz, 2H), 2.39 (s, 3H), 1.43 (t, J=7.2 Hz, 3H); ¹³C NMR (175 MHz, CDCl₃) δ 168.81, 159.84, 159.35, 156.73, 137.87, 137.63, 129.47, 125.24, 62.00, 21.29, 14.18.

### 5-(p-tolyl)-1H-imidazole-2-carboxylic acid

In a round bottom flask, ethyl 5-(p-tolyl)-1H-imidazole-2-carboxylate (1.26 mmol, 1 equiv) was dissolved in ethanol (0.20 M), to which a solution of sodium hydroxide (6.30 mmol, 5 equiv) in water (0.20 M) was added slowly. The flask was heated at reflux for 24 hours. Upon complete consumption of ester, the reaction mixture was acidified to pH 2-3 with hydrochloric acid (3 M) and extracted thrice with 3:1 chloroform:isopropanol. The combined organic layers were dried over sodium sulfate and concentrated in vacuo to afford the title compound as an off-white solid. ¹H NMR (400 MHz, CD₃OD) δ 7.63 (s, 1H), 7.59 (d, J=8.0 Hz, 2H), 7.25 (d, J=8.0 Hz, 2H), 2.33 (s, 3H); ¹³C NMR (125 MHz, (CD₃)₂SO) δ 159.15, 139.90, 138.90, 136.51, 129.53, 129.22, 124.87, 118.11, 20.81.

### 2-imidazole-5-carboxamide, Representative Intermediate

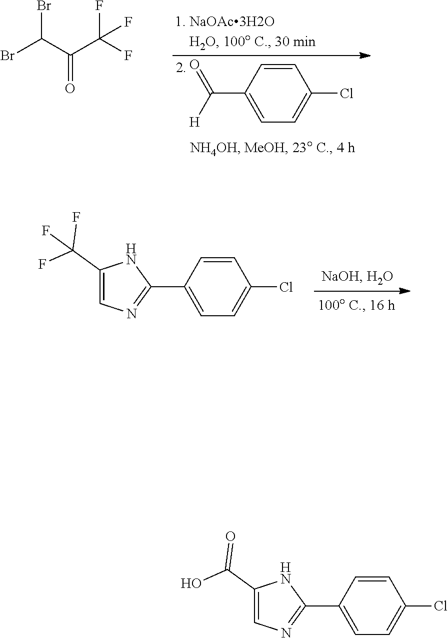 [SMILES: O=C(C(Br)Br)C(F)(F)F ; O=Cc1ccc(Cl)cc1 ; FC(F)(F)c1cnc(-c2ccc(Cl)cc2)[nH]1 ; O=C(O)c1cnc(-c2ccc(Cl)cc2)[nH]1]

### 2-(4-chlorophenyl)-5-(trifluoromethyl)-1H-imidazole

In a 3-necked round bottom flask, sodium acetate trihydrate (4.52 mmol, 1.27 equiv) was dissolved in water (1.42 M), to which 3,3-dibromo-1,1,1-trifluoropropan-2-one (3.73 mmol, 1.05 equiv) was added. The flask was heated at 100° C. for 30 minutes then cooled to room temperature. A solution of 4-chlorobenzaldehyde (3.56 mmol, 1 equiv) and ammonium hydroxide (3.88 mL) dissolved in methanol (0.94 M) was added to the reaction mixture. After 4 hours, the reaction was quenched with water and extracted thrice with dichloromethane. The combined organic layers were dried over sodium sulfate and concentrated in vacuo. The crude product was purified by flash column chromatography with hexanes/ethyl acetate to afford the title compound as a white solid (25%). R_{f} (30% EA/Hex)=0.40; ¹H NMR (500 MHz, CD₃OD) δ 7.87 (d, J=8.6 Hz, 2H), 7.65 (s, 1H), 7.49 (d, J=8.6 Hz, 2H).

### 2-(4-chlorophenyl)-1H-imidazole-5-carboxylic acid

In a round bottom flask, 2-(4-chlorophenyl)-5-(trifluoromethyl)-1H-imidazole (0.770 mmol, 1 equiv) and sodium hydroxide pellets (3.96 mmol, 5.14 equiv) were dissolved in water (0.32 M). The flask was heated at 100° C. for 16 hours then cooled to room temperature. The reaction mixture was further diluted in water and extracted thrice with dichloromethane. The aqueous layer was then acidified to pH 2-3 with hydrochloric acid (3 M) and the formed precipitate was isolated via vacuum filtration. The solids were dissolved in acetone and methanol and concentrated in vacuo to afford the title compound as a dark orange solid (64%). ¹H NMR (400 MHz, CD₃OD) δ 7.87 (d, J=8.7 Hz, 2H), 7.73 (s, 1H), 7.44 (d, J=8.7 Hz, 2H); ¹³C NMR (125 MHz, CD₃OD) δ 163.60, 147.29, 135.22, 131.16, 128.79, 127.24, 127.14.

### 2-oxazole-5-carboxamide, Representative Intermediate

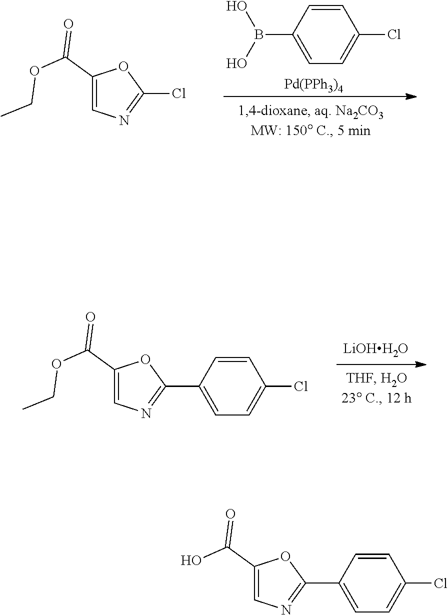 [SMILES: CCOC(=O)c1cnc(Cl)o1 ; OB(O)c1ccc(Cl)cc1 ; CCOC(=O)c1cnc(-c2ccc(Cl)cc2)o1 ; "LiOH·H2O" ; O=C(O)c1cnc(-c2ccc(Cl)cc2)o1 ; "Pd(PPh3)4"]

### ethyl 2-(4-chlorophenyl)oxazole-5-carboxylate

In a microwave vial, while stirring, 4-chlorophenyl boronic acid (0.285 mmol, 1 equiv), aqueous sodium carbonate (2 M), tetrakis(triphenylphosphine)palladium(0) (0.0285 mmol, 10 mol %), and ethyl 2-chlorooxazole-5-carboxylate (0.285 mmol, 1 equiv) were dissolved in 1,4-dioxane (0.14 M). Once well mixed, the reaction mixture was heated at 150° C. for 5 minutes in a microwave reactor (Anton Paar Monowave 400). Once cooled to 70° C., the reaction mixture was filtered through celite and washed with water. The filtrate was transferred to a separatory funnel and the aqueous layer was extracted thrice with 3:1 chloroform:isopropanol. The combined organic layers were washed with brine, dried over sodium sulfate, and concentrated in vacuo. The crude product was purified by flash column chromatography with hexanes/ethyl acetate to afford the title compound as a white solid (25%). R_{f} (10% EA/Hex)=0.21; ¹H NMR (400 MHz, CDCl₃) 8.08 (d, J=8.6 Hz, 2H), 7.83 (s, 1H), 7.47 (d, J=8.6 Hz, 2H), 4.42 (q, J=7.2 Hz, 2H), 1.41 (t, J=7.1 Hz, 3H).

### 5-(4-chlorophenyl)oxazole-2-carboxylic acid

In a round bottom flask, ester (0.282 mmol, 1 equiv) was dissolved in tetrahydrofuran (0.24 M) and water (0.12 M). Following addition of lithium hydroxide monohydrate (0.338 mmol, 1.2 equiv), the reaction mixture was stirred at room temperature for 12 hours. Upon complete consumption of ester, the reaction mixture was basified to pH 12-13 with sodium hydroxide (3 M) and extracted thrice with dichloromethane. The aqueous layer was then acidified to pH 2-3 with hydrochloric acid (3 M). The formed precipitate was isolated via vacuum filtration to afford the title compound as a white solid (69%). ¹H NMR (400 MHz, (CD₃)₂SO) 8.03 (d, J=6.5 Hz, 2H), 8.02 (s, 1H), 7.64 (d, J=8.6 Hz, 2H).

### 5-oxazole-2-carboxamide, Representative Intermediate

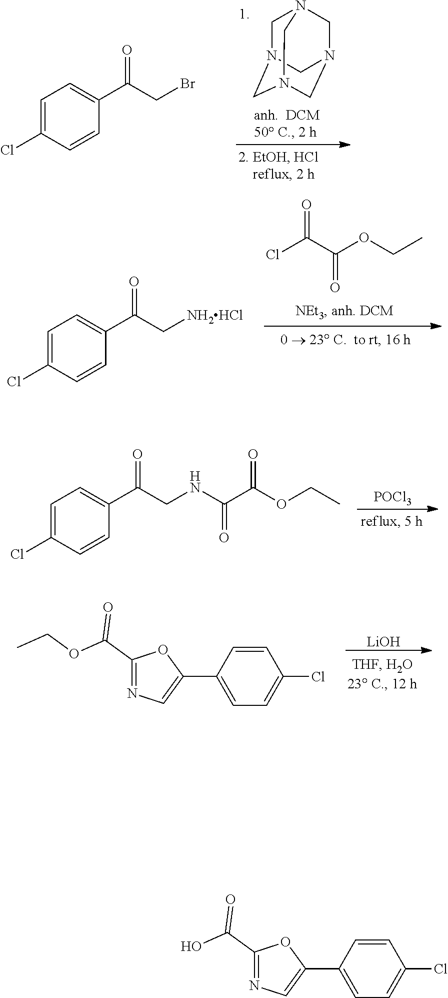

### ethyl 2-oxo-2-((2-oxo-2-(p-tolyl)ethyl)amino)acetate

In a flame-dried round bottom flask under argon, acetophenone (6 mmol, 1 equiv) and 1,3,5,7-tetraazaadamantane (6 mmol, 1 equiv) were dissolved in anhydrous dichloromethane (0.13 M). The flask was equipped with a reflux condenser and the reaction mixture was heated to 50° C. for 2 hours. The reaction mixture was cooled to room temperature and the precipitate was isolated by vacuum filtration, washing the cake with dichloromethane and ethanol. The resulting white solid (urotropinium salt) was transferred to a round bottom flask, dissolved in ethanol (0.04 M) and concentrated hydrochloric acid (0.4 M), and heated to reflux for 2 hours. The reaction mixture was concentrated to half in vacuo. The light-yellow precipitate was isolated by vacuum filtration and carried into the next step without further purification.

In a flame-dried round bottom flask under argon, 2-amino-1-(4-chlorophenyl)ethan-1-one hydrochloride (6 mmol, 1 equiv) was dissolved in anhydrous dichloromethane (0.29 M) and triethylamine (18 mmol, 3 equiv) was added. The reaction mixture was cooled to 0° C. and ethyl 2-chloro-2-oxoacetate (6 mmol, 1 equiv) was added. The reaction mixture was warmed to room temperature and stirred for 16 hours. Upon complete consumption of amine, the reaction mixture was diluted in water and the aqueous layer was extracted thrice with dichloromethane. The combined organic layers were washed with water, brine, dried over sodium sulfate, and concentrated in vacuo. The crude product was purified by flash column chromatography with hexanes/ethyl acetate to afford the title compound as a white solid (41%). R_{f} (50% EA/Hex)=0.28; ¹H NMR (500 MHz, CDCl₃) δ 8.04 (bs, 1H), 7.94 (d, J=8.1 Hz, 2H), 7.51 (d, J=8.1 Hz, 2H), 4.83-4.78 (m, 2H), 4.41 (q, J=7.1 Hz, 2H), 1.42 (t, J=7.1 Hz, 3H); ¹³C NMR (175 MHz, CDCl₃) δ 191.59, 159.85, 156.77, 141.08, 132.28, 129.51, 129.48, 63.59, 46.55, 14.12.

### ethyl 5-(4-chlorophenyl)oxazole-2-carboxylate

In a round bottom flask, ethyl 5-(4-chlorophenyl)oxazole-2-carboxylate (2.41 mmol, 1 equiv) was dissolved in phosphoryl chloride (0.33 M) and heated to reflux for 5 hours. Upon reaction completion via LC-MS, the reaction mixture was diluted in saturated sodium bicarbonate and dichloromethane. The aqueous layer was extracted thrice with dichloromethane. The combined organic layers were dried over sodium sulfate and concentrated in vacuo. The crude product was purified by flash column chromatography with hexanes/ethyl acetate, followed by recrystallization from dichloromethane/hexanes to afford the title compound as a lustrous tan solid (87%). R_{f} (20% EA/Hex)=0.25; ¹H NMR (500 MHz, CDCl₃) δ 7.70 (dt, J=8.5, 2.6, 2.0 Hz, 2H), 7.52 (s, 1H), 7.44 (dt, J=8.5, 2.6, 2.0 Hz, 2H), 4.50 (q, J=7.1 Hz, 2H), 1.46 (t, J=7.2 Hz, 3H); ¹³C NMR (125 MHz, CDCl₃) δ 155.63, 153.29, 151.83, 135.86, 129.44, 126.38, 125.19, 124.19, 62.74, 14.21.

### 5-(4-chlorophenyl)oxazole-2-carboxylic acid

In a round bottom flask, ethyl 5-(4-chlorophenyl)oxazole-2-carboxylate (1.99 mmol, 1 equiv) was dissolved in tetrahydrofuran (0.13 M) and water (0.40 M). Following addition of lithium hydroxide (3.97 mmol, 2 equiv), the reaction mixture was stirred at room temperature for 12 hours. Upon complete consumption of ester, the reaction mixture was concentrated in vacuo to afford the title compound as a white solid (quantitative). ¹H NMR (500 MHz, CD₃OD) δ 7.80 (dt, J=8.6, 2.6, 2.0 Hz, 2H), 7.56 (s, 1H), 7.46 (dt, J=8.6, 2.6, 2.0 Hz, 2H); ¹³C NMR (125 MHz, CD₃OD) δ 161.80, 159.56, 152.48, 135.77, 130.26, 127.77, 127.22, 124.10.

### 5-thiazole-2-carboxamide, Representative Intermediate

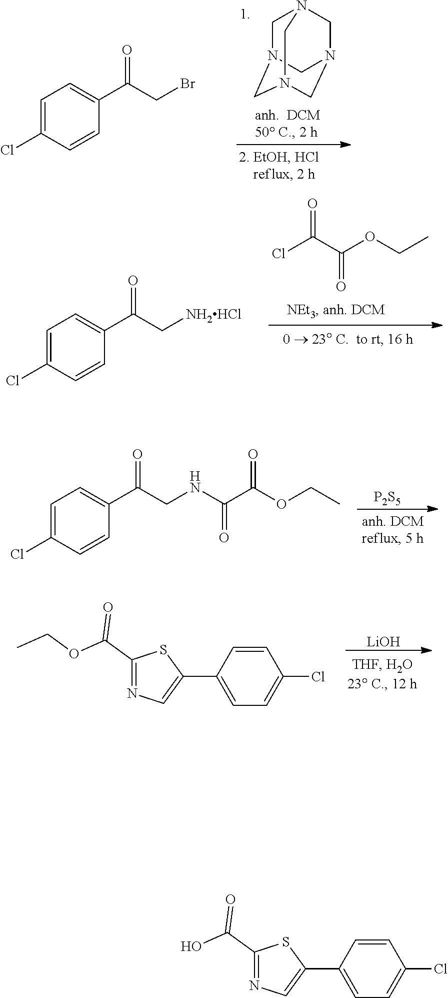

### ethyl 2-oxo-2-((2-oxo-2-(p-tolyl)ethyl)amino)acetate

In a flame-dried round bottom flask under argon, acetophenone (6 mmol, 1 equiv) and 1,3,5,7-tetraazaadamantane (6 mmol, 1 equiv) were dissolved in anhydrous dichloromethane (0.13 M). The flask was equipped with a reflux condenser and the reaction mixture was heated to 50° C. for 2 hours. The reaction mixture was cooled to room temperature and the precipitate was isolated by vacuum filtration, washing the cake with dichloromethane and ethanol. The resulting white solid (urotropinium salt) was transferred to a round bottom flask, dissolved in ethanol (0.04 M) and concentrated hydrochloric acid (0.4 M), and heated to reflux for 2 hours. The reaction mixture was concentrated to half in vacuo. The light-yellow precipitate was isolated by vacuum filtration and carried into the next step without further purification.

In a flame-dried round bottom flask under argon, 2-amino-1-(4-chlorophenyl)ethan-1-one hydrochloride (6 mmol, 1 equiv) was dissolved in anhydrous dichloromethane (0.29 M) and triethylamine (18 mmol, 3 equiv) was added. The flask was cooled to 0° C. and ethyl 2-chloro-2-oxoacetate (6 mmol, 1 equiv) was added. The reaction mixture was warmed to room temperature and stirred for 16 hours. Upon complete consumption of amine, the reaction mixture was diluted in water and the aqueous layer was extracted thrice with dichloromethane. The combined organic layers were washed with water, brine, dried over sodium sulfate, and concentrated in vacuo. The crude product was purified by flash column chromatography with hexanes/ethyl acetate to afford the title compound as a white solid (41%). R_{f} (50% EA/Hex)=0.28; ¹H NMR (500 MHz, CDCl₃) δ 8.04 (bs, 1H), 7.94 (d, J=8.1 Hz, 2H), 7.51 (d, J=8.1 Hz, 2H), 4.83-4.78 (m, 2H), 4.41 (q, J=7.1 Hz, 2H), 1.42 (t, J=7.1 Hz, 3H); ¹³C NMR (175 MHz, CDCl₃) δ 191.59, 159.85, 156.77, 141.08, 132.28, 129.51, 129.48, 63.59, 46.55, 14.12.

### ethyl 5-(p-tolyl)thiazole-2-carboxylate

In a round bottom flask under argon, ethyl 2-oxo-2-((2-oxo-2-(p-tolyl)ethyl)amino)acetate (1.04 mmol, 1 equiv) was dissolved in anhydrous dichloromethane (0.38 M) and phosphorus pentasulfide (2.08 mmol, 2 equiv) was added. The reaction mixture was heated to reflux for 5 hours, then cooled to room temperature and quenched via the slow addition of water. The solution was transferred to a separatory funnel and the aqueous layer was extracted thrice with dichloromethane. The combined organic layers were washed with brine, dried over sodium sulfate, and concentrated in vacuo. The crude product was purified by flash column chromatography with hexanes/ethyl acetate to afford the title compound (77%). R_{f} (10% EA/Hex)=0.13; ¹H NMR (500 MHz, CD₃OD) δ 8.30 (s, 1H), 7.71 (d, J=8.5 Hz, 2H), 7.47 (d, J=8.5 Hz, 2H), 4.46 (q, J=7.1 Hz, 2H), 1.43 (t, J=7.1 Hz, 3H).

### 5-(p-tolyl)thiazole-2-carboxylic acid

In a round bottom flask, ester (ethyl 5-(p-tolyl)thiazole-2-carboxylate) (0.112 mmol, 1 equiv) was dissolved in tetrahydrofuran (0.13 M) and water (0.40 M). Following addition of lithium hydroxide (0.224 mmol, 2 equiv), the reaction mixture was stirred at room temperature for 12 hours. Upon complete consumption of ester, the reaction mixture was concentrated in vacuo to afford the title compound as a light yellow lustrous solid (42%). ¹H NMR (500 MHz, CD₃OD) δ 8.05 (s, 1H), 7.61 (dt, J=8.6, 2.7, 2.0 Hz, 2H), 7.39 (dt, J=8.6, 2.7, 2.0 Hz, 2H); ¹³C NMR (125 MHz, CD₃OD) δ 168.61, 165.97, 143.02, 140.27, 135.56, 131.40, 130.40, 129.23.

### 2-thiazole-5-carboxamide, Representative Intermediate

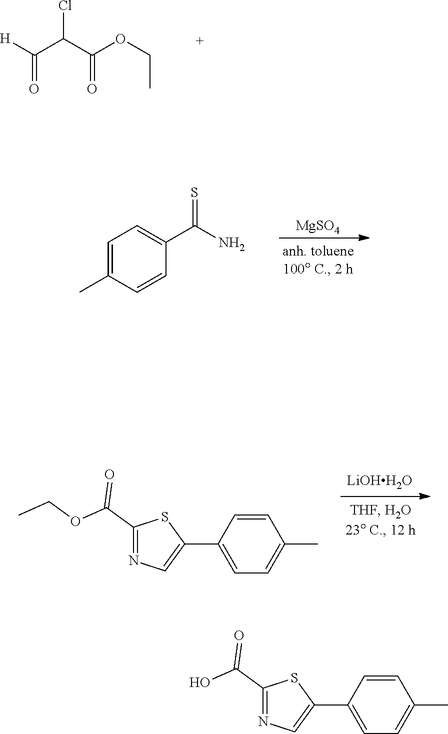

### ethyl 2-(p-tolyl)thiazole-5-carboxylate

In a flame-dried round bottom flask under argon, 4-chlorothiobenzamide (1.98 mmol, 1 equiv) was dissolved in anhydrous toluene (0.30 M). Anhydrous magnesium sulfate (3.97 mmol, 2 equiv) and ethyl 2-chloro-2-formylacetate (3.97 mmol, 2 equiv) were added and the reaction mixture was heated to 100° C. for 2 hours. Upon complete consumption of thioamide, the reaction mixture was cooled to room temperature. The formed precipitate was isolated via vacuum filtration. The crude product was purified by flash column chromatography with hexanes/ethyl acetate, followed by recrystallization in dichloromethane/hexanes to afford the title compound as a lustrous white solid (77%). R_{f} (10% EA/Hex)=0.34; ¹H NMR (500 MHz, CDCl₃) δ 8.39 (s, 1H), 7.88 (d, J=8.2 Hz, 2H), 7.28 (d, J=8.2 Hz, 2H), 4.39 (q, J=7.1 Hz, 2H), 2.41 (s, 3H), 1.40 (t, J=7.1 Hz, 3H); ¹³C NMR (175 MHz, CDCl₃) δ 173.65, 161.65, 149.20, 141.86, 130.30, 129.93, 128.51, 126.86, 61.76, 21.68, 14.44.

### 2-(p-tolyl)thiazole-5-carboxylic acid

In a round bottom flask, ester (1.01 mmol, 1 equiv) was dissolved in tetrahydrofuran (0.13 M) and water (0.40 M). Following addition of lithium hydroxide monohydrate (3.03 mmol, 3 equiv), the reaction mixture was stirred at room temperature for 12 hours. Upon complete consumption of ester, the reaction mixture was basified to pH 12-13 with sodium hydroxide (3 M) and extracted thrice with dichloromethane. The aqueous layer was then acidified to pH 2-3 with hydrochloric acid (3 M). The formed precipitate was isolated via vacuum filtration to afford the title compound as a white solid. ¹H NMR (500 MHz, (CD₃)₂SO) δ 13.57 (bs, 1H), 8.37 (s, 1H), 7.89 (d, J=8.1 Hz, 2H), 7.33 (d, J=8.1 Hz, 2H), 2.36 (s, 3H); ¹³C NMR (125 MHz, (CD₃)₂SO) δ 172.15, 162.08, 148.72, 141.51, 129.93, 129.84, 129.78, 126.57, 21.04.

### 4-picolinamide, Representative Intermediate

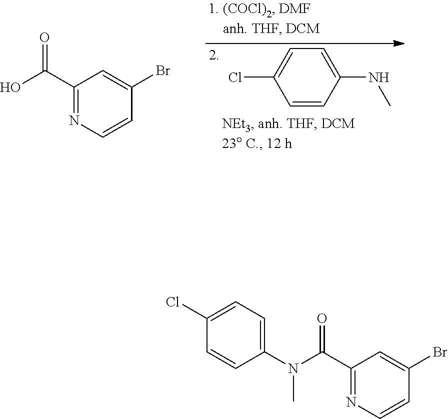

### 4-bromo-N-(4-chlorophenyl)-N-methylpicolinamide

4-bromo-N-(4-chlorophenyl)-N-methylpicolinamide was prepared via General Procedure A at a 1.49 mmol scale. The crude product was purified by flash column chromatography with hexanes/ethyl acetate to afford the title compound as an off-white solid (59%). R_{f} (30% EA/Hex)=0.27; ¹H NMR (500 MHz, CD₃OD) δ 8.26 (s, 1H), 7.61 (s, 1H), 7.38 (s, 1H), 7.26 (s, 2H), 7.15 (s, 2H), 3.46 (s, 3H).

### 5-nicotinamide, Representative Intermediate

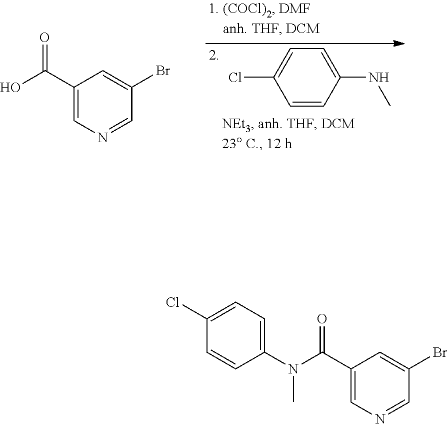

### 5-bromo-N-(4-chlorophenyl)-N-methylnicotinamide

5-bromo-N-(4-chlorophenyl)-N-methylnicotinamide was prepared via General Procedure A at a 4.95 mmol scale. The crude product was purified by flash column chromatography with hexanes/ethyl acetate to afford the title compound as an off-white solid (70%). R_{f} (50% EA/Hex)=0.47; ¹H NMR (500 MHz, CDCl₃) δ 8.55 (d, J=1.9 Hz, 1H), 8.34-8.24 (m, 1H), 7.87 (t, J=1.9 Hz, 1H), 7.27 (d, J=8.3 Hz, 2H), 7.01 (d, J=8.3 Hz, 2H), 3.48 (s, 3H); ¹³C NMR (175 MHz, CDCl₃) δ 166.43, 151.66, 147.33, 142.26, 138.85, 133.38, 132.80, 129.98, 128.24, 120.31, 38.49, 29.71.

### Thiazole Lactam, Representative Intermediate

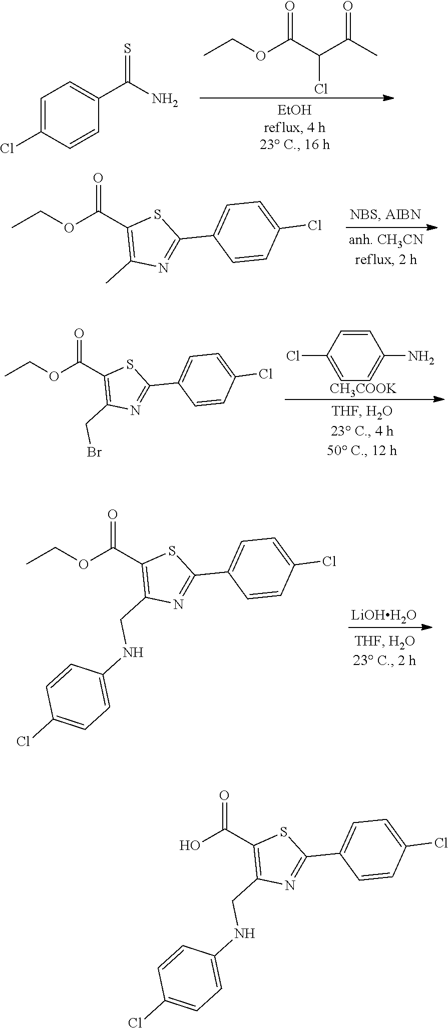

### ethyl 2-(4-chlorophenyl)-4-methylthiazole-5-carboxylate

In a round bottom flask, 4-chlorobenzothioamide (14.6 mmol, 1 equiv) was dissolved in ethanol (0.65 M), and ethyl 2-chloroacetoacetate (17.9 mmol, 1.23 equiv) was added. The reaction mixture was stirred for 4 hours at reflux, then allowed to cool to room temperature and stir for 16 hours. The resulting yellow precipitate was isolated via vacuum filtration and washed with ethanol (0° C.) to afford the title compound as a yellow fluffy solid (81%). R_{f} (30% EA/Hex)=0.80; ¹H NMR (500 MHz, CDCl₃) δ 7.90 (d, J=8.4 Hz, 2H), 7.42 (d, J=8.4 Hz, 2H), 4.36 (q, J=7.1 Hz, 2H), 2.77 (s, 3H), 1.39 (t, J=7.1 Hz, 3H).

### ethyl 4-(bromomethyl)-2-(4-chlorophenyl)thiazole-5-carboxylate

In a flame-dried round bottom flask under argon, ethyl 2-(4-chlorophenyl)-4-methylthiazole-5-carboxylate (3.55 mmol, 1 equiv) was dissolved in anhydrous acetonitrile (0.25 M). N-bromosuccinimide (6.21 mmol, 1.75 equiv) followed by azobisisobutyronitrile (0.355 mmol, 0.1 equiv), were added and the flask was heated at reflux for 2 hours. Upon complete consumption of ester starting material, the reaction mixture was concentrated in vacuo, diluted with water, and extracted thrice with dichloromethane. The combined organic layers were dried over sodium sulfate and concentrated in vacuo. The crude product was purified by flash column chromatography with hexanes/ethyl acetate to afford the title compound as a white solid (32%). R_{f} (30% EA/Hex)=0.81; ¹H NMR (500 MHz, CDCl₃) δ 7.92 (dt, J=8.6, 2.5, 2.0 Hz, 2H), 7.44 (dt, J=8.6, 2.4, 2.0 Hz, 2H), 4.98 (s, 2H), 4.41 (q, J=7.1 Hz, 2H), 1.41 (t, J=7.1 Hz, 3H).

### ethyl 2-(4-chlorophenyl)-4-(((4-chlorophenyl)amino)methyl)thiazole-5-carboxylate

In a round bottom flask, 4-chloroaniline (0.499 mmol, 1.2 equiv) was dissolved in water (0.25 M) and tetrahydrofuran (0.25 M) and potassium acetate (0.499 mmol, 1.2 equiv) was added. The reaction mixture was stirred for 5 minutes at room temperature, then ethyl 4-(bromomethyl)-2-(4-chlorophenyl)thiazole-5-carboxylate (0.416 M, 1 equiv) was added. The reaction mixture was stirred for an additional 4 hours at room temperature, then 12 hours at 50° C. Upon complete consumption of bromo starting material, the reaction mixture was concentrated in vacuo to remove tetrahydrofuran, diluted with water, and extracted thrice with dichloromethane. The combined organic layers were dried over sodium sulfate and concentrated in vacuo. The crude product was purified by flash column chromatography with hexanes/ethyl acetate to afford the title compound as a yellow solid (97%). R_{f} (30% EA/Hex)=0.77; ¹H NMR (500 MHz, CDCl₃) δ 7.93-7.88 (m, 2H), 7.46-7.42 (m, 2H), 7.14-7.10 (m, 2H), 6.75-6.70 (m, 2H), 4.95 (s, 1H), 4.74 (s, 2H), 4.39 (q, J=7.2 Hz, 2H), 1.41 (t, J=7.1 Hz, 3H).

### 2-(4-chlorophenyl)-4-(((4-chlorophenyl)amino)methyl)thiazole-5-carboxylic acid

In a round bottom flask, ethyl 2-(4-chlorophenyl)-4-(((4-chlorophenyl)amino)methyl)thiazole-5-carboxylate (0.491 mmol, 1 equiv) was dissolved in water (0.40 M) and tetrahydrofuran (0.13 M) and lithium hydroxide monohydrate (1.47 mmol, 3 equiv) was added. The reaction mixture was stirred at room temperature for 2 hours. The reaction mixture was concentrated in vacuo to remove tetrahydrofuran and acidified to pH 2 with hydrochloric acid (3 M), where the resulting precipitate was isolated via vacuum filtration to afford the title compound as a yellow-orange solid (97%). ¹H NMR (500 MHz, CD₃OD) δ 7.96 (d, J=8.6 Hz, 2H), 7.47 (d, J=8.6 Hz, 2H), 7.06 (d, J=8.8 Hz, 2H), 6.75 (d, J=8.9 Hz, 2H), 4.72 (s, 2H).

### 4-methylthiazole-5-carboxamide, Representative Intermediate

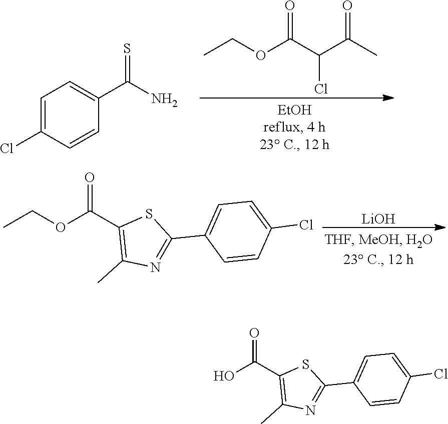

### ethyl 2-(4-chlorophenyl)-4-methylthiazole-5-carboxylate

In a round bottom flask, 4-chlorobenzothioamide (14.6 mmol, 1 equiv) was dissolved in ethanol (0.65 M), and ethyl 2-chloroacetoacetate (17.9 mmol, 1.23 equiv) was added. The reaction mixture was stirred for 4 hours at reflux, then allowed to cool to room temperature and stir for 16 hours. The resulting yellow precipitate was isolated via vacuum filtration and washed with ethanol (0° C.) to afford the title compound as a yellow fluffy solid (81%). R_{f} (30% EA/Hex)=0.80; ¹H NMR (500 MHz, CDCl₃) δ 7.90 (d, J=8.4 Hz, 2H), 7.42 (d, J=8.4 Hz, 2H), 4.36 (q, J=7.1 Hz, 2H), 2.77 (s, 3H), 1.39 (t, J=7.1 Hz, 3H).

### 2-(4-chlorophenyl)-4-methylthiazole-5-carboxylic acid

In a round bottom flask, ethyl 2-(4-chlorophenyl)-4-methylthiazole-5-carboxylate (7.45 mmol, 1 equiv) was dissolved in tetrahydrofuran (1 M), methanol (1.19 M), and water (2 M). Following slow addition of lithium hydroxide (39.5 mmol, 5.3 equiv), the reaction mixture was stirred for 12 hours at room temperature. The reaction mixture was concentrated in vacuo and washed thrice with dichloromethane. The aqueous layer was then acidified to pH 3 with hydrochloric acid (3 M) and extracted thrice with ethyl acetate. The combined organic layers were dried over sodium sulfate and concentrated in vacuo to afford the title compound as a white solid (80%). ¹H NMR (500 MHz, CD₃OD) δ 7.95 (dt, J=8.6, 2.5, 1.8 Hz, 2H), 7.50 (dt, J=8.6, 2.5, 1.9 Hz, 2H), 2.72 (s, 3H).

### 4-methyloxazole-5-carboxamide, Representative Intermediate

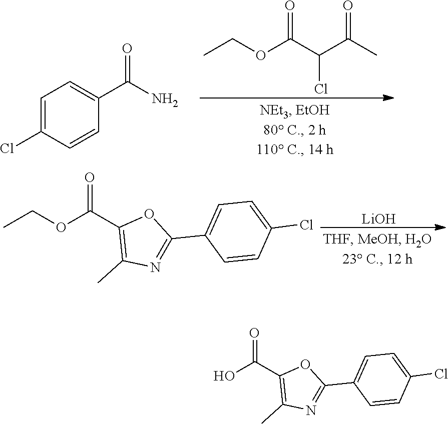

### ethyl 2-(4-chlorophenyl)-4-methyloxazole-5-carboxylate

In a round bottom flask, ethyl 2-chloroacetoacetate (3.04 mmol, 1 equiv) and 4-chlorobenzamide (9.11 mmol, 3 equiv) were dissolved in ethanol (1.20 M). Triethylamine (3.04 mmol, 1 equiv) was added and the reaction mixture was heated to 80° C. for 2 hours, then heated at 110° C. for 14 hours. Upon complete consumption of acetoacetate, the reaction mixture was cooled and the resulting precipitate was removed via vacuum filtration. The filtrate was diluted with ethyl acetate and basified to pH 10 with sodium hydroxide (1 M). The aqueous layer was extracted thrice with ethyl acetate and the combined organic layers were dried over sodium sulfate and concentrated in vacuo. The resulting solids were washed with dichloromethane. The crude product, in the filtrate, was purified by flash column chromatography with hexanes/ethyl acetate, followed by recrystallization in dichloromethane/hexanes to afford the title compound as a light yellow solid (11%). R_{f} (30% EA/Hex)=0.74; ¹H NMR (500 MHz, CD₃OD) δ 8.03 (d, J=8.7 Hz, 2H), 7.54 (d, J=8.7 Hz, 2H), 4.39 (q, J=7.1 Hz, 2H), 2.49 (s, 3H), 1.40 (t, J=7.1 Hz, 3H).

### 2-(4-chlorophenyl)-4-methyloxazole-5-carboxylic acid

In a round bottom flask, ethyl 2-(4-chlorophenyl)-4-methyloxazole-5-carboxylate (0.282 mmol, 1 equiv) was dissolved in tetrahydrofuran (1.20 M), methanol (1.20 M), and water (2.40 M). Following addition of lithium hydroxide (1.50 mmol, 5.3 equiv), the reaction mixture was stirred at room temperature for 12 hours. Upon completion consumption of ester, the reaction mixture was basified to pH 12-13 with sodium hydroxide (3 M) and extracted thrice with dichloromethane. The aqueous layer was then acidified to pH 2-3 with hydrochloric acid (3 M). The formed precipitate was isolated via vacuum filtration to afford the title compound as a white solid (90%). ¹H NMR (500 MHz, CD₃)₂SO) δ 8.00 (dt, J=8.6, 2.6, 2.0 Hz, 2H), 7.62 (dt, J=8.6, 2.6, 2.0 Hz, 2H), 2.44 (s, 3H); ¹³C NMR (175 MHz, CD₃)₂SO) δ 160.03, 159.32, 145.89, 138.00, 136.42, 129.49, 128.37, 124.79, 13.14.

### Weinreb Ketone, Representative Intermediate

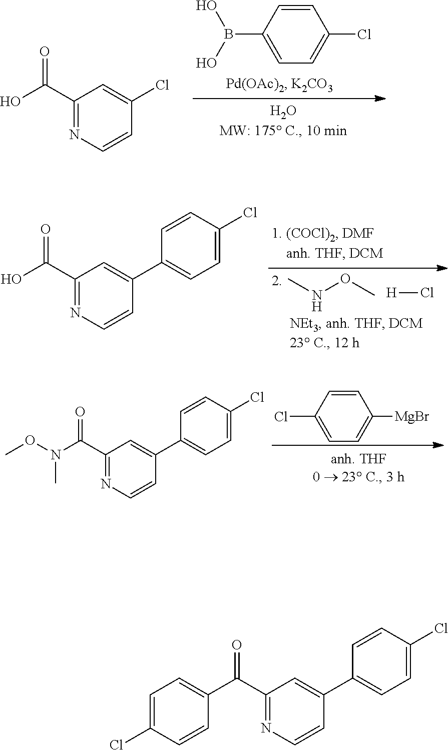

### 4-(4-chlorophenyl)picolinic acid

4-(4-chlorophenyl)picolinic acid was prepared via General Procedure C at a 4.95 mmol scale with potassium carbonate in place of cesium carbonate. The crude product was purified by acid-base extraction, as described, to afford the title compound as a light yellow solid (30%). ¹H NMR (500 MHz, CD₃OD) δ 8.69 (d, J=5.2 Hz, 1H), 8.41 (s, 1H), 7.92 (d, J=5.2 Hz, 1H), 7.79 (d, J=8.2 Hz, 2H), 7.53 (d, J=8.2 Hz, 2H); ¹³C NMR (125 MHz, CD₃OD) δ 166.98, 151.25, 149.99, 149.94, 137.38, 136.59, 130.64, 129.87, 125.84, 123.71.

### 4-(4-chlorophenyl)-N-methoxy-N-methylpicolinamide

4-(4-chlorophenyl)-N-methoxy-N-methylpicolinamide was prepared via General Procedure A from 4-(4-chlorophenyl)picolinic acid and N,O-dimethylhydroxylamine hydrochloride at a 0.60 mmol scale. The crude product was purified by flash column chromatography with hexanes/ethyl acetate to afford the title compound as a white solid (62%). R_{f} (70% EA/Hex)=0.23; ¹H NMR (500 MHz, CDCl₃) δ 8.66 (d, J=5.1 Hz, 1H), 7.86 (s, 1H), 7.61 (dt, J=8.5, 2.6, 2.1 Hz, 2H), 7.54 (dd, J=5.1, 1.9 Hz, 1H), 7.47 (dt, J=8.5, 2.6, 2.1 Hz, 2H), 3.80 (s, 3H), 3.44 (s, 3H).

### (4-chlorophenyl)(4-(4-chlorophenyl)pyridin-2-yl)methanone

In a flame-dried round bottom flask under argon, 4-(4-chlorophenyl)-N-methoxy-N-methylpicolinamide (0.325 mmol, 1 equiv) was dissolved in anhydrous THF (0.20 M). The flask was cooled to 0° C. and (4-chlorophenyl)magnesium bromide (1 M in anhydrous tetrahydrofuran) (0.975 mmol, 3 equiv) was added. The reaction mixture was warmed to room temperature and stirred for 3 hours. Upon complete consumption of the Weinreb amide, the reaction mixture was quenched via slow addition of water at 0° C. This solution was transferred to a separatory funnel and the aqueous layer was extracted thrice with ethyl acetate. The combined organic layers were washed with brine, dried over sodium sulfate, and concentrated in vacuo. The crude product was purified by flash column chromatography with hexanes/ethyl acetate to afford the title compound as a fluffy white solid (45%). R_{f} (20% EA/Hex)=0.47; ¹H NMR (500 MHz, CDCl₃) δ 8.76 (d, J=5.1 Hz, 1H), 8.29-8.24 (m, 1H), 8.10 (d, J=8.5 Hz, 2H), 7.72-7.62 (m, 3H), 7.55-7.44 (m, 4H); ¹³C NMR (125 MHz, CDCl₃) δ 192.54, 155.51, 149.30, 148.68, 139.69, 136.11, 135.88, 134.69, 132.67, 129.71, 128.66, 128.50, 123.99, 122.45.

### 6-picolinamide, Representative Intermediate

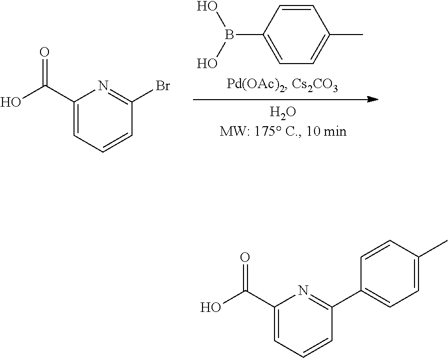

### 6-(p-tolyl)picolinic acid

6-(p-tolyl)picolinic acid was prepared via General Procedure B at a 1.49 mmol scale. The crude product was purified by acid-base extraction to afford the title compound as a white solid (33%). ¹H NMR (500 MHz, CD₃OD) δ 8.08-7.93 (m, 5H), 7.28 (d, J=7.9 Hz, 2H), 2.37 (s, 3H); ¹³C NMR (125 MHz, CD₃OD) δ 166.72, 157.45, 147.55, 139.56, 138.25, 135.26, 129.11, 126.87, 123.56, 122.54, 19.91.

### 2-isonicotinamide, Representative Intermediate

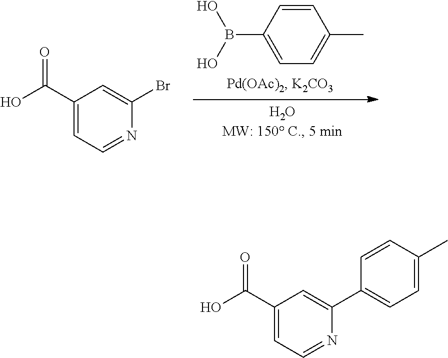

### 2-(p-tolyl)isonicotinic acid

2-(p-tolyl)isonicotinic acid was prepared via General Procedure C at a 1.49 mmol scale in the absence of tetrabutylammonium bromide. The crude product was purified by acid-base extraction (as described in General Procedure B) to afford the title compound as a white solid (82%). H NMR (500 MHz, (CD₃)₂SO) δ 8.83 (d, J=4.9 Hz, 1H), 8.26 (s, 1H), 8.03 (d, J=8.0 Hz, 2H), 7.75 (d, J=4.9 Hz, 1H), 7.33 (d, J=8.0 Hz, 2H), 2.38 (s, 3H).

### 6-pyridazine-4-carboxamide, Representative Intermediate

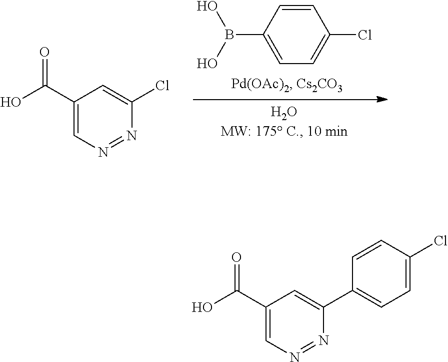 [SMILES: O=C(O)c1cnnc(Cl)c1 ; OB(O)c1ccc(Cl)cc1 ; O=C(O)c1cnnc(-c2ccc(Cl)cc2)c1]

### 6-(4-chlorophenyl)pyridazine-4-carboxylic acid

6-(4-chlorophenyl)pyridazine-4-carboxylic acid was prepared via General Procedure B at a 1.89 mmol scale. The crude product was purified by acid-base extraction to afford the title compound as a dark brown solid (45%). ¹H NMR (500 MHz, CD₃OD) δ 9.55 (d, J=1.6 Hz, 1H), 8.55 (d, J=1.6 Hz, 1H), 8.16 (d, J=8.6 Hz, 2H), 7.59 (d, J=8.6 Hz, 2H).

### 6-pyrazine-2-carboxamide, Representative Intermediate

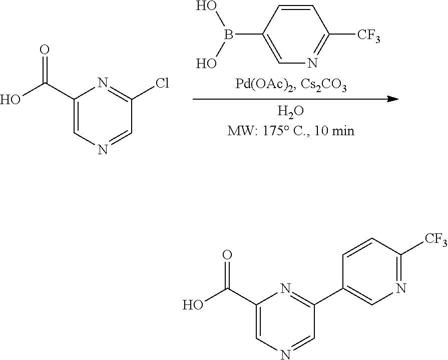 [SMILES: O=C(O)c1cncc(Cl)n1 ; OB(O)c1ccc(C(F)(F)F)nc1 ; O=C(O)c1cncc(-c2ccc(C(F)(F)F)nc2)n1]

### 6-(6-(trifluoromethyl)pyridin-3-yl)pyrazine-2-carboxylic acid

6-(6-(trifluoromethyl)pyridin-3-yl)pyrazine-2-carboxylic acid was prepared via General Procedure B at a 3.15 mmol scale. The crude product was purified by acid-base extraction to afford the title compound as a gray solid (47%). R_{f} (10% MeOH/DCM)=0.21; ¹H NMR (500 MHz, CD₃OD) δ 9.53 (d, J=2.1 Hz, 1H), 9.49 (s, 1H), 9.31 (s, 1H), 8.85 (dd, J=8.2, 2.1 Hz, 1H), 8.01 (d, J=8.2 Hz, 1H); ¹³C NMR (175 MHz, (CD₃)₂CO) 6165.75, 150.69, 150.25 (q, CCF₃, J=34.5 Hz), 149.81, 147.50, 147.04, 144.56, 138.47, 136.08, 123.59 (q, CF₃, J=273.3 Hz), 122.57 (q, ArCF₃, J=3.0 Hz).

### 6-pyrimidine-4-carboxamide, Representative Intermediate

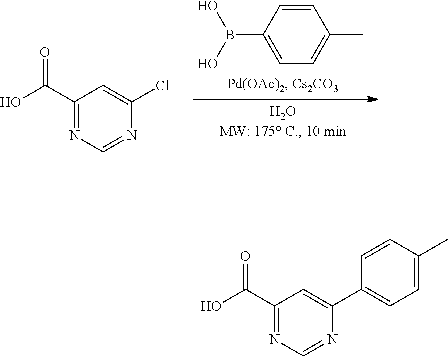

### 6-(p-tolyl)pyrimidine-4-carboxylic acid

6-(p-tolyl)pyrimidine-4-carboxylic acid was prepared via General Procedure B at a 1.58 mmol scale. The crude product was purified by acid-base extraction to afford the title compound as a light pink solid (41%). ¹H NMR (500 MHz, (CD₃)₂SO) δ 9.38-9.32 (m, 1H), 8.47-8.40 (m, 1H), 8.18 (d, J=8.0 Hz, 2H), 7.38 (d, J=8.0 Hz, 2H), 2.39 (s, 3H); ¹³C NMR (125 MHz, (CD₃)₂SO) δ 165.98, 165.25, 159.44, 157.02, 142.33, 133.15, 130.28, 127.68, 116.14, 21.48.

### 2-pyrimidine-4-carboxamide, Representative Intermediate

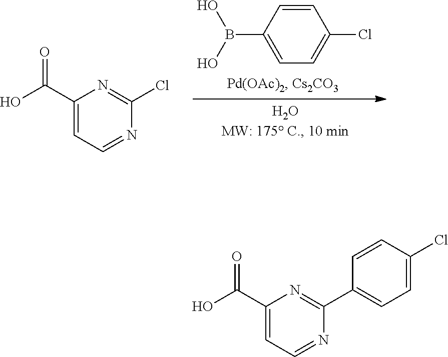 [SMILES: O=C(O)c1ccnc(Cl)n1 ; OB(O)c1ccc(Cl)cc1 ; O=C(O)c1ccnc(-c2ccc(Cl)cc2)n1]

### 2-(4-chlorophenyl)pyrimidine-4-carboxylic acid

2-(4-chlorophenyl)pyrimidine-4-carboxylic acid was prepared via General Procedure B at a 2.21 mmol scale. The crude product was purified by acid-base extraction to afford the title compound as a light yellow solid (5%). ¹H NMR (500 MHz, CD₃OD) δ 9.07 (d, J=4.9 Hz, 1H), 8.53 (d, J=8.5 Hz, 2H), 7.94 (d, J=4.9 Hz, 1H), 7.52 (d, J=8.5 Hz, 2H); ¹³C NMR (125 MHz, CD₃OD) δ 164.94, 162.60, 159.96, 138.04, 137.54, 131.13, 129.68, 119.30.

### 4-pyrimidine-2-carboxamide, Representative Intermediate

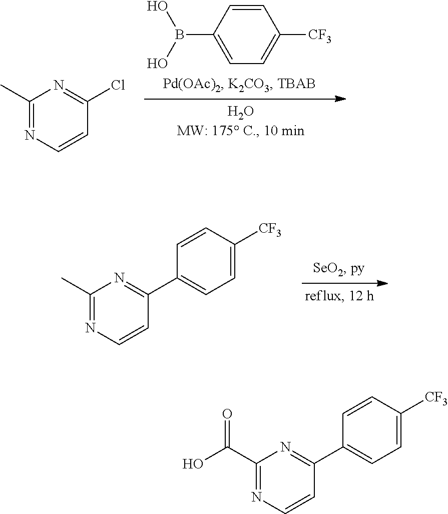

### 2-methyl-4-(4-(trifluoromethyl)phenyl)pyrimidine

In a microwave vial, while stirring, 4-chloro-2-methylpyrimidine (3.89 mmol, 1 equiv) and potassium carbonate (11.7 mmol, 3 equiv) were dissolved in water (0.5 M). Palladium (II) acetate (0.4 mol %), 4-(trifluoromethyl)phenylboronic acid (5.83 mmol, 1.5 equiv), and tetrabutylammonium bromide (3.89 mmol, 1 equiv) were added. Once well mixed, the reaction mixture was heated at 175° C. for 10 minutes in a microwave reactor (Anton Paar Monowave 400). Once cooled to 70° C., the reaction mixture was filtered through celite and washed with ethyl acetate and minimal water. The filtrate was transferred to a separatory funnel and extracted thrice with ethyl acetate. The combined organic layers were dried over sodium sulfate and concentrated in vacuo. The crude product was purified by flash column chromatography with hexanes/ethyl acetate to afford the title compound as a light yellow solid (80%). R_{f} (30% EA/Hex)=0.23; ¹H NMR (500 MHz, CDCl₃) δ 8.74 (d, J=5.3 Hz, 1H), 8.19 (d, J=8.2 Hz, 2H), 7.76 (d, J=8.2 Hz, 2H), 7.54 (d, J=5.3 Hz, 1H), 2.82 (s, 3H); ¹³C NMR (175 MHz, CDCl₃) δ 168.87, 162.65, 158.02, 140.41, 132.61 (q, CCF₃, J=32.6 Hz), 127.67, 126.00 (q, ArCF₃, J=3.8 Hz), 124.03 (q, CF₃, J=272.5 Hz), 114.33, 26.38.

### 4-(4-(trifluoromethyl)phenyl)pyrimidine-2-carboxylic acid

In a flame-dried round bottom flask under argon, 2-methyl-4-(4-(trifluoromethyl)phenyl)pyrimidine (2.94 mmol, 1 equiv) and selenium dioxide (14.7 mmol, 5 equiv) were dissolved in anhydrous pyridine (1 M) and heated to reflux for 12 hours. Upon complete starting material consumption, the reaction mixture was cooled, filtered through a pad of celite, and the filtrate was concentrated in vacuo. The resulting residue was diluted in water, basified to pH 12-13 with sodium hydroxide (3 M), and extracted thrice with dichloromethane. The aqueous layer was then acidified to pH 2-3 with hydrochloric acid (3 M). The formed precipitate was isolated via vacuum filtration to afford the title compound as a light pink solid (90%). ¹H NMR (500 MHz, CD₃OD) δ 8.96 (d, J=5.3 Hz, 1H), 8.41 (d, J=8.2 Hz, 2H), 8.17 (d, J=5.3 Hz, 1H), 7.80 (d, J=8.2 Hz, 2H); ¹³C NMR (125 MHz, CD₃OD) δ 166.39, 164.97, 159.97, 158.26, 140.55, 140.54, 134.09 (q, CCF₃, J=32.6 Hz), 129.41, 127.02 (q, ArCF₃, J=3.8 Hz), 125.38 (q, CF₃, J=271.6 Hz), 120.59.

### 1,3,4-oxadiazole, Representative Intermediate

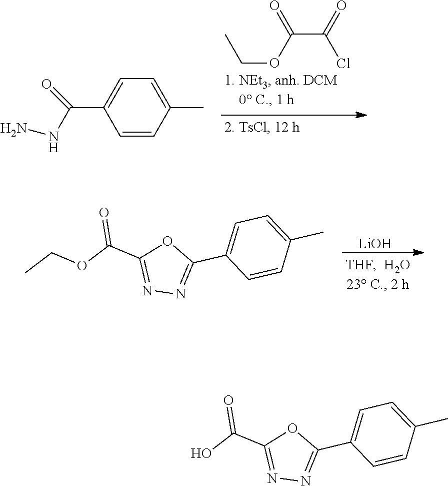

### ethyl 5-(p-tolyl)-1,3,4-oxadiazole-2-carboxylate

In a flame-dried round bottom flask under argon, 4-methylbenzohydrazide (6.66 mmol, 1 equiv) and triethylamine (20.0 mmol, 3 equiv) were dissolved in anhydrous dichloromethane (0.19 M). The flask was cooled to 0° C. and ethyl 2-chloro-2-oxoacetate (6.66 mmol, 1 equiv) was added slowly. After stirring for 1 hour at 0° C., tosyl chloride (6.66 mmol, 1 equiv) was added and the reaction mixture was allowed to warm to room temperature and stir for 12 hours. Upon complete consumption of 4-methylbenzohydrazide, the reaction was diluted with ethyl acetate and washed with saturated aqueous sodium bicarbonate and brine. The organic layer was then dried over sodium sulfate and concentrated in vacuo. The crude product was purified by flash column chromatography with hexanes/ethyl acetate to afford the title compound as a fluffy white solid (86%). R_{f} (30% EA/Hex)=0.52; ¹H NMR (500 MHz, CDCl₃) δ 8.05 (d, J=8.2 Hz, 2H), 7.35 (d, J=8.2 Hz, 2H), 4.55 (q, J=7.1 Hz, 2H), 2.45 (s, 3H), 1.49 (t, J=7.1 Hz, 3H); ¹³C NMR (125 MHz, CDCl₃) δ 166.81, 156.44, 154.65, 143.77, 130.08, 127.74, 120.13, 63.59, 21.88, 14.25.

### 5-(p-tolyl)-1,3,4-oxadiazole-2-carboxylic acid

In a round bottom flask, ethyl 5-(p-tolyl)-1,3,4-oxadiazole-2-carboxylate (3.96 mmol, 1 equiv) was dissolved in tetrahydrofuran (0.13 M) and water (0.40 M). Following addition of lithium hydroxide (7.92 mmol, 2 equiv), the reaction mixture was stirred at room temperature for 12 hours. Upon completion consumption of ester, the reaction mixture was basified to pH 12-13 with sodium hydroxide (3 M) and extracted thrice with dichloromethane. The aqueous layer was then acidified to pH 2-3 with hydrochloric acid (3 M). The formed precipitate was isolated via vacuum filtration to afford the title compound as a white solid (quantitative). ¹H NMR (500 MHz, CD₃OD) δ 7.99 (d, J=8.1 Hz, 2H), 7.38 (d, J=8.1 Hz, 2H), 2.42 (s, 3H); ¹³C NMR (125 MHz, CD₃OD) δ 166.63, 162.71, 159.50, 144.41, 131.00, 128.17, 122.05, 21.61.

### Buchwald Product, Representative Intermediate

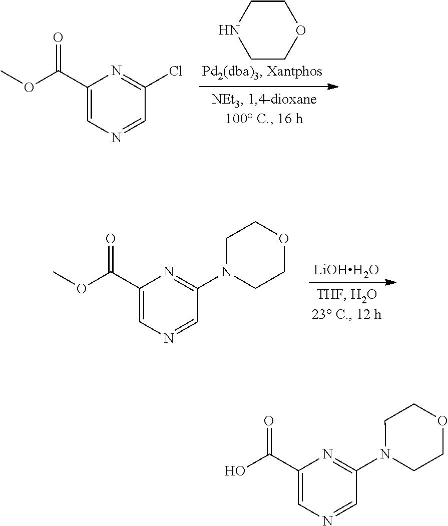

### methyl 6-morpholinopyrazine-2-carboxylate

In a flame-dried round bottom flask, methyl 6-chloropyrazine-2-carboxylate (5.79 mmol, 1 equiv) was dissolved in 1,4-dioxane (0.20 M). Tris(dibenzylideneacetone)dipalladium(0) (0.348 mmol, 6 mol %), Xantphos (0.579 mmol, 0.1 equiv), triethylamine (11.6 mmol, 2 equiv), and morpholine (12.2 mmol, 2.1 equiv) were added, and the reaction mixture was degassed with argon. The flask was heated at 100° C. for 16 hours. Upon complete consumption of the ester, the reaction mixture was filtered through a pad of celite and extracted thrice with ethyl acetate. The combined organic layers were dried over sodium sulfate and concentrated in vacuo. The crude product was purified by flash column chromatography with hexanes/ethyl acetate, followed by recrystallization from dichloromethane/hexanes to afford the title compound as a light orange crystalline solid (41%). R_{f} (50% EA/Hex)=0.31; ¹H NMR (500 MHz, CDCl₃) δ 8.57 (s, 1H), 8.28 (s, 1H), 3.97 (s, 3H), 3.84 (dd, J=5.1, 4.8 Hz, 4H), 3.66 (dd, J=5.1, 4.8 Hz, 4H); ¹³C NMR (175 MHz, CDCl₃) δ 165.34, 154.08, 140.14, 134.42, 133.93, 66.54, 52.96, 44.53.

### 6-morpholinopyrazine-2-carboxylic acid

In a round bottom flask, methyl 6-morpholinopyrazine-2-carboxylate (2.46 mmol, 1 equiv) was dissolved in tetrahydrofuran (0.13 M) and water (0.40 M). Following addition of lithium hydroxide monohydrate (7.39 mmol, 3 equiv), the reaction mixture was stirred at room temperature for 3 hours. Upon completion consumption of the ester, the reaction mixture was basified to pH 12-13 with sodium hydroxide (3 M) and extracted thrice with ether. The aqueous layer was then acidified to pH 2-3 with hydrochloric acid (3 M). The formed precipitate was isolated via vacuum filtration to afford the title compound as a light yellow crystalline solid (76%). ¹H NMR (500 MHz, CDCl₃) δ 10.09 (s, 1H), 8.72 (s, 1H), 8.43 (s, 1H), 3.88 (t, J=5.0 Hz, 4H), 3.65 (t, J=5.0 Hz, 4H); ¹³C NMR (175 MHz, CDCl₃) δ 163.61, 152.70, 137.29, 135.59, 133.14, 66.20, 44.36.

### N-methyl-N,1-di-p-tolyl-1H-1,2,4-triazole-3-carboxamide (APZ-2327)

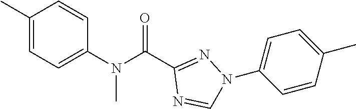

N-methyl-N,1-di-p-tolyl-1H-1,2,4-triazole-3-carboxamide was prepared via General Procedure A from 1-(p-tolyl)-1H-1,2,4-triazole-3-carboxylic acid. The crude product was purified by flash column chromatography with hexanes/ethyl acetate to afford title compound as a white pearlescent solid (47%). mp 177-178° C.; R_{f} (60% EA/Hex)=0.21; ¹H NMR (700 MHz, CDCl₃) δ 8.26 (s, 1H), 7.36 (d, J=8.0 Hz, 2H), 7.23 (d, J=8.0 Hz, 2H), 7.17-6.98 (m, 4H), 3.51 (s, 3H), 2.38 (s, 3H), 2.31 (s, 3H); ¹³C NMR (175 MHz, CDCl₃) δ 161.83, 158.63, 141.65, 140.40, 138.62, 137.06, 134.44, 130.30, 129.81, 126.79, 119.98, 38.19, 21.17, 21.16.

### N-methyl-N-phenyl-1-(p-tolyl)-1H-1,2,4-triazole-3-carboxamide (APZ-2329)

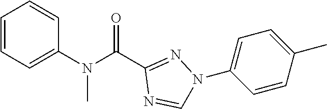 [SMILES: Cc1ccc(-n2cnc(C(=O)N(C)c3ccccc3)n2)cc1]

N-methyl-N-phenyl-1-(p-tolyl)-1H-1,2,4-triazole-3-carboxamide was prepared via General Procedure A from 1-(p-tolyl)-1H-1,2,4-triazole-3-carboxylic acid. The crude product was purified by flash column chromatography with hexanes/ethyl acetate to afford title compound as a fluffy white solid (61%). mp 150-154° C.; R_{f} (60% EA/Hex)=0.22; ¹H NMR (700 MHz, CDCl₃) δ 8.27 (s, 1H), 7.45-7.28 (m, 4H), 7.26-7.10 (m, 5H), 3.54 (s, 3H), 2.37 (s, 3H); ¹³C NMR (175 MHz, CDCl₃) δ 161.68, 158.41, 144.20, 140.47, 138.63, 134.34, 130.26, 129.16, 127.17, 126.97, 119.94, 38.11, 21.12.

### 1-(4-chlorophenyl)-N-methyl-N-phenyl-1H-1,2,4-triazole-3-carboxamide (APZ-2330)

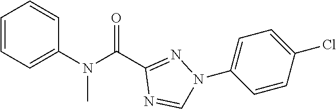 [SMILES: CN(C(=O)c1ncn(-c2ccc(Cl)cc2)n1)c1ccccc1]

1-(4-chlorophenyl)-N-methyl-N-phenyl-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a light yellow solid (57%). mp 178-180° C.; R_{f} (60% EA/Hex)=0.25; ¹H NMR (500 MHz, CD₃OD) δ 8.90 (s, 1H), 7.57-7.43 (m, 4H), 7.37-7.19 (m, 5H), 3.51 (s, 3H); ¹³C NMR (175 MHz, CDCl₃) δ 161.37, 158.77, 144.10, 140.58, 135.15, 134.26, 129.97, 129.23, 127.31, 127.03, 121.18, 38.15.

### 1-(4-chlorophenyl)-N-methyl-N-(p-tolyl)-1H-1,2,4-triazole-3-carboxamide (APZ-2010)

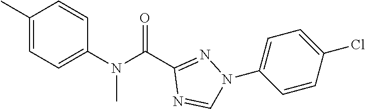 [SMILES: Cc1ccc(N(C)C(=O)c2ncn(-c3ccc(Cl)cc3)n2)cc1]

1-(4-chlorophenyl)-N-methyl-N-(p-tolyl)-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as an off-white solid (53%). mp 177-179° C.; R_{f} (60% EA/Hex)=0.28; ¹H NMR (500 MHz, CDCl₃) δ 8.30 (s, 1H), 7.52-7.36 (m, 4H), 7.16-6.95 (m, 4H), 3.51 (s, 3H), 2.31 (s, 3H); ¹³C NMR (175 MHz, CDCl₃) δ 161.49, 158.91, 141.45, 140.50, 137.18, 135.19, 134.19, 129.95, 129.81, 126.79, 121.16, 38.17, 21.13.

### 1-(4-fluorophenyl)-N-methyl-N-(p-tolyl)-1H-1,2,4-triazole-3-carboxamide (APZ-2011)

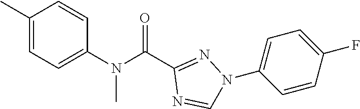 [SMILES: Cc1ccc(N(C)C(=O)c2ncn(-c3ccc(F)cc3)n2)cc1]

1-(4-fluorophenyl)-N-methyl-N-(p-tolyl)-1H-1,2,4-triazole-3-carboxamide was prepared via General Procedure A from 1-(4-fluorophenyl)-1H-1,2,4-triazole-3-carboxylic acid. The crude product was purified by flash column chromatography with hexanes/ethyl acetate to afford title compound as a light yellow solid (99%). mp 177-180° C.; R_{f} (60% EA/Hex)=0.21; ¹H NMR (700 MHz, CDCl₃) δ 8.27 (s, 1H), 7.59-7.38 (m, 2H), 7.22-6.85 (m, 6H), 3.51 (s, 3H), 2.32 (s, 3H); ¹³C NMR (175 MHz, CDCl₃) δ 162.22 (d, CF, J=249.1 Hz), 161.59, 158.87, 141.51, 140.56, 137.14, 132.97 (d, ArF, J=3.1 Hz), 129.81, 126.78, 122.01 (d, ArF, J=8.4 Hz), 116.76 (d, ArF, J=23.3 Hz), 38.16, 21.13.

### 1-(4-chlorophenyl)-N-(4-ethylphenyl)-1H-1,2,4-triazole-3-carboxamide (APZ-2022)

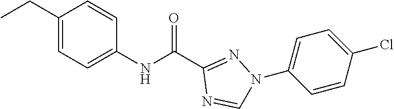 [SMILES: CCc1ccc(NC(=O)c2ncn(-c3ccc(Cl)cc3)n2)cc1]

1-(4-chlorophenyl)-N-(4-ethylphenyl)-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a lustrous white solid (43%). mp 223-228° C.; R_{f} (40% EA/Hex)=0.32; ¹H NMR (700 MHz, CDCl₃) δ 8.92 (s, 1H), 8.59 (s, 1H), 7.74 (dt, J=8.8, 2.9, 2.0 Hz, 2H), 7.65 (dt, J=8.4, 2.7, 2.0, 2H), 7.52 (dt, J=8.8, 2.9, 2.0 Hz, 2H), 7.22 (dt, J=8.4, 2.7, 2.0, 2H), 2.65 (q, J=7.6 Hz, 2H), 1.24 (t, J=7.6 Hz, 3H); ¹³C NMR (175 MHz, CDCl₃) δ 158.21, 156.22, 141.55, 141.11, 135.18, 135.01, 134.97, 130.24, 128.64, 121.68, 120.13, 28.53, 15.79.

### N-(4-(tert-butyl)phenyl)-1-(4-chlorophenyl)-1H-1,2,4-triazole-3-carboxamide (APZ-2023)

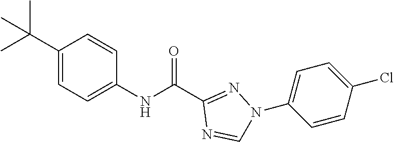 [SMILES: CC(C)(C)c1ccc(NC(=O)c2ncn(-c3ccc(Cl)cc3)n2)cc1]

N-(4-(tert-butyl)phenyl)-1-(4-chlorophenyl)-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a lustrous yellow solid (60%). R_{f} (40% EA/Hex)=0.40; ¹H NMR (500 MHz, CDCl₃) δ 8.94 (s, 1H), 8.60 (s, 1H), 7.77-7.71 (m, 2H), 7.69-7.63 (m, 2H), 7.55-7.49 (m, 2H), 7.43-7.39 (m, 2H), 1.33 (s, 9H); ¹³C NMR (175 MHz, CDCl₃) δ 158.17, 156.25, 148.00, 141.59, 135.16, 134.93, 134.74, 130.21, 126.13, 121.66, 119.82, 34.60, 31.49.

### 1-(4-chlorophenyl)-N-(4-(dimethylamino)phenyl)-1H-1,2,4-triazole-3-carboxamide (APZ-2024)

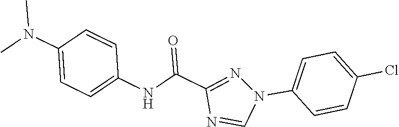 [SMILES: CN(C)c1ccc(NC(=O)c2ncn(-c3ccc(Cl)cc3)n2)cc1]

1-(4-chlorophenyl)-N-(4-(dimethylamino)phenyl)-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a light yellow solid (70%). R_{f} (70% EA/Hex)=0.36; ¹H NMR (500 MHz, CDCl₃) δ 8.82 (s, 1H), 8.58 (s, 1H), 7.74 (dt, J=8.9, 3.0, 2.0 Hz, 2H), 7.60 (dt, J=9.0, 3.3, 2.1 Hz, 2H), 7.52 (dt, J=8.9, 3.0, 2.0 Hz, 2H), 6.76 (dt, J=9.0, 3.3, 2.1 Hz, 2H), 2.95 (s, 6H); ¹³C NMR (175 MHz, CDCl₃) δ 158.42, 155.94, 148.36, 141.47, 135.23, 134.79, 130.18, 127.21, 121.62, 121.58, 113.09, 40.97.

### N,1-bis(4-chlorophenyl)-N-methyl-1H-1,2,4-triazole-3-carboxamide (APZ-2025)

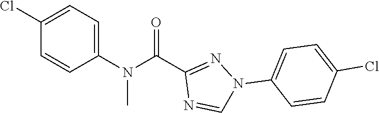 [SMILES: CN(C(=O)c1ncn(-c2ccc(Cl)cc2)n1)c1ccc(Cl)cc1]

N,1-bis(4-chlorophenyl)-N-methyl-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a white solid (96%). R_{f} (60% EA/Hex)=0.21; ¹H NMR (500 MHz, CDCl₃) δ 8.33 (s, 1H), 7.50-7.41 (m, 4H), 7.28 (d, J=8.0 Hz, 1H), 7.18-7.09 (m, 2H), 3.52 (s, 3H); ¹³C NMR (175 MHz, CDCl₃) δ 161.18, 158.52, 142.64, 140.68, 135.08, 134.44, 133.13, 130.07, 129.44, 128.42, 121.20, 38.17.

### 1-(4-chlorophenyl)-N-(4-methoxyphenyl)-N-methyl-1H-1,2,4-triazole-3-carboxamide (APZ-2026)

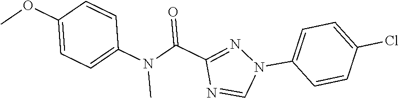 [SMILES: COc1ccc(N(C)C(=O)c2ncn(-c3ccc(Cl)cc3)n2)cc1]

1-(4-chlorophenyl)-N-(4-methoxyphenyl)-N-methyl-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a light yellow pearlescent solid (89%). R_{f} (80% EA/Hex)=0.24; ¹H NMR (500 MHz, CDCl₃) δ 8.29 (s, 1H), 7.44 (q, J=8.8 Hz, 4H), 7.11 (d, J=8.7 Hz, 2H), 6.80 (d, J=8.7 Hz, 2H), 3.77 (s, 3H), 3.49 (s, 3H); ¹³C NMR (175 MHz, CDCl₃) δ 161.60, 158.90, 158.63, 140.50, 136.83, 135.17, 134.16, 129.94, 128.29, 121.13, 114.31, 55.52, 38.31.

### 1-(4-chlorophenyl)-N-(4-ethylphenyl)-N-methyl-1H-1,2,4-triazole-3-carboxamide (APZ-2027)

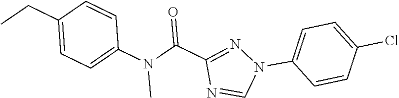 [SMILES: CCc1ccc(N(C)C(=O)c2ncn(-c3ccc(Cl)cc3)n2)cc1]

1-(4-chlorophenyl)-N-(4-ethylphenyl)-N-methyl-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a white pearlescent solid (87%). mp 197-199° C.; R_{f} (60% EA/Hex)=0.20; ¹H NMR (500 MHz, CDCl₃) δ 8.30 (s, 1H), 7.40 (s, 4H), 7.11 (q, J=7.9 Hz, 4H), 3.52 (s, 3H), 2.62 (q, J=7.6 Hz, 2H), 1.21 (t, J=7.6 Hz, 3H); ¹³C NMR (175 MHz, CDCl₃) δ 161.38, 158.83, 143.52, 141.70, 140.53, 135.21, 134.21, 129.94, 128.62, 126.90, 121.18, 38.21, 28.52, 15.67.

### 1-(4-chlorophenyl)-N-(3,4-dimethylphenyl)-N-methyl-1H-1,2,4-triazole-3-carboxamide (APZ-2028)

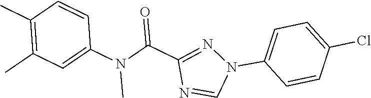 [SMILES: Cc1ccc(N(C)C(=O)c2ncn(-c3ccc(Cl)cc3)n2)cc1C]

1-(4-chlorophenyl)-N-(3,4-dimethylphenyl)-N-methyl-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a white pearlescent solid (85%). mp 196-198° C.; R_{f} (60% EA/Hex)=0.21; ¹H NMR (700 MHz, CDCl₃) δ 8.31 (s, 1H), 7.53-7.30 (m, 4H), 7.07-6.94 (m, 2H), 6.88 (d, J=6.3 Hz, 1H), 3.50 (s, 3H), 2.36-2.09 (m, 6H); ¹³C NMR (175 MHz, CDCl₃) δ 161.53, 159.01, 141.62, 140.48, 137.60, 135.83, 135.22, 134.16, 130.17, 129.95, 127.84, 124.30, 121.16, 38.21, 19.88, 19.45.

### 1-(4-chlorophenyl)-N-(4-fluorophenyl)-1H-1,2,4-triazole-3-carboxamide (APZ-2041)

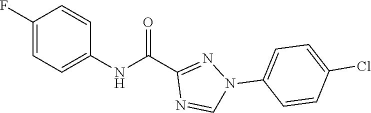 [SMILES: O=C(Nc1ccc(F)cc1)c1ncn(-c2ccc(Cl)cc2)n1]

1-(4-chlorophenyl)-N-(4-fluorophenyl)-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a white solid (85%). mp 224-225° C.; R_{f} (70% EA/Hex)=0.58; ¹H NMR (400 MHz, (CD₃)₂SO) δ 10.63 (s, 1H), 9.53 (s, 1H), 8.00 (d, J=8.8 Hz, 2H), 7.87 (dd, J=9.0, 4.9 Hz, 2H), 7.70 (d, J=8.8 Hz, 2H), 7.21 (t, J=9.0 Hz, 2H); ¹³C NMR (175 MHz, (CD₃)₂SO) δ 158.56 (d, J=240.9 Hz), 157.42, 156.95, 143.82, 135.29, 134.61 (d, J=2.6 Hz), 132.77, 129.83, 122.47 (d, J=8.0 Hz), 121.61, 115.26 (d, J=22.3 Hz).

### N,1-bis(4-chlorophenyl)-1H-1,2,4-triazole-3-carboxamide (APZ-2042)

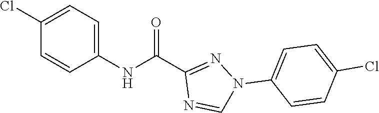 [SMILES: O=C(Nc1ccc(Cl)cc1)c1ncn(-c2ccc(Cl)cc2)n1]

N,1-bis(4-chlorophenyl)-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a tan solid (65%). R_{f} (50% EA/Hex)=0.23; ¹H NMR (400 MHz, CDCl₃) δ 8.98 (s, 1H), 8.60 (s, 1H), 7.72 (t, J=8.5 Hz, 4H), 7.53 (d, J=8.2 Hz, 2H), 7.36 (d, J=8.5 Hz, 2H), 3.35 (s, 1H).

### 1-(4-chlorophenyl)-N-(4-fluorophenyl)-N-methyl-1H-1,2,4-triazole-3-carboxamide (APZ-2043)

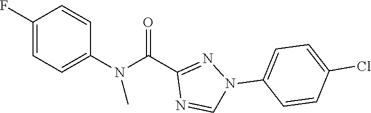 [SMILES: CN(C(=O)c1ncn(-c2ccc(Cl)cc2)n1)c1ccc(F)cc1]

1-(4-chlorophenyl)-N-(4-fluorophenyl)-N-methyl-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a white pearlescent solid (85%). mp 174-176° C.; R_{f} (70% EA/Hex)=0.19; ¹H NMR (500 MHz, CDCl₃) δ 8.33 (s, 1H), 7.56-7.34 (m, 4H), 7.24-7.12 (m, 2H), 7.07-6.89 (m, 2H), 3.51 (s, 3H); ¹³C NMR (175 MHz, CDCl₃) δ 161.56 (d, CF, J=247.8 Hz), 161.32, 158.59, 140.61, 140.08, 135.09, 134.37, 130.04, 128.93 (d, ArF, J=8.5 Hz), 121.14, 116.11 (d, ArF, J=22.7 Hz), 38.31.

### N-(4-(tert-butyl)phenyl)-1-(4-chlorophenyl)-N-methyl-1H-1,2,4-triazole-3-carboxamide (APZ-2044)

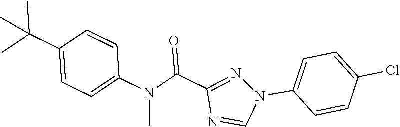 [SMILES: CN(C(=O)c1ncn(-c2ccc(Cl)cc2)n1)c1ccc(C(C)(C)C)cc1]

N-(4-(tert-butyl)phenyl)-1-(4-chlorophenyl)-N-methyl-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a white solid (91%). mp 203-207° C.; R_{f} (70% EA/Hex)=0.22; ¹H NMR (500 MHz, CDCl₃) δ 8.32 (s, 1H), 7.46-7.28 (m, 6H), 7.10 (d, J=8.1 Hz, 2H), 3.52 (s, 3H), 1.30 (s, 9H); ¹³C NMR (175 MHz, CDCl₃) δ 161.20, 158.64, 150.40, 141.55, 140.60, 135.22, 134.22, 129.90, 126.58, 126.07, 121.18, 38.20, 34.70, 31.45.

### 1-(4-chlorophenyl)-N-(4-(trifluoromethyl)phenyl)-1H-1,2,4-triazole-3-carboxamide (APZ-2045)

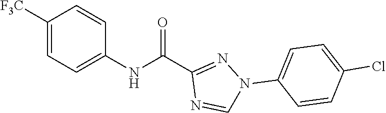 [SMILES: O=C(Nc1ccc(C(F)(F)F)cc1)c1ncn(-c2ccc(Cl)cc2)n1]

1-(4-chlorophenyl)-N-(4-(trifluoromethyl)phenyl)-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a light yellow solid (99%). mp 214-217° C.; R_{f} (40% EA/Hex)=0.32; ¹H NMR (500 MHz, (CD₃)₂CO) δ 10.03 (s, 1H), 9.28 (s, 1H), 8.18 (d, J=8.3 Hz, 2H), 8.04-7.93 (m, 2H), 7.75 (d, J=8.3 Hz, 2H), 7.70-7.52 (m, 2H).

### 1-(4-chlorophenyl)-N-(3,4-dichlorophenyl)-1H-1,2,4-triazole-3-carboxamide (APZ-2046)

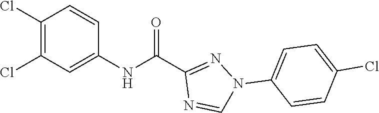 [SMILES: O=C(Nc1ccc(Cl)c(Cl)c1)c1ncn(-c2ccc(Cl)cc2)n1]

1-(4-chlorophenyl)-N-(3,4-dichlorophenyl)-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a white solid (37%). mp 254-258° C.; R_{f} (70% EA/Hex)=0.59; ¹H NMR (400 MHz, (CD₃)₂SO) δ 10.88 (s, 1H), 9.54 (s, 1H), 8.21 (d, J=1.6 Hz, 1H), 8.03-7.95 (m, 2H), 7.86 (dd, J=8.8, 1.6 Hz, 1H), 7.69 (d, J=8.6 Hz, 2H), 7.65-7.59 (m, 1H).

### 1-(4-chlorophenyl)-N-methyl-N-(4-(trifluoromethyl)phenyl)-1H-1,2,4-triazole-3-carboxamide (APZ-2048)

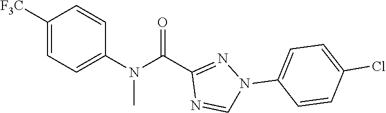 [SMILES: CN(C(=O)c1ncn(-c2ccc(Cl)cc2)n1)c1ccc(C(F)(F)F)cc1]

1-(4-chlorophenyl)-N-methyl-N-(4-(trifluoromethyl)phenyl)-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a light orange solid (82%). mp 173-176° C.; R_{f} (70% EA/Hex)=0.30; ¹H NMR (400 MHz, CDCl₃) δ 8.36 (s, 1H), 7.60 (d, J=8.1 Hz, 2H), 7.42 (s, 4H), 7.33 (d, J=8.1 Hz, 2H), 3.57 (s, 3H); ¹³C NMR (175 MHz, CDCl₃) δ 160.94, 158.22, 147.28, 140.84, 134.99, 134.52, 130.04, 129.31 (q, CCF₃, J=32.8 Hz), 127.26, 126.42 (q, ArCF₃, J=3.7 Hz), 123.88 (q, CF₃, J=272.1 Hz), 121.17, 38.23.

### N-(4-acetylphenyl)-1-(4-chlorophenyl)-N-methyl-1H-1,2,4-triazole-3-carboxamide (APZ-2049)

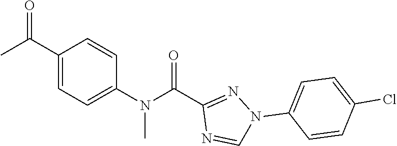 [SMILES: CC(=O)c1ccc(N(C)C(=O)c2ncn(-c3ccc(Cl)cc3)n2)cc1]

N-(4-acetylphenyl)-1-(4-chlorophenyl)-N-methyl-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a yellow solid (85%). mp 166-169° C.; R_{f} (80% EA/Hex)=0.23; ¹H NMR (500 MHz, CDCl₃) δ 8.39 (s, 1H), 7.91 (d, J=8.3 Hz, 2H), 7.48 (d, J=8.5 Hz, 2H), 7.46-7.38 (m, 2H), 7.28 (d, J=8.3 Hz, 2H), 3.57 (s, 3H), 2.58 (s, 3H); ¹³C NMR (175 MHz, CDCl₃) δ 197.01, 161.15, 158.45, 148.15, 140.79, 135.49, 134.97, 134.43, 130.01, 129.42, 126.59, 121.17, 38.07, 26.70.

### 1-(4-chlorophenyl)-N-methyl-N-(4-nitrophenyl)-1H-1,2,4-triazole-3-carboxamide (APZ-2052)

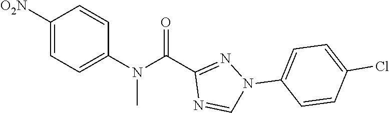 [SMILES: CN(C(=O)c1ncn(-c2ccc(Cl)cc2)n1)c1ccc([N+](=O)[O-])cc1]

1-(4-chlorophenyl)-N-methyl-N-(4-nitrophenyl)-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a yellow solid (73%). R_{f} (70% EA/Hex)=0.24; ¹H NMR (400 MHz, CDCl₃) δ 8.38 (s, 1H), 8.19 (d, J=8.6 Hz, 2H), 7.51 (d, J=8.8 Hz, 2H), 7.45 (d, J=8.8 Hz, 2H), 7.36 (d, J=8.6 Hz, 2H), 3.61 (s, 3H); ¹³C NMR (175 MHz, CDCl₃) δ 160.97, 158.17, 149.75, 146.07, 140.93, 134.89, 134.71, 130.14, 127.10, 124.72, 121.24, 38.20.

### 1-(4-chlorophenyl)-N-(3,4-dichlorophenyl)-N-methyl-1H-1,2,4-triazole-3-carboxamide (APZ-2053)

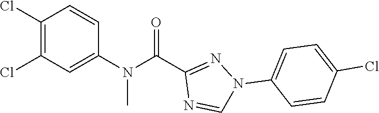 [SMILES: CN(C(=O)c1ncn(-c2ccc(Cl)cc2)n1)c1ccc(Cl)c(Cl)c1]

1-(4-chlorophenyl)-N-(3,4-dichlorophenyl)-N-methyl-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a lustrous light yellow solid (74%). R_{f} (70% EA/Hex)=0.31; ¹H NMR (500 MHz, CDCl₃) δ 8.41 (s, 1H), 7.67-7.33 (m, 6H), 7.15-6.96 (m, 1H), 3.52 (s, 3H); ¹³C NMR (175 MHz, (CD₃)₂SO) δ 160.90, 158.16, 143.33, 140.86, 134.99, 134.51, 132.93, 131.52, 130.79, 130.07, 129.11, 126.52, 121.22, 38.26.

### 1-(4-chlorophenyl)-N-(4-nitrophenyl)-1H-1,2,4-triazole-3-carboxamide (APZ-2054)

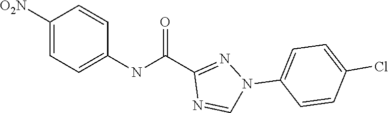 [SMILES: O=C(Nc1ccc([N+](=O)[O-])cc1)c1ncn(-c2ccc(Cl)cc2)n1]

1-(4-chlorophenyl)-N-(4-nitrophenyl)-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a lustrous yellow solid (43%). mp>260° C.; R_{f} (70% EA/Hex)=0.50; ¹H NMR (400 MHz, (CD₃)₂SO) δ 11.18 (s, 1H), 9.59 (s, 1H), 8.29 (d, J=8.8 Hz, 2H), 8.17 (d, J=8.8 Hz, 2H), 8.03 (d, J=8.4 Hz, 2H), 7.72 (d, J=8.4 Hz, 2H); ¹³C NMR (125 MHz, (CD₃)₂SO) δ 157.69, 157.01, 144.55, 144.15, 143.02, 135.31, 133.07, 129.97, 124.85, 121.84, 120.44.

### N-(4-acetylphenyl)-1-(4-chlorophenyl)-1H-1,2,4-triazole-3-carboxamide (APZ-2055)

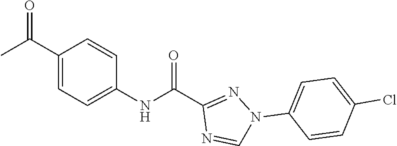 [SMILES: CC(=O)c1ccc(NC(=O)c2ncn(-c3ccc(Cl)cc3)n2)cc1]

N-(4-acetylphenyl)-1-(4-chlorophenyl)-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a tan solid (72%). mp>260° C.; R_{f} (70% EA/Hex)=0.37; ¹H NMR (500 MHz, (CD₃)₂SO) δ 10.84 (s, 1H), 9.54 (s, 1H), 8.04-7.93 (m, 6H), 7.69 (dt, J=8.8, 3.2, 2.1 Hz, 2H), 2.54 (s, 3H ¹³C NMR (175 MHz, (CD₃)₂SO) δ 196.69, 157.37, 157.25, 143.96, 142.71, 135.28, 132.86, 132.43, 129.86, 129.28, 121.66, 119.83, 26.53.

### 1-(4-chlorophenyl)-N-methyl-N-(o-tolyl)-1H-1,2,4-triazole-3-carboxamide (APZ-2061)

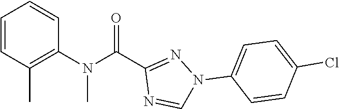 [SMILES: Cc1ccccc1N(C)C(=O)c1ncn(-c2ccc(Cl)cc2)n1]

1-(4-chlorophenyl)-N-methyl-N-(o-tolyl)-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a light yellow solid (56%). mp 166-168° C.; R_{f} (70% EA/Hex)=0.46; ¹H NMR (500 MHz, CDCl₃, rotameric mixture, major form reported) δ 8.29 (s, 1H), 7.42-7.31 (m, 4H), 7.24-7.08 (m, 4H), 3.43 (s, 3H), 2.32 (s, 3H); ¹³C NMR (175 MHz, CDCl₃, rotameric mixture, major form reported) δ 161.40, 158.43, 142.84, 140.52, 136.17, 135.19, 134.15, 130.95, 129.95, 128.40, 128.22, 126.84, 121.00, 37.19, 17.87.

### 1-(4-chlorophenyl)-N-methyl-N-(3-(trifluoromethyl)phenyl)-1H-1,2,4-triazole-3-carboxamide (APZ-2062)

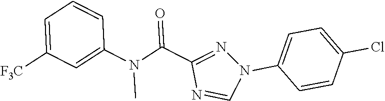 [SMILES: CN(C(=O)c1ncn(-c2ccc(Cl)cc2)n1)c1cccc(C(F)(F)F)c1]

1-(4-chlorophenyl)-N-methyl-N-(3-(trifluoromethyl)phenyl)-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a light yellow solid (62%). mp 143-145° C.; R_{f} (70% EA/Hex)=0.47; ¹H NMR (500 MHz, CDCl₃) δ 8.36 (s, 1H), 7.52 (d, J=8.0 Hz, 2H), 7.49-7.29 (m, 6H), 3.57 (s, 3H); ¹³C NMR (175 MHz, CDCl₃) δ 161.06, 158.21, 144.60, 140.82, 135.01, 134.52, 131.71 (q, CCF₃, J=32.8 Hz), 130.33, 130.05, 129.90, 124.34, 124.07 (q, ArCF₃, J=3.8 Hz), 123.62 (q, CF₃, J=272.5 Hz), 121.20, 38.20.

### 1-(4-chlorophenyl)-N-methyl-N-(4-(methylsulfonyl)phenyl)-1H-1,2,4-triazole-3-carboxamide (APZ-2063)

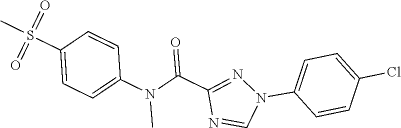 [SMILES: CN(C(=O)c1ncn(-c2ccc(Cl)cc2)n1)c1ccc(S(C)(=O)=O)cc1]

1-(4-chlorophenyl)-N-methyl-N-(4-(methylsulfonyl)phenyl)-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a lustrous light yellow solid (55%). mp 202-204° C.; R_{f} (70% EA/Hex)=0.24; ¹H NMR (500 MHz, CDCl₃) δ 8.39 (s, 1H), 7.91 (d, J=8.4 Hz, 2H), 7.57-7.32 (m, 6H), 3.60 (s, 3H), 3.06 (s, 3H); ¹³C NMR (175 MHz, CDCl₃) δ 160.99, 158.12, 148.95, 140.95, 138.89, 134.92, 134.67, 130.12, 128.65, 127.47, 121.23, 44.54, 38.28.

### 1-(4-chlorophenyl)-N-methyl-N-(m-tolyl)-1H-1,2,4-triazole-3-carboxamide (APZ-2064)

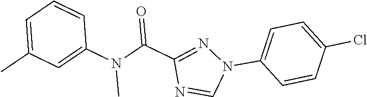 [SMILES: Cc1cccc(N(C)C(=O)c2ncn(-c3ccc(Cl)cc3)n2)c1]

1-(4-chlorophenyl)-N-methyl-N-(m-tolyl)-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as an orange solid (45%). mp 147-149° C.; R_{f} (70% EA/Hex)=0.32; ¹H NMR (500 MHz, CDCl₃) δ 8.32 (s, 1H), 7.42 (s, 4H), 7.16 (m, 1H), 7.04 (d, J=7.2 Hz, 2H), 6.99-6.81 (m, 1H), 3.52 (s, 3H), 2.30 (s, 3H); ¹³C NMR (175 MHz, CDCl₃) δ 161.42, 158.85, 143.93, 140.57, 139.22, 135.19, 134.26, 129.98, 128.94, 128.08, 127.50, 124.11, 121.20, 38.16, 21.36.

### 1-(4-ethylphenyl)-N-methyl-N-(m-tolyl)-1H-1,2,4-triazole-3-carboxamide (APZ-2065)

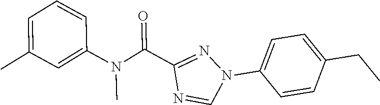 [SMILES: CCc1ccc(-n2cnc(C(=O)N(C)c3cccc(C)c3)n2)cc1]

1-(4-ethylphenyl)-N-methyl-N-(m-tolyl)-1H-1,2,4-triazole-3-carboxamide was prepared via General Procedure A from 1-(4-ethylphenyl)-1H-1,2,4-triazole-3-carboxylic acid. The crude product was purified by flash column chromatography with hexanes/ethyl acetate to afford title compound as a lustrous white solid (99%). mp 64-66° C.; R_{f} (60% EA/Hex)=0.31; ¹H NMR (500 MHz, (CD₃)₂CO) 8.81 (s, 1H), 7.69-7.50 (m, 2H), 7.35 (d, J=8.1 Hz, 2H), 7.16 (t, J=7.3 Hz, 1H), 7.09 (s, 1H), 7.05-6.90 (m, 2H), 3.45 (s, 3H), 2.68 (q, J=7.6 Hz, 2H), 2.26 (s, 3H), 1.22 (t, J=7.6 Hz, 3H); ¹³C NMR (175 MHz, (CD₃)₂CO) δ 162.47, 159.45, 145.30, 144.97, 142.16, 139.50, 135.75, 129.83, 129.46, 128.27 (2 non-equivalent C), 124.78, 120.56, 37.64, 28.86, 21.16, 15.90.

### 1-(4-isopropylphenyl)-N-methyl-N-(m-tolyl)-1H-1,2,4-triazole-3-carboxamide (APZ-2066)

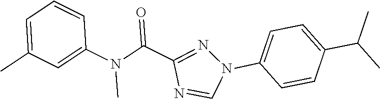 [SMILES: Cc1cccc(N(C)C(=O)c2ncn(-c3ccc(C(C)C)cc3)n2)c1]

1-(4-isopropylphenyl)-N-methyl-N-(m-tolyl)-1H-1,2,4-triazole-3-carboxamide was prepared via General Procedure A from 1-(4-isopropylphenyl)-1H-1,2,4-triazole-3-carboxylic acid. The crude product was purified by flash column chromatography with hexanes/ethyl acetate to afford title compound as a light orange solid (87%). mp 93-96° C.; R_{f} (70% EA/Hex)=0.31; ¹H NMR (500 MHz, (CD₃)₂CO) δ 8.81 (s, 1H), 7.66-7.49 (m, 2H), 7.38 (d, J=8.2 Hz, 2H), 7.16 (t, J=7.8 Hz, 1H), 7.09 (s, 1H), 7.05-6.94 (m, 2H), 3.45 (s, 3H), 2.96 (hept, J=6.9 Hz, 1H), 2.26 (s, 3H), 1.24 (d, J=6.9 Hz, 6H); ¹³C NMR (175 MHz, (CD₃)₂CO) δ 162.47, 159.44, 149.83, 144.96, 142.17, 139.49, 135.79, 129.46, 128.39, 128.26 (2 non-equivalent C), 124.76, 120.57, 37.63, 34.40, 24.13, 21.16.

### 1-(4-ethtlphenyl)-N-methyl-N-(p-tolyl)-1H-1,2,4-triazole-3-carboxamide (APZ-2067)

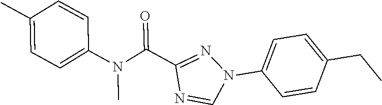 [SMILES: CCc1ccc(-n2cnc(C(=O)N(C)c3ccc(C)cc3)n2)cc1]

1-(4-ethylphenyl)-N-methyl-N-(p-tolyl)-1H-1,2,4-triazole-3-carboxamide was prepared via General Procedure A from 1-(4-ethylphenyl)-1H-1,2,4-triazole-3-carboxylic acid. The crude product was purified by flash column chromatography with hexanes/ethyl acetate to afford title compound as a lustrous white solid (99%). mp 148-151° C.; R_{f} (60% EA/Hex)=0.30; ¹H NMR (500 MHz, (CD₃)₂CO) δ 8.79 (s, 1H), 7.57 (s, 2H), 7.35 (d, J=8.1 Hz, 2H), 7.11 (s, 4H), 3.43 (s, 3H), 2.68 (q, J=7.6 Hz, 2H), 2.26 (s, 3H), 1.22 (t, J=7.6 Hz, 3H); ¹³C NMR (175 MHz, (CD₃)₂CO) δ 162.52, 159.51, 145.30, 142.52, 142.11, 137.32, 135.76, 130.24, 129.84, 127.65, 120.53, 37.60, 28.87, 20.92, 15.91.

### 1-(4-isopropylphenyl)-N-methyl-N-(p-tolyl)-1H-1,2,4-triazole-3-carboxamide (APZ-2068)

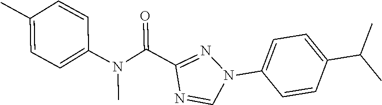 [SMILES: Cc1ccc(N(C)C(=O)c2ncn(-c3ccc(C(C)C)cc3)n2)cc1]

1-(4-isopropylphenyl)-N-methyl-N-(p-tolyl)-1H-1,2,4-triazole-3-carboxamide was prepared via General Procedure A from 1-(4-isopropylphenyl)-1H-1,2,4-triazole-3-carboxylic acid. The crude product was purified by flash column chromatography with hexanes/ethyl acetate to afford title compound as a lustrous light yellow solid (89%). R_{f} (70% EA/Hex)=0.59; ¹H NMR (500 MHz, CDCl₃) δ 8.27 (s, 1H), 7.40 (d, J=7.8 Hz, 2H), 7.29 (d, J=7.8 Hz, 3H), 7.17-6.99 (m, 4H), 3.51 (s, 3H), 2.94 (hept, J=7.0 Hz, 1H), 2.32 (s, 3H), 1.26 (d, J=7.0 Hz, 6H).

### N-(3,4-dimethylphenyl)-1-(4-isopropylphenyl)-N-methyl-1H-1,2,4-triazole-3-carboxamide (APZ-2070)

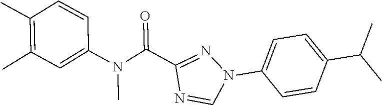 [SMILES: Cc1ccc(N(C)C(=O)c2ncn(-c3ccc(C(C)C)cc3)n2)cc1C]

N-(3,4-dimethylphenyl)-1-(4-isopropylphenyl)-N-methyl-1H-1,2,4-triazole-3-carboxamide was prepared via General Procedure A from 1-(4-isopropylphenyl)-1H-1,2,4-triazole-3-carboxylic acid. The crude product was purified by flash column chromatography with hexanes/ethyl acetate to afford title compound as a lustrous white solid (67%). mp 109-113° C.; R_{f} (70% EA/Hex)=0.39; ¹H NMR (500 MHz, CDCl₃) δ 8.28 (s, 1H), 7.47-7.34 (m, 2H), 7.29 (d, J=7.6 Hz, 2H), 7.08-6.80 (m, 3H), 3.50 (s, 3H), 2.94 (hept, J=6.9 Hz, 1H), 2.32-2.11 (m, 6H), 1.26 (d, J=6.9 Hz, 6H); ¹³C NMR (175 MHz, CDCl₃) δ 161.88, 158.65, 149.52, 141.74, 140.42, 137.51, 135.66, 134.62, 130.13, 127.81, 127.70, 124.25, 120.06, 38.14, 33.86, 23.96, 19.86, 19.43.

### 1-(4-isopropylphenyl)-N-(4-methoxyphenyl)-N-methyl-1H-1,2,4-triazole-3-carboxamide (APZ-2071)

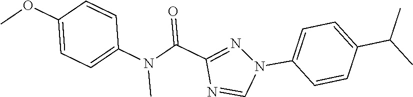 [SMILES: COc1ccc(N(C)C(=O)c2ncn(-c3ccc(C(C)C)cc3)n2)cc1]

1-(4-isopropylphenyl)-N-(4-methoxyphenyl)-N-methyl-1H-1,2,4-triazole-3-carboxamide was prepared via General Procedure A from 1-(4-isopropylphenyl)-1H-1,2,4-triazole-3-carboxylic acid. The crude product was purified by flash column chromatography with hexanes/ethyl acetate to afford title compound as a tan solid (84%). mp 105-107° C.; R_{f} (70% EA/Hex)=0.29; ¹H NMR (500 MHz, CDCl₃, rotameric mixture, major form reported) δ 8.28 (s, 1H), 7.40 (d, J=8.1 Hz, 2H), 7.28 (d, J=8.6 Hz, 2H), 7.11 (d, J=8.4 Hz, 2H), 6.80 (d, J=8.4 Hz, 2H), 3.77 (s, 3H), 3.49 (s, 3H), 2.94 (hept, J=6.9 Hz, 1H), 1.25 (d, J=6.9 Hz, 6H); ¹³C NMR (175 MHz, CDCl₃) δ 161.96, 158.55, 158.54, 149.52, 140.42, 136.99, 134.57, 128.25, 127.70, 120.01, 114.27, 55.51, 38.25, 33.85, 23.95.

### N-(4-chlorophenyl)-1-(4-isopropylphenyl)-N-methyl-1H-1,2,4-triazole-3-carboxamide (APZ-2072)

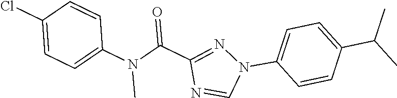 [SMILES: CC(C)c1ccc(-n2cnc(C(=O)N(C)c3ccc(Cl)cc3)n2)cc1]

N-(4-chlorophenyl)-1-(4-isopropylphenyl)-N-methyl-1H-1,2,4-triazole-3-carboxamide was prepared via General Procedure A from 1-(4-isopropylphenyl)-1H-1,2,4-triazole-3-carboxylic acid. The crude product was purified by flash column chromatography with hexanes/ethyl acetate to afford title compound as a light yellow solid (92%). mp 151-152° C.; R_{f} (70% EA/Hex)=0.40; ¹H NMR (500 MHz, CDCl₃) δ 8.32 (s, 1H), 7.52-7.35 (m, 2H), 7.35-7.22 (m, 4H), 7.21-7.06 (m, 2H), 3.52 (s, 3H), 2.95 (hept, J=6.9 Hz, 1H), 1.26 (d, J=6.9 Hz, 6H); ¹³C NMR (175 MHz, CDCl₃) δ 161.53, 158.15, 149.78, 142.78, 140.63, 134.47, 132.96, 129.38, 128.37, 127.82, 120.09, 38.14, 33.89, 23.97.

### 1-(4-ethylphenyl)-N-(4-methoxyphenyl)-N-methyl-1H-1,2,4-triazole-3-carboxamide (APZ-2303)

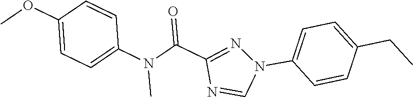 [SMILES: CCc1ccc(-n2cnc(C(=O)N(C)c3ccc(OC)cc3)n2)cc1]

1-(4-ethylphenyl)-N-(4-methoxyphenyl)-N-methyl-1H-1,2,4-triazole-3-carboxamide was prepared via General Procedure A from 1-(4-ethylphenyl)-1H-1,2,4-triazole-3-carboxylic acid. The crude product was purified by flash column chromatography with hexanes/ethyl acetate to afford title compound as a white solid (28%). mp 124-126° C.; R_{f} (80% EA/Hex)=0.51; ¹H NMR (500 MHz, CDCl₃, rotameric mixture, major form reported) δ 8.28 (s, 1H), 7.40 (d, J=8.0 Hz, 2H), 7.25 (d, J=8.0 Hz, 2H), 7.12 (d, J=8.3 Hz, 2H), 6.80 (d, J=8.3 Hz, 2H), 3.77 (s, 3H), 3.49 (s, 3H), 2.67 (q, J=7.6 Hz, 2H), 1.24 (t, J=7.6 Hz, 3H); ¹³C NMR (175 MHz, CDCl₃) δ 161.94, 158.56, 144.92, 140.42 (2 non-equivalent C), 137.01, 134.54, 129.12, 128.27, 120.02, 114.29, 55.53, 38.28, 28.50, 15.56.

### N-(4-chlorophenyl)-1-(4-ethylphenyl)-N-methyl-1H-1,2,4-triazole-3-carboxamide (APZ-2304)

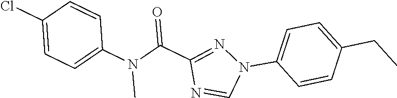 [SMILES: CCc1ccc(-n2cnc(C(=O)N(C)c3ccc(Cl)cc3)n2)cc1]

N-(4-chlorophenyl)-1-(4-ethylphenyl)-N-methyl-1H-1,2,4-triazole-3-carboxamide was prepared via General Procedure A from 1-(4-ethylphenyl)-1H-1,2,4-triazole-3-carboxylic acid. The crude product was purified by flash column chromatography with hexanes/ethyl acetate to afford title compound as a white solid (62%). mp 154-156° C.; R_{f} (70% EA/Hex)=0.29; ¹H NMR (500 MHz, CDCl₃) δ 8.31 (s, 1H), 7.46-7.34 (m, 2H), 7.28 (d, J=7.6 Hz, 4H), 7.21-7.08 (m, 2H), 3.52 (s, 3H), 2.69 (q, J=7.6 Hz, 2H), 1.25 (t, J=7.6 Hz, 3H); ¹³C NMR (175 MHz, CDCl₃) δ 161.50, 158.14, 145.15, 142.77, 140.63, 134.43, 132.96, 129.37, 129.21, 128.37, 120.07, 38.14, 28.52, 15.54.

### 1-(4-chlorophenyl)-N-cyclohexyl-N-methyl-1H-1,2,4-triazole-3-carboxamide (APZ-2305)

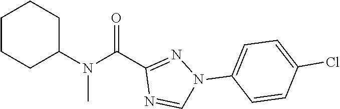 [SMILES: CN(C(=O)c1ncn(-c2ccc(Cl)cc2)n1)C1CCCCC1]

1-(4-chlorophenyl)-N-cyclohexyl-N-methyl-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate, followed by preparatory TLC to afford title compound as a white oil (44%). R_{f} (70% EA/Hex)=0.26.

### 1-(4-chlorophenyl)-N-cyclopentyl-N-methyl-1H-1,2,4-triazole-3-carboxamide (APZ-2306)

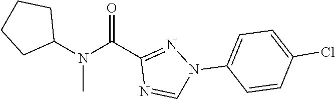 [SMILES: CN(C(=O)c1ncn(-c2ccc(Cl)cc2)n1)C1CCCC1]

1-(4-chlorophenyl)-N-cyclopentyl-N-methyl-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate to afford title compound as a white oil (24%). R_{f} (70% EA/Hex)=0.22; ¹H NMR (500 MHz, CDCl₃, rotameric mixture, both forms reported) δ 8.57 (s, 1H), 7.74-7.62 (m, 2H), 7.55-7.44 (m, 2H), 5.10 (p, J=8.2 Hz, 0.4H), 4.49 (p, J=8.2 Hz, 0.6H), 3.07 (s, 1.2H), 3.03 (s, 1.8H), 2.07-1.41 (m, 8H); ¹³C NMR (175 MHz, CDCl₃, rotameric mixture, both forms reported) δ 162.04, 161.94, 159.48, 159.34, 140.92, 140.88, 135.32, 135.29, 134.50, 134.40, 130.14, 121.48, 121.36, 59.70, 55.31, 31.43, 29.54, 28.23, 28.10, 24.65, 24.46.

### N-methyl-N-(pyridin-4-yl)-1-(p-tolyl)-1H-1,2,4-triazole-3-carboxamide (APZ-2084)

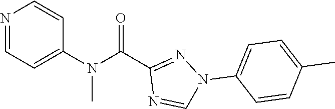 [SMILES: Cc1ccc(-n2cnc(C(=O)N(C)c3ccncc3)n2)cc1]

N-methyl-N-(pyridin-4-yl)-1-(p-tolyl)-1H-1,2,4-triazole-3-carboxamide was prepared via General Procedure A from 1-(p-tolyl)-1H-1,2,4-triazole-3-carboxylic acid. The crude product was purified by flash column chromatography with dichloromethane/methanol acetate to afford title compound as a lustrous light yellow solid (91%). mp 121-124° C.; R_{f} (5% MeOH/DCM)=0.26; ¹H NMR (500 MHz, CD₃OD) δ 8.96 (s, 1H), 8.52-8.46 (m, 2H), 7.48 (d, J=8.3 Hz, 2H), 7.36-7.32 (m, 2H), 7.30 (d, J=8.3 Hz, 3H), 3.59 (s, 3H), 2.38 (s, 3H); ¹³C NMR (175 MHz, CD₃OD) δ 162.80, 158.23, 153.45, 151.18, 143.39, 140.28, 135.55, 131.30, 122.42, 120.92, 37.79, 21.00.

### N-cyclohexyl-N-methyl-1-(p-tolyl)-1H-1,2,4-triazole-3-carboxamide (APZ-2085)

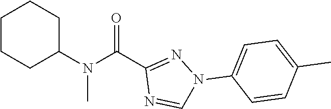 [SMILES: Cc1ccc(-n2cnc(C(=O)N(C)C3CCCCC3)n2)cc1]

N-cyclohexyl-N-methyl-1-(p-tolyl)-1H-1,2,4-triazole-3-carboxamide was prepared via General Procedure A from 1-(p-tolyl)-1H-1,2,4-triazole-3-carboxylic acid. The crude product was purified by flash column chromatography with hexanes/ethyl acetate to afford title compound as a white oil (95%). R_{f} (80% EA/Hex)=0.16; ¹H NMR (500 MHz, CDCl₃, rotameric mixture, both forms reported) δ 8.54 (s, 0.55H), 8.53 (s, 0.45H), 7.64-7.53 (m, 2H), 7.37-7.28 (m, 2H), 4.59 (m, 0.45H), 3.96 (tt, J=11.8, 3.7 Hz, 0.55H), 3.06 (s, 1.35H), 3.03 (s, 1.65H), 2.41 (s, 3H), 2.07-1.39 (m, 8H), 1.30-1.03 (m, 2H); ¹³C NMR (175 MHz, CDCl₃, rotameric mixture, both forms reported) δ 162.03, 161.68, 159.13, 158.82, 140.77, 138.76, 138.66, 134.50, 134.45, 130.38, 130.36, 120.23, 120.00, 57.97, 53.29, 31.29, 31.03, 29.54, 27.97, 25.69, 25.66, 25.63, 25.34, 21.14.

### N-cyclopentyl-N-methyl-1-(p-tolyl)-1H-1,2,4-triazole-3-carboxamide (APZ-2186)

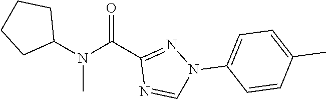 [SMILES: Cc1ccc(-n2cnc(C(=O)N(C)C3CCCC3)n2)cc1]

N-cyclopentyl-N-methyl-1-(p-tolyl)-1H-1,2,4-triazole-3-carboxamide was prepared via General Procedure A from 1-(p-tolyl)-1H-1,2,4-triazole-3-carboxylic acid. The crude product was purified by flash column chromatography with hexanes/ethyl acetate to afford title compound as a white oil (55%). R_{f} (80% EA/Hex)=0.32; ¹H NMR (500 MHz, CDCl₃, rotameric mixture, both forms reported) δ 8.53 (s, 1H), 7.59 (dt, J=8.5, 2.6, 2.0 Hz, 2H), 7.34-7.28 (m, 2H), 5.11 (p, J=8.1 Hz, 0.4H), 4.54 (p, J=8.1 Hz, 0.6H), 3.08 (s, 1H), 3.02 (s, 2H), 2.42 (s, 3H), 2.05-1.45 (m, 9H); ¹³C NMR (175 MHz, CDCl₃) δ 162.31, 162.20, 159.08, 158.92, 140.82, 140.80, 138.83, 138.74, 134.51, 134.47, 130.41, 120.25, 120.10, 59.65, 55.22, 31.41, 29.80, 29.50, 28.19, 28.06, 24.62, 24.43, 21.18.

### N-methyl-N-(thiazol-2-yl)-1-(p-tolyl)-1H-1,2,4-triazole-3-carboxamide (APZ-2086)

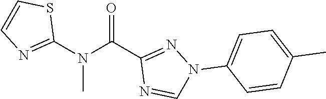 [SMILES: Cc1ccc(-n2cnc(C(=O)N(C)c3nccs3)n2)cc1]

N-methyl-N-(thiazol-2-yl)-1-(p-tolyl)-1H-1,2,4-triazole-3-carboxamide was prepared via General Procedure A from 1-(p-tolyl)-1H-1,2,4-triazole-3-carboxylic acid. The crude product was purified by flash column chromatography with dichloromethane/methanol to afford title compound as a lustrous light yellow solid (61%). mp 111-113° C.; R_{f} (80% EA/Hex)=0.47; ¹H NMR (500 MHz, CDCl₃) δ 8.62 (s, 1H), 7.69-7.53 (m, 3H), 7.34 (d, J=8.1 Hz, 2H), 7.12 (d, J=3.6 Hz, 1H), 4.07 (s, 3H), 2.44 (s, 3H); ¹³C NMR (175 MHz, CDCl₃) δ 160.39, 160.34, 157.38, 141.32, 139.33, 137.43, 134.23, 130.53, 120.29, 115.82, 37.28, 21.21.

### 1-(4-chlorophenyl)-N-methyl-N-(pyridin-4-yl)-1H-1,2,4-triazole-3-carboxamide (APZ-2087)

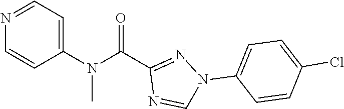 [SMILES: CN(C(=O)c1ncn(-c2ccc(Cl)cc2)n1)c1ccncc1]

1-(4-chlorophenyl)-N-methyl-N-(pyridin-4-yl)-1H-1,2,4-triazole-3-carboxamide was prepared via General Procedure A from 1-(4-chlorophenyl)-1H-1,2,4-triazole-3-carboxylic acid. The crude product was purified by flash column chromatography with dichloromethane/methanol to afford title compound as a white solid (63%). mp 96-104° C.; R_{f} (100% EA/Hex)=0.22; ¹H NMR (500 MHz, CD₃OD) δ 9.02 (s, 1H), 8.58-8.43 (m, 2H), 7.64 (dt, J=8.9, 2.9, 2.1 Hz, 2H), 7.50 (dt, J=8.9, 2.9, 2.1 Hz, 2H), 7.39-7.26 (m, 2H), 3.59 (s, 3H); ¹³C NMR (175 MHz, CD₃OD) δ 162.63, 158.55, 153.36, 151.23, 143.76, 136.55, 135.38, 130.97, 122.47, 122.43, 37.81.

### 1-(4-chlorophenyl)-N-methyl-N-(thiazol-2-yl)-1H-1,2,4-triazole-3-carboxamide (APZ-2088)

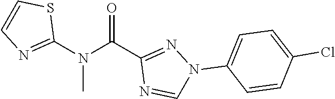 [SMILES: CN(C(=O)c1ncn(-c2ccc(Cl)cc2)n1)c1nccs1]

1-(4-chlorophenyl)-N-methyl-N-(thiazol-2-yl)-1H-1,2,4-triazole-3-carboxamide was prepared via General Procedure A from 1-(4-chlorophenyl)-1H-1,2,4-triazole-3-carboxylic acid. The crude product was purified by flash column chromatography with dichloromethane/methanol to afford title compound as a lustrous orange solid (50%). mp 183-185° C.; R_{f} (40% EA/Hex)=0.28; ¹H NMR (500 MHz, CDCl₃) δ 8.66 (s, 1H), 7.75-7.67 (m, 2H), 7.62 (d, J=3.6 Hz, 1H), 7.56-7.49 (m, 2H), 7.13 (d, J=3.6 Hz, 1H), 4.06 (s, 3H); ¹³C NMR (175 MHz, CDCl₃) δ 160.29, 160.11, 157.79, 141.41, 137.52, 135.04, 134.99, 130.29, 121.58, 115.96, 37.29.

### N,1-bis(4-chlorophenyl)-N-methyl-1H-1,2,3-triazole-4-carboxamide (APZ-2089)

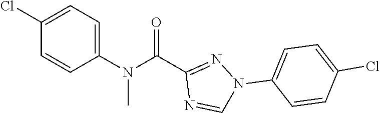 [SMILES: CN(C(=O)c1ncn(-c2ccc(Cl)cc2)n1)c1ccc(Cl)cc1]

N,1-bis(4-chlorophenyl)-N-methyl-1H-1,2,3-triazole-4-carboxamide was prepared via General Procedure A from 1-(4-chlorophenyl)-1H-1,2,3-triazole-4-carboxylic acid. The crude product was purified by flash column chromatography with hexanes/ethyl acetate to afford title compound as a lustrous white solid (82%). mp 162-165° C.; R_{f} (40% EA/Hex)=0.32; ¹H NMR (500 MHz, CDCl₃) δ 8.21 (bs, 1H), 7.62 (d, J=6.3 Hz, 2H), 7.49 (d, J=8.3 Hz, 2H), 7.36 (d, J=8.3 Hz, 2H), 7.19 (d, J=6.3 Hz, 2H), 3.51 (bs, 3H); ¹³C NMR (175 MHz, CDCl₃) δ 160.74, 144.29, 142.57, 135.09, 134.83, 133.42, 130.07, 129.62, 128.62, 124.98, 121.64, 38.67.

### N-(4-chlorophenyl)-N-methyl-1-(p-tolyl)-1H-1,2,3-triazole-4-carboxamide (APZ-2090)

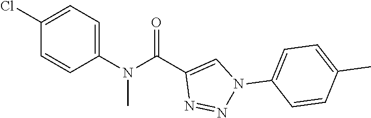 [SMILES: Cc1ccc(-n2cc(C(=O)N(C)c3ccc(Cl)cc3)nn2)cc1]

N-(4-chlorophenyl)-N-methyl-1-(p-tolyl)-1H-1,2,3-triazole-4-carboxamide was prepared via General Procedure A from 1-(p-tolyl)-1H-1,2,3-triazole-4-carboxylic acid. The crude product was purified by flash column chromatography with hexanes/ethyl acetate to afford title compound as a white solid (72%). mp 146-148° C.; R_{f} (70% EA/Hex)=0.56; ¹H NMR (500 MHz, CDCl₃) δ 8.16 (bs, 1H), 7.53 (d, J=7.1 Hz, 2H), 7.36 (d, J=8.2 Hz, 2H), 7.30 (t, J=8.2 Hz, 2H), 7.20 (d, J=7.1 Hz, 2H), 3.53 (bs, 3H), 2.41 (s, 3H); ¹³C NMR (175 MHz, CDCl₃) δ 161.13, 144.04, 142.81, 139.53, 134.19, 133.40, 130.46, 129.70, 128.71, 125.13, 120.48, 38.83, 21.22.

### N,2-bis(4-chlorophenyl)-N-methylthiazole-5-carboxamide (APZ-2091)

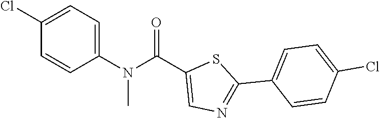 [SMILES: CN(C(=O)c1cnc(-c2ccc(Cl)cc2)s1)c1ccc(Cl)cc1]

N,2-bis(4-chlorophenyl)-N-methylthiazole-5-carboxamide was prepared via General Procedure A from 2-(4-chlorophenyl)thiazole-5-carboxylic acid. The crude product was purified by flash column chromatography with hexanes/ethyl acetate to afford title compound as a white solid (80%). mp 148-150° C.; R_{f} (30% EA/Hex)=0.45; ¹H NMR (500 MHz, CDCl₃) δ 7.77 (dt, J=8.6, 2.5, 1.9 Hz, 2H), 7.43 (dt, J=8.6, 3.0, 2.1 Hz, 2H), 7.38 (dt, J=8.6, 2.5, 1.9 Hz, 2H), 7.35 (s, 1H), 7.22 (dt, J=8.6, 3.0, 2.1 Hz, 2H), 3.44 (s, 3H); ¹³C NMR (175 MHz, CDCl₃) δ 170.41, 161.14, 147.60, 142.06, 137.06, 134.85, 133.40, 131.43, 130.53, 129.44, 129.42, 127.97, 38.99.

### N-(4-chlorophenyl)-N-methyl-2-(p-tolyl)thiazole-5-carboxamide (APZ-2092)

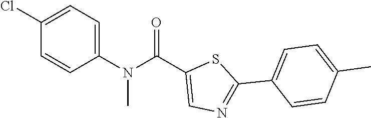 [SMILES: Cc1ccc(-c2ncc(C(=O)N(C)c3ccc(Cl)cc3)s2)cc1]

N-(4-chlorophenyl)-N-methyl-2-(p-tolyl)thiazole-5-carboxamide was prepared via General Procedure A from 2-(p-tolyl)thiazole-5-carboxylic acid. The crude product was purified by flash column chromatography with hexanes/ethyl acetate to afford title compound as a fluffy white solid (67%). mp 116-119° C.; R_{f} (30% EA/Hex)=0.41; ¹H NMR (500 MHz, CDCl₃) δ 7.76-7.69 (m, 2H), 7.42 (dt, J=8.6, 3.0, 2.0 Hz, 2H), 7.34 (s, 1H), 7.25-7.15 (m, 4H), 3.44 (s, 3H), 2.37 (s, 3H); ¹³C NMR (175 MHz, CDCl₃) δ 172.07, 161.41, 147.51, 142.19, 141.47, 134.70, 132.53, 130.45, 130.31, 129.83, 129.44, 126.70, 38.95, 21.59.

### N-(4-chlorophenyl)-N-methyl-5-(p-tolyl)thiazole-2-carboxamide (APZ-2094)

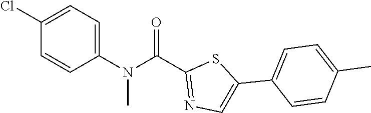 [SMILES: Cc1ccc(-c2cnc(C(=O)N(C)c3ccc(Cl)cc3)s2)cc1]

N-(4-chlorophenyl)-N-methyl-5-(p-tolyl)thiazole-2-carboxamide was prepared via General Procedure A from 5-(p-tolyl)thiazole-2-carboxylic acid. The crude product was purified by flash column chromatography with hexanes/ethyl acetate to afford title compound as a light yellow solid (89%). mp 142-146° C.; R_{f} (30% EA/Hex)=0.47; ¹H NMR (500 MHz, CDCl₃) δ 7.75 (bs, 1H), 7.43 (d, J=7.7 Hz, 2H), 7.34 (d, J=8.2 Hz, 2H), 7.24-7.12 (m, 4H), 3.56 (bs, 3H), 2.37 (s, 3H); ¹³C NMR (175 MHz, CDCl₃) δ 161.31, 161.03, 144.29, 142.93, 139.40, 138.58, 133.09, 130.00, 129.55, 128.33, 127.86, 127.02, 39.53, 21.40.

### 1-(4-chlorophenyl)-N-methyl-N-(p-tolyl)-1H-1,2,3-triazole-4-carboxamide (APZ-2188)

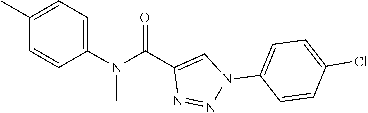 [SMILES: Cc1ccc(N(C)C(=O)c2cn(-c3ccc(Cl)cc3)nn2)cc1]

1-(4-chlorophenyl)-N-methyl-N-(p-tolyl)-1H-1,2,3-triazole-4-carboxamide was prepared via General Procedure A from 1-(4-chlorophenyl)-1H-1,2,3-triazole-4-carboxylic acid. The crude product was purified by flash column chromatography with hexanes/ethyl acetate to afford title compound as a lustrous white solid (68%). R_{f} (50% EA/Hex)=0.40; ¹H NMR (500 MHz, CDCl₃) δ 7.79 (bs, 1H), 7.54 (s, 2H), 7.46 (d, J=7.9 Hz, 2H), 7.20 (d, J=7.5 Hz, 2H), 7.16-7.08 (m, 2H), 3.49 (bs, 2H), 2.38 (s, 3H); ¹³C NMR (175 MHz, CDCl₃) δ 160.93, 144.17, 141.50, 137.92, 135.04, 135.01, 130.25, 130.10, 127.33, 124.32, 121.73, 38.65, 21.27.

### N-methyl-N,1-di-p-tolyl-1H-1,2,3-triazole-4-carboxamide (APZ-2095)

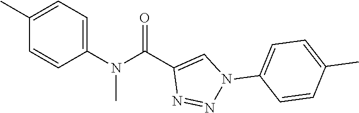

N-methyl-N,1-di-p-tolyl-1H-1,2,3-triazole-4-carboxamide was prepared via General Procedure A from 1-(p-tolyl)-1H-1,2,3-triazole-4-carboxylic acid. The crude product was purified by flash column chromatography with hexanes/ethyl acetate to afford title compound as a white solid (24%). mp 130-134° C.; R_{f} (50% EA/Hex)=0.42; ¹H NMR (500 MHz, (CD₃)₂CO) δ 8.31 (bs, 1H), 7.66 (d, J=7.6 Hz, 2H), 7.37 (d, J=8.1 Hz, 2H), 7.25-7.12 (m, 4H), 3.45 (s, 3H), 2.39 (bs, 3H), 2.32 (s, 3H); ¹³C NMR (175 MHz, (CD₃)₂CO) δ 161.66, 144.93, 143.01, 139.86, 137.67, 135.37, 131.10, 130.57, 128.08, 125.41, 121.08, 38.49, 21.01, 20.97.

### N,5-bis(4-chlorophenyl)-N-methyl-1H-imidazole-2-carboxamide (APZ-2308)

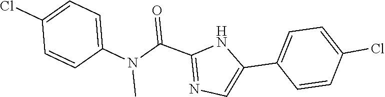 [SMILES: CN(C(=O)c1ncc(-c2ccc(Cl)cc2)[nH]1)c1ccc(Cl)cc1]

N,5-bis(4-chlorophenyl)-N-methyl-1H-imidazole-2-carboxamide was prepared via General Procedure A from 5-(4-chlorophenyl)-1H-imidazole-2-carboxylic acid. The crude product was purified by flash column chromatography with hexanes/ethyl acetate to afford title compound as a white solid (65%). mp 198-202° C.; R_{f} (40% EA/Hex)=0.29; ¹H NMR (500 MHz, (CD₃)₂CO, tautomeric mixture, major form reported) δ 12.09 (bs, 1H), 7.71 (s, 1H), 7.68-7.50 (m, 2H), 7.45 (dt, J=8.6, 2.9, 2.0 Hz, 2H), 7.38 (d, J=8.6 Hz, 2H), 7.31 (d, J=8.1 Hz, 2H), 3.65 (bs, 3H); ¹³C NMR (175 MHz, (CD₃)₂CO, tautomeric mixture, major form reported) δ 159.42, 144.85, 142.02, 141.46, 134.18, 132.79, 132.55, 129.95, 129.65, 129.26, 127.06, 115.78, 39.13.

### N-(4-chlorophenyl)-N-methyl-5-(p-tolyl)-1H-imidazole-2-carboxamide (APZ-2309)

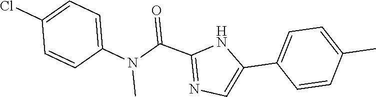 [SMILES: Cc1ccc(-c2cnc(C(=O)N(C)c3ccc(Cl)cc3)[nH]2)cc1]

N-(4-chlorophenyl)-N-methyl-5-(p-tolyl)-1H-imidazole-2-carboxamide was prepared via General Procedure A from 5-(p-tolyl)-1H-imidazole-2-carboxylic acid. The crude product was purified by flash column chromatography with hexanes/ethyl acetate to afford title compound as a white solid (76%). mp 170-175° C.; R_{f} (40% EA/Hex)=0.35; ¹H NMR (700 MHz, (CD₃)₂CO, tautomeric mixture, major form reported) δ 12.02 (bs, 1H), 7.60 (s, 1H), 7.51 (s, 2H), 7.44 (dt, J=8.6, 2.8, 2.0 Hz, 2H), 7.38 (d, J=8.2 Hz, 2H), 7.11 (d, J=7.7 Hz, 2H), 3.65 (bs, 3H), 2.29 (s, 3H); ¹³C NMR (175 MHz, (CD₃)₂CO, tautomeric mixture, major form reported) δ 159.58, 144.94, 141.68, 136.98, 132.67, 132.57, 130.38, 129.91, 129.82, 129.61, 127.08, 125.54, 114.80, 39.16, 21.14.

### N,5-bis(4-chlorophenyl)-N-methylthiazole-2-carboxamide (APZ-2310)

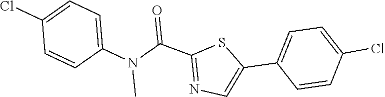 [SMILES: CN(C(=O)c1ncc(-c2ccc(Cl)cc2)s1)c1ccc(Cl)cc1]

N,5-bis(4-chlorophenyl)-N-methylthiazole-2-carboxamide was prepared via General Procedure A from 5-(4-chlorophenyl)thiazole-2-carboxylic acid. The crude product was purified by flash column chromatography with hexanes/ethyl acetate to afford title compound as a light pink solid (75%). mp 133-137° C.; R_{f} (20% EA/Hex)=0.35; ¹H NMR (500 MHz, CDCl₃) δ 7.75 (s, 1H), 7.46 (d, J=8.2 Hz, 2H), 7.40-7.29 (m, 4H), 7.17 (d, J=6.4 Hz, 2H), 3.55 (s, 3H); ¹³C NMR (175 MHz, CDCl₃) δ 162.18, 160.75, 142.77, 139.24, 135.17, 133.22, 129.58, 129.55, 129.22, 128.29 (2 non-equivalent C), 39.54.

### N-(4-chlorophenyl)-N-methyl-2-(p-tolyl)-1H-imidazole-5-carboxamide (APZ-2311)

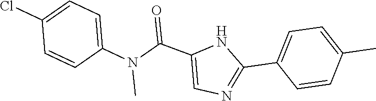 [SMILES: Cc1ccc(-c2ncc(C(=O)N(C)c3ccc(Cl)cc3)[nH]2)cc1]

N-(4-chlorophenyl)-N-methyl-2-(p-tolyl)-1H-imidazole-5-carboxamide was prepared via General Procedure A from 2-(p-tolyl)-1H-imidazole-5-carboxylic acid. The crude product was purified by flash column chromatography with hexanes/ethyl acetate, followed by preparatory TLC to afford title compound as a white solid (51%). R_{f} (50% EA/Hex)=0.29; ¹H NMR (400 MHz, CDCl₃, rotameric mixture, major form reported) δ 10.96 (bs, 1H), 7.79 (d, J=7.9 Hz, 2H), 7.47 (d, J=8.4 Hz, 2H), 7.33-7.18 (m, 4H), 5.79 (bs, 1H), 3.44 (s, 3H), 2.38 (s, 3H).

### N,2-bis(4-chlorophenyl)-N-methyl-1H-imidazole-5-carboxamide (APZ-2312)

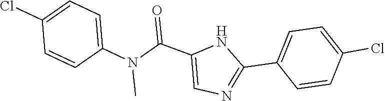 [SMILES: CN(C(=O)c1cnc(-c2ccc(Cl)cc2)[nH]1)c1ccc(Cl)cc1]

N,2-bis(4-chlorophenyl)-N-methyl-1H-imidazole-5-carboxamide was prepared via General Procedure A from 2-(4-chlorophenyl)-1H-imidazole-5-carboxylic acid. The crude product was purified by flash column chromatography with hexanes/ethyl acetate, followed by preparatory TLC to afford title compound as a white solid (32%). R_{f} (50% EA/Hex)=0.32; ¹H NMR (400 MHz, CDCl₃) δ 11.43 (bs, 1H), 7.89 (d, J=8.2 Hz, 2H), 7.48 (d, J=8.0 Hz, 2H), 7.39 (d, J=8.2 Hz, 2H), 7.28 (d, J=8.0 Hz, 2H), 5.83 (bs, 1H), 3.44 (bs, 3H).

### (1-(4-chlorophenyl)-1H-1,2,4-triazol-3-yl)(pyrrolidin-1-yl)methanone (APZ-2313)

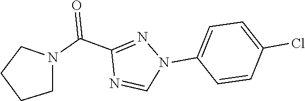 [SMILES: O=C(c1ncn(-c2ccc(Cl)cc2)n1)N1CCCC1]

(1-(4-chlorophenyl)-1H-1,2,4-triazol-3-yl)(pyrrolidin-1-yl)methanone was prepared via General Procedure A from 1-(4-chlorophenyl)-1H-1,2,4-triazole-3-carboxylic acid. The crude product was purified by flash column chromatography with hexanes/ethyl acetate then dichloromethane/methanol, to afford title compound as a tan solid (40%). mp 158-163° C.; R_{f} (100% EA/Hex)=0.28; ¹H NMR (700 MHz, CDCl₃) δ 8.59 (s, 1H), 7.68 (d, J=8.2 Hz, 2H), 7.47 (d, J=8.2 Hz, 2H), 3.91 (t, J=6.7 Hz, 2H), 3.71 (t, J=6.7 Hz, 2H), 1.95 (dp, J=30.5, 7.0 Hz, 4H); ¹³C NMR (175 MHz, CDCl₃) δ 159.23, 159.03, 141.06, 135.30, 134.44, 130.09, 121.45, 48.95, 47.05, 26.55, 24.08.

### N,2-bis(4-chlorophenyl)-N,4-dimethyloxazole-5-carboxamide (APZ-2314)

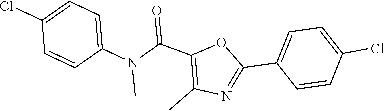 [SMILES: Cc1nc(-c2ccc(Cl)cc2)oc1C(=O)N(C)c1ccc(Cl)cc1]

N,2-bis(4-chlorophenyl)-N,4-dimethyloxazole-5-carboxamide was prepared via General Procedure A from 2-(4-chlorophenyl)-4-methyloxazole-5-carboxylic acid. The crude product was purified by flash column chromatography with hexanes/ethyl acetate to afford title compound as a lustrous white solid (67%). mp 146-152° C.; R_{f} (30% EA/Hex)=0.50; ¹H NMR (700 MHz, CDCl₃) δ 7.41 (dt, J=8.5, 3.1, 2.1 Hz, 2H), 7.37-7.30 (m, 4H), 7.18 (dt, J=8.5, 3.1, 2.1 Hz, 2H), 3.45 (s, 3H), 2.53 (s, 3H); ¹³C NMR (175 MHz, CDCl₃) δ 159.30, 158.82, 146.90, 142.89, 139.26, 137.41, 133.49, 129.70, 129.25, 128.48, 127.80, 124.96, 38.44, 13.82.

### N-(4-chlorophenyl)-N,4-dimethyl-2-(p-tolyl)oxazole-5-carboxamide (APZ-2315)

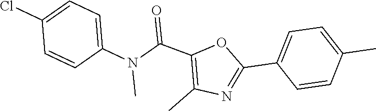 [SMILES: Cc1ccc(-c2nc(C)c(C(=O)N(C)c3ccc(Cl)cc3)o2)cc1]

N-(4-chlorophenyl)-N,4-dimethyl-2-(p-tolyl)oxazole-5-carboxamide was prepared via General Procedure A from 4-methyl-2-(p-tolyl)oxazole-5-carboxylic acid. The crude product was purified by flash column chromatography with hexanes/ethyl acetate to afford title compound as a lustrous white solid (quantitative). mp 103-105° C.; R_{f} (30% EA/Hex)=0.46; ¹H NMR (700 MHz, CDCl₃) δ 7.40 (dt, J=8.6, 3.1, 2.1 Hz, 2H), 7.31 (d, J=7.9 Hz, 2H), 7.18 (dt, J=8.6, 3.1, 2.1 Hz, 2H), 7.15 (d, J=7.9 Hz, 2H), 3.44 (s, 3H), 2.53 (s, 3H), 2.35 (s, 3H); ¹³C NMR (175 MHz, CDCl₃) δ 160.52, 159.03, 146.82, 142.99, 141.68, 138.76, 133.33, 129.64, 129.59, 128.43, 126.54, 123.73, 38.41, 21.63, 13.84.

### N,5-bis(4-chlorophenyl)-N-methyloxazole-2-carboxamide (APZ-2161)

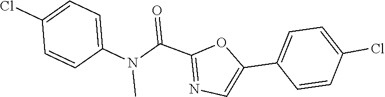 [SMILES: CN(C(=O)c1ncc(-c2ccc(Cl)cc2)o1)c1ccc(Cl)cc1]

N,5-bis(4-chlorophenyl)-N-methyloxazole-2-carboxamide was prepared via General Procedure A from 5-(4-chlorophenyl)oxazole-2-carboxylic acid. The crude product was purified by flash column chromatography with hexanes/ethyl acetate to afford title compound as a white solid (82%). mp 139-141° C.; R_{f} (30% EA/Hex)=0.30; ¹H NMR (500 MHz, (CD₃)₂SO) δ 7.75 (bs, 1H), 7.64-7.51 (m, 4H), 7.45 (d, J=8.2 Hz, 2H), 7.37 (d, J=8.2 Hz, 2H), 3.43 (bs, 3H); ¹³C NMR (175 MHz, CDCl₃) δ 156.63, 154.01, 151.33, 142.21, 135.39, 133.68, 129.75, 129.38, 128.07, 126.08, 125.43, 123.09, 38.63.

### N-(4-chlorophenyl)-N-methyl-5-(p-tolyl)oxazole-2-carboxamide (APZ-2119)

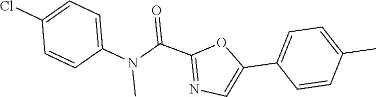 [SMILES: Cc1ccc(-c2cnc(C(=O)N(C)c3ccc(Cl)cc3)o2)cc1]

N-(4-chlorophenyl)-N-methyl-5-(p-tolyl)oxazole-2-carboxamide was prepared via General Procedure A from 5-(p-tolyl)oxazole-2-carboxylic acid. The crude product was purified by flash column chromatography with hexanes/ethyl acetate to afford title compound as a white solid (90%). mp 126-132° C.; R_{f} (30% EA/Hex)=0.36; ¹H NMR (700 MHz, CDCl₃) δ 7.36 (d, J=8.3 Hz, 4H), 7.24-7.06 (m, 5H), 3.51 (bs, 3H), 2.37 (s, 3H); ¹³C NMR (175 MHz, CDCl₃) δ 156.87, 153.59, 152.65, 142.38, 139.73, 133.58, 129.78, 129.74, 128.08, 124.84, 124.23, 122.22, 38.69, 21.54.

### (1-(4-chlorophenyl)-1H-1,2,4-triazol-3-yl)(4-methylpiperazin-1-yl)methanone (APZ-2121)

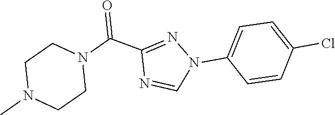 [SMILES: CN1CCN(C(=O)c2ncn(-c3ccc(Cl)cc3)n2)CC1]

(1-(4-chlorophenyl)-1H-1,2,4-triazol-3-yl)(4-methylpiperazin-1-yl)methanone was prepared via General Procedure A from 1-(4-chlorophenyl)-1H-1,2,4-triazole-3-carboxylic acid. The crude product was purified by flash column chromatography with hexanes/ethyl acetate, followed by preparatory TLC (7% MeOH/DCM) to afford title compound as a light yellow solid (66%). mp 127-128° C.; R_{f} (7% MeOH/DCM)=0.17; ¹H NMR (700 MHz, CDCl₃) δ 8.56 (s, 1H), 7.66 (dt, J=8.7, 3.0, 2.0 Hz, 2H), 7.51 (dt, J=8.7, 3.0, 2.0 Hz, 2H), 3.95-3.80 (m, 4H), 2.53 (t, J=5.1 Hz, 2H), 2.48 (t, J=5.1 Hz, 2H), 2.35 (s, 3H); ¹³C NMR (175 MHz, CDCl₃) δ 160.01, 158.54, 140.97, 135.19, 134.63, 130.17, 121.52, 55.46, 54.72, 47.04, 46.12, 42.53.

### (1-(4-chlorophenyl)-1H-1,2,4-triazol-3-yl)(piperidin-1-yl)methanone (APZ-2122)

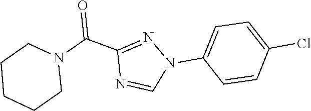 [SMILES: O=C(c1ncn(-c2ccc(Cl)cc2)n1)N1CCCCC1]

(1-(4-chlorophenyl)-1H-1,2,4-triazol-3-yl)(piperidin-1-yl)methanone was prepared via General Procedure A from 1-(4-chlorophenyl)-1H-1,2,4-triazole-3-carboxylic acid. The crude product was purified by flash column chromatography with hexanes/ethyl acetate to afford title compound as a yellow solid (64%). mp 156-158° C.; R_{f} (70% EA/Hex)=0.22; ¹H NMR (500 MHz, CD₃OD) δ 9.16 (s, 1H), 7.87 (d, J=8.6 Hz, 2H), 7.60 (d, J=8.6 Hz, 2H), 3.77 (t, J=5.5 Hz, 2H), 3.72 (t, J=5.5 Hz, 2H), 1.83-1.61 (m, 6H); ¹³C NMR (175 MHz, CDCl₃) δ 160.19, 158.89, 140.94, 135.24, 134.40, 130.08, 121.43, 48.27, 43.62, 26.69, 25.64, 24.64.

### 4-chloro-N-(1-(p-tolyl)-1H-1,2,4-triazol-3-yl)benzamide (APZ-2162)

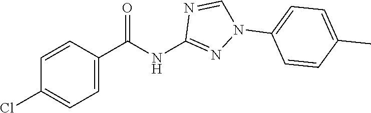 [SMILES: Cc1ccc(-n2cnc(NC(=O)c3ccc(Cl)cc3)n2)cc1]

4-chloro-N-(1-(p-tolyl)-1H-1,2,4-triazol-3-yl)benzamide was prepared from 1-(p-tolyl)-1H-1,2,4-triazol-3-amine and 4-chlorobenzoyl chloride as described herein. The crude product was purified by flash column chromatography with hexanes/ethyl acetate to afford title compound as a fluffy white solid (19%). mp 194-196° C.; R_{f} (50% EA/Hex)=0.19; ¹H NMR (500 MHz, (CD₃)₂SO) δ 11.05 (s, 1H), 9.18 (s, 1H), 8.01 (d, J=8.5 Hz, 2H), 7.73 (d, J=8.3 Hz, 2H), 7.60 (d, J=8.5 Hz, 2H), 7.37 (d, J=8.3 Hz, 2H), 2.36 (s, 3H); ¹³C NMR (125 MHz, (CD₃)₂SO) δ 164.76, 157.23, 142.15, 137.61, 137.36, 134.94, 132.84, 130.61, 130.34, 129.03, 119.37, 21.00.

### 4-chloro-N-(1-(4-chlorophenyl)-1H-1,2,4-triazol-3-yl)benzamide (APZ-2163)

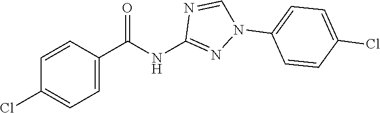 [SMILES: O=C(Nc1ncn(-c2ccc(Cl)cc2)n1)c1ccc(Cl)cc1]

4-chloro-N-(1-(4-chlorophenyl)-1H-1,2,4-triazol-3-yl)benzamide was prepared from 1-(4-chlorophenyl)-1H-1,2,4-triazol-3-amine and 4-chlorobenzoyl chloride as previously described for 4-chloro-N-(1-(p-tolyl)-1H-1,2,4-triazol-3-yl)benzamide. The crude product was purified by flash column chromatography with hexanes/ethyl acetate to afford title compound (20%). ¹H NMR (500 MHz, (CD₃)₂SO) δ 11.12 (s, 1H), 9.27 (s, 1H), 8.01 (d, J=8.5 Hz, 2H), 7.89 (d, J=8.8 Hz, 2H), 7.65 (d, J=8.8 Hz, 2H), 7.60 (d, J=8.5 Hz, 2H); ¹³C NMR (125 MHz, (CD₃)₂SO) δ 164.68, 157.55, 142.63, 137.41, 136.01, 132.79, 132.17, 130.37, 130.23, 129.04, 121.08.

### 4-methyl-N-(1-(p-tolyl)-1H-1,2,4-triazol-3-yl)benzamide (APZ-2164)

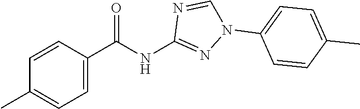

4-methyl-N-(1-(p-tolyl)-1H-1,2,4-triazol-3-yl)benzamide was prepared from 1-(p-tolyl)-1H-1,2,4-triazol-3-amine and 4-methylbenzoyl chloride as previously described for 4-chloro-N-(1-(p-tolyl)-1H-1,2,4-triazol-3-yl)benzamide. The crude product was purified by flash column chromatography with hexanes/ethyl acetate to afford title compound as a white solid (25%). mp 189-194° C.; R_{f} (60% EA/Hex)=0.15; ¹H NMR (500 MHz, (CD₃)₂SO) δ 10.83 (s, 1H), 9.17 (s, 1H), 7.90 (d, J=7.8 Hz, 2H), 7.73 (d, J=8.0 Hz, 2H), 7.36 (d, J=8.0 Hz, 2H), 7.32 (d, J=7.8 Hz, 2H), 2.38 (s, 3H), 2.35 (s, 3H); ¹³C NMR (125 MHz, (CD₃)₂SO) δ 165.67, 157.43, 142.57, 142.08, 137.55, 134.98, 131.25, 130.61, 129.46, 128.42, 119.33, 21.51, 20.99.

### N-(1-(4-chlorophenyl)-1H-1,2,4-triazol-3-yl)-4-methylbenzamide (APZ-2165)

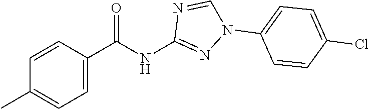 [SMILES: Cc1ccc(C(=O)Nc2ncn(-c3ccc(Cl)cc3)n2)cc1]

N-(1-(4-chlorophenyl)-1H-1,2,4-triazol-3-yl)-4-methylbenzamide was prepared from 1-(4-chlorophenyl)-1H-1,2,4-triazol-3-amine and 4-methylbenzoyl chloride as previously described for 4-chloro-N-(1-(p-tolyl)-1H-1,2,4-triazol-3-yl)benzamide. The crude product was purified by flash column chromatography with hexanes/ethyl acetate to afford title compound as a yellow solid (70%). mp 228-232° C.; R_{f} (60% EA/Hex)=0.18; ¹H NMR (500 MHz, (CD₃)₂SO) δ 10.90 (s, 1H), 9.25 (s, 1H), 7.90 (t, J=8.9 Hz, 4H), 7.64 (d, J=8.6 Hz, 2H), 7.32 (d, J=7.9 Hz, 2H), 2.38 (s, 3H); ¹³C NMR (125 MHz, (CD₃)₂SO) δ 165.58, 157.77, 142.62, 142.56, 136.04, 132.11, 131.20, 130.22, 129.47, 128.45, 121.04, 21.51.

### N-(1-(4-chlorophenyl)-1H-1,2,4-triazol-3-yl)-N,4-dimethylbenzamide (APZ-2331)

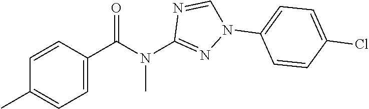 [SMILES: Cc1ccc(C(=O)N(C)c2ncn(-c3ccc(Cl)cc3)n2)cc1]

N-(1-(4-chlorophenyl)-1H-1,2,4-triazol-3-yl)-N,4-dimethylbenzamide was prepared via General Procedure A from N-(1-(4-chlorophenyl)-1H-1,2,4-triazol-3-yl)-4-methylbenzamide. The crude product was purified by flash column chromatography with hexanes/ethyl acetate to afford title compound as a white solid (60%). mp 158-162° C.; R_{f} (50% EA/Hex)=0.32; ¹H NMR (500 MHz, (CD₃)₂SO) δ 9.17 (s, 1H), 7.68 (dt, J=8.9, 2.9, 2.1 Hz, 2H), 7.59 (dt, J=8.9, 2.9, 2.1 Hz, 2H), 7.24 (d, J=8.0 Hz, 2H), 7.10 (d, J=8.0 Hz, 2H), 3.41 (s, 3H), 2.26 (s, 3H); ¹³C NMR (125 MHz, (CD₃)₂SO) δ 169.72, 161.78, 142.67, 140.22, 135.14, 132.85, 132.02, 129.72, 128.48, 127.84, 120.57, 35.72, 20.88.

### 4-chloro-N-(1-(4-chlorophenyl)-1H-1,2,4-triazol-3-yl)-N-methylbenzamide (APZ-2332)

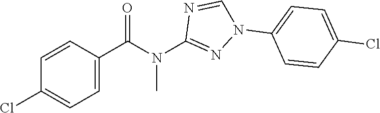 [SMILES: CN(C(=O)c1ccc(Cl)cc1)c1ncn(-c2ccc(Cl)cc2)n1]

4-chloro-N-(1-(4-chlorophenyl)-1H-1,2,4-triazol-3-yl)-N-methylbenzamide was prepared via General Procedure A from 4-chloro-N-(1-(4-chlorophenyl)-1H-1,2,4-triazol-3-yl)benzamide. The crude product was purified by flash column chromatography with hexanes/ethyl acetate to afford title compound as a white solid (62%). mp 95-98° C.; R_{f} (50% EA/Hex)=0.21; ¹H NMR (500 MHz, (CD₃)₂SO) δ 9.18 (s, 1H), 7.69 (dt, J=8.9, 2.9, 2.1 Hz, 2H), 7.60 (dt, J=8.9, 2.9, 2.1 Hz, 2H), 7.38 (ddt, J=10.7, 4.2, 1.9 Hz, 4H), 3.44 (s, 3H); ¹³C NMR (125 MHz, (CD₃)₂SO) δ 168.70, 161.36, 142.73, 135.09, 134.99, 134.62, 132.08, 129.73, 129.59, 128.12, 120.56, 35.55.

### N,4-dimethyl-N-(1-(p-tolyl)-1H-1,2,4-triazol-3-yl)benzamide (APZ-2333)

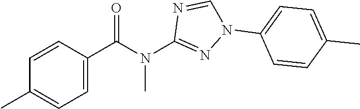

N,4-dimethyl-N-(1-(p-tolyl)-1H-1,2,4-triazol-3-yl)benzamide was prepared via General Procedure A from 4-methyl-N-(1-(p-tolyl)-1H-1,2,4-triazol-3-yl)benzamide. The crude product was purified by flash column chromatography with hexanes/ethyl acetate to afford title compound as a lustrous white solid (86%). mp 128-131° C.; R_{f} (50% EA/Hex)=0.32; ¹H NMR (500 MHz, (CD₃)₂SO) δ 9.09 (s, 1H), 7.52 (d, J=8.4 Hz, 2H), 7.30 (d, J=8.4 Hz, 2H), 7.24 (d, J=8.1 Hz, 2H), 7.10 (d, J=8.1 Hz, 2H), 3.41 (s, 3H), 2.33 (s, 3H), 2.26 (s, 3H); ¹³C NMR (125 MHz, (CD₃)₂SO) δ 169.72, 161.50, 142.18, 140.11, 137.45, 134.08, 132.92, 130.07, 128.43, 127.82, 118.86, 35.74, 20.86, 20.46.

### 4-chloro-N-methyl-N-(1-(p-tolyl)-1H-1,2,4-triazol-3-yl)benzamide (APZ-2334)

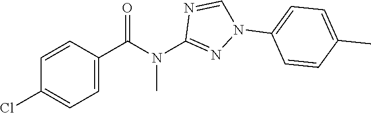 [SMILES: Cc1ccc(-n2cnc(N(C)C(=O)c3ccc(Cl)cc3)n2)cc1]

4-chloro-N-methyl-N-(1-(p-tolyl)-1H-1,2,4-triazol-3-yl)benzamide was prepared via General Procedure A from 4-chloro-N-(1-(p-tolyl)-1H-1,2,4-triazol-3-yl)benzamide. The crude product was purified by flash column chromatography with hexanes/ethyl acetate to afford title compound as a white solid (52%). mp 86-88° C.; R_{f} (50% EA/Hex)=0.37; ¹H NMR (500 MHz, (CD₃)₂SO) δ 9.10 (s, 1H), 7.51 (d, J=8.4 Hz, 2H), 7.37 (ddt, J=10.8, 4.2, 2.2 Hz, 4H), 7.31 (d, J=8.4 Hz, 2H), 3.44 (s, 3H), 2.33 (s, 3H); ¹³C NMR (125 MHz, (CD₃)₂SO) δ 168.72, 161.06, 142.25, 137.53, 134.90, 134.72, 134.03, 130.08, 129.56, 128.08, 118.85, 35.54, 20.47.

### N-(3,4-dimethylphenyl)-1-(4-ethylphenyl)-N-methyl-1H-1,2,4-triazole-3-carboxamide (APZ-2335)

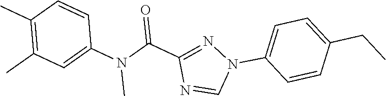 [SMILES: CCc1ccc(-n2cnc(C(=O)N(C)c3ccc(C)c(C)c3)n2)cc1]

N-(3,4-dimethylphenyl)-1-(4-ethylphenyl)-N-methyl-1H-1,2,4-triazole-3-carboxamide was prepared via General Procedure A from 1-(4-ethylphenyl)-1H-1,2,4-triazole-3-carboxylic acid. The crude product was purified by flash column chromatography with hexanes/ethyl acetate to afford title compound as a white solid (50%). mp 122-124° C.; R_{f} (70% EA/Hex)=0.27; ¹H NMR (500 MHz, (CD₃)₂SO) δ 9.08 (s, 1H), 7.57 (s, 2H), 7.36 (d, J=8.0 Hz, 2H), 7.15-6.95 (m, 2H), 6.87 (s, 1H), 3.38 (s, 3H), 2.65 (q, J=7.6 Hz, 2H), 2.15 (s, 6H), 1.19 (t, J=7.6 Hz, 3H); ¹³C NMR (125 MHz, (CD₃)₂SO) δ 161.30, 157.86, 144.01, 142.00, 141.08, 136.86, 134.93, 134.22, 129.70, 128.99, 127.44, 123.92, 119.38, 37.23, 27.61, 19.25, 18.83, 15.46.

### N-methyl-N-(m-tolyl)-1-(p-tolyl)-1H-1,2,4-triazole-3-carboxamide (APZ-2336)

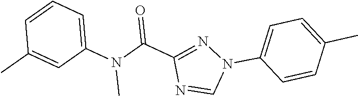

N-methyl-N-(m-tolyl)-1-(p-tolyl)-1H-1,2,4-triazole-3-carboxamide was prepared via General Procedure A from 1-(p-tolyl)-1H-1,2,4-triazole-3-carboxylic acid. The crude product was purified by flash column chromatography with hexanes/ethyl acetate to afford title compound as a lustrous off-white solid (78%). mp 100-105° C.; R_{f} (90% EA/Hex)=0.39; ¹H NMR (500 MHz, (CD₃)₂SO) δ 9.11 (bs, 1H), 7.55 (s, 2H), 7.33 (d, J=8.0 Hz, 2H), 7.18 (t, J=7.1 Hz, 1H), 7.09 (s, 1H), 7.03 (d, J=7.1 Hz, 1H), 7.01-6.87 (m, 1H), 3.40 (s, 3H), 2.34 (s, 3H), 2.25 (s, 3H); ¹³C NMR (175 MHz, CDCl₃) δ 161.74, 158.53, 144.05, 140.46, 139.13, 138.63, 134.40, 130.28, 128.89, 127.95, 127.46, 124.05, 119.98, 38.10, 21.35, 21.13.

### 1-(4-methoxyphenyl)-N-methyl-N-(m-tolyl)-1H-1,2,4-triazole-3-carboxamide (APZ-2337)

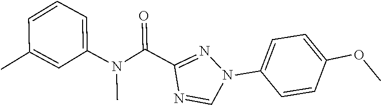

1-(4-methoxyphenyl)-N-methyl-N-(m-tolyl)-1H-1,2,4-triazole-3-carboxamide was prepared via General Procedure A from 1-(4-methoxyphenyl)-1H-1,2,4-triazole-3-carboxylic acid. The crude product was purified by flash column chromatography with hexanes/ethyl acetate to afford title compound as a lustrous white solid (89%). mp 112-115° C.; R_{f} (80% EA/Hex)=0.28; ¹H NMR (500 MHz, (CD₃)₂SO) δ 9.04 (s, 1H), 7.57 (s, 2H), 7.18 (t, J=7.6 Hz, 1H), 7.13-7.05 (m, 3H), 7.04 (d, J=7.6 Hz, 1H), 7.01-6.90 (m, 1H), 3.80 (s, 3H), 3.40 (s, 3H), 2.25 (s, 3H); ¹³C NMR (175 MHz, CDCl₃) δ 161.75, 159.65, 158.44, 144.06, 140.46, 139.11, 130.09, 128.87, 127.91, 127.42, 124.03, 121.76, 114.81, 55.69, 38.09, 21.34.

### 1-(4-chlorophenyl)-N-cyclopropyl-N-methyl-1H-1,2,4-triazole-3-carboxamide (APZ-2338)

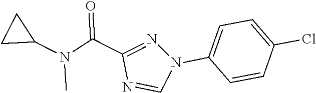 [SMILES: CN(C(=O)c1ncn(-c2ccc(Cl)cc2)n1)C1CC1]

1-(4-chlorophenyl)-N-cyclopropyl-N-methyl-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate, followed by preparatory TLC to afford title compound as a light yellow solid (59%). mp 93-98° C.; R_{f} (80% EA/Hex)=0.27; ¹H NMR (500 MHz, CDCl₃, rotameric mixture, both forms reported) δ 8.56 (s, 1H), 7.68 (d, J=8.8 Hz, 2H), 7.50 (dt, J=8.8, 2.9, 2.0 Hz, 2H), 3.19 (s, 0.55H), 3.16 (s, 2.45H), 3.14-3.06 (m, 0.80H), 2.93 (s, 0.20H), 0.96-0.90 (m, 0.40H), 0.84-0.76 (m, 0.40H), 0.69-0.61 (m, 1.60H), 0.59 f-0.50 (m, 1.60H); ¹³C NMR (125 MHz, CDCl₃, rotameric mixture, major form reported) δ 163.68, 159.61, 140.61, 135.26, 134.25, 130.01, 121.26, 34.70, 32.85, 8.99.

### 1-(4-chlorophenyl)-N-cyclobutyl-N-methyl-1H-1,2,4-triazole-3-carboxamide (APZ-2339)

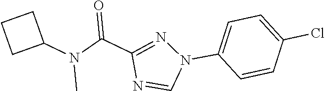 [SMILES: CN(C(=O)c1ncn(-c2ccc(Cl)cc2)n1)C1CCC1]

1-(4-chlorophenyl)-N-cyclobutyl-N-methyl-1H-1,2,4-triazole-3-carboxamide was prepared via General Procedure A from 1-(4-chlorophenyl)-1H-1,2,4-triazole-3-carboxylic acid. The crude product was purified by flash column chromatography with hexanes/ethyl acetate, followed by preparatory TLC to afford title compound as a white solid (61%). mp 97-101° C.; R_{f} (70% EA/Hex)=0.19; ¹H NMR (500 MHz, CDCl₃, rotameric mixture, both forms reported) δ 8.62-8.53 (m, 1H), 7.73-7.63 (m, 2H), 7.54-7.46 (m, 2H), 5.10 (p, J=8.6 Hz, 0.40H), 4.66 (p, J=8.6 Hz, 0.60H), 3.17 (s, 1.15H), 3.14 (s, 1.85H), 2.36-2.20 (m, 2.80H), 2.15-2.07 (m, 1.20H), 1.80-1.65 (m, 1.60H), 1.62-1.50 (m, 0.40H); ¹³C NMR (125 MHz, CDCl₃, rotameric mixture, major form reported) δ 161.69, 159.18, 140.98, 135.29, 134.42, 130.13, 121.38, 53.14, 28.57, 28.38, 14.50.

### N,5-bis(4-chlorophenyl)-N-(methyl-d₃)oxazole-2-carboxamide (APZ-2179)

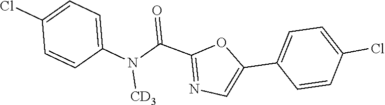 [SMILES: [2H]C([2H])([2H])N(C(=O)c1ncc(-c2ccc(Cl)cc2)o1)c1ccc(Cl)cc1]

N,5-bis(4-chlorophenyl)-N-(methyl-d₃)oxazole-2-carboxamide was prepared via General Procedure D from N,5-bis(4-chlorophenyl)oxazole-2-carboxamide with iodomethane-d₃ in place of iodomethane. The crude product was purified by flash column chromatography with hexanes/ethyl acetate to afford title compound as a light yellow solid (64%). mp 140-142° C.; R_{f} (30% EA/Hex)=0.24; ¹H NMR (500 MHz, (CD₃)₂SO) δ 7.75 (s, 1H), 7.67-7.56 (m, 2H), 7.54 (d, J=8.2 Hz, 2H), 7.45 (d, J=8.4 Hz, 2H), 7.38 (d, J=8.2 Hz, 2H); ¹³C NMR (125 MHz, (CD₃)₂SO) δ 155.85, 153.75, 150.21, 142.16, 133.88, 131.83, 129.29, 129.17, 128.49, 126.11, 125.35, 123.88.

### (4-chlorophenyl)(5-(4-chlorophenyl)oxazol-2-yl)methanone (APZ-2180)

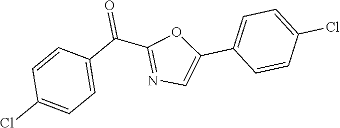 [SMILES: O=C(c1ccc(Cl)cc1)c1ncc(-c2ccc(Cl)cc2)o1]

(4-chlorophenyl)(5-(4-chlorophenyl)oxazol-2-yl)methanone was prepared from 5-(4-chlorophenyl)-N-methoxy-N-methyloxazole-2-carboxamide as previously described for (4-chlorophenyl)(4-(4-chlorophenyl)pyridin-2-yl)methanone. The crude product was purified by flash column chromatography with hexanes/ethyl acetate to afford title compound as a lustrous light yellow solid (63%). mp 186-189° C.; R_{f} (5% EA/Hex)=0.23; ¹H NMR (500 MHz, (CD₃)₂SO) δ 8.41 (d, J=8.2 Hz, 2H), 8.20 (s, 1H), 7.93 (d, J=8.2 Hz, 2H), 7.71 (d, J=8.2 Hz, 2H), 7.66 (d, J=8.2 Hz, 2H); ¹³C NMR (125 MHz, (CD₃)₂SO) δ 176.79, 156.50, 152.42, 139.00, 134.66, 133.62, 132.31, 129.54, 128.75, 126.91, 125.61, 125.18.

### (4-chlorophenyl)(4-(4-chlorophenyl)pyridin-2-yl)methanone (APZ-2182)

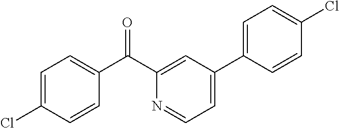 [SMILES: O=C(c1ccc(Cl)cc1)c1cc(-c2ccc(Cl)cc2)ccn1]

(4-chlorophenyl)(4-(4-chlorophenyl)pyridin-2-yl)methanone was prepared from 4-(4-chlorophenyl)-N-methoxy-N-methylpicolinamide as described herein. The crude product was purified by flash column chromatography with hexanes/ethyl acetate to afford title compound as a fluffy white solid (45%). mp 161-166° C.; R_{f} (20% EA/Hex)=0.47; ¹H NMR (500 MHz, CDCl₃) δ 8.76 (d, J=5.1 Hz, 1H), 8.29-8.24 (m, 1H), 8.10 (d, J=8.5 Hz, 2H), 7.72-7.62 (m, 3H), 7.55-7.44 (m, 4H); ¹³C NMR (125 MHz, CDCl₃) δ 192.54, 155.51, 149.30, 148.68, 139.69, 136.11, 135.88, 134.69, 132.67, 129.71, 128.66, 128.50, 123.99, 122.45.

### N,4-bis(4-chlorophenyl)-N-(methyl-d₃)picolinamide (APZ-2183)

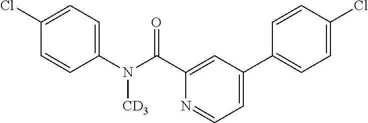 [SMILES: [2H]C([2H])([2H])N(C(=O)c1cc(-c2ccc(Cl)cc2)ccn1)c1ccc(Cl)cc1]

N,4-bis(4-chlorophenyl)-N-(methyl-d₃)picolinamide was prepared via General Procedure D from N,4-bis(4-chlorophenyl)picolinamide with iodomethane-d₃ in place of iodomethane. The crude product was purified by flash column chromatography with hexanes/ethyl acetate to afford title compound as a white solid (22%). mp 121-123° C.; R_{f} (40% EA/Hex)=0.18; ¹H NMR (500 MHz, CDCl₃) δ 8.36 (s, 1H), 7.77 (s, 1H), 7.51 (d, J=8.2 Hz, 2H), 7.45 (d, J=8.2 Hz, 2H), 7.41-7.29 (m, 1H), 7.25-7.14 (m, 2H), 7.14-6.90 (m, 2H); ¹³C NMR (125 MHz, CDCl₃) δ 168.66, 154.63, 149.07, 147.88, 143.17, 135.93, 135.86, 132.30, 129.57, 129.32, 128.35, 127.99, 121.95, 121.78.

### N-methyl-N,5-di-p-tolyloxazole-2-carboxamide (APZ-2340)

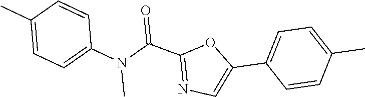

N-methyl-N,5-di-p-tolyloxazole-2-carboxamide was prepared via General Procedure A from 5-(p-tolyl)oxazole-2-carboxylic acid. The crude product was purified by flash column chromatography with hexanes/ethyl acetate, followed by recrystallization in dichloromethane/hexanes to afford title compound as a white solid (73%). mp 116-119° C.; R_{f} (30% EA/Hex)=0.22; ¹H NMR (500 MHz, (CD₃)₂SO) δ 7.70-7.48 (bs, 1H), 7.48-7.29 (m, 2H), 7.24 (d, J=7.7 Hz, 2H), 7.21-7.11 (m, 4H), 3.45 (bs, 3H), 2.31 (s, 3H), 2.28 (s, 3H); ¹³C NMR (125 MHz, (CD₃)₂SO) δ 156.72, 154.10, 151.62, 141.22, 139.51, 137.31, 130.16, 130.12, 126.77, 124.77, 124.32, 122.97, 38.35, 21.36, 21.02.

### N-(4-chlorophenyl)-N-methyl-2-(p-tolyl)pyrimidine-4-carboxamide (APZ-2341)

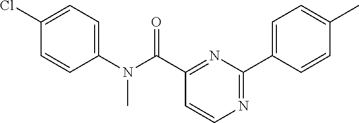 [SMILES: Cc1ccc(-c2nccc(C(=O)N(C)c3ccc(Cl)cc3)n2)cc1]

N-(4-chlorophenyl)-N-methyl-2-(p-tolyl)pyrimidine-4-carboxamide was prepared via General Procedure A from 2-(p-tolyl)pyrimidine-4-carboxylic acid. The crude product was purified by flash column chromatography with hexanes/ethyl acetate, followed by recrystallization in dichloromethane/hexanes to afford title compound as a white solid (48%). mp 114-117° C.; R_{f} (30% EA/Hex)=0.19; ¹H NMR (500 MHz, CDCl₃) δ 8.80 (d, J=5.0 Hz, 1H), 7.88 (d, J=7.2 Hz, 2H), 7.46 (d, J=5.0 Hz, 1H), 7.27-7.16 (m, 4H), 7.09 (d, J=7.2 Hz, 2H), 3.53 (s, 3H), 2.39 (s, 3H); ¹³C NMR (125 MHz, (CD₃)₂SO) δ 166.17, 162.74, 161.35, 159.48, 143.09, 141.54, 134.21, 131.80, 129.69, 129.45, 129.22, 128.12, 118.69, 37.81, 21.46.

### 5-(4-chlorophenyl)-N-methyl-N-(p-tolyl)oxazole-2-carboxamide (APZ-2342)

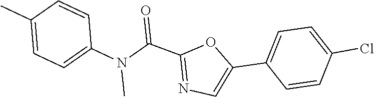 [SMILES: Cc1ccc(N(C)C(=O)c2ncc(-c3ccc(Cl)cc3)o2)cc1]

5-(4-chlorophenyl)-N-methyl-N-(p-tolyl)oxazole-2-carboxamide was prepared via General Procedure A from 5-(4-chlorophenyl)oxazole-2-carboxylic acid. The crude product was purified by flash column chromatography with hexanes/ethyl acetate, followed by recrystallization in dichloromethane/hexanes to afford title compound as a white solid (51%). mp 134-137° C.; R_{f} (30% EA/Hex)=0.18; ¹H NMR (500 MHz, CDCl₃) δ 7.43-7.27 (m, 4H), 7.21 (bs, 1H), 7.17 (d, J=7.5 Hz, 2H), 7.08 (d, J=7.5 Hz, 2H), 3.49 (s, 3H), 2.36 (s, 3H); ¹³C NMR (125 MHz, (CD₃)₂SO) δ 156.13, 154.10, 149.88, 140.61, 136.93, 133.76, 129.69, 129.22, 126.30, 126.03, 125.41, 123.75, 37.81, 20.52.

### N,2-bis(4-chlorophenyl)-N-methylpyrimidine-4-carboxamide (APZ-2343)

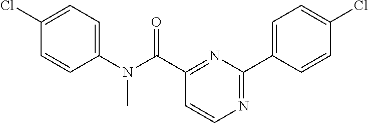 [SMILES: CN(C(=O)c1ccnc(-c2ccc(Cl)cc2)n1)c1ccc(Cl)cc1]

N,2-bis(4-chlorophenyl)-N-methylpyrimidine-4-carboxamide was prepared via General Procedure A from 2-(4-chlorophenyl)pyrimidine-4-carboxylic acid. The crude product was purified by flash column chromatography with hexanes/ethyl acetate, followed by recrystallization in dichloromethane/hexanes to afford title compound as a white solid (35%). mp 80-83° C.; R_{f} (30% EA/Hex)=0.20; ¹H NMR (500 MHz, CDCl₃) δ 8.82 (d, J=5.0 Hz, 1H), 7.93 (d, J=8.2 Hz, 2H), 7.51 (d, J=5.0 Hz, 1H), 7.36 (d, J=8.2 Hz, 2H), 7.28-7.19 (m, 3H), 7.09 (d, J=8.0 Hz, 2H), 3.53 (s, 3H); ¹³C NMR (125 MHz, (CD₃)₂SO) δ 165.93, 161.65, 161.45, 159.72, 143.02, 136.57, 135.66, 131.90, 129.83, 129.50, 129.28, 129.20, 119.28, 37.80.

### N-(4-chlorophenyl)-N-methyl-5-(p-tolyl)-1,3,4-oxadiazole-2-carboxamide (APZ-2344)

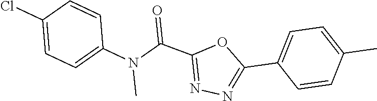 [SMILES: Cc1ccc(-c2nnc(C(=O)N(C)c3ccc(Cl)cc3)o2)cc1]

N-(4-chlorophenyl)-N-methyl-5-(p-tolyl)-1,3,4-oxadiazole-2-carboxamide was prepared via General Procedure A from 5-(p-tolyl)-1,3,4-oxadiazole-2-carboxylic acid. The crude product was purified by flash column chromatography with hexanes/ethyl acetate, followed by recrystallization in dichloromethane/hexanes to afford title compound as a white solid (67%). mp 125-127° C.; R_{f} (30% EA/Hex)=0.32; ¹H NMR (500 MHz, CDCl₃, rotameric mixture, major form reported) δ 7.86 (d, J=7.7 Hz, 2H), 7.36 (d, J=8.1 Hz, 2H), 7.29 (d, J=7.7 Hz, 3H), 7.18 (d, J=8.1 Hz, 2H), 3.51 (s, 3H), 2.42 (s, 3H); ¹³C NMR (125 MHz, CDCl₃) δ 165.10, 157.70, 155.02, 143.28, 141.08, 134.53, 129.98, 129.94, 128.42, 127.39, 120.14, 38.53, 21.79.

### N,5-bis(4-chlorophenyl)-N-methyl-1,3,4-oxadiazole-2-carboxamide (APZ-2345)

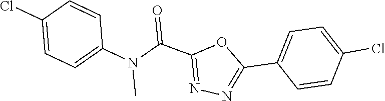 [SMILES: CN(C(=O)c1nnc(-c2ccc(Cl)cc2)o1)c1ccc(Cl)cc1]

N,5-bis(4-chlorophenyl)-N-methyl-1,3,4-oxadiazole-2-carboxamide was prepared via General Procedure A from 5-(4-chlorophenyl)-1,3,4-oxadiazole-2-carboxylic acid. The crude product was purified by flash column chromatography with hexanes/ethyl acetate, followed by recrystallization in dichloromethane/hexanes to afford title compound as a lustrous off-white solid (71%). mp 137-140° C.; R_{f} (30% EA/Hex)=0.42; ¹H NMR (500 MHz, CDCl₃, rotameric mixture, major form reported) δ 7.92 (d, J=8.2 Hz, 2H), 7.48 (d, J=8.2 Hz, 2H), 7.37 (d, J=8.1 Hz, 2H), 7.18 (d, J=8.1 Hz, 2H), 3.51 (s, 3H); ¹³C NMR (125 MHz, CDCl₃ₙ) δ 164.16, 158.00, 154.78, 140.97, 139.00, 134.68, 130.06, 129.69, 128.71, 128.45, 121.41, 38.59.

### N-(4-chlorophenyl)-N-methyl-6-(p-tolyl)pyrimidine-4-carboxamide (APZ-2346)

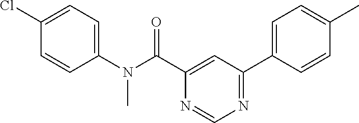 [SMILES: Cc1ccc(-c2cc(C(=O)N(C)c3ccc(Cl)cc3)ncn2)cc1]

N-(4-chlorophenyl)-N-methyl-6-(p-tolyl)pyrimidine-4-carboxamide was prepared via General Procedure A from 6-(p-tolyl)pyrimidine-4-carboxylic acid. The crude product was purified by flash column chromatography with hexanes/ethyl acetate to afford title compound as a white solid (45%). mp 123-125° C.; R_{f} (30% EA/Hex)=0.19; ¹H NMR (500 MHz, CDCl₃) δ 8.93 (s, 1H), 7.95 (d, J=7.8 Hz, 2H), 7.89 (s, 1H), 7.32 (d, J=7.8 Hz, 2H), 7.25-7.13 (m, 2H), 7.13-6.95 (m, 2H), 3.51 (s, 3H), 2.43 (s, 3H); ¹³C NMR (125 MHz, (CD₃)₂SO) δ 166.21, 163.39, 162.21, 157.81, 141.89, 141.75, 132.66, 131.39, 129.74, 129.02, 128.88, 126.97, 115.15, 36.83, 20.98.

### N,6-bis(4-chlorophenyl)-N-methylpyrimidine-4-carboxamide (APZ-2273)

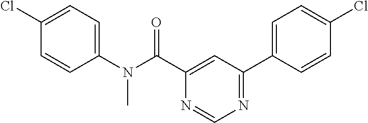 [SMILES: CN(C(=O)c1cc(-c2ccc(Cl)cc2)ncn1)c1ccc(Cl)cc1]

N,6-bis(4-chlorophenyl)-N-methylpyrimidine-4-carboxamide was prepared via General Procedure A from 6-(4-chlorophenyl)pyrimidine-4-carboxylic acid. The crude product was purified by flash column chromatography with hexanes/ethyl acetate to afford title compound as a white solid (25%). mp 126-129° C.; R_{f} (30% EA/Hex)=0.23; ¹H NMR (500 MHz, (CD₃)₂SO) δ 9.02 (s, 1H), 8.27 (s, 1H), 8.24-8.06 (m, 2H), 7.63 (d, J=8.2 Hz, 2H), 7.43-7.15 (m, 4H), 3.37 (s, 3H); ¹³C NMR (125 MHz, (CD₃)₂SO) δ 166.54, 163.02, 162.80, 158.35, 142.30, 137.02, 134.74, 131.91, 129.72 (2 non-equivalent C), 129.53, 129.35, 116.12, 37.33.

### N-(4-chlorophenyl)-N-methyl-6-(p-tolyl)picolinamide (APZ-2191)

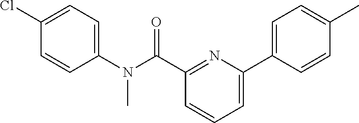 [SMILES: Cc1ccc(-c2cccc(C(=O)N(C)c3ccc(Cl)cc3)n2)cc1]

N-(4-chlorophenyl)-N-methyl-6-(p-tolyl)picolinamide was prepared via General Procedure A from 6-(p-tolyl)picolinic acid. The crude product was purified by flash column chromatography with hexanes/ethyl acetate to afford title compound as an orange solid (64%). mp 79-83° C.; R_{f} (30% EA/Hex)=0.22; ¹H NMR (500 MHz, CDCl₃) δ 7.75 (t, J=7.7 Hz, 1H), 7.66 (d, J=7.4 Hz, 1H), 7.58 (d, J=7.0 Hz, 1H), 7.44-7.27 (m, 2H), 7.24 (d, J=7.7 Hz, 2H), 7.17 (d, J=7.7 Hz, 2H), 7.13-7.02 (m, 2H), 3.54 (s, 3H), 2.37 (s, 3H); ¹³C NMR (125 MHz, CDCl₃) δ 168.18, 155.85, 152.79, 144.06, 139.48, 137.65, 135.59, 132.11, 129.42, 129.26, 128.02, 126.91, 122.63, 121.06, 38.78, 21.39.

### N,6-bis(4-chlorophenyl)-N-methylpicolinamide (APZ-2317)

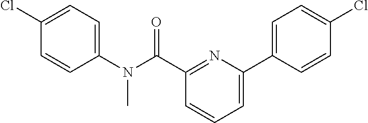 [SMILES: CN(C(=O)c1cccc(-c2ccc(Cl)cc2)n1)c1ccc(Cl)cc1]

N,6-bis(4-chlorophenyl)-N-methylpicolinamide was prepared via General Procedure A from 6-(4-chlorophenyl)picolinic acid. The crude product was purified by flash column chromatography with hexanes/ethyl acetate, followed by recrystallization in dichloromethane/hexanes to afford title compound as a white solid (47%). mp 131-135° C.; R_{f} (30% EA/Hex)=0.23; ¹H NMR (500 MHz, CDCl₃) δ 7.77 (t, J=7.8 Hz, 1H), 7.74-7.70 (m, 1H), 7.56 (d, J=7.8 Hz, 1H), 7.49-7.28 (m, 4H), 7.24 (d, J=8.2 Hz, 2H), 7.14-6.95 (m, 2H), 3.54 (s, 3H); ¹³C NMR (125 MHz, CDCl₃) δ 167.96, 154.57, 153.01, 144.07, 137.76, 136.88, 135.49, 132.17, 129.26, 128.85, 128.21, 127.99, 123.21, 121.02, 38.77.

### 6-(4-chlorophenyl)-N-methyl-N-(p-tolyl)pyrazine-2-carboxamide (APZ-2192)

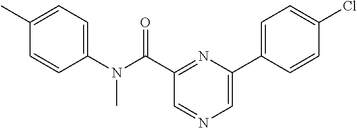 [SMILES: Cc1ccc(N(C)C(=O)c2cncc(-c3ccc(Cl)cc3)n2)cc1]

6-(4-chlorophenyl)-N-methyl-N-(p-tolyl)pyrazine-2-carboxamide was prepared via General Procedure A from 6-(4-chlorophenyl)pyrazine-2-carboxylic acid. The crude product was purified by flash column chromatography with hexanes/ethyl acetate, followed by recrystallization in dichloromethane/hexanes to afford title compound as a lustrous light yellow solid (52%). mp 123-125° C.; R_{f} (30% EA/Hex)=0.23; ¹H NMR (500 MHz, CDCl₃) δ 8.88-8.75 (m, 2H), 7.50 (d, J=8.2 Hz, 2H), 7.36 (d, J=8.2 Hz, 2H), 7.07 (d, J=7.9 Hz, 2H), 7.00 (d, J=7.9 Hz, 2H), 3.54 (s, 3H), 2.28 (s, 3H); ¹³C NMR (125 MHz, CDCl₃) δ 166.19, 149.26, 148.33, 143.55, 141.88, 141.50, 137.12, 136.46, 134.13, 129.97, 129.14, 128.37, 126.86, 38.55, 21.06.

### N-methyl-N,6-di-p-tolylpyrazine-2-carboxamide (APZ-2193)

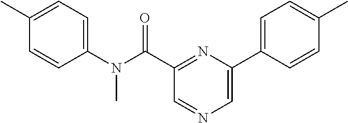

N-methyl-N,6-di-p-tolylpyrazine-2-carboxamide was prepared via General Procedure A from 6-(p-tolyl)pyrazine-2-carboxylic acid. The crude product was purified by flash column chromatography with hexanes/ethyl acetate to afford title compound as a lustrous white solid (78%). mp 119-121° C.; R_{f} (30% EA/Hex)=0.27; ¹H NMR (500 MHz, CDCl₃) δ 8.81 (s, 1H), 8.74 (s, 1H), 7.46 (d, J=7.6 Hz, 2H), 7.19 (d, J=7.6 Hz, 2H), 7.06 (d, J=7.7 Hz, 2H), 7.01 (d, J=7.7 Hz, 2H), 3.54 (s, 3H), 2.38 (s, 3H), 2.27 (s, 3H); ¹³C NMR (125 MHz, CDCl₃) δ 166.53, 150.52, 148.27, 142.84, 141.90, 141.59, 140.38, 136.99, 132.94, 129.93, 129.62, 127.03, 126.85, 38.49, 21.45, 21.04.

### N,4-bis(4-chlorophenyl)-N-methylpyrimidine-2-carboxamide (APZ-2194)

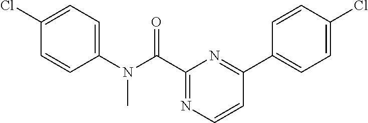 [SMILES: CN(C(=O)c1nccc(-c2ccc(Cl)cc2)n1)c1ccc(Cl)cc1]

N,4-bis(4-chlorophenyl)-N-methylpyrimidine-2-carboxamide was prepared via General Procedure A from 4-(4-chlorophenyl)pyrimidine-2-carboxylic acid. The crude product was purified by flash column chromatography with hexanes/ethyl acetate, followed by recrystallization in dichloromethane/hexanes to afford title compound as a lustrous white solid (53%). mp 133-135° C.; R_{f} (50% EA/Hex)=0.25; ¹H NMR (500 MHz, CDCl₃, rotameric mixture, major form reported) δ 8.59 (d, J=5.0 Hz, 1H), 7.78 (d, J=7.8 Hz, 2H), 7.48 (d, J=5.0 Hz, 1H), 7.30-7.23 (m, 2H), 7.15 (d, J=8.1 Hz, 2H), 7.11 (d, J=8.1 Hz, 2H), 3.54 (s, 3H), 2.41 (s, 3H); ¹³C NMR (125 MHz, CDCl₃) δ 166.68, 163.81, 162.29, 157.49, 142.36, 142.11, 133.05, 132.71, 129.81, 129.31, 128.23, 127.29, 115.80, 37.38, 21.60.

### N-(4-chlorophenyl)-N-methyl-4-(p-tolyl)pyrimidine-2-carboxamide (APZ-2195)

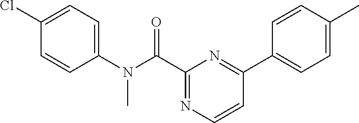 [SMILES: Cc1ccc(-c2ccnc(C(=O)N(C)c3ccc(Cl)cc3)n2)cc1]

N-(4-chlorophenyl)-N-methyl-4-(p-tolyl)pyrimidine-2-carboxamide was prepared via General Procedure A from 4-(p-tolyl)pyrimidine-2-carboxylic acid. The crude product was purified by flash column chromatography with hexanes/ethyl acetate, followed by recrystallization in dichloromethane/hexanes to afford title compound as a fluffy white solid (69%). mp 142-144° C.; R_{f} (50% EA/Hex)=0.16; ¹H NMR (500 MHz, CDCl₃, rotameric mixture, major form reported) δ 8.64 (d, J=5.0 Hz, 1H), 7.83 (d, J=8.2 Hz, 2H), 7.48 (d, J=5.0 Hz, 1H), 7.43 (d, J=8.2 Hz, 2H), 7.16 (d, J=8.3 Hz, 2H), 7.10 (d, J=8.3 Hz, 2H), 3.55 (s, 3H); ¹³C NMR (125 MHz, CDCl₃) δ 166.42, 162.65, 162.42, 157.87, 142.25, 137.92, 134.21, 132.84, 129.36 (2 non-equivalent C), 128.62, 128.24, 115.90, 37.39.

### N-(4-chlorophenyl)-5-(4-ethylphenyl)-N-methyloxazole-2-carboxamide (AW-02-2196)

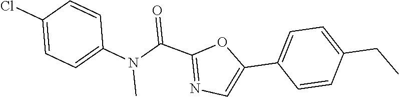 [SMILES: CCc1ccc(-c2cnc(C(=O)N(C)c3ccc(Cl)cc3)o2)cc1]

N-(4-chlorophenyl)-5-(4-ethylphenyl)-N-methyloxazole-2-carboxamide was prepared via General Procedure A from 5-(4-ethylphenyl)oxazole-2-carboxylic acid. The crude product was purified by flash column chromatography with hexanes/ethyl acetate, followed by recrystallization in dichloromethane/hexanes to afford title compound as a lustrous white solid (73%). mp 103-104° C.; R_{f} (30% EA/Hex)=0.22; ¹H NMR (500 MHz, CDCl₃) δ 7.54-7.27 (m, 4H), 7.24-7.19 (m, 3H), 7.19-7.09 (m, 2H), 3.52 (s, 3H), 2.66 (q, J=7.6 Hz, 2H), 1.24 (t, J=7.6 Hz, 3H); ¹³C NMR (125 MHz, CDCl₃) δ 156.86, 153.61, 152.66, 146.03, 142.36, 133.54, 129.73, 128.59, 128.05, 124.93, 124.44, 122.24, 38.72, 28.86, 15.46.

### N-(4-chlorophenyl)-5-(4-isopropylphenyl)-N-methyloxazole-2-carboxamide (APZ-2197)

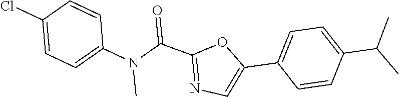 [SMILES: CC(C)c1ccc(-c2cnc(C(=O)N(C)c3ccc(Cl)cc3)o2)cc1]

N-(4-chlorophenyl)-5-(4-isopropylphenyl)-N-methyloxazole-2-carboxamide was prepared via General Procedure A from 5-(4-isopropylphenyl)oxazole-2-carboxylic acid. The crude product was purified by flash column chromatography with hexanes/ethyl acetate, followed by recrystallization in dichloromethane/hexanes to afford title compound as a lustrous white solid (57%). mp 111-113° C.; R_{f} (30% EA/Hex)=0.20; ¹H NMR (500 MHz, CDCl₃) δ 7.47-7.37 (m, 2H), 7.36 (d, J=8.5 Hz, 2H), 7.24 (d, J=8.1 Hz, 2H), 7.22-7.01 (m, 3H), 3.52 (s, 3H), 2.92 (hept, J=6.9 Hz, 1H), 1.25 (d, J=6.9 Hz, 6H ¹³C NMR (125 MHz, CDCl₃) δ 156.88, 153.63, 152.64, 150.64, 142.37, 133.54, 129.73, 128.04, 127.18, 124.96, 124.57, 122.25, 38.71, 34.15, 23.90.

### N-(4-chlorophenyl)-N-methyl-5-(4-(trifluoromethyl)phenyl)oxazole-2-carboxamide (APZ-2198)

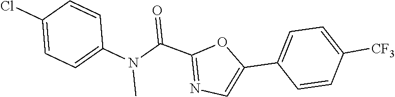 [SMILES: CN(C(=O)c1ncc(-c2ccc(C(F)(F)F)cc2)o1)c1ccc(Cl)cc1]

N-(4-chlorophenyl)-N-methyl-5-(4-(trifluoromethyl)phenyl)oxazole-2-carboxamide was prepared via General Procedure A from 5-(4-(trifluoromethyl)phenyl)oxazole-2-carboxylic acid. The crude product was purified by flash column chromatography with hexanes/ethyl acetate, followed by recrystallization in dichloromethane/hexanes to afford title compound as a lustrous white solid (49%). mp 117-118° C.; R_{f} (30% EA/Hex)=0.28; ¹H NMR (500 MHz, CDCl₃) δ 7.87-7.47 (m, 4H), 7.44-7.28 (m, 3H), 7.24-7.06 (m, 2H), 3.52 (s, 3H); ¹³C NMR (125 MHz, CDCl₃) δ 156.60, 154.60, 150.85, 142.11, 133.82, 131.17 (q, CCF₃, J=32.9 Hz), 130.20, 129.83, 128.09, 126.17 (q, ArCF₃, J=3.8 Hz), 125.03, 123.87 (q, CF₃, J=272.1 Hz), 38.66.

### N-methyl-N,4-di-p-tolylpyrimidine-2-carboxamide (APZ-2211)

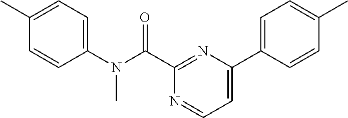

N-methyl-N,4-di-p-tolylpyrimidine-2-carboxamide was prepared via General Procedure A from 4-(p-tolyl)pyrimidine-2-carboxylic acid. The crude product was purified by flash column chromatography with hexanes/ethyl acetate, followed by recrystallization in dichloromethane/hexanes to afford title compound as a lustrous white solid (72%). mp 107-109° C.; R_{f} (50% EA/Hex)=0.25; ¹H NMR (500 MHz, CDCl₃, rotameric mixture, major form reported) δ 8.58 (d, J=5.3 Hz, 1H), 7.77 (d, J=8.0 Hz, 2H), 7.43 (d, J=5.3 Hz, 1H), 7.24 (d, J=8.0 Hz, 2H), 7.05 (d, J=8.0 Hz, 2H), 6.97 (d, J=8.0 Hz, 2H), 3.54 (s, 3H), 2.40 (s, 3H), 2.20 (s, 3H); ¹³C NMR (125 MHz, CDCl₃) δ 166.89, 163.61, 162.74, 157.34, 141.82, 141.07, 136.79, 133.27, 129.67, 127.30, 126.70, 115.55, 37.38, 21.55, 21.00.

### 4-(4-chlorophenyl)-N-methyl-N-(p-tolyl)pyrimidine-2-carboxamide (APZ-2212)

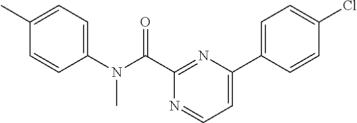 [SMILES: Cc1ccc(N(C)C(=O)c2nccc(-c3ccc(Cl)cc3)n2)cc1]

4-(4-chlorophenyl)-N-methyl-N-(p-tolyl)pyrimidine-2-carboxamide was prepared via General Procedure A from 4-(4-chlorophenyl)pyrimidine-2-carboxylic acid. The crude product was purified by flash column chromatography with hexanes/ethyl acetate, followed by recrystallization in dichloromethane/hexanes to afford title compound as a white solid (40%). mp 152-153° C.; R_{f} (60% EA/Hex)=0.21; ¹H NMR (500 MHz, CDCl₃, rotameric mixture, major form reported) δ 8.63 (d, J=5.3 Hz, 1H), 7.81 (d, J=8.3 Hz, 2H), 7.45 (d, J=5.3 Hz, 1H), 7.41 (d, J=8.3 Hz, 2H), 7.04 (d, J=8.1 Hz, 2H), 6.98 (d, J=8.1 Hz, 2H), 3.54 (s, 3H), 2.20 (s, 3H); ¹³C NMR (125 MHz, CDCl₃) δ 166.62, 162.83, 162.43, 157.74, 140.98, 137.66, 136.93, 134.42, 129.70, 129.21, 128.63, 126.70, 115.65, 37.40, 21.00.

### N-(4-chlorophenyl)-6-(4-ethylphenyl)-N-methylpyrazine-2-carboxamide (APZ-2213)

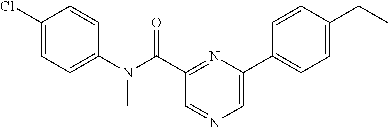 [SMILES: CCc1ccc(-c2cncc(C(=O)N(C)c3ccc(Cl)cc3)n2)cc1]

N-(4-chlorophenyl)-6-(4-ethylphenyl)-N-methylpyrazine-2-carboxamide was prepared via General Procedure A from 6-(4-ethylphenyl)pyrazine-2-carboxylic acid. The crude product was purified by flash column chromatography with hexanes/ethyl acetate, followed by recrystallization in dichloromethane/hexanes to afford title compound as a lustrous tan solid (80%). mp 151-154° C.; R_{f} (30% EA/Hex)=0.23; ¹H NMR (500 MHz, CDCl₃) δ 8.94-8.77 (m, 2H), 7.58-7.33 (m, 2H), 7.34-7.17 (m, 4H), 7.17-6.98 (m, 2H), 3.54 (s, 3H), 2.69 (q, J=7.6 Hz, 2H), 1.26 (t, J=7.6 Hz, 3H); ¹³C NMR (125 MHz, CDCl₃) δ 166.11, 150.34, 147.38, 146.82, 143.19, 143.09, 141.99, 132.80, 132.71, 129.43, 128.48, 128.17, 126.92, 38.59, 28.72, 15.41.

### N-(4-chlorophenyl)-N-methyl-6-(4-(trifluoromethyl)phenyl)pyrazine-2-carboxamide (APZ-2214)

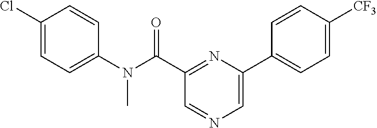 [SMILES: CN(C(=O)c1cncc(-c2ccc(C(F)(F)F)cc2)n1)c1ccc(Cl)cc1]

N-(4-chlorophenyl)-N-methyl-6-(4-(trifluoromethyl)phenyl)pyrazine-2-carboxamide was prepared via General Procedure A from 6-(4-(trifluoromethyl)phenyl)pyrazine-2-carboxylic acid. The crude product was purified by flash column chromatography with hexanes/ethyl acetate, followed by recrystallization in dichloromethane/hexanes to afford title compound as a lustrous tan solid (81%). mp 144-146° C.; R_{f} (30% EA/Hex)=0.20; ¹H NMR (500 MHz, CDCl₃) δ 8.97 (s, 1H), 8.93 (bs, 1H), 7.74-7.53 (m, 4H), 7.28 (d, J=8.0 Hz, 2H), 7.22-6.95 (m, 2H), 3.56 (s, 3H); ¹³C NMR (125 MHz, CDCl₃) δ 165.71, 148.79, 147.77, 144.57, 143.18, 142.37, 138.75, 133.04, 132.09 (q, CCF₃, J=32.6 Hz), 129.62, 128.33, 127.32, 125.98 (q, ArCF₃, J=3.8 Hz), 123.97 (q, CF₃, J=272.3 Hz), 38.73.

### N-(4-chlorophenyl)-4-(4-ethylphenyl)-N-methylpyrimidine-2-carboxamide (APZ-2215)

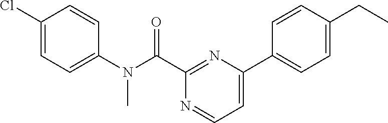 [SMILES: CCc1ccc(-c2ccnc(C(=O)N(C)c3ccc(Cl)cc3)n2)cc1]

N-(4-chlorophenyl)-4-(4-ethylphenyl)-N-methylpyrimidine-2-carboxamide was prepared via General Procedure A from 4-(4-ethylphenyl)pyrimidine-2-carboxylic acid. The crude product was purified by flash column chromatography with hexanes/ethyl acetate, followed by recrystallization in dichloromethane/hexanes to afford title compound as a lustrous off-white solid (81%). mp 149-151° C.; R_{f} (50% EA/Hex)=0.30; ¹H NMR (500 MHz, CDCl₃, rotameric mixture, major form reported) δ 8.60 (d, J=5.4 Hz, 1H), 7.80 (d, J=7.8 Hz, 2H), 7.48 (d, J=5.4 Hz, 1H), 7.28 (d, J=7.8 Hz, 2H), 7.15 (d, J=8.3 Hz, 2H), 7.11 (d, J=8.3 Hz, 2H), 3.54 (s, 3H), 2.70 (q, J=7.6 Hz, 2H), 1.26 (t, J=7.6 Hz, 3H); ¹³C NMR (125 MHz, CDCl₃) δ 166.67, 163.83, 162.27, 157.46, 148.34, 142.33, 133.26, 132.69, 129.29, 128.61, 128.20, 127.38, 115.84, 37.35, 28.90, 15.43.

### N-(4-chlorophenyl)-N-methyl-4-(4-(trifluoromethyl)phenyl)pyrimidine-2-carboxamide (APZ-2216)

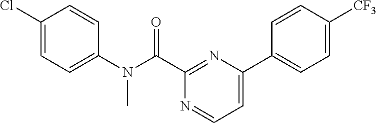 [SMILES: CN(C(=O)c1nccc(-c2ccc(C(F)(F)F)cc2)n1)c1ccc(Cl)cc1]

N-(4-chlorophenyl)-N-methyl-4-(4-(trifluoromethyl)phenyl)pyrimidine-2-carboxamide was prepared via General Procedure A from 4-(4-(trifluoromethyl)phenyl)pyrimidine-2-carboxylic acid. The crude product was purified by flash column chromatography with hexanes/ethyl acetate, followed by recrystallization in dichloromethane/hexanes to afford title compound as a fluffy white solid (78%). mp 184-187° C.; R_{f} (50% EA/Hex)=0.31; ¹H NMR (500 MHz, CDCl₃, rotameric mixture, major form reported) δ 8.71 (d, J=5.3 Hz, 1H), 7.99 (d, J=8.0 Hz, 2H), 7.72 (d, J=8.0 Hz, 2H), 7.56 (d, J=5.3 Hz, 1H), 7.17 (d, J=8.3 Hz, 2H), 7.11 (d, J=8.3 Hz, 2H), 3.56 (s, 3H); ¹³C NMR (125 MHz, CDCl₃) δ 166.29, 162.58, 162.36, 158.17, 142.16, 139.11, 133.13 (q, CCF₃, J=33.1 Hz), 132.93, 129.39, 128.27, 127.71, 126.04 (q, ArCF₃, J=3.8 Hz), 123.87 (q, CF₃, J=272.4 Hz), 116.51, 37.39.

### N-(4-chlorophenyl)-6-(4-isopropylphenyl)-N-methylpyrazine-2-carboxamide (APZ-2217)

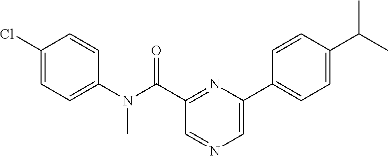 [SMILES: CC(C)c1ccc(-c2cncc(C(=O)N(C)c3ccc(Cl)cc3)n2)cc1]

N-(4-chlorophenyl)-6-(4-isopropylphenyl)-N-methylpyrazine-2-carboxamide was prepared via General Procedure A from 6-(4-isopropylphenyl)pyrazine-2-carboxylic acid. The crude product was purified by flash column chromatography with hexanes/ethyl acetate, followed by recrystallization in dichloromethane/hexanes to afford title compound as a lustrous off-white solid (68%). mp 113-115° C.; R_{f} (30% EA/Hex)=0.26; ¹H NMR (500 MHz, CDCl₃) δ 8.98-8.66 (m, 2H), 7.63-7.33 (m, 2H), 7.31-7.23 (m, 4H), 7.17-6.93 (m, 2H), 3.55 (s, 3H), 2.95 (hept, J=6.9 Hz, 1H), 1.27 (d, J=6.9 Hz, 6H); ¹³C NMR (125 MHz, CDCl₃) δ 166.19, 151.49, 150.43, 147.47, 143.24, 143.12, 142.07, 133.00, 132.79, 129.51, 128.24, 127.13, 127.02, 34.08, 23.88.

### N-(4-chlorophenyl)-4-(4-isopropylphenyl)-N-methylpyrimidine-2-carboxamide (APZ-2218)

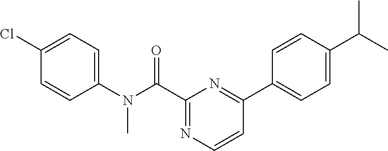 [SMILES: CC(C)c1ccc(-c2ccnc(C(=O)N(C)c3ccc(Cl)cc3)n2)cc1]

N-(4-chlorophenyl)-4-(4-isopropylphenyl)-N-methylpyrimidine-2-carboxamide was prepared via General Procedure A from 4-(4-isopropylphenyl)pyrimidine-2-carboxylic acid. The crude product was purified by flash column chromatography with hexanes/ethyl acetate, followed by recrystallization in dichloromethane/hexanes to afford title compound as a white solid (73%). mp 134-136° C.; R_{f} (50% EA/Hex)=0.32; ¹H NMR (500 MHz, CDCl₃) δ 8.60 (d, J=4.9 Hz, 1H), 7.81 (d, J=7.9 Hz, 2H), 7.48 (d, J=4.9 Hz, 1H), 7.31 (d, J=7.9 Hz, 2H), 7.16 (d, J=8.2 Hz, 2H), 7.11 (d, J=8.2 Hz, 2H), 3.54 (s, 3H), 2.96 (hept, J=7.0 Hz, 1H), 1.28 (d, J=7.0 Hz, 6H); ¹³C NMR (125 MHz, CDCl₃) δ 166.67, 163.84, 162.28, 157.46, 152.92, 142.32, 133.41, 132.69, 129.30, 128.20, 127.42, 127.19, 115.86, 37.35, 34.21, 23.87.

### morpholino(6-(4-(trifluoromethyl)phenyl)pyrazin-2-yl)methanone (APZ-2233)

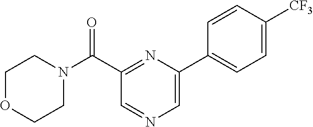 [SMILES: O=C(c1cncc(-c2ccc(C(F)(F)F)cc2)n1)N1CCOCC1]

morpholino(6-(4-(trifluoromethyl)phenyl)pyrazin-2-yl)methanone was prepared via General Procedure A from 6-(4-(trifluoromethyl)phenyl)pyrazine-2-carboxylic acid. The crude product was purified by flash column chromatography with hexanes/ethyl acetate, followed by recrystallization in dichloromethane/hexanes to afford title compound as a fluffy white solid (85%). mp 102-104° C.; R_{f} (70% EA/Hex)=0.31; ¹H NMR (500 MHz, CDCl₃) δ 9.14 (s, 1H), 8.97 (s, 1H), 8.14 (d, J=8.2 Hz, 2H), 7.80 (d, J=8.2 Hz, 2H), 3.92-3.83 (m, 4H), 3.75 (s, 4H); ¹³C NMR (125 MHz CDCl₃) δ 165.05, 148.83, 148.27, 144.68, 142.66, 138.91, 132.31 (q, CCF₃, J=32.7 Hz), 127.42, 126.31 (q, ArCF₃, J=3.8 Hz), 123.96 (q, CF₃, J=272.4 Hz), 67.07, 66.95, 47.95, 43.05.

### piperidin-1-yl(6-(4-(trifluoromethyl)phenyl)pyrazin-2-yl)methanone (APZ-2234)

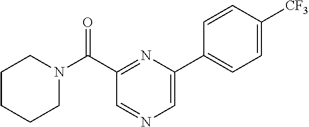 [SMILES: O=C(c1cncc(-c2ccc(C(F)(F)F)cc2)n1)N1CCCCC1]

piperidin-1-yl(6-(4-(trifluoromethyl)phenyl)pyrazin-2-yl)methanone was prepared via General Procedure A from 6-(4-(trifluoromethyl)phenyl)pyrazine-2-carboxylic acid. The crude product was purified by flash column chromatography with hexanes/ethyl acetate, followed by recrystallization in dichloromethane/hexanes to afford title compound as a lustrous light yellow solid 54%). mp 78-80° C.; R_{f} (50% EA/Hex)=0.28; ¹H NMR (500 MHz, CDCl₃) δ 9.11 (s, 1H), 8.87 (s, 1H), 8.17 (d, J=8.2 Hz, 2H), 7.79 (d, J=8.2 Hz, 2H), 3.86-3.74 (m, 2H), 3.59-3.48 (m, 2H), 1.80-1.73 (m, 4H), 1.71-1.63 (m, 2H); ¹³C NMR (175 MHz, CDCl₃) δ 165.09, 149.39, 148.98, 143.97, 142.16, 139.13, 132.12 (q, CCF₃, J=32.8 Hz), 127.45, 126.20 (q, ArCF₃, J=3.8 Hz), 124.01 (q, CF₃, J=272.3 Hz), 48.58, 43.71, 26.71, 25.75, 24.61.

### (4-methylpiperidin-1-yl)(6-(4-(trifluoromethyl)phenyl)pyrazin-2-yl)methanone (APZ-2235)

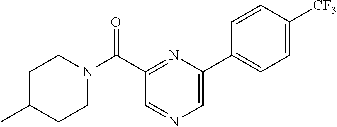 [SMILES: CC1CCN(C(=O)c2cncc(-c3ccc(C(F)(F)F)cc3)n2)CC1]

(4-methylpiperidin-1-yl)(6-(4-(trifluoromethyl)phenyl)pyrazin-2-yl)methanone was prepared via General Procedure A from 6-(4-(trifluoromethyl)phenyl)pyrazine-2-carboxylic acid. The crude product was purified by flash column chromatography with hexanes/ethyl acetate, followed by recrystallization in dichloromethane/hexanes to afford title compound as a lustrous white solid (59%). mp 75-77° C.; R_{f} (50% EA/Hex)=0.39; ¹H NMR (500 MHz, CDCl₃) δ 9.10 (s, 1H), 8.87 (s, 1H), 8.16 (d, J=8.1 Hz, 2H), 7.79 (d, J=8.1 Hz, 2H), 4.82-4.66 (m, 1H), 4.03-3.90 (m, 1H), 3.14 (td, J=12.9, 2.8 Hz, 1H), 2.87 (td, J=12.9, 2.8 Hz, 1H), 1.88-1.79 (m, 1H), 1.74-1.63 (m, 2H), 1.41-1.25 (m, 2H), 1.02 (d, J=6.4 Hz, 3H); ¹³C NMR (125 MHz, CDCl₃) δ 164.97, 149.27, 148.88, 143.87, 142.07, 139.02, 132.02 (q, CCF₃, J=32.7 Hz), 127.34, 126.10 (q, ArCF₃, J=3.7 Hz), 123.90 (q, CF₃, J=272.4 Hz), 47.71, 42.95, 34.71, 33.77, 31.11, 21.70.

### N-cyclopentyl-N-methyl-6-(4-(trifluoromethyl)phenyl)pyrazine-2-carboxamide (APZ-2236)

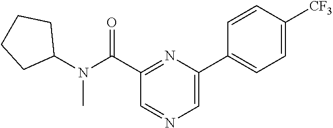 [SMILES: CN(C(=O)c1cncc(-c2ccc(C(F)(F)F)cc2)n1)C1CCCC1]

N-cyclopentyl-N-methyl-6-(4-(trifluoromethyl)phenyl)pyrazine-2-carboxamide was prepared via General Procedure A from 6-(4-(trifluoromethyl)phenyl)pyrazine-2-carboxylic acid. The crude product was purified by flash column chromatography with hexanes/ethyl acetate, followed by preparatory TLC (40% EA/Hex) to afford title compound as a white solid (36%). mp 95-96° C.; R_{f} (50% EA/Hex)=0.39; ¹H NMR (500 MHz, CDCl₃, rotameric mixture, both forms reported) δ 9.11 (s, 0.65H), 9.09 (s, 0.35H), 8.90 (s, 0.35H), 8.84 (s, 0.65H), 8.23-8.12 (m, 2H), 7.84-7.72 (m, 2H), 5.11 (p, J=8.2 Hz, 0.35H), 4.24 (p, J=8.2 Hz, 0.65H), 3.06 (s, 2H), 3.01 (s, 1H), 2.07-1.33 (m, 8H); ¹³C NMR (125 MHz, CDCl₃, rotameric mixture, major form reported) δ 166.89, 149.98, 149.14, 143.61, 142.13, 139.18, 132.13 (q, CCF₃, J=32.9 Hz), 127.42, 126.22 (q, ArCF₃, J=4.0 Hz), 124.03 (q, CF₃, J=272.5 Hz), 60.07, 55.47, 29.62, 28.03, 24.62.

### (4-methyl-1,4-diazepan-1-yl)(6-(4-(trifluoromethyl)phenyl)pyrazin-2-yl)methanone (APZ-2237)

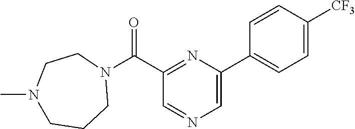 [SMILES: CN1CCCN(C(=O)c2cncc(-c3ccc(C(F)(F)F)cc3)n2)CC1]

(4-methyl-1,4-diazepan-1-yl)(6-(4-(trifluoromethyl)phenyl)pyrazin-2-yl)methanone was prepared via General Procedure A from 6-(4-(trifluoromethyl)phenyl)pyrazine-2-carboxylic acid. The crude product was purified by flash column chromatography with dichloromethane/methanol, followed by preparatory TLC (4% MeOH/DCM) to afford title compound as a light yellow oil (38%). R_{f} (5% MeOH/DCM)=0.26; ¹H NMR (500 MHz, CDCl₃) δ 9.11 (s, 1H), 8.92 (d, J=5.7 Hz, 1H), 8.15 (d, J=8.1 Hz, 2H), 7.79 (d, J=8.1 Hz, 2H), 3.94-3.81 (m, 2H), 3.75 (dd, J=4.6 Hz, 1H), 3.68 (t, J=6.3 Hz, 1H), 2.82 (dd, J=4.9 Hz, 1H), 2.74 (dd, J=4.6 Hz, 1H), 2.71 (dd, J=5.4 Hz, 1H), 2.67 (dd, J=5.4 Hz, 1H), 2.45 and 2.40 (s, 3H), 2.08 (p, J=6.0 Hz, 1H), 1.97 (p, J=6.0 Hz, 1H).

### N-methyl-N-(1-methylpyrrolidin-3-yl)-6-(4-(trifluoromethyl)phenyl)pyrazine-2-carboxamide (APZ-2238)

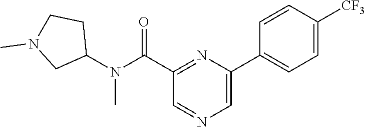 [SMILES: CN1CCC(N(C)C(=O)c2cncc(-c3ccc(C(F)(F)F)cc3)n2)C1]

N-methyl-N-(1-methylpyrrolidin-3-yl)-6-(4-(trifluoromethyl)phenyl)pyrazine-2-carboxamide was prepared via General Procedure A from 6-(4-(trifluoromethyl)phenyl)pyrazine-2-carboxylic acid. The crude product was purified by flash column chromatography with dichloromethane/methanol, followed by preparatory TLC (8% MeOH/DCM) to afford title compound as a yellow oil (47%). R_{f} (10% MeOH/DCM)=0.22; ¹H NMR (500 MHz, CDCl₃, rotameric mixture, both forms reported) δ 9.11 (s, 0.6H), 9.10 (s, 0.4H), 8.89 (s, 0.4H), 8.86 (s, 0.6H), 8.24-8.09 (m, 2H), 7.84-7.74 (m, 2H), 5.44-5.34 (m, 0.4H), 4.55 (tt, J=9.9, 5.4 Hz, 0.6H), 3.18 (s, 2H), 3.15 (s, 1H), 2.99-2.92 (m, 0.4H), 2.89 (dd, J=10.6, 3.7 Hz, 0.4H), 2.81-2.72 (m, 1.2H), 2.66-2.58 (m, 0.4H), 2.58-2.51 (m, 0.6H), 2.41-2.26 (m, 4.45H), 2.17-2.10 (m, 0.55H), 2.08-1.98 (m, 0.6H), 1.98-1.90 (m, 0.4H); ¹³C NMR (125 MHz, CDCl₃, rotameric mixture, major form reported) δ 166.69, 149.60, 149.05, 143.92, 142.29, 139.09, 132.14 (q, CCF₃, J=32.8 Hz), 127.44, 126.21 (q, ArCF₃, J=3.8 Hz), 123.99 (q, CF₃, J=272.3 Hz), 59.65, 58.34, 55.94, 42.05, 30.05, 28.87.

### N-methyl-N-(tetrahydrofuran-3-yl)-6-(4-(trifluoromethyl)phenyl)pyrazine-2-carboxamide (APZ-2239)

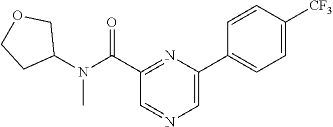 [SMILES: CN(C(=O)c1cncc(-c2ccc(C(F)(F)F)cc2)n1)C1CCOC1]

N-methyl-N-(tetrahydrofuran-3-yl)-6-(4-(trifluoromethyl)phenyl)pyrazine-2-carboxamide was prepared via General Procedure A from 6-(4-(trifluoromethyl)phenyl)pyrazine-2-carboxylic acid. The crude product was purified by flash column chromatography with hexanes/ethyl acetate, followed by recrystallization in dichloromethane/hexanes to afford title compound as a light yellow solid (51%). mp 108-110° C.; R_{f} (70% EA/Hex)=0.30; ¹H NMR (500 MHz, CDCl₃, rotameric mixture, both forms reported) δ 9.14 (s, 0.55H), 9.12 (s, 0.45H), 8.92 (s, 0.55H), 8.91 (s, 0.45H), 8.21-8.10 (m, 2H), 7.85-7.74 (m, 2H), 5.47 (tt, J=8.8, 4.9 Hz, 0.45H), 4.72 (tt, J=8.8, 4.9 Hz, 0.55H), 4.11 (dtd, J=21.5, 8.7, 4.6 Hz, 1H), 3.98 (td, J=10.1, 3.6 Hz, 1H), 3.94-3.86 (m, 0.5H), 3.82-3.69 (m, 1H), 3.63 (q, J=8.3 Hz, 0.5H), 3.15 (s, 1.65H), 3.12 (s, 1.35H), 2.44 (dtd, J=13.2, 8.4, 4.7 Hz, 0.5H), 2.29-2.18 (m, 0.5H), 2.18-2.10 (m, 0.5H), 2.05 (dtd, J=13.2, 8.4, 4.7 Hz, 0.5H); ¹³C NMR (125 MHz, CDCl₃, rotameric mixture, major form reported) δ 166.61, 149.23, 148.90, 144.41, 142.50, 138.98, 132.25 (q, CCF₃, J=32.6 Hz), 127.39, 126.29 (q, ArCF₃, J=3.9 Hz), 123.95 (q, CF₃, J=272.5 Hz), 70.87, 68.06, 59.03, 30.79, 28.69.

### N-(5-chloropyridin-2-yl)-N-methyl-6-(4-(trifluoromethyl)phenyl)pyrazine-2-carboxamide (APZ-2240)

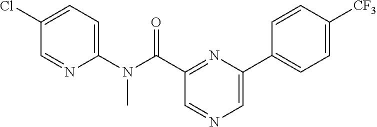 [SMILES: CN(C(=O)c1cncc(-c2ccc(C(F)(F)F)cc2)n1)c1ccc(Cl)cn1]

N-(5-chloropyridin-2-yl)-N-methyl-6-(4-(trifluoromethyl)phenyl)pyrazine-2-carboxamide was prepared via General Procedure A from 6-(4-(trifluoromethyl)phenyl)pyrazine-2-carboxylic acid. The crude product was purified by flash column chromatography with hexanes/ethyl acetate to afford title compound as a white solid (52%). mp 116-118° C.; R_{f} (30% EA/Hex)=0.24; ¹H NMR (500 MHz, CDCl₃) δ 9.08 (s, 1H), 9.01 (s, 1H), 8.23 (d, J=2.6 Hz, 1H), 7.67 (s, 4H), 7.64 (dd, J=8.6, 2.6 Hz, 1H), 7.18 (d, J=8.6 Hz, 1H), 3.64 (s, 3H); ¹³C NMR (125 MHz, CDCl₃) δ 166.58, 154.95, 148.38, 147.75, 147.24, 144.84, 142.60, 138.65, 137.75, 132.14 (q, CCF₃, J=32.8 Hz), 129.54, 127.12, 126.02 (q, ArCF₃, J=3.8 Hz), 123.94 (q, CF₃, J=272.5 Hz), 120.53, 36.59.

### N-(6-chloropyridazin-3-yl)-N-methyl-6-(4-(trifluoromethyl)phenyl)pyrazine-2-carboxamide (APZ-2241)

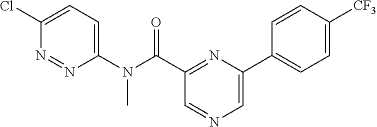 [SMILES: CN(C(=O)c1cncc(-c2ccc(C(F)(F)F)cc2)n1)c1ccc(Cl)nn1]

N-(6-chloropyridazin-3-yl)-N-methyl-6-(4-(trifluoromethyl)phenyl)pyrazine-2-carboxamide was prepared via General Procedure A from 6-(4-(trifluoromethyl)phenyl)pyrazine-2-carboxylic acid. The crude product was purified by flash column chromatography with hexanes/ethyl acetate to afford title compound as a fluffy white solid (66%). mp 155-158° C.; R_{f} (40% EA/Hex)=0.19; ¹H NMR (500 MHz, CDCl₃) δ 9.16 (s, 1H), 9.08 (s, 1H), 7.86-7.67 (m, 4H), 7.65 (d, J=9.1 Hz, 1H), 7.50 (d, J=9.1 Hz, 1H), 3.72 (s, 3H); ¹³C NMR (125 MHz, CDCl₃) δ 166.75, 159.03, 154.31, 148.45, 146.78, 145.18, 143.30, 138.45, 132.31 (q, CCF₃, J=32.7 Hz), 129.45, 127.14, 126.15 (q, ArCF₃, J=3.8 Hz), 126.01, 123.89 (q, CF₃, J=272.4 Hz), 36.93.

### N-(1,1-dioxidotetrahydrothiophen-3-yl)-N-methyl-6-(4-(trifluoromethyl)phenyl)pyrazine-2-carboxamide (APZ-2242)

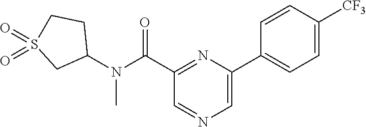 [SMILES: CN(C(=O)c1cncc(-c2ccc(C(F)(F)F)cc2)n1)C1CCS(=O)(=O)C1]

N-(1,1-dioxidotetrahydrothiophen-3-yl)-N-methyl-6-(4-(trifluoromethyl)phenyl)pyrazine-2-carboxamide was prepared via General Procedure A from 6-(4-(trifluoromethyl)phenyl)pyrazine-2-carboxylic acid. The crude product was purified by flash column chromatography with hexanes/ethyl acetate, followed by recrystallization in dichloromethane/hexanes to afford title compound as a light yellow solid (77%). mp 170-174° C.; R_{f} (50% EA/Hex)=0.15; ¹H NMR (500 MHz, CDCl₃, rotameric mixture, both forms reported) δ 9.16 (s, 1H), 9.04 (s, 0.4H), 8.96 (s, 0.6H), 8.15 (d, J=8.1 Hz, 1.2H), 8.09 (d, J=8.1 Hz, 0.8H), 7.88-7.75 (m, 2H), 5.43 (tt, J=17.8, 9.1 Hz, 0.6H), 5.13 (tt, J=17.8, 9.1 Hz, 0.4H), 3.56-3.46 (m, 1H), 3.46-3.36 (m, 0.6H), 3.35-3.27 (m, 0.4H), 3.28-3.09 (m, 4.55H), 3.01-2.91 (m, 0.45H), 2.65-2.40 (m, 2H); ¹³C NMR (175 MHz, CDCl₃, rotameric mixture, major form reported) δ 167.05, 148.99, 148.05, 144.56, 143.09, 138.76, 132.37 (q, CCF₃, J=32.7 Hz), 127.51, 126.29 (q, ArCF₃, J=3.8 Hz), 123.92 (q, CF₃, J=272.5 Hz), 51.42, 51.09, 50.76, 33.11, 25.86.

### N-methyl-N-(pyrimidin-4-yl)-6-(4-(trifluoromethyl)phenyl)pyrazine-2-carboxamide (APZ-2243)

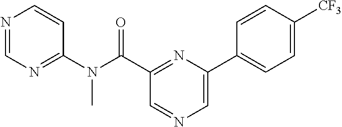 [SMILES: CN(C(=O)c1cncc(-c2ccc(C(F)(F)F)cc2)n1)c1ccncn1]

N-methyl-N-(pyrimidin-4-yl)-6-(4-(trifluoromethyl)phenyl)pyrazine-2-carboxamide was prepared via General Procedure A from 6-(4-(trifluoromethyl)phenyl)pyrazine-2-carboxylic acid. The crude product was purified by flash column chromatography with hexanes/ethyl acetate, followed by preparatory TLC (40% EA/Hex) to afford title compound as a light yellow solid (59%). mp 74-77° C.; R_{f} (50% EA/Hex)=0.30; ¹H NMR (500 MHz, CDCl₃) δ 9.11 (s, 1H), 9.08 (s, 1H), 8.83 (s, 1H), 8.64 (d, J=5.6 Hz, 1H), 7.80 (d, J=8.3 Hz, 2H), 7.68 (d, J=8.3 Hz, 2H), 7.38 (d, J=5.6 Hz, 1H), 3.68 (s, 3H); ¹³C NMR (125 MHz, CDCl₃) δ 167.79, 162.21, 158.12, 157.80, 148.55, 147.63, 144.39, 143.11, 138.45, 132.17 (q, CCF₃, J=32.7 Hz), 127.10, 126.10 (q, ArCF₃, J=3.8 Hz), 123.85 (q, CF₃, J=272.2 Hz), 114.35, 35.36.

### N-methyl-N-(pyridin-4-yl)-6-(4-(trifluoromethyl)phenyl)pyrazine-2-carboxamide (APZ-2244)

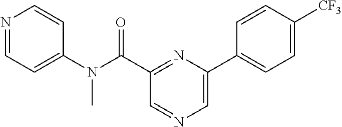 [SMILES: CN(C(=O)c1cncc(-c2ccc(C(F)(F)F)cc2)n1)c1ccncc1]

N-methyl-N-(pyridin-4-yl)-6-(4-(trifluoromethyl)phenyl)pyrazine-2-carboxamide was prepared via General Procedure A from 6-(4-(trifluoromethyl)phenyl)pyrazine-2-carboxylic acid. The crude product was purified by flash column chromatography with hexanes/ethyl acetate to afford title compound as a fluffy white solid (53%). mp 97-100° C.; R_{f} (70% EA/Hex)=0.31; ¹H NMR (500 MHz, CDCl₃) δ 9.08 (s, 1H), 9.02 (s, 1H), 8.55 (d, J=5.5 Hz, 2H), 7.66 (d, J=8.1 Hz, 2H), 7.56 (d, J=8.1 Hz, 2H), 7.06 (d, J=5.5 Hz, 2H), 3.61 (s, 3H); ¹³C NMR (125 MHz, CDCl₃) δ 165.75, 151.95, 150.94, 148.58, 146.81, 144.66, 142.90, 138.32, 132.11 (q, CCF₃, J=32.8 Hz), 127.05, 125.97 (q, ArCF₃, J=3.8 Hz), 123.78 (q, CF₃, J=272.5 Hz), 120.75, 37.93.

### N-methyl-N-(thiazol-5-yl)-6-(4-(trifluoromethyl)phenyl)pyrazine-2-carboxamide (APZ-2249)

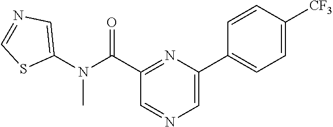 [SMILES: CN(C(=O)c1cncc(-c2ccc(C(F)(F)F)cc2)n1)c1cncs1]

N-methyl-N-(thiazol-5-yl)-6-(4-(trifluoromethyl)phenyl)pyrazine-2-carboxamide was prepared via General Procedure D from N-(thiazol-5-yl)-6-(4-(trifluoromethyl)phenyl)pyrazine-2-carboxamide. The crude product was purified by flash column chromatography with hexanes/ethyl acetate to afford title compound as an off-white solid (29%). mp 156-159° C.; R_{f} (50% EA/Hex)=0.24; ¹H NMR (500 MHz, CDCl₃, rotameric mixture, both forms reported) δ 9.22 (s, 0.6H), 9.08 (s, 0.6H), 9.05-8.89 (m, 0.8H), 8.61 (s, 1H), 8.20 (d, J=8.1 Hz, 1H), 7.95-7.62 (m, 3.3H), 7.56-7.40 (m, 0.4H), 3.78 (s, 1.8H), 3.58 (s, 1.2H); ¹³C NMR (175 MHz, CDCl₃, rotameric mixture, major form reported) δ 163.96, 152.09, 148.93, 147.41, 145.03, 143.33, 138.57, 132.55 (q, CCF₃, J=32.2 Hz), 132.11, 130.12, 127.53, 126.37, 123.93 (q, CF₃, J=272.5 Hz), 39.00.

### 4-chlorophenyl 6-(4-(trifluoromethyl)phenyl)pyrazine-2-carboxylate (APZ-2269)

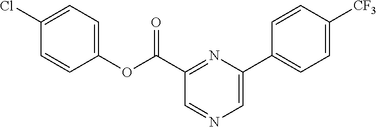 [SMILES: O=C(Oc1ccc(Cl)cc1)c1cncc(-c2ccc(C(F)(F)F)cc2)n1]

4-chlorophenyl 6-(4-(trifluoromethyl)phenyl)pyrazine-2-carboxylate was prepared via General Procedure A from 6-(4-(trifluoromethyl)phenyl)pyrazine-2-carboxylic acid and 4-chlorophenol. The crude product was purified by flash column chromatography with hexanes/ethyl acetate to afford title compound as an off-white solid (quantitative). mp 86-88° C.; R_{f} (20% EA/Hex)=0.49; ¹H NMR (500 MHz, (CD₃)₂CO) δ 9.59 (s, 1H), 9.40 (s, 1H), 8.50 (d, J=8.2 Hz, 2H), 7.95 (d, J=8.2 Hz, 2H), 7.55 (dt, J=8.9, 3.2, 2.2 Hz, 2H), 7.44 (dt, J=8.9, 3.2, 2.2 Hz, 2H); ¹³C NMR (175 MHz, (CD₃)₂CO) δ 163.08, 151.29, 150.56, 146.61, 146.16, 143.16, 140.07, 132.35 (q, CCF₃, J=32.3 Hz), 132.02, 130.48, 128.83, 126.89 (q, ArCF₃, J=3.8 Hz), 125.15 (q, CF₃, J=271.5 Hz), 124.43.

### (4-(4-chlorophenyl)piperazin-1-yl)(6-(4-(trifluoromethyl)phenyl)pyrazin-2-yl)methanone (APZ-2270)

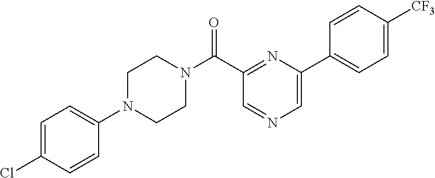 [SMILES: O=C(c1cncc(-c2ccc(C(F)(F)F)cc2)n1)N1CCN(c2ccc(Cl)cc2)CC1]

(4-(4-chlorophenyl)piperazin-1-yl)(6-(4-(trifluoromethyl)phenyl)pyrazin-2-yl)methanone was prepared via General Procedure A from 6-(4-(trifluoromethyl)phenyl)pyrazine-2-carboxylic acid. The crude product was purified by flash column chromatography with hexanes/ethyl acetate to afford title compound as a fluffy yellow solid (53%). mp 53-56° C.; R_{f} (50% EA/Hex)=0.21; ¹H NMR (500 MHz, CDCl₃) δ 9.15 (s, 1H), 8.98 (s, 1H), 8.16 (d, J=8.2 Hz, 2H), 7.80 (d, J=8.2 Hz, 2H), 7.24 (dt, J=9.0, 3.3, 2.1 Hz, 2H), 6.87 (dt, J=9.0, 3.3, 2.1 Hz, 2H), 4.03 (dd, J=5.1 Hz, 2H), 3.88 (dd, J=5.0 Hz, 2H), 3.33 (dd, J=5.1 Hz, 2H), 3.22 (dd, J=5.0 Hz, 2H); ¹³C NMR (175 MHz, (CD₃)₂CO) δ 165.55, 150.99, 149.63, 149.26, 145.16, 143.63, 140.52, 132.03 (q, CCF₃, J=32.3 Hz), 129.67, 128.67, 126.84 (q, ArCF₃, J=3.8 Hz), 125.18 (q, CF₃, J=271.5 Hz), 124.88, 118.59, 50.32, 49.60, 47.64, 42.77.

### N-methyl-N-(1-methyl-1H-pyrazol-4-yl)-6-(4-(trifluoromethyl)phenyl)pyrazine-2-carboxamide (APZ-2271)

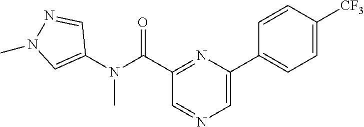 [SMILES: CN(C(=O)c1cncc(-c2ccc(C(F)(F)F)cc2)n1)c1cnn(C)c1]

N-methyl-N-(1-methyl-1H-pyrazol-4-yl)-6-(4-(trifluoromethyl)phenyl)pyrazine-2-carboxamide was prepared via General Procedure E from N-(1-methyl-1H-pyrazol-4-yl)-6-(4-(trifluoromethyl)phenyl)pyrazine-2-carboxamide. The crude product was purified by flash column chromatography with hexanes/ethyl acetate to afford title compound as a tan solid (42%). mp 144-145° C.; R_{f} (100% EA)=0.30; ¹H NMR (500 MHz, CDCl₃, rotameric mixture, major form reported) δ 8.95 (s, 1H), 8.76 (s, 1H), 7.93 (d, J=8.1 Hz, 2H), 7.74 (d, J=8.1 Hz, 2H), 7.28 (s, 1H), 7.21 (s, 1H), 3.75 (s, 3H), 3.48 (s, 3H); ¹³C NMR (125 MHz, CDCl₃, rotameric mixture, major form reported) δ 166.58, 149.23, 148.89, 143.44, 142.07, 139.03, 136.60, 132.08 (q, CCF₃, J=32.6 Hz), 127.38, 126.71, 126.09 (q, ArCF₃, J=3.8 Hz), 123.99 (q, CF₃, J=272.5 Hz), 123.61, 39.52, 39.11.

### N-methyl-N-(5-methylisoxazol-3-yl)-6-(4-(trifluoromethyl)phenyl)pyrazine-2-carboxamide (APZ-2272)

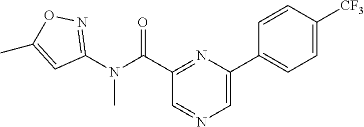 [SMILES: Cc1cc(N(C)C(=O)c2cncc(-c3ccc(C(F)(F)F)cc3)n2)no1]

N-methyl-N-(5-methylisoxazol-3-yl)-6-(4-(trifluoromethyl)phenyl)pyrazine-2-carboxamide was prepared via General Procedure E from N-(5-methylisoxazol-3-yl)-6-(4-(trifluoromethyl)phenyl)pyrazine-2-carboxamide. The crude product was purified by flash column chromatography with hexanes/ethyl acetate to afford title compound as a lustrous white solid (59%). mp 144-146° C.; R_{f} (30% EA/Hex)=0.22; ¹H NMR (500 MHz, (CD₃)₂CO) δ 9.38 (s, 1H), 8.99 (s, 1H), 8.46-8.03 (m, 2H), 7.89 (d, J=8.2 Hz, 2H), 6.52 (bs, 1H), 3.53 (s, 3H), 2.39 (s, 3H); ¹³C NMR (175 MHz, (CD₃)₂CO) 6171.52, 166.80, 149.25, 148.58, 145.00 (2 non-equivalent C), 144.27, 140.26, 132.12 (q, CCF₃, J=32.3 Hz), 128.58, 126.78 (q, ArCF₃, J=3.9 Hz), 125.16 (q, CF₃, J=271.5 Hz), 99.38, 36.38, 12.42.

### N-(4-chlorophenyl)-N-methyl-1-(4-(trifluoromethyl)phenyl)-1H-1,2,4-triazole-3-carboxamide (APZ-2301)

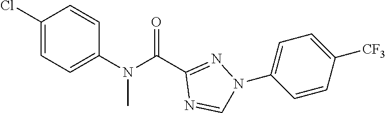 [SMILES: CN(C(=O)c1ncn(-c2ccc(C(F)(F)F)cc2)n1)c1ccc(Cl)cc1]

N-(4-chlorophenyl)-N-methyl-1-(4-(trifluoromethyl)phenyl)-1H-1,2,4-triazole-3-carboxamide was prepared via General Procedure F from 1-(4-(trifluoromethyl)phenyl)-1H-1,2,4-triazole-3-carboxylic acid. The crude product was purified by flash column chromatography with hexanes/ethyl acetate, followed by recrystallization in dichloromethane/hexanes to afford title compound as a fluffy tan solid (31%). mp 165-167° C.; R_{f} (70% EA/Hex)=0.50; ¹H NMR (500 MHz, CDCl₃) δ 8.56 (s, 1H), 7.72 (d, J=8.1 Hz, 2H), 7.67-7.49 (m, 2H), 7.31 (d, J=8.1 Hz, 2H), 7.23-7.04 (m, 2H), 3.53 (s, 3H); ¹³C NMR (125 MHz, CDCl₃) δ 160.97, 158.56, 142.51, 141.15, 138.99, 133.39, 130.66 (q, CCF₃, J=32.8 Hz), 129.51, 128.54, 127.26 (q, ArCF₃, J=3.7 Hz), 123.57 (q, CF₃, J=272.4 Hz), 119.88, 38.34.

### N-methyl-N-(3-methylisoxazol-5-yl)-6-(4-(trifluoromethyl)phenyl)pyrazine-2-carboxamide (APZ-2247)

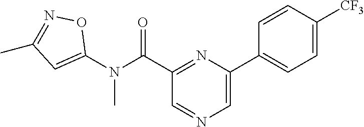 [SMILES: Cc1cc(N(C)C(=O)c2cncc(-c3ccc(C(F)(F)F)cc3)n2)on1]

N-methyl-N-(3-methylisoxazol-5-yl)-6-(4-(trifluoromethyl)phenyl)pyrazine-2-carboxamide was prepared via General Procedure D from N-(3-methylisoxazol-5-yl)-6-(4-(trifluoromethyl)phenyl)pyrazine-2-carboxamide. The crude product was purified by flash column chromatography with hexanes/ethyl acetate to afford title compound as a white solid (41%). mp 110-113° C.; R_{f} (40% EA/Hex)=0.36; ¹H NMR (500 MHz, CDCl₃) δ 9.13 (s, 1H), 9.08 (s, 1H), 8.18-7.81 (m, 2H), 7.74 (d, J=8.0 Hz, 2H), 5.94 (bs, 1H), 3.59 (s, 3H), 2.24 (s, 3H); ¹³C NMR (175 MHz, CDCl₃) δ 164.96, 161.56, 148.89, 146.37, 144.51, 143.58 (2 non-equivalent C), 138.53, 132.27 (q, CCF₃, J=32.7 Hz), 127.39, 126.11 (q, ArCF₃, J=3.8 Hz), 123.92 (q, CF₃, J=272.5 Hz), 95.20, 36.86, 12.02.

### N-methyl-N-(5-methylthiazol-2-yl)-6-(4-(trifluoromethyl)phenyl)pyrazine-2-carboxamide (APZ-2261)

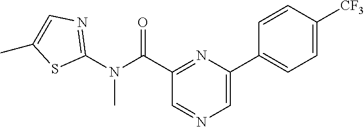 [SMILES: Cc1cnc(N(C)C(=O)c2cncc(-c3ccc(C(F)(F)F)cc3)n2)s1]

N-methyl-N-(5-methylthiazol-2-yl)-6-(4-(trifluoromethyl)phenyl)pyrazine-2-carboxamide was prepared via General Procedure D from N-(5-methylthiazol-2-yl)-6-(4-(trifluoromethyl)phenyl)pyrazine-2-carboxamide. The crude product was purified by flash column chromatography with hexanes/ethyl acetate to afford title compound as a white solid (16%). mp 222-224° C.; R_{f} (80% EA/Hex)=0.35; ¹H NMR (500 MHz, CDCl₃) δ 9.56 (s, 1H), 9.14 (s, 1H), 8.30 (d, J=8.1 Hz, 2H), 7.77 (d, J=8.1 Hz, 2H), 6.78 (s, 1H), 3.87 (s, 3H), 2.35 (s, 3H); ¹³C NMR (175 MHz, CDCl₃) δ 170.56, 168.04, 150.43, 148.81, 145.05, 142.98, 139.66, 131.75 (q, CCF₃, J=32.5 Hz), 127.70, 125.94 (q, ArCF₃, J=3.8 Hz), 124.14 (q, CF₃, J=272.2 Hz), 123.45, 123.26, 35.76, 12.78.

### N-methyl-N-(thiazol-2-yl)-6-(4-(trifluoromethyl)phenyl)pyrazine-2-carboxamide (APZ-2258)

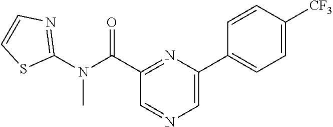 [SMILES: CN(C(=O)c1cncc(-c2ccc(C(F)(F)F)cc2)n1)c1nccs1]

N-methyl-N-(thiazol-2-yl)-6-(4-(trifluoromethyl)phenyl)pyrazine-2-carboxamide was prepared via General Procedure D from N-(thiazol-2-yl)-6-(4-(trifluoromethyl)phenyl)pyrazine-2-carboxamide. The crude product was purified by flash column chromatography with hexanes/ethyl acetate to afford title compound as a yellow solid (36%). mp 187-193° C.; R_{f} (80% EA/Hex)=0.30; ¹H NMR (500 MHz, CDCl₃) δ 9.58 (s, 1H), 9.14 (s, 1H), 8.30 (d, J=8.0 Hz, 2H), 7.77 (d, J=8.0 Hz, 2H), 7.11 (d, J=4.6 Hz, 1H), 6.82 (d, J=4.6 Hz, 1H), 3.94 (s, 3H); ¹³C NMR (175 MHz, CDCl₃) δ 170.97, 168.74, 150.53, 148.64, 145.07, 143.14, 139.64, 131.84 (q, CCF₃, J=32.5 Hz), 127.73, 127.30, 125.99 (q, ArCF₃, J=3.8 Hz), 124.15 (q, CF₃, J=272.3 Hz), 110.50, 36.20.

### N-(4-chlorophenyl)-N-methyl-6-(2-methyl-4-(trifluoromethyl)phenyl)pyrazine-2-carboxamide (APZ-2274)

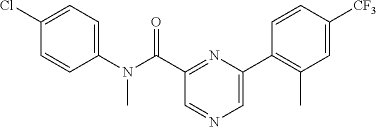 [SMILES: Cc1cc(C(F)(F)F)ccc1-c1cncc(C(=O)N(C)c2ccc(Cl)cc2)n1]

N-(4-chlorophenyl)-N-methyl-6-(2-methyl-4-(trifluoromethyl)phenyl)pyrazine-2-carboxamide was prepared via General Procedure A from 6-(2-methyl-4-(trifluoromethyl)phenyl)pyrazine-2-carboxylic acid. The crude product was purified by flash column chromatography with hexanes/ethyl acetate, followed by recrystallization in dichloromethane/hexanes to afford title compound as a fluffy white solid (49%). mp 120-121° C.; R_{f} (40% EA/Hex)=0.33; ¹H NMR (500 MHz, (CD₃)₂CO) δ 8.88 (s, 1H), 8.75 (s, 1H), 7.64 (s, 1H), 7.58 (d, J=8.0 Hz, 1H), 7.34 (d, J=8.3 Hz, 2H), 7.32-7.10 (m, 3H), 3.50 (s, 3H), 2.19 (s, 3H); ¹³C NMR (175 MHz, CDCl₃) δ 166.22, 151.92, 148.40, 145.03, 143.68, 142.79, 139.32, 137.01, 133.00, 131.50 (q, CCF₃, J=32.5 Hz), 130.54, 129.65, 128.37, 127.70 (q, ArCF₃, J=3.0 Hz), 123.98 (q, CF₃, J=272.6 Hz), 123.01 (q, ArCF₃, J=3.9 Hz), 38.36, 19.97.

### N-(4-chloro-2-methylphenyl)-N-methyl-6-(6-(trifluoromethyl)pyridin-3-yl)pyrazine-2-carboxamide (APZ-2294)

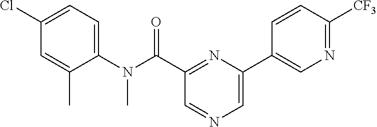 [SMILES: Cc1cc(Cl)ccc1N(C)C(=O)c1cncc(-c2ccc(C(F)(F)F)nc2)n1]

N-(4-chloro-2-methylphenyl)-N-methyl-6-(6-(trifluoromethyl)pyridin-3-yl)pyrazine-2-carboxamide was prepared via General Procedure E from N-(4-chloro-2-methylphenyl)-6-(6-(trifluoromethyl)pyridin-3-yl)pyrazine-2-carboxamide. The crude product was purified by flash column chromatography with hexanes/ethyl acetate to afford title compound as a fluffy white solid (66%). mp 99-101° C.; R_{f} (50% EA/Hex)=0.51; ¹H NMR (500 MHz, CDCl₃, rotameric mixture, major form reported) δ 9.05 (s, 1H), 8.99 (d, J=2.2 Hz, 1H), 8.97 (s, 1H), 7.91 (dd, J=8.2, 2.2 Hz, 1H), 7.75 (d, J=8.2 Hz, 1H), 7.23 (d, J=2.4 Hz, 1H), 7.09 (dd, J=8.3, 2.4 Hz, 1H), 7.00 (d, J=8.3 Hz, 1H), 3.44 (s, 3H), 2.31 (s, 3H); ¹³C NMR (125 MHz, CDCl₃) δ 165.52, 149.21 (q, CCF₃, J=35.1 Hz), 148.25, 148.10, 146.43, 145.04, 142.38, 141.64, 137.51, 135.85, 133.94, 133.85, 131.20, 129.45, 127.31, 121.42 (q, CF₃, J=274.3 Hz), 120.62 (q, ArCF₃, J=2.7 Hz), 37.73, 17.96.

### N-(5-chloro-3-methylpyridin-2-yl)-N-methyl-6-(6-(trifluoromethyl)pyridin-3-yl)pyrazine-2-carboxamide (APZ-2295)

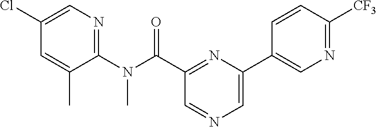 [SMILES: Cc1cc(Cl)cnc1N(C)C(=O)c1cncc(-c2ccc(C(F)(F)F)nc2)n1]

N-(5-chloro-3-methylpyridin-2-yl)-N-methyl-6-(6-(trifluoromethyl)pyridin-3-yl)pyrazine-2-carboxamide was prepared via General Procedure E from N-(5-chloro-3-methylpyridin-2-yl)-6-(6-(trifluoromethyl)pyridin-3-yl)pyrazine-2-carboxamide. The crude product was purified by flash column chromatography with hexanes/ethyl acetate to afford title compound as a white solid (46%). mp 120-122° C.; R_{f} (50% EA/Hex)=0.34; ¹H NMR (500 MHz, CDCl₃, rotameric mixture, major form reported) δ 9.21 (s, 1H), 9.00 (s, 1H), 8.88 (d, J=2.2 Hz, 1H), 8.08 (d, J=2.5 Hz, 1H), 7.92 (dd, J=8.1, 2.2 Hz, 1H), 7.74 (d, J=8.1 Hz, 1H), 7.59 (d, J=2.5 Hz, 1H), 3.46 (s, 3H), 2.38 (s, 3H); ¹³C NMR (125 MHz, CDCl₃) δ 165.96, 154.29, 149.24 (q, CCF₃, J=35.2 Hz), 148.07, 147.60, 146.02, 145.92, 145.41, 142.67, 139.44, 135.42, 133.80, 131.56, 131.16, 121.39 (q, CF₃, J=274.3 Hz), 120.55 (q, ArCF₃, J=2.8 Hz), 35.97, 17.65.

### N-(4-chloro-2-methoxyphenyl)-N-methyl-6-(6-(trifluoromethyl)pyridin-3-yl)pyrazine-2-carboxamide (APZ-2296)

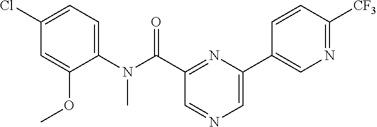 [SMILES: COc1cc(Cl)ccc1N(C)C(=O)c1cncc(-c2ccc(C(F)(F)F)nc2)n1]

N-(4-chloro-2-methoxyphenyl)-N-methyl-6-(6-(trifluoromethyl)pyridin-3-yl)pyrazine-2-carboxamide was prepared via General Procedure E from N-(4-chloro-2-methoxyphenyl)-6-(6-(trifluoromethyl)pyridin-3-yl)pyrazine-2-carboxamide. The crude product was purified by flash column chromatography with hexanes/ethyl acetate to afford title compound as a light yellow solid (80%). mp 135-137° C.; R_{f} (50% EA/Hex)=0.35; ¹H NMR (500 MHz, CDCl₃) δ 9.01 (d, J=2.2 Hz, 1H), 8.99 (s, 1H), 8.96 (s, 1H), 7.97 (dd, J=8.3, 2.2 Hz, 1H), 7.74 (d, J=8.2 Hz, 1H), 7.17 (d, J=8.2 Hz, 1H), 6.92 (dd, J=8.3, 2.2 Hz, 1H), 6.77 (d, J=2.2 Hz, 1H), 3.69 (s, 3H), 3.44 (s, 3H); ¹³C NMR (125 MHz, CDCl₃) δ 166.33, 154.75, 149.13 (q, CCF₃, J=35.1 Hz), 148.68, 148.10, 146.02, 144.78, 142.02, 135.75, 134.47, 133.98, 131.78, 129.47, 121.43 (q, CF₃, J=274.3 Hz), 121.14, 120.58 (q, ArCF₃, J=2.7 Hz), 112.65, 55.95, 37.53.

### N-(4-chloro-2-methoxyphenyl)-N-(methyl-d₃)-6-(6-(trifluoromethyl)pyridin-3-yl)pyrazine-2-carboxamide (APZ-2297)

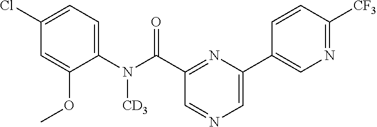 [SMILES: [2H]C([2H])([2H])N(C(=O)c1cncc(-c2ccc(C(F)(F)F)nc2)n1)c1ccc(Cl)cc1OC]

N-(4-chloro-2-methoxyphenyl)-N-(methyl-d₃)-6-(6-(trifluoromethyl)pyridin-3-yl)pyrazine-2-carboxamide was prepared via General Procedure E from N-(4-chloro-2-methoxyphenyl)-6-(6-(trifluoromethyl)pyridin-3-yl)pyrazine-2-carboxamide with iodomethane-d₃ in place of iodomethane. The crude product was purified by flash column chromatography with hexanes/ethyl acetate to afford title compound as a light yellow solid (79%). mp 139-140° C.; R_{f} (50% EA/Hex)=0.38; ¹H NMR (500 MHz, CDCl₃) δ 9.01 (d, J=2.2 Hz, 1H), 8.99 (s, 1H), 8.96 (s, 1H), 7.99 (dd, J=8.3, 2.2 Hz, 1H), 7.74 (d, J=8.3 Hz, 1H), 7.17 (d, J=8.3 Hz, 1H), 6.92 (dd, J=8.3, 2.2 Hz, 1H), 6.77 (d, J=2.2 Hz, 1H), 3.69 (s, 3H); ¹³C NMR (125 MHz, CDCl₃) δ 166.35, 154.76, 149.13 (q, CCF₃, J=35.0 Hz), 148.68, 148.10, 146.02, 144.78, 142.03, 135.75, 134.46, 133.99, 131.75, 129.46, 121.44 (q, CF₃, J=274.1 Hz), 121.14, 120.58 (q, ArCF₃, J=2.8 Hz), 112.64, 55.95, 37.03-36.58 (m, CD₃).

### N-(4-chlorophenyl)-N-(methyl-d₃)-6-(4-(trifluoromethoxy)phenyl)pyrazine-2-carboxamide (APZ-2298)

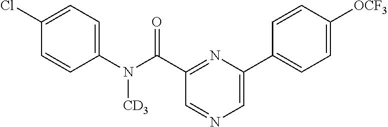 [SMILES: [2H]C([2H])([2H])N(C(=O)c1cncc(-c2ccc(OC(F)(F)F)cc2)n1)c1ccc(Cl)cc1]

N-(4-chlorophenyl)-N-(methyl-d₃)-6-(4-(trifluoromethoxy)phenyl)pyrazine-2-carboxamide was prepared via General Procedure E from N-(4-chlorophenyl)-6-(4-(trifluoromethoxy)phenyl)pyrazine-2-carboxamide with iodomethane-d₃ in place of iodomethane. The crude product was purified by flash column chromatography with hexanes/ethyl acetate to afford title compound as a lustrous light yellow solid (92%). mp 116-118° C.; R_{f} (50% EA/Hex)=0.35; ¹H NMR (500 MHz, CDCl₃) δ 8.93 (s, 1H), 8.88 (s, 1H), 7.68-7.42 (m, 2H), 7.36-7.16 (m, 4H), 7.17-6.94 (m, 2H); ¹³C NMR (125 MHz, CDCl₃) δ 165.83, 150.89-150.75 (m, COCF₃), 148.94, 147.58, 143.98, 143.20, 142.04, 133.96, 132.95, 129.59, 128.59, 128.28, 121.26, 120.50 (q, OCF₃, J=258.2 Hz).

### N-(4-chloro-2-methoxyphenyl)-N-methyl-6-(4-(trifluoromethoxy)phenyl)pyrazine-2-carboxamide (APZ-2299)

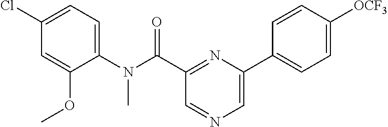 [SMILES: COc1cc(Cl)ccc1N(C)C(=O)c1cncc(-c2ccc(OC(F)(F)F)cc2)n1]

N-(4-chloro-2-methoxyphenyl)-N-methyl-6-(4-(trifluoromethoxy)phenyl)pyrazine-2-carboxamide was prepared via General Procedure E from N-(4-chloro-2-methoxyphenyl)-6-(4-(trifluoromethoxy)phenyl)pyrazine-2-carboxamide with iodomethane-d₃ in place of iodomethane. The crude product was purified by flash column chromatography with hexanes/ethyl acetate, followed by recrystallization in dichloromethane/hexanes to afford title compound as a lustrous white solid (95%). mp 124-125° C.; R_{f} (50% EA/Hex)=0.42; ¹H NMR (500 MHz, CDCl₃) δ 8.91 (s, 1H), 8.86 (s, 1H), 7.58 (d, J=8.7 Hz, 2H), 7.29-7.22 (m, 2H), 7.16 (d, J=8.3 Hz, 1H), 6.92 (dd, J=8.3, 2.2 Hz, 1H), 6.77 (d, J=2.2 Hz, 1H), 3.66 (s, 3H), 3.43 (s, 3H); ¹³C NMR (125 MHz, CDCl₃) δ 166.60, 154.77, 150.79-150.63 (m, COCF₃), 148.55, 147.98, 143.50, 141.86, 134.20, 134.14, 132.20, 129.37, 128.55, 121.19, 121.09, 120.50 (q, OCF₃, J=257.9 Hz), 112.66, 55.89, 37.67.

### N-(4-chloro-2-methoxyphenyl)-N-(methyl-d₃)-6-(4-(trifluoromethoxy)phenyl)pyrazine-2-carboxamide (APZ-2300)

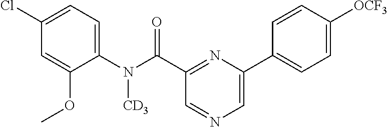 [SMILES: [2H]C([2H])([2H])N(C(=O)c1cncc(-c2ccc(OC(F)(F)F)cc2)n1)c1ccc(Cl)cc1OC]

N-(4-chloro-2-methoxyphenyl)-N-(methyl-d₃)-6-(4-(trifluoromethoxy)phenyl)pyrazine-2-carboxamide was prepared via General Procedure E from N-(4-chloro-2-methoxyphenyl)-6-(4-(trifluoromethoxy)phenyl)pyrazine-2-carboxamide with iodomethane-d₃ in place of iodomethane. The crude product was purified by flash column chromatography with hexanes/ethyl acetate, followed by recrystallization in dichloromethane/hexanes to afford title compound as a lustrous white solid (90%). mp 123-124° C.; R_{f} (30% EA/Hex)=0.12; ¹H NMR (500 MHz, CDCl₃) δ 8.91 (s, 1H), 8.86 (s, 1H), 7.58 (dt, J=8.7, 2.9, 2.1 Hz, 2H), 7.29-7.22 (m, 2H), 7.16 (d, J=8.3 Hz, 1H), 6.92 (dd, J=8.3, 2.2 Hz, 1H), 6.78 (d, J=2.2 Hz, 1H), 3.66 (s, 3H); ¹³C NMR (125 MHz, CDCl₃) δ 166.62, 154.78, 150.78-150.66 (m, COCF₃), 148.55, 147.98, 143.51, 141.87, 134.19, 134.15, 132.17, 129.36, 128.55, 121.20, 121.09, 120.51 (q, OCF₃, J=257.9 Hz), 112.66, 55.89, 37.38-36.47 (m, CD₃).

### N-(4-chlorophenyl)-N-methyl-6-((4-(trifluoromethyl)phenyl)amino)pyrazine-2-carboxamide (APZ-2302)

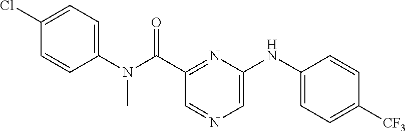 [SMILES: CN(C(=O)c1cncc(Nc2ccc(C(F)(F)F)cc2)n1)c1ccc(Cl)cc1]

N-(4-chlorophenyl)-N-methyl-6-((4-(trifluoromethyl)phenyl)amino)pyrazine-2-carboxamide was prepared via General Procedure A from 6-((4-(trifluoromethyl)phenyl)amino)pyrazine-2-carboxylic acid. The crude product was purified by flash column chromatography with hexanes/ethyl acetate to afford title compound as a yellow solid (30%). mp 209-211° C.; R_{f} (70% EA/Hex)=0.35; ¹H NMR (500 MHz, CD₃OD) δ 8.17 (s, 1H), 8.10 (s, 1H), 7.55 (d, J=8.4 Hz, 2H), 7.48 (s, 2H), 7.26-7.13 (m, 4H), 3.52 (s, 3H); ¹³C NMR (175 MHz, (CD₃)₂SO) δ 166.08, 149.38, 145.28, 143.42, 142.73, 136.14, 134.47, 130.67, 129.00, 128.09, 125.88 (q, ArCF₃, J=3.7 Hz), 124.64 (q, CF₃, J=270.9 Hz), 121.41 (q, CCF₃, J=31.8 Hz), 117.77, 37.47.

### N-(4-chlorophenyl)-N-methyl-1-(p-tolyl)-1H-1,2,4-triazole-3-carboxamide (APZ-2078)

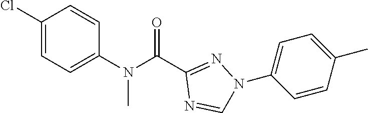 [SMILES: Cc1ccc(-n2cnc(C(=O)N(C)c3ccc(Cl)cc3)n2)cc1]

N-(4-chlorophenyl)-N-methyl-1-(p-tolyl)-1H-1,2,4-triazole-3-carboxamide was prepared using General Procedure A from 1-(p-tolyl)-1H-1,2,4-triazole-3-carboxylic acid. The crude product was purified by flash column chromatography with hexanes/ethyl acetate to afford the title compound as a white solid (11%). mp 189-192° C.; R_{f} (70% EA/Hex)=0.40; ¹H NMR (500 MHz, CD₃OD) δ 8.87 (s, 1H), 7.43 (s, 2H), 7.36 (d, J=6.9 Hz, 2H), 7.30 (d, J=7.3 Hz, 2H), 7.25 (s, 2H), 3.51 (s, 3H), 2.38 (s, 3H).

### N-(4-chlorophenyl)-1-(4-methoxyphenyl)-N-methyl-1H-1,2,4-triazole-3-carboxamide (APZ-2079)

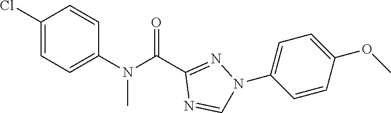 [SMILES: COc1ccc(-n2cnc(C(=O)N(C)c3ccc(Cl)cc3)n2)cc1]

N-(4-chlorophenyl)-1-(4-methoxyphenyl)-N-methyl-1H-1,2,4-triazole-3-carboxamide was prepared using General Procedure A from 1-(4-methoxyphenyl)-1H-1,2,4-triazole-3-carboxylic acid. The crude product was purified by flash column chromatography with hexanes/ethyl acetate to afford the title compound as an off-white solid (31%). mp 143-145° C.; R_{f} (70% EA/Hex)=0.28; ¹H NMR (500 MHz, CDCl₃) δ 8.25 (s, 1H), 7.39 (bs, 2H), 7.27 (bs, 2H), 7.14 (bs, 2H), 6.95 (d, J=8.7 Hz, 2H), 3.84 (s, 3H), 3.52 (s, 3H); ¹³C NMR (175 MHz, CDCl₃) δ 161.41, 159.68, 157.98, 142.69, 140.48, 132.85, 129.88, 129.27, 128.26, 121.67, 114.81, 55.63, 38.05.

### N-(4-chlorophenyl)-1-(p-tolyl)-1H-1,2,4-triazole-3-carboxamide (APZ-2080)

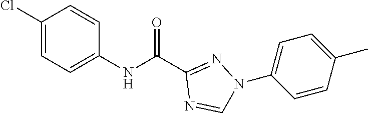 [SMILES: Cc1ccc(-n2cnc(C(=O)Nc3ccc(Cl)cc3)n2)cc1]

N-(4-chlorophenyl)-1-(p-tolyl)-1H-1,2,4-triazole-3-carboxamide was prepared using General Procedure A from 1-(p-tolyl)-1H-1,2,4-triazole-3-carboxylic acid. The crude product was purified by flash column chromatography with hexanes/ethyl acetate to afford the title compound as a white solid (10%). mp>260° C.; R_{f} (70% EA/Hex)=0.74; ¹H NMR (500 MHz, CDCl₃) δ 8.98 (s, 1H), 8.56 (s, 1H), 7.71 (d, J=8.8 Hz, 2H), 7.64 (d, J=8.4 Hz, 2H), 7.35 (t, J=8.1 Hz, 4H), 2.44 (s, 3H); ¹³C NMR (175 MHz, CDCl₃) δ 157.41, 156.47, 141.48, 139.35, 135.95, 134.18, 130.42, 129.71, 129.19, 121.09, 120.33, 21.13.

### 1-(4-methoxyphenyl)-N-methyl-N-(p-tolyl)-1H-1,2,4-triazole-3-carboxamide (APZ-2081)

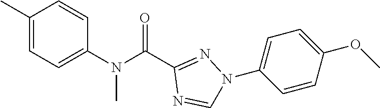

1-(4-methoxyphenyl)-N-methyl-N-(p-tolyl)-1H-1,2,4-triazole-3-carboxamide was prepared using General Procedure A from 1-(4-methoxyphenyl)-1H-1,2,4-triazole-3-carboxylic acid. The crude product was purified by flash column chromatography with hexanes/ethyl acetate to afford the title compound as a white solid (63%). mp 129-134° C.; R_{f} (70% EA/Hex)=0.25; ¹H NMR (500 MHz, CDCl₃) δ 8.21 (s, 1H), 7.49-7.31 (m, 2H), 7.08 (s, 4H), 6.93 (d, J=8.5 Hz, 2H), 3.83 (s, 3H), 3.51 (s, 3H), 2.32 (s, 3H); ¹³C NMR (175 MHz, CDCl₃) δ 161.73, 159.55, 158.40, 141.52, 140.29, 136.92, 129.68, 126.65, 121.63, 114.71, 55.62, 38.05, 21.03.

### N-(3,4-dimethylphenyl)-1-(4-methoxyphenyl)-N-methyl-1H-1,2,4-triazole-3-carboxamide (APZ-2082)

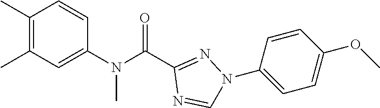

N-(3,4-dimethylphenyl)-1-(4-methoxyphenyl)-N-methyl-1H-1,2,4-triazole-3-carboxamide was prepared using General Procedure A from 1-(4-methoxyphenyl)-1H-1,2,4-triazole-3-carboxylic acid. The crude product was purified by flash column chromatography with hexanes/ethyl acetate to afford the title compound as a white solid (47%). mp 104-106° C.; R_{f} (10% EA/Hex)=0.28; ¹H NMR (500 MHz, CDCl₃) δ 8.21 (s, 1H), 7.39 (d, J=8.4 Hz, 2H), 7.04-6.97 (m, 2H), 6.94 (d, J=8.4 Hz, 2H), 6.88 (d, J=7.9 Hz, 1H), 3.84 (s, 3H), 3.50 (s, 3H), 2.21 (s, 3H), 2.20 (s, 3H); ¹³C NMR (175 MHz, CDCl₃) δ 161.78, 159.54, 158.51, 141.70, 140.29, 137.43, 135.56, 130.06, 130.05, 127.71, 124.16, 121.65, 114.72, 55.62, 38.08, 19.78, 19.35.

### N-(3,4-dimethylphenyl)-N-methyl-1-(p-tolyl)-1H-1,2,4-triazole-3-carboxamide (APZ-2083)

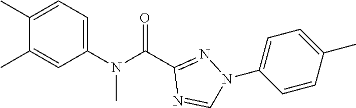

N-(3,4-dimethylphenyl)-N-methyl-1-(p-tolyl)-1H-1,2,4-triazole-3-carboxamide was prepared using General Procedure A from 1-(p-tolyl)-1H-1,2,4-triazole-3-carboxylic acid. The crude product was purified by flash column chromatography with hexanes/ethyl acetate to afford the title compound as a white solid (11%). mp 144-147° C.; R_{f} (70% EA/Hex)=0.36; ¹H NMR (500 MHz, CDCl₃) δ 8.26 (s, 1H), 7.45-7.30 (m, 2H), 7.24 (d, J=7.0 Hz, 2H), 7.08-6.94 (m, 2H), 6.93-6.81 (m, 1H), 3.50 (s, 3H), 2.38 (s, 3H), 2.20 (d, J=8.0 Hz, 6H).

### N,1-bis(4-methoxyphenyl)-N-methyl-1H-1,2,4-triazole-3-carboxamide (APZ-2318)

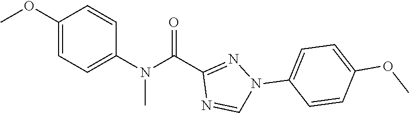

N,1-bis(4-methoxyphenyl)-N-methyl-1H-1,2,4-triazole-3-carboxamide was prepared using General Procedure A from 1-(4-methoxyphenyl)-1H-1,2,4-triazole-3-carboxylic acid. The crude product was purified by flash column chromatography with hexanes/ethyl acetate to afford the title compound as an off-white solid (71%). mp 129-131° C.; R_{f} (70% EA/Hex)=0.19; ¹H NMR (500 MHz, CD₃OD) δ 8.76 (s, 1H), 7.46 (d, J=8.7 Hz, 2H), 7.14 (d, J=8.6 Hz, 2H), 7.00 (d, J=8.8 Hz, 2H), 6.86 (d, J=8.6 Hz, 2H), 3.82 (s, 3H), 3.75 (s, 3H), 3.46 (s, 3H); ¹³C NMR (175 MHz, CD₃OD) δ 163.79, 161.34, 160.54, 158.75, 142.73, 137.70, 131.26, 129.52, 122.68, 115.84, 115.36, 56.15, 55.99, 38.45.

### N-(4-methoxyphenyl)-N-methyl-1-(p-tolyl)-1H-1,2,4-triazole-3-carboxamide (APZ-2319)

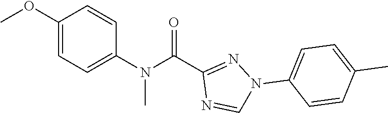

N-(4-methoxyphenyl)-N-methyl-1-(p-tolyl)-1H-1,2,4-triazole-3-carboxamide was prepared using General Procedure A from 1-(p-tolyl)-1H-1,2,4-triazole-3-carboxylic acid. The crude product was purified by flash column chromatography with hexanes/ethyl acetate to afford the title compound as a white solid (25%). mp 150-152° C.; R_{f} (70% EA/Hex)=0.29; ¹H NMR (500 MHz, CD₃OD) δ 8.83 (s, 1H), 7.43 (d, J=8.2 Hz, 2H), 7.28 (d, J=8.1 Hz, 2H), 7.15 (d, J=8.6 Hz, 2H), 6.86 (d, J=8.6 Hz, 2H), 3.75 (s, 3H), 3.46 (s, 3H), 2.37 (s, 3H); ¹³C NMR (175 MHz, CD₃OD) δ 163.70, 160.53, 158.82, 142.81, 140.07, 137.68, 135.67, 131.26, 129.52, 120.86, 115.36, 55.98, 38.46, 21.02.

### N-(4-chlorophenyl)-N,4-dimethyl-2-(p-tolyl)thiazole-5-carboxamide (APZ-2320)

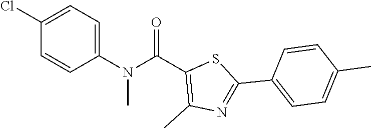 [SMILES: Cc1ccc(-c2nc(C)c(C(=O)N(C)c3ccc(Cl)cc3)s2)cc1]

N-(4-chlorophenyl)-N,4-dimethyl-2-(p-tolyl)thiazole-5-carboxamide was prepared using General Procedure A from 4-methyl-2-(p-tolyl)thiazole-5-carboxylic acid. The crude product was purified by flash column chromatography with hexanes/ethyl acetate to afford the title compound (46%). ¹H NMR (500 MHz, CD₃OD) δ 7.65 (d, J=8.1 Hz, 2H), 7.38 (d, J=8.5 Hz, 2H), 7.27 (d, J=8.6 Hz, 2H), 7.24 (d, J=8.0 Hz, 2H), 3.45 (s, 3H), 2.43 (s, 3H), 2.36 (s, 3H).

### N,5-bis(4-chlorophenyl)-N-methylnicotinamide (APZ-2321)

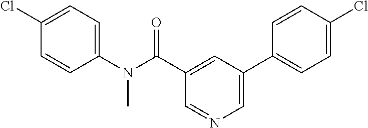 [SMILES: CN(C(=O)c1cncc(-c2ccc(Cl)cc2)c1)c1ccc(Cl)cc1]

N,5-bis(4-chlorophenyl)-N-methylnicotinamide was prepared using General Procedure C from 5-bromo-N-(4-chlorophenyl)-N-methylnicotinamide. The crude product was purified by flash column chromatography with hexanes/ethyl acetate to afford the title compound as a white solid (70%). mp 110-113° C.; R_{f} (50% EA/Hex)=0.19; ¹H NMR (500 MHz, CDCl₃) δ 8.69 (s, 1H), 8.43 (s, 1H), 7.81 (s, 1H), 7.43 (d, J=8.4 Hz, 2H), 7.34 (d, J=8.3 Hz, 2H), 7.30-7.25 (m, 2H), 7.04 (d, J=8.3 Hz, 2H), 3.52 (s, 3H); ¹³C NMR (175 MHz, CDCl₃) δ 167.72, 148.80, 148.39, 142.74, 135.21, 134.85, 134.52, 133.16, 131.31, 129.87, 129.42, 128.35, 128.32, 38.47.

### N,4-bis(4-chlorophenyl)-N-methylpicolinamide (APZ-2123)

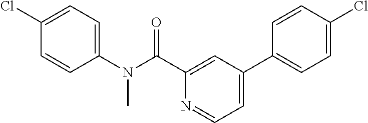 [SMILES: CN(C(=O)c1cc(-c2ccc(Cl)cc2)ccn1)c1ccc(Cl)cc1]

N,4-bis(4-chlorophenyl)-N-methylpicolinamide was prepared using General Procedure C from 5-bromo-N-(4-chlorophenyl)-N-methylnicotinamide. The crude product was purified by flash column chromatography with hexanes/ethyl acetate to afford the title compound as a white solid (5%). mp 134-136° C.; R_{f} (70% EA/Hex)=0.58; ¹H NMR (500 MHz, CDCl₃) δ 8.36 (bs, 1H), 7.77 (s, 1H), 7.51 (d, J=8.2 Hz, 2H), 7.45 (d, J=8.4 Hz, 2H), 7.36 (bs, 1H), 7.20 (bs, 2H), 7.04 (bs, 2H), 3.52 (s, 3H); ¹³C NMR (175 MHz, CDCl₃) δ 168.53, 154.50, 148.95, 147.78, 143.12, 135.81, 135.75, 132.21, 129.46, 129.21, 128.23, 127.91, 121.82, 121.66, 38.19.

### N-(4-chlorophenyl)-N-methyl-4-(p-tolyl)picolinamide (APZ-2124)

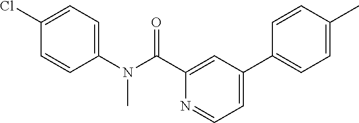 [SMILES: Cc1ccc(-c2ccnc(C(=O)N(C)c3ccc(Cl)cc3)c2)cc1]

N-(4-chlorophenyl)-N-methyl-4-(p-tolyl)picolinamide was prepared using General Procedure A from 5-bromo-N-(4-chlorophenyl)-N-methylnicotinamide. The crude product was purified by flash column chromatography with hexanes/ethyl acetate to afford the title compound as a white solid (56%). mp 123-130° C.; R_{f} (30% EA/Hex)=0.13; ¹H NMR (500 MHz, CDCl₃) δ 8.34 (s, 1H), 7.78 (s, 1H), 7.48 (d, J=6.9 Hz, 2H), 7.38 (s, 1H), 7.31-7.25 (m, 2H), 7.20 (s, 2H), 7.05 (s, 2H), 3.52 (s, 3H), 2.41 (s, 3H); ¹³C NMR (175 MHz, CDCl₃) δ 168.80, 154.27, 148.85, 148.80, 143.13, 139.64, 134.37, 132.10, 129.91, 129.16, 127.87, 126.76, 121.78, 121.54, 38.14, 21.23.

### N,2-bis(4-chlorophenyl)-N,4-dimethylthiazole-5-carboxamide (APZ-2125)

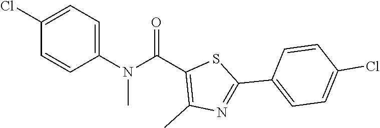 [SMILES: Cc1nc(-c2ccc(Cl)cc2)sc1C(=O)N(C)c1ccc(Cl)cc1]

N,2-bis(4-chlorophenyl)-N,4-dimethylthiazole-5-carboxamide was prepared using General Procedure A from 2-(4-chlorophenyl)-4-methylthiazole-5-carboxylic acid. The crude product was purified by flash column chromatography with hexanes/ethyl acetate to afford the title compound as an orange solid (94%). mp 111-113° C.; R_{f} (50% EA/Hex)=0.65; ¹H NMR (500 MHz, CD₃OD) δ 7.76 (d, J=8.1 Hz, 2H), 7.43 (d, J=8.1 Hz, 2H), 7.38 (d, J=8.0 Hz, 2H), 7.27 (d, J=8.0 Hz, 2H), 3.45 (s, 3H), 2.44 (s, 3H); ¹³C NMR (175 MHz, CD₃OD) δ 168.56, 164.84, 157.36, 143.59, 137.91, 134.73, 132.47, 130.79, 130.42, 130.04, 128.95, 126.31, 38.67, 17.14.

### N-(4-chlorophenyl)-N-methyl-5-(p-tolyl)nicotinamide (APZ-2160)

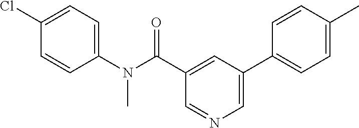 [SMILES: Cc1ccc(-c2cncc(C(=O)N(C)c3ccc(Cl)cc3)c2)cc1]

N-(4-chlorophenyl)-N-methyl-5-(p-tolyl)nicotinamide was prepared using General Procedure A from 5-bromo-N-(4-chlorophenyl)-N-methylnicotinamide. The crude product was purified by flash column chromatography with hexanes/ethyl acetate to afford the title compound as a white solid (26%). mp 128-131° C.; R_{f} (50% EA/Hex)=0.24; ¹H NMR (500 MHz, CDCl₃) δ 8.71 (s, 1H), 8.41 (s, 1H), 7.82 (s, 1H), 7.31 (d, J=7.8 Hz, 2H), 7.29-7.23 (m, 4H), 7.04 (d, J=8.2 Hz, 2H), 3.52 (s, 3H), 2.40 (s, 3H); ¹³C NMR (175 MHz, CDCl₃) δ 168.03, 148.91, 147.81, 142.82, 138.52, 135.87, 134.35, 133.84, 133.03, 131.18, 129.89, 129.81, 128.34, 126.89, 38.45, 21.16.

### 2,5-bis(4-chlorophenyl)-4,5-dihydro-6H-pyrrolo[3,4-d]thiazol-6-one (APZ-2324)

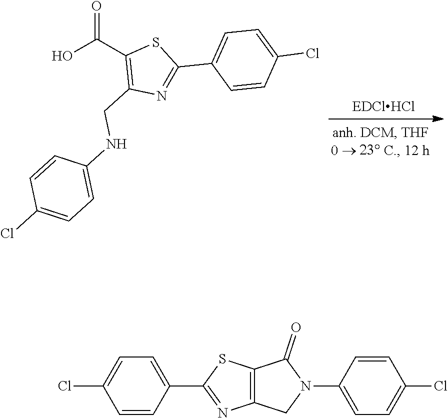 [SMILES: O=C(O)c1sc(-c2ccc(Cl)cc2)nc1CNc1ccc(Cl)cc1 ; O=C1c2sc(-c3ccc(Cl)cc3)nc2CN1c1ccc(Cl)cc1]

2,5-bis(4-chlorophenyl)-4,5-dihydro-6H-pyrrolo[3,4-d]thiazol-6-one was prepared using the following procedure on a 0.264 mmol scale. In a round bottom flask, 2-(4-chlorophenyl)-4-(((4-chlorophenyl)amino)methyl)thiazole-5-carboxylic acid (0.264 mmol, 1.0 equiv) was dissolved in anhydrous dichloromethane (0.1 M) and anhydrous tetrahydrofuran (0.1 M). The reaction mixture was then cooled to 0° C. and EDC HCl (0.791 mmol, 3.0 equiv) was added and stirred for 1 hour at 0° C. After 1 hour, the reaction was allowed to warm to room temperature and stirred for 23 hours. The reaction mixture was concentrated in vacuo, diluted with water, and extracted thrice with dichloromethane. The combined organic layers were dried over sodium sulfate, filtered, and concentrated in vacuo. The crude product was purified by flash column chromatography with hexanes/ethyl acetate to afford the title compound as a yellow solid (33%). mp>260° C.; R_{f} (20% EA/Hex)=0.47; ¹H NMR (500 MHz, (CD₃)₂SO) δ 8.09 (d, J=8.5 Hz, 2H), 7.87 (d, J=8.9 Hz, 2H), 7.65 (d, J=8.5 Hz, 2H), 7.50 (d, J=8.9 Hz, 2H), 5.17 (s, 2H).

### N-(4-chlorophenyl)-N-(methyl-d₃)-5-(4-(trifluoromethyl)phenyl)nicotinamide (APZ-2178)

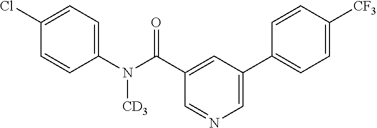 [SMILES: [2H]C([2H])([2H])N(C(=O)c1cncc(-c2ccc(C(F)(F)F)cc2)c1)c1ccc(Cl)cc1]

N-(4-chlorophenyl)-N-(methyl-d₃)-5-(4-(trifluoromethyl)phenyl)nicotinamide was prepared using General Procedure E from N-(4-chlorophenyl)-5-(4-(trifluoromethyl)phenyl)nicotinamide. The crude product was purified by flash column chromatography with hexanes/ethyl acetate to afford the title compound as an off-white solid (16%). mp 227-232° C.; R_{f} (10% MeOH/DCM)=0.15; ¹H NMR (500 MHz, CD₃OD) δ 9.54 (s, 1H), 9.52 (s, 1H), 9.42 (s, 1H), 8.15 (d, J=8.1 Hz, 2H), 7.96 (d, J=8.1 Hz, 2H), 7.83 (d, J=8.6 Hz, 2H), 7.41 (d, J=8.6 Hz, 2H); ¹³C NMR (175 MHz, CD₃OD) δ 161.66, 147.09, 146.04, 142.63, 140.75, 138.20, 138.08, 136.48, 133.44 (q, CCF₃, J=32.6 Hz), 131.40, 130.08, 129.76, 127.73 (q, ArCF₃, J=3.7 Hz), 125.37 (q, CF₃, J=271.5 Hz), 123.40.

### 4-(4-chlorophenyl)-N-methyl-N-(p-tolyl)picolinamide (APZ-2322)

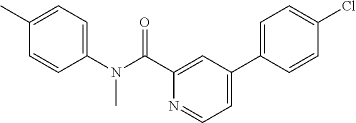 [SMILES: Cc1ccc(N(C)C(=O)c2cc(-c3ccc(Cl)cc3)ccn2)cc1]

4-(4-chlorophenyl)-N-methyl-N-(p-tolyl)picolinamide was prepared using General Procedure C from 4-bromo-N-methyl-N-(p-tolyl)picolinamide. The crude product was purified by flash column chromatography with hexanes/ethyl acetate to afford the title compound as a white solid (30%). mp 126-128° C.; R_{f} (60% EA/Hex)=0.39; ¹H NMR (500 MHz, CDCl₃) δ 8.38 (s, 1H), 7.61 (s, 1H), 7.49-7.39 (m, 4H), 7.30 (s, 1H), 7.08-6.89 (m, 4H), 3.52 (s, 3H), 2.26 (s, 3H); ¹³C NMR (125 MHz, CDCl₃) δ 168.70, 155.08, 149.09, 147.31, 141.81, 136.44, 136.00, 135.57, 129.68, 129.37, 128.20, 126.51, 121.46, 121.40, 38.07, 20.94.

### 5-(3-chlorophenyl)-N-(4-chlorophenyl)-N-methylnicotinamide (APZ-2323)

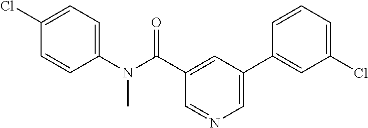 [SMILES: CN(C(=O)c1cncc(-c2cccc(Cl)c2)c1)c1ccc(Cl)cc1]

5-(3-chlorophenyl)-N-(4-chlorophenyl)-N-methylnicotinamide was prepared using General Procedure A from 5-(3-chlorophenyl)nicotinic acid. The crude product was purified by flash column chromatography with hexanes/ethyl acetate to afford the title compound as an off-white solid (14%). mp 123-125° C.; R_{f} (EA/Hex)=0.48; ¹H NMR (700 MHz, CDCl₃) δ 8.69 (s, 1H), 8.47 (s, 1H), 7.81 (s, 1H), 7.41-7.35 (m, 3H), 7.32-7.29 (m, 1H), 7.28 (d, J=8.6 Hz, 2H), 7.05 (d, J=8.4 Hz, 2H), 3.52 (s, 3H); ¹³C NMR (175 MHz, CDCl₃) δ 167.61, 148.88, 148.67, 142.71, 138.56, 135.13, 134.69, 134.63, 133.18, 131.32, 130.42, 129.88, 128.53, 128.37, 127.28, 125.18, 38.45.

### N-methyl-N-(p-tolyl)-5-(4-(trifluoromethyl)phenyl)nicotinamide (APZ-2325)

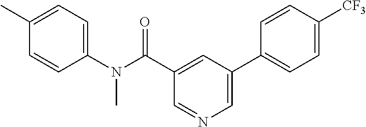 [SMILES: Cc1ccc(N(C)C(=O)c2cncc(-c3ccc(C(F)(F)F)cc3)c2)cc1]

N-methyl-N-(p-tolyl)-5-(4-(trifluoromethyl)phenyl)nicotinamide was prepared using General Procedure A from 5-(4-(trifluoromethyl)phenyl)nicotinic acid. The crude product was purified by flash column chromatography with hexanes/ethyl acetate to afford the title compound as a white solid (50%). mp 110-112° C.; R_{f} (60% EA/Hex)=0.53; ¹H NMR (500 MHz, CD₃OD) δ 8.71 (s, 1H), 8.46 (s, 1H), 7.97 (s, 1H), 7.75 (d, J=8.1 Hz, 2H), 7.64 (d, J=7.0 Hz, 2H), 7.17-7.09 (m, 4H), 3.50 (s, 3H), 2.26 (s, 3H); ¹³C NMR (175 MHz, CD₃OD) δ 169.37, 149.35, 149.17, 142.75, 141.63, 139.09, 136.49, 136.00, 133.95, 131.58 (q, CCF₃, J=32.4 Hz), 131.28, 128.82, 128.55, 127.13 (q, ArCF₃, J=3.8 Hz), 125.58 (q, CF₃, J=271.3 Hz), 38.62, 20.96.

### N-(4-chlorophenyl)-N-methyl-2-(p-tolyl)isonicotinamide (APZ-2326)

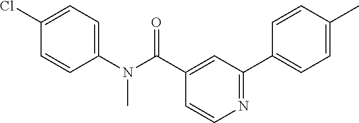 [SMILES: Cc1ccc(-c2cc(C(=O)N(C)c3ccc(Cl)cc3)ccn2)cc1]

N-(4-chlorophenyl)-N-methyl-2-(p-tolyl)isonicotinamide was prepared using General Procedure A from 2-(p-tolyl)isonicotinic acid. The crude product was purified by flash column chromatography with hexanes/ethyl acetate to afford the title compound as a white solid (64%). mp 149-152° C.; R_{f} (60% EA/Hex)=0.69; ¹H NMR (700 MHz, CDCl₃) δ 8.51 (s, 1H), 7.74 (d, J=7.6 Hz, 2H), 7.61 (s, 1H), 7.23 (t, J=8.1 Hz, 4H), 7.05-7.00 (m, 2H), 6.98 (s, 1H), 3.47 (s, 3H), 2.38 (s, 3H); ¹³C NMR (175 MHz, CDCl₃) δ 168.36, 157.64, 149.47, 143.80, 142.35, 139.43, 135.73, 133.05, 129.69, 129.51, 128.15, 126.69, 120.40, 119.14, 38.19, 21.24.

### N,2-bis(4-chlorophenyl)-N-methylisonicotinamide (APZ-2177)

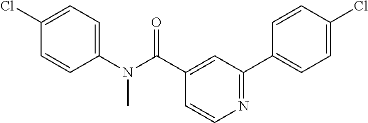 [SMILES: CN(C(=O)c1ccnc(-c2ccc(Cl)cc2)c1)c1ccc(Cl)cc1]

N,2-bis(4-chlorophenyl)-N-methylisonicotinamide was prepared using General Procedure A from 2-(4-chlorophenyl)isonicotinic acid. The crude product was purified by flash column chromatography with hexanes/ethyl acetate to afford the title compound as a white solid (15%). mp 135-136° C.; R_{f} (60% EA/Hex)=0.70; ¹H NMR (500 MHz, CDCl₃) δ 8.52 (d, J=4.3 Hz, 1H), 7.79 (d, J=8.2 Hz, 2H), 7.61 (s, 1H), 7.41 (d, J=8.5 Hz, 2H), 7.26 (d, J=8.1 Hz, 2H), 7.10-6.96 (m, 3H), 3.50 (s, 3H); ¹³C NMR (125 MHz, CDCl₃) δ 168.12, 156.51, 149.65, 144.02, 142.32, 136.97, 135.58, 133.25, 129.81, 129.01, 128.17, 128.12, 121.00, 119.33, 38.30.

### N-(4-chlorophenyl)-N-methyl-6-(p-tolyl)pyrazine-2-carboxamide (APZ-2184)

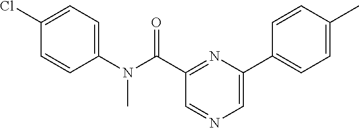 [SMILES: Cc1ccc(-c2cncc(C(=O)N(C)c3ccc(Cl)cc3)n2)cc1]

N-(4-chlorophenyl)-N-methyl-6-(p-tolyl)pyrazine-2-carboxamide was prepared using General Procedure A from 6-(p-tolyl)pyrazine-2-carboxylic acid. The crude product was purified by flash column chromatography with hexanes/ethyl acetate to afford the title compound as a white solid (96%). mp 150-153° C.; R_{f} (60% EA/Hex)=0.71; ¹H NMR (700 MHz, CD₃OD) δ 8.95 (s, 1H), 8.74 (s, 1H), 7.55 (s, 2H), 7.31 (d, J=6.2 Hz, 2H), 7.27-7.19 (m, 4H), 3.53 (s, 3H), 2.37 (s, 3H); ¹³C NMR (175 MHz, CD₃OD) δ 167.98, 152.03, 149.24, 144.43, 143.64, 142.88, 142.00, 134.07, 133.75, 130.67, 130.51, 129.86, 128.01, 38.67, 21.38.

### N,6-bis(4-chlorophenyl)-N-methylpyrazine-2-carboxamide (APZ-2199)

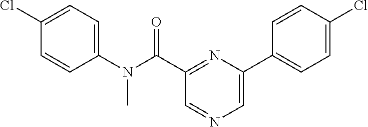 [SMILES: CN(C(=O)c1cncc(-c2ccc(Cl)cc2)n1)c1ccc(Cl)cc1]

N,6-bis(4-chlorophenyl)-N-methylpyrazine-2-carboxamide was prepared using General Procedure A from 6-(4-chlorophenyl)pyrazine-2-carboxylic acid. The crude product was purified by flash column chromatography with hexanes/ethyl acetate to afford the title compound as a white solid (55%). mp 135-138° C.; R_{f} (60% EA/Hex)=0.70; ¹H NMR (700 MHz, CD₃OD) δ 9.01 (s, 1H), 8.81 (s, 1H), 7.75-7.60 (m, 2H), 7.43 (d, J=7.5 Hz, 2H), 7.32 (d, J=7.0 Hz, 2H), 7.29-7.18 (m, 2H), 3.53 (s, 3H); ¹³C NMR (175 MHz, CD₃OD) δ 167.69, 150.64, 149.25, 144.45, 143.06, 137.67, 135.22, 134.13, 130.56, 130.16, 129.91, 129.58, 38.68.

### N-(4-chlorophenyl)-N-methyl-6-(p-tolyl)pyridazine-4-carboxamide (APZ-2316)

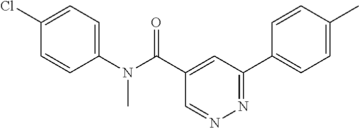 [SMILES: Cc1ccc(-c2cc(C(=O)N(C)c3ccc(Cl)cc3)cnn2)cc1]

N-(4-chlorophenyl)-N-methyl-6-(p-tolyl)pyridazine-4-carboxamide was prepared using General Procedure A from 6-(p-tolyl)pyridazine-4-carboxylic acid. The crude product was purified by flash column chromatography with hexanes/ethyl acetate to afford the title compound as an off-white solid (55%). mp 120-122° C.; R_{f} (60% EA/Hex)=0.51; ¹H NMR (700 MHz, CDCl₃) δ 8.83 (s, 1H), 7.84 (d, J=7.5 Hz, 2H), 7.74 (s, 1H), 7.32-7.26 (m, 4H), 7.06 (d, J=6.6 Hz, 2H), 3.51 (s, 3H), 2.41 (s, 3H); ¹³C NMR (175 MHz, CDCl₃) δ 165.79, 159.21, 147.44, 141.59, 140.83, 134.01, 133.95, 132.69, 130.19, 129.85, 128.30, 126.93, 122.45, 38.32, 21.36.

### N,6-bis(4-chlorophenyl)-N-methylpyridazine-4-carboxamide (APZ-2185)

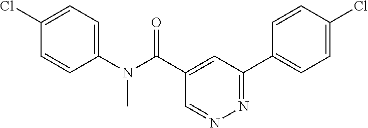 [SMILES: CN(C(=O)c1cnnc(-c2ccc(Cl)cc2)c1)c1ccc(Cl)cc1]

N,6-bis(4-chlorophenyl)-N-methylpyridazine-4-carboxamide was prepared using General Procedure A from 6-(4-chlorophenyl)pyridazine-4-carboxylic acid. The crude product was purified by flash column chromatography with hexanes/ethyl acetate to afford the title compound as a white solid (63%). mp 151-154° C.; R_{f} (60% EA/Hex)=0.54; ¹H NMR (500 MHz, CD₃OD) δ 8.95 (s, 1H), 7.99 (s, 1H), 7.93 (d, J=7.0 Hz, 2H), 7.53 (d, J=8.5 Hz, 2H), 7.35 (d, J=8.2 Hz, 2H), 7.31 (d, J=7.7 Hz, 2H), 3.51 (s, 3H); ¹³C NMR (125 MHz, CD₃OD) δ 167.19, 159.77, 149.06, 143.08, 138.05, 137.12, 135.41, 135.05, 130.97, 130.40, 129.73, 124.67, 47.99.

### N-(4-chlorophenyl)-N-methyl-6-(4-(trifluoromethoxy)phenyl)pyrazine-2-carboxamide (APZ-2254)

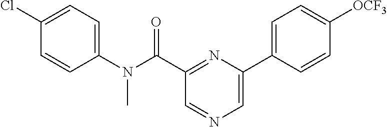 [SMILES: CN(C(=O)c1cncc(-c2ccc(OC(F)(F)F)cc2)n1)c1ccc(Cl)cc1]

N-(4-chlorophenyl)-N-methyl-6-(4-(trifluoromethoxy)phenyl)pyrazine-2-carboxamide was prepared using General Procedure A from 6-(4-(trifluoromethoxy)phenyl)pyrazine-2-carboxylic acid. The crude product was purified by flash column chromatography with hexanes/ethyl acetate to afford the title compound as a white solid (63%). mp 118-120° C.; R_{f} (60% EA/Hex)=0.71; ¹H NMR (700 MHz, CDCl₃) δ 8.93 (s, 1H), 8.88 (s, 1H), 7.60-7.49 (m, 2H), 7.33-7.21 (m, 4H), 7.13-7.03 (m, 2H), 3.55 (s, 3H); ¹³C NMR (175 MHz, CDCl₃) δ 165.70, 150.71, 148.84, 147.47, 143.88, 143.13, 141.93, 133.86, 132.87, 129.49, 128.48, 128.20, 121.15, 120.39 (q, OCF₃, J=258.1 Hz).

### N-(4-chlorophenyl)-6-(4-(difluoromethyl)phenyl)-N-methylpyrazine-2-carboxamide (APZ-2280)

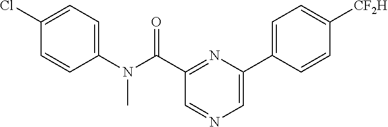 [SMILES: CN(C(=O)c1cncc(-c2ccc(C(F)F)cc2)n1)c1ccc(Cl)cc1]

N-(4-chlorophenyl)-6-(4-(difluoromethyl)phenyl)-N-methylpyrazine-2-carboxamide was prepared using General Procedure A from 6-(4-(difluoromethyl)phenyl)pyrazine-2-carboxylic acid. The crude product was purified by flash column chromatography with hexanes/ethyl acetate to afford the title compound as a white solid (12%). mp 134-138° C.; R_{f} (60% EA/Hex)=0.64; ¹H NMR (700 MHz, CDCl₃) δ 8.95 (s, 1H), 8.92 (s, 1H), 7.63-7.52 (m, 4H), 7.28 (s, 2H), 7.13-7.05 (m, 2H), 6.69 (t, J=56.3 Hz, 1H), 3.55 (s, 3H); ¹³C NMR (175 MHz, CDCl₃) δ 165.70, 149.13, 147.56, 144.14, 143.10, 142.22, 137.59, 135.92 (t, CCF₂H, J=22.5 Hz), 132.86, 129.48, 128.22, 127.20, 126.15 (t, ArCF₂H, J=5.8 Hz), 114.22 (t, CF₂H, J=239.2 Hz), 38.61.

### N-(tert-butyl)-N-(4-chlorophenyl)-6-(4-(trifluoromethyl)phenyl)pyrazine-2-carboxamide (APZ-2348)

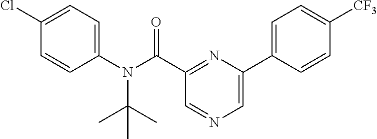 [SMILES: CC(C)(C)N(C(=O)c1cncc(-c2ccc(C(F)(F)F)cc2)n1)c1ccc(Cl)cc1]

N-(tert-butyl)-N-(4-chlorophenyl)-6-(4-(trifluoromethyl)phenyl)pyrazine-2-carboxamide was prepared using General Procedure A from 6-(4-(trifluoromethyl)phenyl)pyrazine-2-carboxylic acid. The crude product was purified by flash column chromatography with hexanes/ethyl acetate to afford the title compound as an off-white solid (81%). mp 117-120° C.; R_{f} (60% EA/Hex)=0.86; ¹H NMR (500 MHz, CDCl₃) δ 8.78 (s, 1H), 8.62 (s, 1H), 7.90 (d, J=8.1 Hz, 2H), 7.73 (d, J=8.3 Hz, 2H), 7.15 (dt, J=8.7, 2.3, 2.1 Hz, 2H), 7.10 (dt, J=8.7, 2.4, 2.2 Hz, 2H), 1.55 (s, 9H); ¹³C NMR (125 MHz, CDCl₃) δ 166.81, 150.80, 148.75, 142.94, 140.94, 139.27, 138.96, 133.96, 132.51, 131.81 (q, CCF₃, J=32.6 Hz), 128.58, 127.27, 125.85 (q, ArCF₃, J=3.7 Hz), 123.93 (q, CF₃, J=272.1 Hz), 59.77, 28.94.

### (5-chloro-1H-indol-1-yl)(6-(4-(trifluoromethyl)phenyl)pyrazin-2-yl)methanone (APZ-2255)

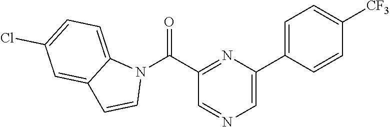 [SMILES: O=C(c1cncc(-c2ccc(C(F)(F)F)cc2)n1)n1ccc2cc(Cl)ccc21]

(5-chloro-1H-indol-1-yl)(6-(4-(trifluoromethyl)phenyl)pyrazin-2-yl)methanone was prepared using General Procedure A from 6-(4-(trifluoromethyl)phenyl)pyrazine-2-carboxylic acid. The crude product was purified by flash column chromatography with hexanes/ethyl acetate to afford the title compound as a yellow-white solid (63%). mp 170-172° C.; R_{f} (40% EA/Hex)=0.75; ¹H NMR (500 MHz, CDCl₃) δ 9.32 (s, 1H), 9.31 (s, 1H), 8.53 (d, J=8.8 Hz, 1H), 8.21 (d, J=8.2 Hz, 2H), 8.03 (d, J=3.8 Hz, 1H), 7.83 (d, J=8.3 Hz, 2H), 7.60 (d, J=2.0 Hz, 1H), 7.39 (dd, J=8.8, 2.1 Hz, 1H), 6.64 (d, J=3.8 Hz, 1H); ¹³C NMR (125 MHz, CDCl₃) δ 163.45, 149.16, 146.47, 145.76, 144.06, 138.29, 134.70, 132.57 (q, CCF₃, J=32.8 Hz), 131.87, 130.26, 128.86, 127.48, 126.36 (q, ArCF₃, J=3.7 Hz), 125.54, 123.79 (q, CF₃, J=272.6 Hz), 120.66, 118.02, 109.34.

### (5-chloroindolin-1-yl)(6-(4-(trifluoromethyl)phenyl)pyrazin-2-yl)methanone (APZ-2256)

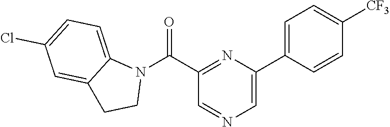 [SMILES: O=C(c1cncc(-c2ccc(C(F)(F)F)cc2)n1)N1CCc2cc(Cl)ccc21]

(5-chloroindolin-1-yl)(6-(4-(trifluoromethyl)phenyl)pyrazin-2-yl)methanone was prepared using General Procedure A from 6-(4-(trifluoromethyl)phenyl)pyrazine-2-carboxylic acid. The crude product was purified by flash column chromatography with hexanes/ethyl acetate to afford the title compound as an off-white solid (78%). mp 195-198° C.; R_{f} (40% EA/Hex)=0.59; ¹H NMR (500 MHz, CDCl₃) δ 9.18 (s, 1H), 9.17 (s, 1H), 8.29 (d, J=8.6 Hz, 1H), 8.17 (d, J=8.1 Hz, 2H), 7.81 (d, J=8.2 Hz, 2H), 7.29-7.24 (m, 1H), 7.24 (s, 1H), 4.51 (t, J=8.3 Hz, 2H), 3.20 (t, J=8.3 Hz, 2H); ¹³C NMR (125 MHz, CDCl₃) δ 163.47, 148.61, 148.49, 144.93, 142.96, 141.67, 138.89, 133.91, 132.22 (q, CCF₃, J=32.7 Hz), 129.88, 127.62, 127.37, 126.23 (q, ArCF₃, J=3.6 Hz), 123.85 (q, CF₃, J=272.7 Hz), 118.96, 50.78, 28.61.

### (6-chloro-3,4-dihydroquinolin-1(2H)-yl)(6-(4-(trifluoromethyl)phenyl)pyrazin-2-yl)methanone (APZ-2257)

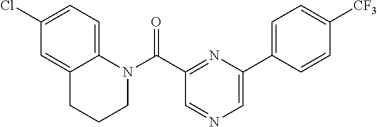 [SMILES: O=C(c1cncc(-c2ccc(C(F)(F)F)cc2)n1)N1CCCc2cc(Cl)ccc21]

(6-chloro-3,4-dihydroquinolin-1(2H)-yl)(6-(4-(trifluoromethyl)phenyl)pyrazin-2-yl)methanone was prepared using General Procedure A from 6-(4-(trifluoromethyl)phenyl)pyrazine-2-carboxylic acid. The crude product was purified by flash column chromatography with hexanes/ethyl acetate to afford the title compound as a white solid (80%). mp 92-96° C.; R_{f} (40% EA/Hex)=0.48; ¹H NMR (500 MHz, CDCl₃) δ 9.04 (s, 1H), 8.92 (s, 1H), 8.09-7.34 (m, 4H), 7.26 (s, 1H), 6.88 (s, 1H), 6.39 (s, 1H), 3.98 (s, 2H), 2.88 (t, J=6.6 Hz, 2H), 2.19-2.04 (m, 2H).

### N-(4-chloro-2-methylphenyl)-6-(4-(trifluoromethyl)phenyl)pyrazine-2-carboxamide (APZ-2275)

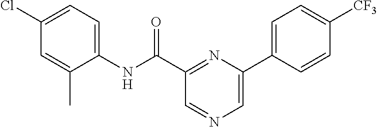 [SMILES: Cc1cc(Cl)ccc1NC(=O)c1cncc(-c2ccc(C(F)(F)F)cc2)n1]

N-(4-chloro-2-methylphenyl)-6-(4-(trifluoromethyl)phenyl)pyrazine-2-carboxamide was prepared using General Procedure A from 6-(4-(trifluoromethyl)phenyl)pyrazine-2-carboxylic acid. The crude product was purified by flash column chromatography with hexanes/ethyl acetate to afford the title compound as a yellow solid (24%). mp 205-209° C.; R_{f} (40% EA/Hex)=0.70; ¹H NMR (400 MHz, (CD₃)₂SO) δ 10.52 (s, 1H), 9.66 (s, 1H), 9.29 (s, 1H), 8.68 (d, J=8.1 Hz, 2H), 7.96 (d, J=8.4 Hz, 2H), 7.66 (d, J=8.4 Hz, 1H), 7.44 (s, 1H), 7.35 (d, J=8.8 Hz, 1H), 2.33 (s, 3H).

### N-(4-chloro-2-methoxyphenyl)-N-methyl-6-(4-(trifluoromethyl)phenyl)pyrazine-2-carboxamide (APZ-2276)

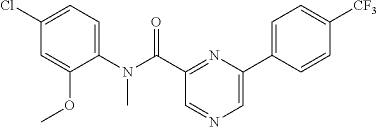 [SMILES: COc1cc(Cl)ccc1N(C)C(=O)c1cncc(-c2ccc(C(F)(F)F)cc2)n1]

N-(4-chloro-2-methoxyphenyl)-N-methyl-6-(4-(trifluoromethyl)phenyl)pyrazine-2-carboxamide was prepared using General Procedure F from N-(4-chloro-2-methoxyphenyl)-6-(4-(trifluoromethyl)phenyl)pyrazine-2-carboxamide. The crude product was purified by flash column chromatography with hexanes/ethyl acetate to afford the title compound as a white solid (80%). mp 148-151° C.; R_{f} (40% EA/Hex)=0.41; ¹H NMR (700 MHz, CDCl₃) δ 8.96 (s, 1H), 8.93 (s, 1H), 7.71-7.64 (m, 4H), 7.18 (d, J=8.3 Hz, 1H), 6.94 (d, J=8.3 Hz, 1H), 6.80 (s, 1H), 3.68 (s, 3H), 3.45 (s, 3H); ¹³C NMR (175 MHz, CDCl₃) δ 166.43, 154.48, 148.23, 147.93, 143.90, 142.11, 138.67, 134.09, 131.79, 131.70 (q, CCF₃, J=32.6 Hz), 129.20, 127.12, 125.78 (q, ArCF₃, J=3.4 Hz), 123.82 (q, CF₃, J=272.3 Hz), 120.93, 112.42, 55.76, 37.56.

### N-(4-chloro-2,6-dimethylphenyl)-N-methyl-6-(4-(trifluoromethyl)phenyl)pyrazine-2-carboxamide (APZ-2277)

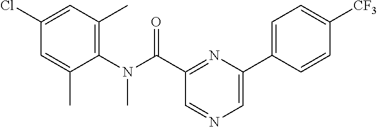 [SMILES: Cc1cc(Cl)cc(C)c1N(C)C(=O)c1cncc(-c2ccc(C(F)(F)F)cc2)n1]

N-(4-chloro-2,6-dimethylphenyl)-N-methyl-6-(4-(trifluoromethyl)phenyl)pyrazine-2-carboxamide was prepared using General Procedure F from N-(4-chloro-2,6-dimethylphenyl)-6-(4-(trifluoromethyl)phenyl)pyrazine-2-carboxamide. The crude product was purified by flash column chromatography with hexanes/ethyl acetate to afford the title compound as a white solid (25%). mp 155-157° C.; R_{f} (40% EA/Hex)=0.50; ¹H NMR (700 MHz, CDCl₃) δ 9.09 (s, 1H), 8.95 (s, 1H), 7.69 (d, J=6.3 Hz, 2H), 7.58 (d, J=6.1 Hz, 2H), 7.27 (s, 1H), 7.06 (s, 2H), 3.37 (s, 3H), 2.23 (s, 6H); ¹³C NMR (175 MHz, CDCl₃) δ 165.57, 148.62, 147.45, 143.88, 142.79, 140.62, 138.67, 137.28, 133.11, 131.74 (q, CCF₃, J=32.4 Hz), 128.44, 127.27, 125.77 (d, ArCF₃, J=3.6 Hz), 123.80 (q, CF₃, J=272.5 Hz), 36.40, 18.15.

### (3,4-dihydroquinolin-1(2H)-yl)(6-(4-(trifluoromethyl)phenyl)pyrazin-2-yl)methanone (APZ-2278)

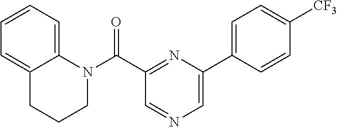 [SMILES: O=C(c1cncc(-c2ccc(C(F)(F)F)cc2)n1)N1CCCc2ccccc21]

(3,4-dihydroquinolin-1(2H)-yl)(6-(4-(trifluoromethyl)phenyl)pyrazin-2-yl)methanone was prepared using General Procedure A from 6-(4-(trifluoromethyl)phenyl)pyrazine-2-carboxylic acid. The crude product was purified by flash column chromatography with hexanes/ethyl acetate to afford the title compound as a yellow solid (63%). mp 142-144° C.; R_{f} (40% EA/Hex)=0.49; ¹H NMR (700 MHz, CDCl₃) δ 8.99 (s, 1H), 8.88 (s, 1H), 7.62 (d, J=20.8 Hz, 4H), 7.28 (s, 1H), 7.07 (s, 1H), 6.81 (s, 1H), 6.37 (s, 1H), 4.05 (s, 2H), 2.91 (s, 2H), 2.14 (s, 2H); ¹³C NMR (175 MHz, CDCl₃) δ 165.71, 148.91, 148.33, 144.00, 142.30, 139.01, 138.69, 133.40, 131.69 (q, CCF₃, J=34.1 Hz), 128.21, 127.26, 125.94, 125.80, 125.31, 125.17, 123.83 (q, CF₃, J=272.3 Hz), 43.69, 26.81, 23.88.

### N-(4-chloro-2-methylphenyl)-N-methyl-6-(4-(trifluoromethyl)phenyl)pyrazine-2-carboxamide (APZ-2279)

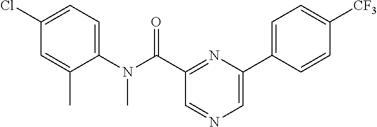 [SMILES: Cc1cc(Cl)ccc1N(C)C(=O)c1cncc(-c2ccc(C(F)(F)F)cc2)n1]

N-(4-chloro-2-methylphenyl)-N-methyl-6-(4-(trifluoromethyl)phenyl)pyrazine-2-carboxamide was prepared using General Procedure F from N-(4-chloro-2-methylphenyl)-6-(4-(trifluoromethyl)phenyl)pyrazine-2-carboxamide. The crude product was purified by flash column chromatography with hexanes/ethyl acetate to afford the title compound as a white solid (51%). mp 92-95° C.; R_{f} (40% EA/Hex)=0.48; ¹H NMR (700 MHz, CDCl₃) δ 9.03 (s, 1H), 8.93 (s, 1H), 7.69 (d, J=8.0 Hz, 2H), 7.61 (d, J=8.0 Hz, 2H), 7.25 (s, 1H), 7.11 (d, J=8.2 Hz, 1H), 7.01 (d, J=8.3 Hz, 1H), 3.44 (s, 3H), 2.30 (s, 3H); ¹³C NMR (175 MHz, CDCl₃) δ 165.60, 148.64, 147.47, 144.22, 142.41, 141.73, 138.60, 137.32, 133.45, 131.77 (q, CCF₃, J=32.6 Hz), 131.04, 129.18, 127.19, 127.12, 125.79 (q, ArCF₃, J=3.5 Hz), 123.80 (q, CF₃, J=272.4 Hz), 37.81, 17.94.

### ((4aR,8aS)-octahydroquinolin-1(2H)-yl)(6-(4-(trifluoromethyl)phenyl)pyrazin-2-yl)methanone (APZ-2281)

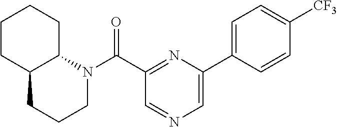 [SMILES: O=C(c1cncc(-c2ccc(C(F)(F)F)cc2)n1)N1CCC[C@H]2CCCC[C@@H]21]

((4aR,8aS)-octahydroquinolin-1(2H)-yl)(6-(4-(trifluoromethyl)phenyl)pyrazin-2-yl)methanone was prepared using General Procedure A from 6-(4-(trifluoromethyl)phenyl)pyrazine-2-carboxylic acid. The crude product was purified by flash column chromatography with hexanes/ethyl acetate to afford the title compound as a white solid (91%). mp 107-108° C.; R_{f} (40% EA/Hex)=0.50; ¹H NMR (500 MHz, CDCl₃) δ 9.09 (s, 1H), 8.86 (s, 1H), 8.16 (d, J=8.2 Hz, 2H), 7.78 (d, J=8.3 Hz, 2H), 3.70 (s, 1H), 3.61 (td, J=10.7, 3.1 Hz, 1H), 3.48-3.38 (m, 1H), 2.31 (s, 1H), 2.00-1.88 (m, 1H), 1.87-1.65 (m, 6H), 1.53-1.39 (m, 2H), 1.38-1.24 (m, 2H), 1.14 (qd, J=12.9, 3.5 Hz, 1H); ¹³C NMR (175 MHz, CDCl₃) δ 166.08, 149.85, 148.74, 143.81, 142.00, 139.04, 131.78 (q, CCF₃, J=32.6 Hz), 127.36, 126.05 (q, ArCF₃, J=3.3 Hz), 123.91 (q, CF₃, J=272.3 Hz), 62.11, 41.68, 38.06, 32.96, 30.12, 26.21, 26.09, 25.46, 23.42.

### (6-chloro-3,4-dihydroquinoxalin-1(2H)-yl)(6-(4-(trifluoromethyl)phenyl)pyrazin-2-yl)methanone (APZ-2282)

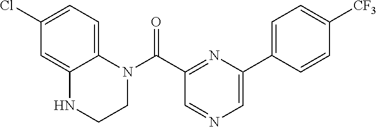 [SMILES: O=C(c1cncc(-c2ccc(C(F)(F)F)cc2)n1)N1CCNc2cc(Cl)ccc21]

(6-chloro-3,4-dihydroquinoxalin-1(2H)-yl)(6-(4-(trifluoromethyl)phenyl)pyrazin-2-yl)methanone was prepared using General Procedure A from 6-(4-(trifluoromethyl)phenyl)pyrazine-2-carboxylic acid. The crude product was purified by flash column chromatography with hexanes/ethyl acetate to afford the title compound as a yellow solid (25%). mp 152-155° C.; R_{f} (40% EA/Hex)=0.32; ¹H NMR (700 MHz, CDCl₃, rotameric mixture, major form reported) δ 9.02 (s, 1H), 8.88 (s, 1H), 7.76-7.64 (m, 4H), 6.74-6.66 (m, 1H), 6.22 (d, J=8.0 Hz, 1H), 6.13 (d, J=8.3 Hz, 1H), 4.43 (s, 1H), 4.10 (s, 2H), 3.66 (s, 2H).

### N-(4-chloro-2-(trifluoromethoxy)phenyl)-N-methyl-6-(4-(trifluoromethyl)phenyl)pyrazine-2-carboxamide (APZ-2283)

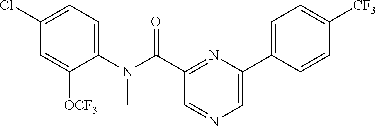 [SMILES: CN(C(=O)c1cncc(-c2ccc(C(F)(F)F)cc2)n1)c1ccc(Cl)cc1OC(F)(F)F]

N-(4-chloro-2-(trifluoromethoxy)phenyl)-N-methyl-6-(4-(trifluoromethyl)phenyl)pyrazine-2-carboxamide was prepared using General Procedure F from N-(4-chloro-2-(trifluoromethoxy)phenyl)-6-(4-(trifluoromethyl)phenyl)pyrazine-2-carboxamide. The crude product was purified by flash column chromatography with hexanes/ethyl acetate to afford the title compound as a white solid (83%). mp 133-135° C.; R_{f} (40% EA/Hex)=0.61; ¹H NMR (700 MHz, CDCl₃) δ 9.16 (s, 1H), 9.01 (s, 1H), 7.67 (d, J=8.2 Hz, 2H), 7.56 (d, J=8.2 Hz, 2H), 7.38 (d, J=8.5 Hz, 1H), 7.35 (dd, J=8.5, 2.1 Hz, 1H), 7.22 (s, 1H), 3.51 (s, 3H); ¹³C NMR (175 MHz, CDCl₃) δ 165.40, 147.95, 146.61, 144.85, 144.16, 142.77, 138.32, 135.80, 133.80, 131.88 (q, CCF₃, J=32.7 Hz), 129.93, 127.70, 126.96, 125.81 (q, ArCF₃, J=3.5 Hz), 123.79 (q, CF₃, J=272.4 Hz), 121.47, 120.00 (q, OCF₃, J=260.8 Hz), 38.43.

### N-(4-chloro-2-hydroxyphenyl)-N-methyl-6-(4-(trifluoromethyl)phenyl)pyrazine-2-carboxamide (APZ-2284)

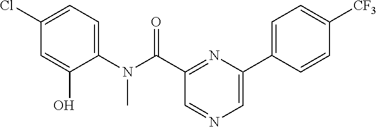 [SMILES: CN(C(=O)c1cncc(-c2ccc(C(F)(F)F)cc2)n1)c1ccc(Cl)cc1O]

N-(4-chloro-2-hydroxyphenyl)-N-methyl-6-(4-(trifluoromethyl)phenyl)pyrazine-2-carboxamide was prepared using General Procedure F from N-(2-((tert-butyldimethylsilyl)oxy)-4-chlorophenyl)-6-(4-(trifluoromethyl)phenyl)pyrazine-2-carboxamide. The crude product was purified by flash column chromatography with hexanes/ethyl acetate to afford the title compound as an off-white solid (37%). mp 166-169° C.; R_{f} (40% EA/Hex)=0.27; ¹H NMR (700 MHz, CDCl₃) δ 8.83 (s, 1H), 8.69 (s, 1H), 8.57 (s, 1H), 7.66 (d, J=8.0 Hz, 2H), 7.60 (d, J=7.9 Hz, 2H), 6.93 (d, J=2.0 Hz, 1H), 6.91 (d, J=8.4 Hz, 1H), 6.72 (dd, J=8.4, 2.0 Hz, 1H), 3.44 (s, 3H); ¹³C NMR (175 MHz, CDCl₃) δ 167.26, 152.91, 148.47, 147.53, 143.16, 142.01, 137.88, 134.64, 132.00 (q, CCF₃, J=32.5 Hz), 129.88, 128.75, 127.01, 125.88 (d, ArCF₃, J=3.3 Hz), 123.71 (q, CF₃, J=272.4 Hz), 120.82, 117.57, 37.60.

### (7-chloro-2,3-dihydro-4H-benzo[b][1,4]oxazin-4-yl)(6-(4-(trifluoromethyl)phenyl)pyrazin-2-yl)methanone (APZ-2347)

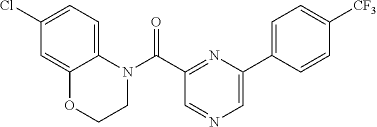 [SMILES: O=C(c1cncc(-c2ccc(C(F)(F)F)cc2)n1)N1CCOc2cc(Cl)ccc21]

(7-chloro-2,3-dihydro-4H-benzo[b][1,4]oxazin-4-yl)(6-(4-(trifluoromethyl)phenyl)pyrazin-2-yl)methanone was prepared using General Procedure A from 6-(4-(trifluoromethyl)phenyl)pyrazine-2-carboxylic acid. The crude product was purified by flash column chromatography with hexanes/ethyl acetate to afford the title compound as a yellow-white solid (56%). mp 107-110° C.; R_{f} (40% EA/Hex)=0.50; ¹H NMR (700 MHz, CDCl₃, rotameric mixture, major form reported) δ 9.36-8.85 (m, 2H), 8.35-7.99 (m, 1.5H), 7.99-7.51 (m, 3H), 7.17-6.76 (m, 1.5H), 6.49 (s, 0.45H), 6.19 (s, 0.45H), 4.54 (s, 0.9H), 4.34 (s, 1.1H), 4.14 (d, J=54.2 Hz, 2H).

### (6-chloro-2H-benzo[d][1,3]oxazin-1(4H)-yl)(6-(4-(trifluoromethyl)phenyl)pyrazin-2-yl)methanone (APZ-2349)

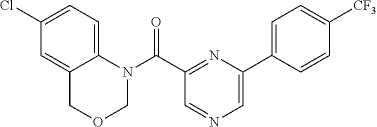 [SMILES: O=C(c1cncc(-c2ccc(C(F)(F)F)cc2)n1)N1COCc2cc(Cl)ccc21]

(6-chloro-2H-benzo[d][1,3]oxazin-1(4H)-yl)(6-(4-(trifluoromethyl)phenyl)pyrazin-2-yl)methanone was prepared using General Procedure A from 6-(4-(trifluoromethyl)phenyl)pyrazine-2-carboxylic acid. The crude product was purified by flash column chromatography with hexanes/ethyl acetate to afford the title compound as a yellow solid (22%). mp 169-172° C.; R_{f} (40% EA/Hex)=0.68; ¹H NMR (500 MHz, (CD₃)₂CO) δ 9.44 (s, 1H), 9.06 (s, 1H), 8.35 (s, 2H), 7.89 (d, 3H), 7.37-7.18 (m, 2H), 5.47 (s, 2H), 5.04 (s, 2H).

### N-(4-chlorophenyl)-6-(4,4-difluoropiperidin-1-yl)-N-methylpyrazine-2-carboxamide (APZ-2356)

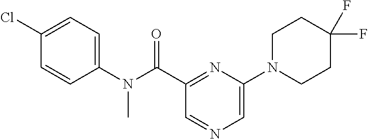 [SMILES: CN(C(=O)c1cncc(N2CCC(F)(F)CC2)n1)c1ccc(Cl)cc1]

N-(4-chlorophenyl)-6-(4,4-difluoropiperidin-1-yl)-N-methylpyrazine-2-carboxamide was prepared using General Procedure A from 6-(4,4-difluoropiperidin-1-yl)pyrazine-2-carboxylic acid. The crude product was purified by flash column chromatography with hexanes/ethyl acetate to afford the title compound as a yellow solid (60%). mp 124-125° C.; R_{f} (40% EA/Hex)=0.22; ¹H NMR (700 MHz, CDCl₃) δ 8.26 (s, 1H), 8.07 (s, 1H), 7.23 (d, J=7.9 Hz, 2H), 7.02 (d, J=7.8 Hz, 2H), 3.49 (s, 3H), 3.42 (s, 4H), 1.82 (s, 4H); ¹³C NMR (175 MHz, CDCl₃) δ 166.45, 151.30, 145.03, 143.30, 133.45, 132.21, 131.25, 129.19, 127.76, 121.55 (t, CF₂, J=242.1 Hz), 41.33, 38.42, 33.15 (t, CCF₂, J=23.1 Hz).

### N-(4-chlorophenyl)-N-methyl-6-(4-(trifluoromethyl)piperidin-1-yl)pyrazine-2-carboxamide (APZ-2357)

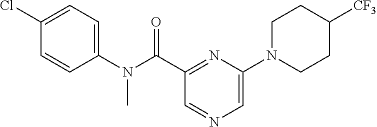 [SMILES: CN(C(=O)c1cncc(N2CCC(C(F)(F)F)CC2)n1)c1ccc(Cl)cc1]

N-(4-chlorophenyl)-N-methyl-6-(4-(trifluoromethyl)piperidin-1-yl)pyrazine-2-carboxamide was prepared using General Procedure A from 6-(4-(trifluoromethyl)piperidin-1-yl)pyrazine-2-carboxylic acid. The crude product was purified by flash column chromatography with hexanes/ethyl acetate to afford the title compound as a yellow solid (49%). mp 125-127° C.; R_{f} (40% EA/Hex)=0.26; ¹H NMR (500 MHz, CDCl₃) δ 8.23 (s, 1H), 8.02 (s, 1H), 7.22 (d, J=8.3 Hz, 2H), 7.01 (d, J=6.8 Hz, 2H), 3.99 (d, J=10.3 Hz, 2H), 3.47 (s, 3H), 2.65 (t, J=12.5 Hz, 2H), 2.30-2.16 (m, 1H), 1.84 (d, J=12.6 Hz, 2H), 1.35 (q, J=10.6 Hz, 2H); ¹³C NMR (125 MHz, CDCl₃) δ 166.67, 151.89, 145.13, 143.49, 133.21, 132.31, 131.31, 129.20, 127.84, 126.94 (q, CF₃, J=278.2 Hz), 43.32, 40.48 (q, CHCF₃, J=27.6 Hz), 38.44, 23.86 (q, CH₂CHCF₃, J=2.6 Hz).

### N-(2-amino-4-chlorophenyl)-N-methyl-6-(4-(trifluoromethyl)phenyl)pyrazine-2-carboxamide (APZ-2358)

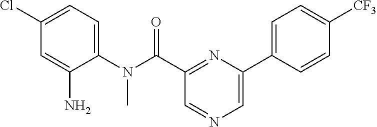 [SMILES: CN(C(=O)c1cncc(-c2ccc(C(F)(F)F)cc2)n1)c1ccc(Cl)cc1N]

N-(2-amino-4-chlorophenyl)-N-methyl-6-(4-(trifluoromethyl)phenyl)pyrazine-2-carboxamide was prepared using General Procedure A from 6-(4-(trifluoromethyl)phenyl)pyrazine-2-carboxylic acid. The crude product was purified by flash column chromatography with hexanes/ethyl acetate to afford the title compound as a yellow-white solid (1%). mp 217-219° C.; R_{f} (40% EA/Hex)=0.19; ¹H NMR (500 MHz, CDCl₃) δ 8.98 (s, 1H), 8.93 (s, 1H), 7.74 (d, J=8.4 Hz, 2H), 7.70 (d, J=8.4 Hz, 2H), 6.79-6.75 (m, 2H), 6.56 (dd, J=8.3, 2.2 Hz, 1H), 4.09 (s, 2H), 3.40 (s, 3H); ¹³C NMR (125 MHz, CDCl₃) δ 166.91, 148.96, 147.50, 143.99, 143.80, 142.72, 138.89, 134.49, 131.91 (q, CCF₃, J=32.6 Hz), 129.33, 128.80, 127.37, 125.85 (q, ArCF₃, J=3.7 Hz), 123.88 (q, CF₃, J=272.3 Hz), 118.77, 115.87, 36.29.

### 5-chloro-1-methyl-2-(6-(4-(trifluoromethyl)phenyl)pyrazin-2-yl)-1H-benzo[d]imidazole (APZ-2359)

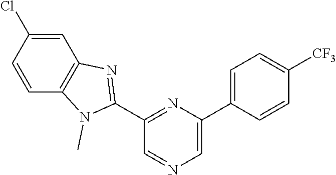 [SMILES: Cn1c(-c2cncc(-c3ccc(C(F)(F)F)cc3)n2)nc2cc(Cl)ccc21]

5-chloro-1-methyl-2-(6-(4-(trifluoromethyl)phenyl)pyrazin-2-yl)-1H-benzo[d]imidazole was prepared using General Procedure F from N-(2-amino-4-chlorophenyl)-N-methyl-6-(4-(trifluoromethyl)phenyl)pyrazine-2-carboxamide. The crude product was purified by flash column chromatography with hexanes/ethyl acetate to afford the title compound as a white solid (48%). mp 218-226° C.; R_{f} (40% EA/Hex)=0.70; ¹H NMR (500 MHz, CDCl₃) δ 9.62 (s, 1H), 9.11 (s, 1H), 8.20 (d, J=8.1 Hz, 2H), 7.86-7.80 (m, 3H), 7.39 (d, J=8.6 Hz, 1H), 7.35 (dd, J=8.7, 1.7 Hz, 1H), 4.34 (s, 3H); ¹³C NMR (125 MHz, CDCl₃) δ 149.18, 148.43, 145.26, 144.97, 143.33, 141.48, 139.27, 135.88, 132.10 (q, CCF₃, J=32.7 Hz), 128.81, 127.30, 126.23 (q, ArCF₃, J=3.7 Hz), 124.69, 123.89 (q, CF₃, J=272.4 Hz), 120.17, 110.82, 33.14.

### N-(4-chlorophenyl)-N-isopropyl-6-(4-(trifluoromethoxy)phenyl)pyrazine-2-carboxamide (APZ-2352)

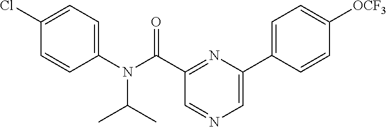 [SMILES: CC(C)N(C(=O)c1cncc(-c2ccc(OC(F)(F)F)cc2)n1)c1ccc(Cl)cc1]

N-(4-chlorophenyl)-N-isopropyl-6-(4-(trifluoromethoxy)phenyl)pyrazine-2-carboxamide was prepared using General Procedure A from 6-(4-(trifluoromethoxy)phenyl)pyrazine-2-carboxylic acid. The crude product was purified by flash column chromatography with hexanes/ethyl acetate to afford the title compound as a white solid (38%). mp 120-121° C.; R_{f} (30% EA/Hex)=0.36; ¹H NMR (500 MHz, (CD₃)₂CO) δ 9.04 (s, 1H), 8.78 (s, 1H), 7.98 (d, J=7.6 Hz, 2H), 7.44 (d, J=8.2 Hz, 2H), 7.40-7.32 (m, 4H), 5.16-4.99 (m, 1H), 1.25 (d, J=6.8 Hz, 6H); ¹³C NMR (175 MHz, (CD₃)₂CO) δ 166.12, 151.26-151.16 (m, COCF₃), 150.21, 149.16, 144.35, 142.29, 138.91, 135.56, 133.98, 133.42, 129.80, 129.62, 122.07, 121.40 (q, OCF₃, J=256.2 Hz), 48.62, 20.96.

### 1-(4-chlorophenyl)-N-(3,4-dimethylphenyl)-1H-1,2,4-triazole-3-carboxamide (APZ-2029)

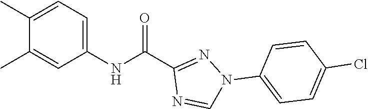 [SMILES: Cc1ccc(NC(=O)c2ncn(-c3ccc(Cl)cc3)n2)cc1C]

1-(4-chlorophenyl)-N-(3,4-dimethylphenyl)-1H-1,2,4-triazole-3-carboxamide was prepared via General Procedure A from 1-(4-chlorophenyl)-1H-1,2,4-triazole-3-carboxylic acid and 3,4-dimethylaniline. The crude product was purified via recrystallization from ethyl acetate to afford the title compound as a white solid (57%). mp>260° C.; R_{f} (50% EA/Hex)=0.38; ¹H NMR (700 MHz, CDCl₃) δ 8.92 (s, 1H), 8.61 (s, 1H), 7.75 (d, J=8.1 Hz, 2H), 7.61-7.41 (m, 5H), 7.23-7.08 (m, 1H), 2.30 (s, 3H), 2.26 (s, 3H); ¹³C NMR (175 MHz, CDCl₃) δ 158.04, 156.09, 141.45, 137.53, 134.98, 134.90, 134.79, 133.37, 130.18, 130.14, 121.53, 121.12, 117.38, 20.12, 19.44.

### 1-(4-chlorophenyl)-N-(4-cyanophenyl)-N-methyl-1H-1,2,4-triazole-3-carboxamide (APZ-2030)

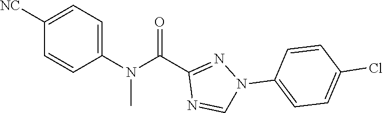 [SMILES: CN(C(=O)c1ncn(-c2ccc(Cl)cc2)n1)c1ccc(C#N)cc1]

1-(4-chlorophenyl)-N-(4-cyanophenyl)-N-methyl-1H-1,2,4-triazole-3-carboxamide was prepared via General Procedure A from 1-(4-chlorophenyl)-1H-1,2,4-triazole-3-carboxylic acid. The crude product was purified via recrystallization from dichloromethane/hexanes to afford the title compound as a beige solid (10%). mp 191-194° C.; R_{f} (80% EA/Hex)=0.30; ¹H NMR (500 MHz, CDCl₃) δ 8.38 (s, 1H), 7.63 (d, J=8.3 Hz, 2H), 7.55-7.44 (m, 4H), 7.33 (d, J=8.3 Hz, 2H), 3.59 (s, 3H).

### N-methyl-1-phenyl-N-(p-tolyl)-1H-1,2,4-triazole-3-carboxamide (APZ-2056)

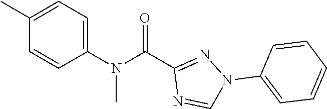 [SMILES: Cc1ccc(N(C)C(=O)c2ncn(-c3ccccc3)n2)cc1]

N-methyl-1-phenyl-N-(p-tolyl)-1H-1,2,4-triazole-3-carboxamide was prepared via General Procedure A from 1-phenyl-1H-1,2,4-triazole-3-carboxylic acid. The crude product was purified by flash column chromatography with hexanes/ethyl acetate to afford the title compound as a pale-yellow solid (94%). mp 159-161° C.; R_{f} (50% EA/Hex)=0.14; ¹H NMR (500 MHz, CDCl₃) δ 8.32 (s, 1H), 7.49 (bs, 1H), 7.44 (t, J=6.9 Hz, 1H), 7.36 (t, J=6.9 Hz, 1H), 7.09 (bs, 4H), 3.51 (s, 3H), 2.31 (s, 3H); ¹³C NMR (125 MHz, CDCl₃) δ 161.63, 158.67, 141.45, 140.43, 136.99, 136.61, 129.70 (2 non-equivalent C), 128.39, 126.67, 119.91, 38.05, 21.02.

### N-methyl-N,1-diphenyl-1H-1,2,4-triazole-3-carboxamide (APZ-2057)

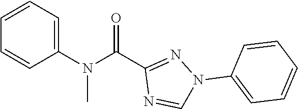 [SMILES: CN(C(=O)c1ncn(-c2ccccc2)n1)c1ccccc1]

N-methyl-N,1-diphenyl-1H-1,2,4-triazole-3-carboxamide was prepared via General Procedure A from (1-phenyl-1H-1,2,4-triazole-3-carboxylic acid. The crude product was purified by flash column chromatography with hexanes/ethyl acetate to afford title compound as a white solid (69%). mp 154-157° C.; R_{f} (70% EA/Hex)=0.26; ¹H NMR (500 MHz, CDCl₃) δ 8.33 (s, 1H), 7.49-7.40 (m, 4H), 7.40-7.27 (m, 3H), 7.26-7.13 (m, 3H), 3.54 (s, 3H); ¹³C NMR (125 MHz, CDCl₃) δ 161.51, 158.51, 144.06, 140.51, 136.56, 129.70, 129.10, 128.44, 127.14, 126.90, 119.92, 38.05.

### N,2-bis(4-chlorophenyl)-N-methyloxazole-5-carboxamide (APZ-2117)

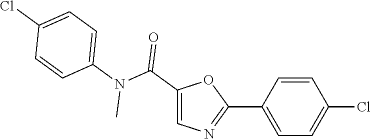 [SMILES: CN(C(=O)c1cnc(-c2ccc(Cl)cc2)o1)c1ccc(Cl)cc1]

N,2-bis(4-chlorophenyl)-N-methyloxazole-5-carboxamide was prepared via General Procedure A from 2-(4-chlorophenyl)oxazole-5-carboxylic acid. The crude product was purified by flash column chromatography with hexanes/ethyl acetate to afford the title compound as a yellow solid (88%). mp 163-166° C.; R_{f} (50% EA/Hex)=0.50; ¹H NMR (700 MHz, CDCl₃) δ 7.69 (d, J=8.2 Hz, 2H), 7.46 (d, J=8.3 Hz, 2H), 7.38 (d, J=8.3 Hz, 1H), 7.24 (d, J=8.2 Hz, 1H), 6.85 (s, 1H), 3.45 (s, 3H); ¹³C NMR (175 MHz, CDCl₃) δ 161.63, 157.39, 144.54, 141.77, 137.54, 134.46, 134.36, 130.09, 129.19, 128.98, 128.02, 124.81, 38.30.

### N-(4-chlorophenyl)-N-methyl-2-(p-tolyl)oxazole-5-carboxamide (APZ-2118)

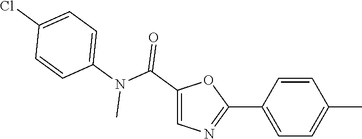 [SMILES: Cc1ccc(-c2ncc(C(=O)N(C)c3ccc(Cl)cc3)o2)cc1]

N-(4-chlorophenyl)-N-methyl-2-(p-tolyl)oxazole-5-carboxamide was prepared via General Procedure A from 2-(p-tolyl)oxazole-5-carboxylic acid. The crude product was purified by flash column chromatography with hexanes/ethyl acetate to afford the title compound as a pale-yellow solid (78%). mp 142-145° C.; R_{f} (50% EA/Hex)=0.58; ¹H NMR (700 MHz, CDCl₃) δ 7.64 (d, J=8.1 Hz, 2H), 7.47-7.42 (m, 2H), 7.25-7.21 (m, 2H), 7.21 (d, J=8.1 Hz, 2H), 6.88 (s, 1H), 3.44 (s, 3H), 2.38 (s, 3H); ¹³C NMR (175 MHz, CDCl₃) δ 162.90, 157.67, 144.11, 141.94, 141.84, 134.44, 134.34, 130.04, 129.57, 128.97, 126.77, 123.65, 38.29, 21.58.

### (4-methylpiperazin-1-yl)(6-(4-(trifluoromethyl)phenyl)pyrazin-2-yl)methanone (APZ-2232)

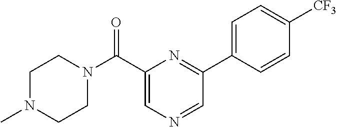 [SMILES: CN1CCN(C(=O)c2cncc(-c3ccc(C(F)(F)F)cc3)n2)CC1]

(4-methylpiperazin-1-yl)(6-(4-(trifluoromethyl)phenyl)pyrazin-2-yl)methanone was prepared via General Procedure A from 6-(4-(trifluoromethyl)phenyl)pyrazine-2-carboxylic acid. The crude product was purified by flash column chromatography with dichloromethane/methanol to afford the title compound as a white solid (66%). mp 111-113° C.; R_{f} (10% MeOH/DCM)=0.25; ¹H NMR (700 MHz, CDCl₃) δ 9.13 (s, 1H), 8.93 (s, 1H), 8.16 (d, J=7.9 Hz, 2H), 7.80 (d, J=7.9 Hz, 2H), 3.90 (t, J=4.7 Hz, 2H), 3.71 (t, J=4.7 Hz, 2H), 2.58 (t, J=4.7 Hz, 2H), 2.49 (t, J=4.7 Hz, 2H); ¹³C NMR (175 MHz, CDCl₃) δ 164.92, 148.77, 148.61, 144.31, 142.34, 138.90, 132.12 (q, CCF₃, J=32.7 Hz), 126.16 (q, ArCF₃, J=3.8 Hz), 124.96 (q, CF₃, J=272.5 Hz), 55.26, 54.63, 47.22, 46.04, 42.47.

### N-(4-chlorophenyl)-N-isopropyl-4-(4-(trifluoromethyl)phenyl)pyrimidine-2-carboxamide (APZ-2250)

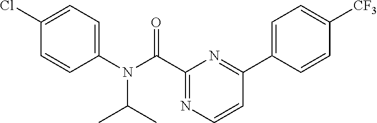 [SMILES: CC(C)N(C(=O)c1nccc(-c2ccc(C(F)(F)F)cc2)n1)c1ccc(Cl)cc1]

N-(4-chlorophenyl)-N-isopropyl-4-(4-(trifluoromethyl)phenyl)pyrimidine-2-carboxamide was prepared via General Procedure A from 4-(4-(trifluoromethyl)phenyl)pyrimidine-2-carboxylic acid. The crude product was purified by flash column chromatography with hexanes/ethyl acetate to afford the title compound as a fluffy, white crystal (68%). mp 119-122° C.; R_{f} (50% EA/Hex)=0.20; ¹H NMR (700 MHz, CDCl₃) δ 8.65 (d, J=5.3 Hz, 1H), 8.05 (d, J=8.1 Hz, 2H), 7.73 (d, J=8.1 Hz, 2H), 7.48 (d, J=5.3 Hz, 1H), 7.17-7.11 (m, 4H), 5.19 (hept, J=6.7 Hz, 1H), 1.26 (d, J=6.7 Hz, 6H); ¹³C NMR (175 MHz, CDCl₃) δ 165.92, 163.10, 162.10, 157.85, 139.11, 136.31, 133.96, 132.93 (q, CCF₃, J=32.8 Hz), 132.29, 128.65, 127.58, 125.90 (q, ArCF₃, J=3.8 Hz), 123.79 (q, CF₃, J=272.3 Hz), 115.94, 47.19, 20.81.

### N-(4-chlorophenyl)-N-ethyl-4-(4-(trifluoromethyl)phenyl)pyrimidine-2-carboxamide (APZ-2251)

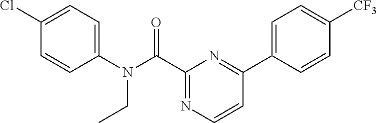 [SMILES: CCN(C(=O)c1nccc(-c2ccc(C(F)(F)F)cc2)n1)c1ccc(Cl)cc1]

N-(4-chlorophenyl)-N-ethyl-4-(4-(trifluoromethyl)phenyl)pyrimidine-2-carboxamide was prepared via General Procedure A from 4-(4-(trifluoromethyl)phenyl)pyrimidine-2-carboxylic acid. The crude product was purified by flash column chromatography with hexanes/ethyl acetate to afford the title compound as a fluffy, white crystal (54%). mp 141-143° C.; R_{f} (50% EA/Hex)=0.22; ¹H NMR (700 MHz, CDCl₃) δ 8.70 (d, J=5.3 Hz, 1H), 8.00 (d, J=8.0 Hz, 2H), 7.72 (d, J=8.0 Hz, 2H), 7.54 (d, J=5.3 Hz, 1H), 7.17 (d, J=8.4 Hz, 2H), 7.12 (d, J=8.4 Hz, 2H), 4.05 (q, J=7.2 Hz, 2H), 1.28 (t, J=7.2 Hz, 3H); ¹³C NMR (175 MHz, CDCl₃) δ 165.75, 162.63, 162.21, 158.01, 140.25, 139.05, 133.09 (q, CCF₃, J=34.9 Hz), 129.41, 129.21, 127.60, 125.89 (q, ArCF₃, J=3.5 Hz), 123.77 (q, CF₃, J=272.4 Hz), 116.30, 44.49, 12.78.

### N-(4-chlorophenyl)-N-isopropyl-6-(4-(trifluoromethyl)phenyl)pyrazine-2-carboxamide (APZ-2266)

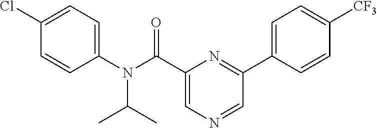 [SMILES: CC(C)N(C(=O)c1cncc(-c2ccc(C(F)(F)F)cc2)n1)c1ccc(Cl)cc1]

N-(4-chlorophenyl)-N-isopropyl-6-(4-(trifluoromethyl)phenyl)pyrazine-2-carboxamide was prepared via General Procedure A from 6-(4-(trifluoromethyl)phenyl)pyrazine-2-carboxylic acid. The crude product was purified by flash column chromatography with hexanes/ethyl acetate to afford the title compound as a fluffy, white crystal (21%). mp 143-144° C.; R_{f} (50% EA/Hex)=0.59; ¹H NMR (700 MHz, CDCl₃) δ 8.84 (d, J=19.8 Hz, 2H), 7.76 (d, J=8.0 Hz, 2H), 7.70 (d, J=8.0 Hz, 2H), 7.25 (d, J=8.3 Hz, 2H), 7.09 (d, J=8.2 Hz, 2H), 5.17 (hept, J=6.9 Hz, 1H), 1.25 (d, J=6.9 Hz, 6H); ¹³C NMR (125 MHz, CDCl₃) δ 165.44, 149.04, 148.81, 143.98, 141.66, 138.81, 137.27, 133.96, 131.82, 132.35-131.42 (m, CCF₃), 128.95, 127.28, 125.83 (q, ArCF₃, J=4.0 Hz), 123.89 (q, CF₃, J=272.2 Hz), 48.16, 20.80.

### N-(4-chlorophenyl)-N-ethyl-6-(4-(trifluoromethyl)phenyl)pyrazine-2-carboxamide (APZ-2267)

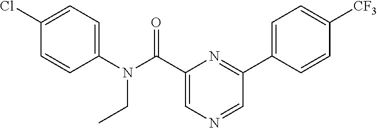 [SMILES: CCN(C(=O)c1cncc(-c2ccc(C(F)(F)F)cc2)n1)c1ccc(Cl)cc1]

N-(4-chlorophenyl)-N-ethyl-6-(4-(trifluoromethyl)phenyl)pyrazine-2-carboxamide was prepared via General Procedure A from 6-(4-(trifluoromethyl)phenyl)pyrazine-2-carboxylic acid. The crude product was purified by flash column chromatography with hexanes/ethyl acetate to afford the title compound as a fluffy, white crystal (33%). mp 113-116° C.; R_{f} (50% EA/Hex)=0.54; ¹H NMR (700 MHz, CDCl₃) δ 8.95 (s, 1H), 8.91 (s, 1H), 7.68 (d, J=8.1 Hz, 2H), 7.65 (d, J=8.1 Hz, 2H), 7.28 (d, J=8.0 Hz, 2H), 7.08 (d, J=8.0 Hz, 2H), 4.03 (q, J=7.3 Hz, 2H), 1.28 (t, J=7.3 Hz, 3H); ¹³C NMR (175 MHz, CDCl₃) δ 165.12, 148.71, 144.46, 142.10, 141.33, 138.70, 133.12, 131.94 (q, CCF₃, J=32.8 Hz), 129.46, 129.24, 127.23, 125.84 (q, ArCF₃, J=4.1 Hz), 122.78 (q, CF₃, J=271.3 Hz), 45.81, 12.71.

### N-(4-chlorophenyl)-N-(methyl-d₃)-6-(4-(trifluoromethyl)phenyl)pyrazine-2-carboxamide (APZ-2268)

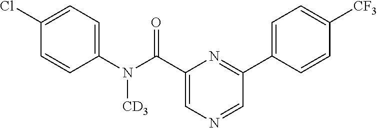 [SMILES: [2H]C([2H])([2H])N(C(=O)c1cncc(-c2ccc(C(F)(F)F)cc2)n1)c1ccc(Cl)cc1]

N-(4-chlorophenyl)-N-(methyl-d₃)-6-(4-(trifluoromethyl)phenyl)pyrazine-2-carboxamide was prepared via General Procedure E from N-(4-chlorophenyl)-6-(4-(trifluoromethyl)phenyl)pyrazine-2-carboxamide. The crude product was purified by flash column chromatography with hexanes/ethyl acetate to afford title compound as a fluffy, white crystal (13%). mp 144-146° C.; R_{f} (50% EA/Hex)=0.44; ¹H NMR (500 MHz, CDCl₃) δ 8.99 (s, 1H), 8.95 (s, 1H), 7.69 (d, J=7.4 Hz, 2H), 7.64 (d, J=7.4 Hz, 2H), 7.30 (d, J=7.7 Hz, 2H), 7.11 (d, J=7.7 Hz, 2H).

### N-(4-chlorophenyl)-6-(4-cyclobutylphenyl)-N-methylpyrazine-2-carboxamide (APZ-2291)

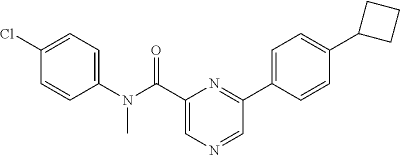 [SMILES: CN(C(=O)c1cncc(-c2ccc(C3CCC3)cc2)n1)c1ccc(Cl)cc1]

N-(4-chlorophenyl)-6-(4-cyclobutylphenyl)-N-methylpyrazine-2-carboxamide was prepared via General Procedure F from 6-(4-cyclobutylphenyl)pyrazine-2-carboxylic acid. The crude product was purified by flash column chromatography with hexanes/ethyl acetate, followed by recrystallization from dichloromethane/hexanes to afford the title compound as a fluffy, white crystal (47%). mp 145-148° C.; R_{f} (30% EA/Hex)=0.20; ¹H NMR (500 MHz, CDCl₃) δ 8.87 (s, 1H), 8.85 (s, 1H), 7.59-7.00 (m, 8H), 3.66-3.51 (m, 1H), 3.54 (s, 3H), 2.42-2.32 (m, 2H), 2.21-2.11 (m, 2H), 2.10-2.00 (m, 1H), 1.93-1.84 (m, 1H).

### N-(4-chlorophenyl)-6-(4-cyclohexylphenyl)-N-methylpyrazine-2-carboxamide (APZ-2292)

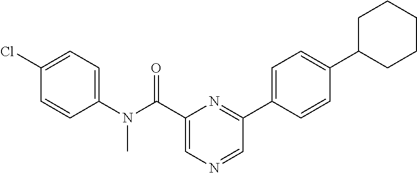 [SMILES: CN(C(=O)c1cncc(-c2ccc(C3CCCCC3)cc2)n1)c1ccc(Cl)cc1]

N-(4-chlorophenyl)-6-(4-cyclohexylphenyl)-N-methylpyrazine-2-carboxamide was prepared via General Procedure F from 6-(4-cyclohexylphenyl)pyrazine-2-carboxylic acid. The crude product was purified by flash column chromatography with hexanes/ethyl acetate, followed by recrystallization from dichloromethane/hexanes to afford the title compound as a fluffy, white crystal (34%). mp 137-140° C.; R_{f} (30% EA/Hex)=0.23; ¹H NMR (700 MHz, CDCl₃) δ 8.85 (s, 2H), 7.59-6.80 (m, 8H), 3.54 (s, 3H), 2.57-2.51 (m, 1H), 1.90-1.83 (m, 4H), 1.79-1.74 (m, 1H), 1.48-1.36 (m, 4H), 1.32-1.24 (m, 1H); ¹³C NMR (175 MHz, CDCl₃) δ 166.09, 150.59, 150.35, 147.34, 143.20, 143.06, 142.00, 132.90, 132.69, 129.42, 128.15, 127.43, 126.90, 44.42, 38.55, 34.23, 26.79, 26.07.

### N-(4-chlorophenyl)-6-(4-cycloheptylphenyl)-N-methylpyrazine-2-carboxamide (APZ-2293)

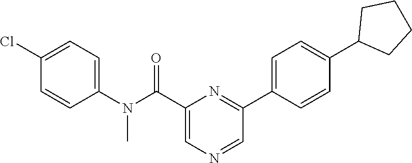 [SMILES: CN(C(=O)c1cncc(-c2ccc(C3CCCC3)cc2)n1)c1ccc(Cl)cc1]

N-(4-chlorophenyl)-6-(4-cyclopentylphenyl)-N-methylpyrazine-2-carboxamide was prepared via General Procedure F from 6-(4-cyclopentylphenyl)pyrazine-2-carboxylic acid. The crude product was purified by flash column chromatography with hexanes/ethyl acetate, followed by recrystallization from dichloromethane/hexanes to afford the title compound as a fluffy, white crystal (26%). mp 149-151° C.; R_{f} (50% EA/Hex)=0.47; ¹H NMR (700 MHz, CDCl₃) δ 8.85 (s, 2H), 7.50-6.92 (m, 8H), 3.54 (s, 3H), 3.03 (p, J=9.3 Hz, 1H), 2.13-2.06 (m, 2H), 1.87-1.78 (m, 2H), 1.77-1.66 (m, 2H), 1.65-1.56 (m, 2H); ¹³C NMR (175 MHz, CDCl₃) δ 166.10, 150.32, 149.20, 147.36, 143.18, 142.85, 141.96, 132.80, 132.69, 129.58, 128.16, 127.69, 126.81, 45.65, 38.56, 34.44, 25.62.

### N-(4-chlorophenyl)-N-methyl-5-(4-(trifluoromethyl)phenyl)nicotinamide (APZ-2157)

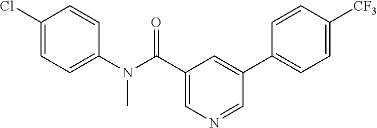 [SMILES: CN(C(=O)c1cncc(-c2ccc(C(F)(F)F)cc2)c1)c1ccc(Cl)cc1]

N-(4-chlorophenyl)-N-methyl-5-(4-(trifluoromethyl)phenyl)nicotinamide was prepared via General Procedure C from 5-bromo-N-(4-chlorophenyl)-N-methylnicotinamide. The crude product was purified by flash column chromatography with hexanes/ethyl acetate to afford the title compound as a white solid (34%). mp 112-115° C.; R_{f} (50% E A/Hex)=0.39; ¹H NMR (500 MHz, CDCl₃) δ 8.73 (d, J=2.2 Hz, 1H), 8.49 8.51-8.45 (m, 1H), 7.86 (t, J=2.2 Hz, 1H), 7.72 (d, J=8.1 Hz, 2H), 7.53 (d, J=8.1 Hz, 2H), 7.29 (d, J=8.6 Hz, 3H), 7.05 (d, J=8.6 Hz, 2H), 3.53 (s, 3H); ¹³C NMR (175 MHz, CDCl₃) δ 167.55, 149.02, 148.97, 142.69, 140.31, 134.86, 134.63, 133.23, 131.42, 130.62 (q, CCF₃, J=33.1 Hz), 129.91, 128.37, 127.46, 126.15 (q, ArCF₃, J=3.4 Hz), 124.91 (q, CF₃, J=272.1 Hz), 38.49.

### N-(4-chlorophenyl)-5-(4-ethylphenyl)-N-methylnicotinamide (APZ-2158)

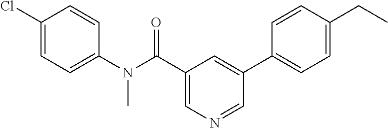 [SMILES: CCc1ccc(-c2cncc(C(=O)N(C)c3ccc(Cl)cc3)c2)cc1]

N-(4-chlorophenyl)-5-(4-ethylphenyl)-N-methylnicotinamide was prepared via General Procedure C from 5-bromo-N-(4-chlorophenyl)-N-methylnicotinamide. The crude product was purified by flash column chromatography with hexanes/ethyl acetate to afford the title compound as a cloudy white oil (3%). R_{f} (50% EA/Hex)=0.49; ¹H NMR (500 MHz, CDCl₃) δ 8.71 (d, J=2.3 Hz, 1H), 8.41 (d, J=2.3 Hz, 1H), 7.82 (t, J=2.2 Hz, 1H), 7.34 (d, J=8.0 Hz, 2H), 7.31-7.24 (m, 4H), 7.04 (d, J=8.4 Hz, 2H), 3.52 (s, 3H), 2.70 (q, J=7.7 Hz, 2H), 1.27 (t, J=7.7 Hz, 3H); ¹³C NMR (125 MHz, CDCl₃) δ 168.05, 148.94, 147.81, 144.86, 142.82, 135.88, 134.36, 134.07, 133.03, 131.19, 129.81, 128.72, 128.34, 126.99, 38.45, 28.55, 15.50.

### 5-(2-chlorophenyl)-N-(4-chlorophenyl)-N-methylnicotinamide (APZ-2190)

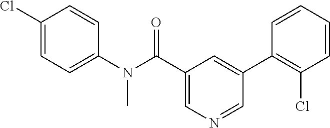 [SMILES: CN(C(=O)c1cncc(-c2ccccc2Cl)c1)c1ccc(Cl)cc1]

5-(2-chlorophenyl)-N-(4-chlorophenyl)-N-methylnicotinamide was prepared via General Procedure C from 5-bromo-N-(4-chlorophenyl)-N-methylnicotinamide. The crude product was purified by flash column chromatography with hexanes/ethyl acetate to afford the title compound as an off-white solid (6%). mp 65-67° C.; R_{f} (50% EA/Hex)=0.27; ¹H NMR (700 MHz, CDCl₃) δ 8.57 (s, 1H), 8.55 (s, 1H), 7.67 (s, 1H), 7.49-7.44 (m, 1H), 7.35-7.30 (m, 2H), 7.28-7.25 (m, 1H), 7.16-7.12 (m, 1H), 7.04 (d, J=8.2 Hz, 2H), 3.51 (s, 3H); ¹³C NMR (175 MHz, CDCl₃) δ 167.90, 150.71, 148.54, 142.67, 136.90, 135.92, 134.22, 132.97, 132.64, 131.10, 130.78, 130.12, 129.86, 129.76, 128.35, 127.21, 38.41.

### N-(6-chloropyridin-3-yl)-N-methyl-6-(4-(trifluoromethyl)phenyl)pyrazine-2-carboxamide (APZ-2252)

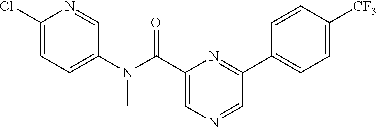 [SMILES: CN(C(=O)c1cncc(-c2ccc(C(F)(F)F)cc2)n1)c1ccc(Cl)nc1]

N-(6-chloropyridin-3-yl)-N-methyl-6-(4-(trifluoromethyl)phenyl)pyrazine-2-carboxamide was prepared via General Procedure D from N-(6-chloropyridin-3-yl)-6-(4-(trifluoromethyl)phenyl)pyrazine-2-carboxamide. The crude product was purified by flash column chromatography with hexanes/ethyl acetate to afford the title compound as a white solid (36%). mp 122-125° C.; R_{f} (50% EA/Hex)=0.31; ¹H NMR (500 MHz, CDCl₃) δ 9.09 (s, 1H), 9.00 (s, 1H), 8.24 (s, 1H), 7.70 (d, J=7.8 Hz, 2H), 7.67-7.55 (m, 2H), 7.51 (bs, 1H), 7.31 (d, J=7.8 Hz, 1H), 3.59 (s, 3H); ¹³C NMR (125 MHz, CDCl₃) δ 165.33, 149.44, 148.54, 147.80, 146.64, 144.98, 142.83, 138.31, 136.63, 132.15 (q, CCF₃, J=32.7 Hz), 127.05, 126.01 (q, ArCF₃, J=3.8 Hz), 124.63, 122.71 (q, CF₃, J=271.9 Hz), 38.93.

### N-(4-chlorophenyl)-N-methyl-6-(6-(trifluoromethyl)pyridin-3-yl)pyrazine-2-carboxamide (APZ-2253)

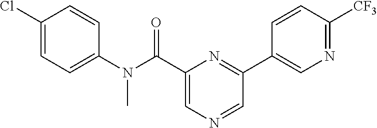 [SMILES: CN(C(=O)c1cncc(-c2ccc(C(F)(F)F)nc2)n1)c1ccc(Cl)cc1]

N-(4-chlorophenyl)-N-methyl-6-(6-(trifluoromethyl)pyridin-3-yl)pyrazine-2-carboxamide was prepared via General Procedure F from 6-(6-(trifluoromethyl)pyridin-3-yl)pyrazine-2-carboxylic acid. The crude product was purified by flash column chromatography with hexanes/ethyl acetate to afford the title compound as a white solid (51%). mp 102-105° C.; R_{f} (50% EA/Hex)=0.37; ¹H NMR (500 MHz, (CD₃)₂CO) δ 9.29 (s, 1H), 9.10 (bs, 1H), 8.97 (s, 1H), 8.34 (bs, 1H), 7.94 (d, J=8.2 Hz, 1H), 7.37 (s, 4H), 3.53 (s, 3H); ¹³C NMR (175 MHz, CDCl₃) δ 165.48, 149.25 (q, CCF₃, J=35.2 Hz), 148.29, 148.10, 146.44, 145.23, 142.87, 142.17, 135.81, 133.81, 133.22, 129.69, 128.37, 121.42 (q, CF₃, J=271.6 Hz), 120.65, 38.63.

### N-methyl-N-(6-methylpyridazin-3-yl)-6-(4-(trifluoromethyl)phenyl)pyrazine-2-carboxamide (APZ-2263)

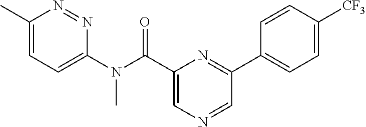 [SMILES: Cc1ccc(N(C)C(=O)c2cncc(-c3ccc(C(F)(F)F)cc3)n2)nn1]

N-methyl-N-(6-methylpyridazin-3-yl)-6-(4-(trifluoromethyl)phenyl)pyrazine-2-carboxamide was prepared via General Procedure D from N-(6-methylpyridazin-3-yl)-6-(4-(trifluoromethyl)phenyl)pyrazine-2-carboxamide. The crude product was purified by flash column chromatography with hexanes/ethyl acetate to afford the title compound as a brown solid (15%). mp 177-180° C.; R_{f} (80% EA/Hex)=0.14; ¹H NMR (500 MHz, CDCl₃) δ 9.52 (s, 1H), 9.10 (s, 1H), 8.81 (d, J=9.4 Hz, 1H), 8.24 (d, J=8.1 Hz, 2H), 7.76 (d, J=8.1 Hz, 2H), 7.37 (d, J=9.4 Hz, 1H), 4.17 (s, 3H), 2.47 (s, 3H); ¹³C NMR (175 MHz, CDCl₃) δ 171.22, 158.06, 150.33, 149.74, 149.69, 145.15, 142.89, 139.68, 132.48, 131.63 (q, CCF₃, J=32.5 Hz), 129.33, 127.64, 125.86 (q, ArCF₃, J=3.7 Hz), 124.82 (q, CF₃, J=272.2 Hz), 44.46, 21.05.

### N-methyl-N-(5-methylpyridin-2-yl)-6-(4-(trifluoromethyl)phenyl)pyrazine-2-carboxamide (APZ-2264)

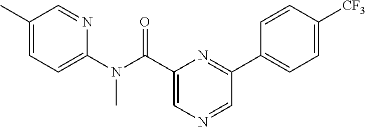 [SMILES: Cc1ccc(N(C)C(=O)c2cncc(-c3ccc(C(F)(F)F)cc3)n2)nc1]

N-methyl-N-(5-methylpyridin-2-yl)-6-(4-(trifluoromethyl)phenyl)pyrazine-2-carboxamide was prepared via General Procedure E from N-(5-methylpyridin-2-yl)-6-(4-(trifluoromethyl)phenyl)pyrazine-2-carboxamide. The crude product was purified by flash column chromatography with hexanes/ethyl acetate to afford the title compound as a white solid (12%). mp 119-121° C.; R_{f} (50% EA/Hex)=0.26; ¹H NMR (500 MHz, CDCl₃) δ 9.04 (s, 1H), 8.98 (s, 1H), 8.15-8.10 (m, 1H), 7.74-7.61 (m, 4H), 7.47 (dd, J=8.1, 1.9 Hz, 1H), 7.06 (d, J=5.9 Hz, 1H), 3.64 (s, 3H), 2.26 (s, 3H); ¹³C NMR (125 MHz, CDCl₃) δ 166.41, 148.66, 148.18, 148.14, 144.60, 142.04, 138.75, 138.50, 131.84 (q, CCF₃, J=32.6 Hz), 131.42, 127.07, 125.73 (q, ArCF₃, J=3.6 Hz), 124.97 (q, CF₃, J=271.8 Hz), 119.50, 36.40, 17.78.

### N-methyl-N-(6-methylpyridin-3-yl)-6-(4-(trifluoromethyl)phenyl)pyrazine-2-carboxamide (APZ-2265)

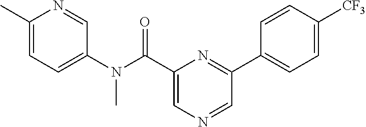 [SMILES: Cc1ccc(N(C)C(=O)c2cncc(-c3ccc(C(F)(F)F)cc3)n2)cn1]

N-methyl-N-(6-methylpyridin-3-yl)-6-(4-(trifluoromethyl)phenyl)pyrazine-2-carboxamide was prepared via General Procedure E from N-(6-methylpyridin-3-yl)-6-(4-(trifluoromethyl)phenyl)pyrazine-2-carboxamide. The crude product was purified by flash column chromatography (hexanes/ethyl acetate) to afford the title compound as a white solid (26%). mp 137-140° C.; R_{f} (80% EA/Hex)=0.18; ¹H NMR (500 MHz, CDCl₃) δ 9.02 (s, 1H), 8.96 (s, 1H), 8.33 (s, 1H), 7.74-7.58 (m, 4H), 7.41 (d, J=8.2 Hz, 1H), 7.13 (d, J=8.2 Hz, 1H), 3.59 (s, 3H), 2.50 (s, 3H); ¹³C NMR (175 MHz, CDCl₃) δ 165.69, 157.24, 148.52, 147.45, 147.38, 144.64, 142.32, 138.54, 134.21, 131.88 (q, CCF₃, J=32.0 Hz), 127.12, 125.82 (q, ArCF₃, J=2.8 Hz), 123.83 (q, CF₃, J=272.3 Hz), 123.37, 38.70, 23.96.

### N-(4-chlorophenyl)-5-(4-isopropylphenyl)-N-methylnicotinamide (APZ-2290)

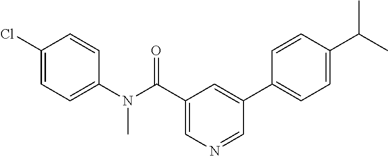 [SMILES: CC(C)c1ccc(-c2cncc(C(=O)N(C)c3ccc(Cl)cc3)c2)cc1]

N-(4-chlorophenyl)-5-(4-isopropylphenyl)-N-methylnicotinamide was prepared via General Procedure F 5-bromo-N-(4-chlorophenyl)-N-methylnicotinamide. The crude product was purified by flash column chromatography with hexanes/ethyl acetate to afford the title compound as an oil (20%). R_{f} ((50% EA/Hex)=0.18; ¹H NMR (500 MHz, CDCl₃) δ 8.71 (s, 1H), 8.42 (s, 1H), 7.84-7.79 (m, 1H), 7.38-7.29 (m, 4H), 7.29-7.24 (m, 2H), 7.04 (d, J=8.5 Hz, 2H), 3.52 (s, 3H), 2.95 (hept, J=7.0 Hz, 1H), 1.28 (d, J=7.0 Hz, 6H); ¹³C NMR (175 MHz, CDCl₃) δ 168.07, 149.47, 148.96, 147.82, 142.83, 135.86, 134.34, 134.21, 133.03, 131.20, 129.81, 128.35, 127.30, 127.01, 38.45, 33.87, 23.92.

### N-methyl-N-(2-methylthiazol-5-yl)-6-(4-(trifluoromethyl)phenyl)pyrazine-2-carboxamide (APZ-2248)

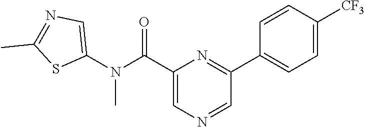 [SMILES: Cc1ncc(N(C)C(=O)c2cncc(-c3ccc(C(F)(F)F)cc3)n2)s1]

N-methyl-N-(2-methylthiazol-5-yl)-6-(4-(trifluoromethyl)phenyl)pyrazine-2-carboxamide was prepared via General Procedure D from N-(2-methylthiazol-5-yl)-6-(4-(trifluoromethyl)phenyl)pyrazine-2-carboxamide. The crude product was purified by flash column chromatography with hexanes/ethyl acetate to afford title compound as a light yellow solid (21%). mp: 161-163° C.; R_{f} (100% EA)=0.31; ¹H NMR (700 MHz, CDCl₃, rotameric mixture, major form reported) δ 9.00 (s, 1H), 8.91 (s, 1H), 7.91 (d, J=8.0 Hz, 2H), 7.73 (d, J=8.0 Hz, 2H), 7.17 (s, 1H), 3.54 (s, 3H), 2.56 (s, 3H).

### (4-(4-fluorophenyl)piperazin-1-yl)(6-(4-(trifluoromethyl)phenyl)pyrazin-2-yl)methanone (APZ-2259)

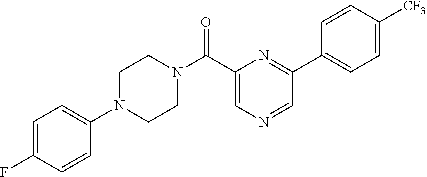 [SMILES: O=C(c1cncc(-c2ccc(C(F)(F)F)cc2)n1)N1CCN(c2ccc(F)cc2)CC1]

(4-(4-fluorophenyl)piperazin-1-yl)(6-(4-(trifluoromethyl)phenyl)pyrazin-2-yl)methanone was prepared via General Procedure F from 6-(4-(trifluoromethyl)phenyl)pyrazine-2-carboxylic acid. The crude product was purified by flash column chromatography with hexanes/ethyl acetate to afford title compound as a fluffy yellow solid (43%). mp 141-143° C.; R_{f} (50% EA/Hex)=0.40; ¹H NMR (500 MHz, CDCl₃) δ 9.15 (s, 1H), 8.98 (s, 1H), 8.16 (t, J=8.1 Hz, 2H), 7.80 (d, J=8.1 Hz, 2H), 7.05-6.95 (m, 2H), 6.95-6.87 (m, 2H), 4.03 (t, J=5.1 Hz, 2H), 3.88 (t, J=5.2 Hz, 2H), 3.26 (t, J=5.2 Hz, 2H), 3.18 (t, J=5.1 Hz, 2H); ¹³C NMR (500 MHz, CDCl₃) δ 165.06, 158.82, 156.91, 148.87, 148.47, 147.64 (d, J=2.4 Hz), 144.64, 142.65, 138.97, 132.31 (q, CCF₃, J=32.7 Hz), 127.45, 126.27 (q, ArCF₃, J=3.8 Hz), 123.98 (q, CF₃, J=272.1 Hz), 118.89, 118.82, 116.01, 115.83, 51.28, 50.48, 47.42, 42.68.

### N-(4-chlorophenyl)-6-(4-(difluoromethoxy)phenyl)-N-methylpyrazine-2-carboxamide (APZ-2288)

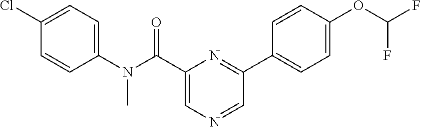 [SMILES: CN(C(=O)c1cncc(-c2ccc(OC(F)F)cc2)n1)c1ccc(Cl)cc1]

N-(4-chlorophenyl)-6-(4-(difluoromethoxy)phenyl)-N-methylpyrazine-2-carboxamide was prepared via General Procedure B from 6-chloro-N-(4-chlorophenyl)-N-methylpyrazine-2-carboxamide. The crude product was purified by flash column chromatography with hexanes/ethyl acetate to afford title compound as a light yellow solid (28%). R_{f} (50% EA/Hex)=0.34; ¹H NMR (500 MHz, CDCl₃) δ 8.90 (s, 1H), 8.87 (s, 1H), 7.52 (d, J=5.9 Hz, 2H), 7.28 (d, J=8.3 Hz, 2H), 7.16 (d, J=8.3 Hz, 2H), 7.07 (d, J=5.9 Hz, 2H), 6.57 (t, CHF₂, J=73.4 Hz, 1H), 3.55 (s, 3H).); ¹³C NMR (500 MHz, CDCl₃) δ 165.92, 152.82 (t, COCF₂, J=2.8 Hz), 149.21, 147.55, 143.71, 143.26, 141.94, 132.93, 132.54, 129.58, 128.63, 128.30, 119.77, 115.71 (t, CF₂, J=216.8 Hz), 38.72.

### N-(4-ethynylphenyl)-N-methyl-6-(4-(trifluoromethyl)phenyl)pyrazine-2-carboxamide (APZ-2289)

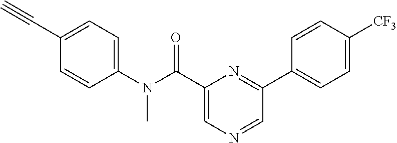 [SMILES: C#Cc1ccc(N(C)C(=O)c2cncc(-c3ccc(C(F)(F)F)cc3)n2)cc1]

N-(4-ethynylphenyl)-N-methyl-6-(4-(trifluoromethyl)phenyl)pyrazine-2-carboxamide was prepared via General Procedure D from N-(4-ethynylphenyl)-6-(4-(trifluoromethyl)phenyl)pyrazine-2-carboxamide. The crude product was purified by flash column chromatography with hexanes/ethyl acetate to afford title compound as a fluffy light orange solid (63%). mp 168-170° C.; R_{f} (30% EA/Hex)=0.38; ¹H NMR (500 MHz, CDCl₃) δ 8.98 (s, 1H), 8.93 (s, 1H), 7.66 (d, J=8.0 Hz, 2H), 7.63-7.54 (m, 2H), 7.43 (d, J=8.0 Hz, 2H), 7.10 (d, J=8.0 Hz, 2H), 3.57 (s, 3H), 3.09 (s, 1H); ¹³C NMR (500 MHz, CDCl₃) δ 165.71, 148.80, 147.73, 144.91, 144.61, 142.40, 138.74, 133.21, 132.05 (q, CCF₃, J=32.7 Hz), 127.36, 126.89, 125.99 (q, ArCF₃, J=3.8 Hz), 123.99 (q, CF₃, J=272.2 Hz), 121.14, 82.46, 78.54, 38.65.

### N-(4-cyclopropylphenyl)-N-methyl-6-(4-(trifluoromethyl)phenyl)pyrazine-2-carboxamide (APZ-2350)

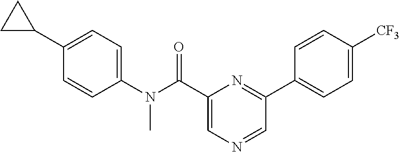 [SMILES: CN(C(=O)c1cncc(-c2ccc(C(F)(F)F)cc2)n1)c1ccc(C2CC2)cc1]

N-(4-cyclopropylphenyl)-N-methyl-6-(4-(trifluoromethyl)phenyl)pyrazine-2-carboxamide was prepared via General Procedure E from N-(4-cyclopropylphenyl)-6-(4-(trifluoromethyl)phenyl)pyrazine-2-carboxamide. The crude product was purified by flash column chromatography with hexanes/ethyl acetate to afford title compound as a yellow solid (6.4%). mp 127-129° C.; R_{f} (40% EA/Hex)=0.40; ¹H NMR (500 MHz, CDCl₃) δ 8.91 (s, 1H), 8.88 (s, 1H), 7.63 (q, J=8.2 Hz, 4H), 6.99 (q, J=8.2 Hz, 4H), 3.55 (s, 3H), 1.90-1.75 (m, 1H), 0.98-0.91 (m, 2H), 0.61-0.55 (m, 2H). NMR (700 MHz, CDCl₃) δ 165.84, 148.84, 148.34, 144.39, 143.47, 142.00, 141.82, 139.03, 131.90 (q, CCF₃, J=32.5 Hz), 127.47, 126.88, 126.51, 125.82 (q, ArCF₃, J=3.7 Hz), 124.03 (q, CF₃, J=272.5 Hz), 38.67, 15.08, 9.65.

### N-(4-chlorophenyl)-N-methyl-6-(4-methylpiperazin-1-yl)pyrazine-2-carboxamide (APZ-2360)

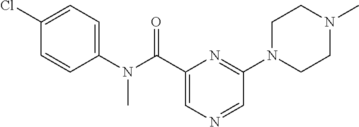 [SMILES: CN1CCN(c2cncc(C(=O)N(C)c3ccc(Cl)cc3)n2)CC1]

N-(4-chlorophenyl)-N-methyl-6-(4-methylpiperazin-1-yl)pyrazine-2-carboxamide was prepared via General Procedure A from 6-(4-methylpiperazin-1-yl)pyrazine-2-carboxylic acid. The crude product was purified by flash column chromatography with hexanes/ethyl acetate to afford title compound as a fluffy light orange solid (14%). mp 153-155° C.; R_{f} (10% MeOH/DCM)=0.40; ¹H NMR (500 MHz, CDCl₃) δ 8.20 (s, 1H), 7.98 (s, 1H), 7.21 (d, J=8.1 Hz, 2H), 7.00 (d, J=8.1 Hz, 2H), 3.46 (s, 3H), 3.36-3.19 (m, 4H), 2.39-2.31 (m, 4H), 2.30 (s, 3H); ¹³C NMR (500 MHz, CDCl₃) δ 166.85, 152.25, 145.17, 143.65, 133.12, 132.28, 131.33, 129.28, 127.97, 54.49, 46.24, 44.10, 38.54.

### N-(4-chlorophenyl)-N-methyl-6-(3-methyl-4-(trifluoromethyl)phenyl)pyrazine-2-carboxamide (APZ-2285)

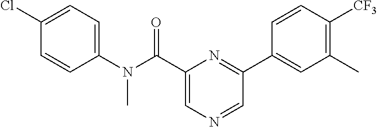 [SMILES: Cc1cc(-c2cncc(C(=O)N(C)c3ccc(Cl)cc3)n2)ccc1C(F)(F)F]

N-(4-chlorophenyl)-N-methyl-6-(3-methyl-4-(trifluoromethyl)phenyl)pyrazine-2-carboxamide was prepared via General Procedure A from 6-(3-methyl-4-(trifluoromethyl)phenyl)pyrazine-2-carboxylic acid. The crude product was purified by flash column chromatography with hexanes/ethyl acetate to afford the title compound as a white pearlescent solid (76%). mp 115-118° C.; R_{f} (30% EA/Hex)=0.29; ¹H NMR (500 MHz, CDCl₃) δ 8.97 (s, 1H), 8.91 (s, 1H), 7.65 (d, J=8.1 Hz, 1H), 7.45 (d, J=8.3 Hz, 1H), 7.32 (s, 1H), 7.28 (d, J=8.1 Hz, 2H), 7.09 (d, J=8.3 Hz, 2H), 3.56 (s, 3H), 2.53 (s, 3H); ¹³C NMR (175 MHz, CDCl₃) δ 165.80, 148.98, 147.66, 144.54, 143.28, 142.54, 138.52, 137.76, 132.90, 130.46 (q, CCF₃, J=30.5 Hz), 130.37, 129.64, 128.26, 126.55 (q, ArCF₃, J=5.7 Hz), 124.38 (q, CF₃, J=273.7 Hz), 124.20, 38.73, 19.47.

### (6-fluoro-3,4-dihydroquinolin-1(2H)-yl)(6-(4-(trifluoromethyl)phenyl)pyrazin-2-yl)methanone (APZ-2286o)

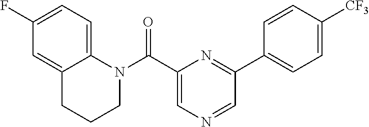 [SMILES: O=C(c1cncc(-c2ccc(C(F)(F)F)cc2)n1)N1CCCc2cc(F)ccc21]

(6-fluoro-3,4-dihydroquinolin-1(2H)-yl)(6-(4-(trifluoromethyl)phenyl)pyrazin-2-yl)methanone was prepared via General Procedure A from 6-(4-(trifluoromethyl)phenyl)pyrazine-2-carboxylic acid The crude product was purified by flash column chromatography with hexanes/ethyl acetate to afford the title compound as white pearlescent needles (53%). mp 124-128° C.; R_{f} (30% EA/Hex)=0.29; ¹H NMR (500 MHz, CDCl₃) δ 9.02 (s, 1H), 8.89 (s, 1H), 8.32-7.50 (m, 4H), 6.96 (d, J=8.6 Hz, 1H), 6.80-6.00 (m, 2H), 4.20-3.70 (m, 2H), 2.89 (t, J=6.6 Hz, 2H), 2.27-1.97 (m, 2H); ¹³C NMR (175 MHz, CDCl₃) δ 165.60, 160.73, 159.34, 148.98, 148.43, 144.08, 142.39, 138.72, 135.31, 132.04 (q, CCF₃, J=33.2 Hz), 127.23, 126.31 (d, ArF, J=8.3 Hz), 125.97, 123.86 (q, CF₃, J=272.4 Hz), 114.98 (d, ArF, J=22.4 Hz), 112.94 (d, ArF, J=22.9 Hz), 43.57, 26.93, 23.66.

### N-(4-fluorophenyl)-N-methyl-6-(4-(trifluoromethyl)phenyl)pyrazine-2-carboxamide (APZ-2287)

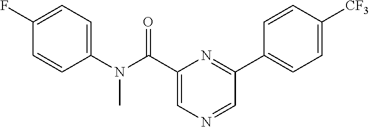 [SMILES: CN(C(=O)c1cncc(-c2ccc(C(F)(F)F)cc2)n1)c1ccc(F)cc1]

N-(4-fluorophenyl)-N-methyl-6-(4-(trifluoromethyl)phenyl)pyrazine-2-carboxamide was prepared via General Procedure E from N-(4-fluorophenyl)-6-(4-(trifluoromethyl)phenyl)pyrazine-2-carboxamide. The crude product was purified by flash column chromatography with hexanes/ethyl acetate to afford the title compound as a white pearlescent solid (57%). mp 141-145° C.; R_{f} (30% EA/Hex)=0.26; ¹H NMR (500 MHz, CDCl₃) δ 8.92 (s, 2H), 7.73-7.64 (m, 4H), 7.13 (dd, J=8.4, 4.6 Hz, 2H), 6.98 (t, J=8.3 Hz, 2H), 3.55 (s, 3H); ¹³C NMR (175 MHz, CDCl₃) δ 165.88, 161.97, 160.56, 148.67, 148.06, 144.26, 141.99, 140.38, 138.65, 131.97 (q, CCF₃, J=32.7 Hz), 128.72 (d, ArF, J=8.4 Hz), 127.16, 125.85 (q, ArCF₃, J=3.8 Hz), 123.86 (q, CF₃, J=272.4 Hz), 116.24 (d, ArF, J=22.6 Hz), 38.65.

### N-(4-chloro-2-methoxyphenyl)-N-(methyl-d₃)-6-(4-(trifluoromethyl)phenyl)pyrazine-2-carboxamide (APZ-2351)

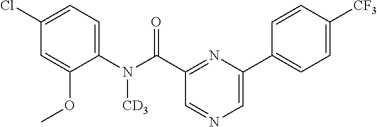 [SMILES: [2H]C([2H])([2H])N(C(=O)c1cncc(-c2ccc(C(F)(F)F)cc2)n1)c1ccc(Cl)cc1OC]

N-(4-chloro-2-methoxyphenyl)-N-(methyl-d₃)-6-(4-(trifluoromethyl)phenyl)pyrazine-2-carboxamide was prepared via General Procedure E from N-(4-chloro-2-methoxyphenyl)-6-(4-(trifluoromethyl)phenyl)pyrazine-2-carboxamide with iodomethane-d₃ in place of iodomethane. The crude product was purified by flash column chromatography with hexanes/ethyl acetate to afford the title compound as a white pearlescent solid (50%). mp 142-145° C.; R_{f} (30% EA/Hex)=0.29; ¹H NMR (500 MHz, CDCl₃) δ 8.96 (s, 1H), 8.91 (s, 1H), 7.70-7.63 (m, 4H), 7.16 (d, J=8.3 Hz, 1H), 6.92 (dd, J=8.3, 2.2 Hz, 1H), 6.78 (d, J=2.2 Hz, 1H), 3.67 (s, 3H); ¹³C NMR (175 MHz, CDCl₃) δ 166.54, 154.80, 148.40, 148.16, 144.11, 142.20, 138.93, 134.27, 132.10, 131.96 (q, CCF₃, J=32.6 Hz), 129.40, 127.29, 125.91 (q, ArCF₃, J=3.8 Hz), 124.00 (q, CF₃, J=272.3 Hz), 121.12, 112.68, 55.92, 37.34-36.55 (m, CD₃).

### N-(4-chlorophenyl)-N-methyl-6-morpholinopyrazine-2-carboxamide (APZ-2353)

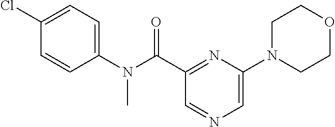 [SMILES: CN(C(=O)c1cncc(N2CCOCC2)n1)c1ccc(Cl)cc1]

N-(4-chlorophenyl)-N-methyl-6-morpholinopyrazine-2-carboxamide was prepared via General Procedure A from 6-morpholinopyrazine-2-carboxylic acid. The crude product was purified by flash column chromatography with hexanes/ethyl acetate to afford the title compound as a buff pearlescent solid (78%). mp 132-135° C.; R_{f} (50% EA/Hex)=0.22; ¹H NMR (500 MHz, CDCl₃) δ 8.24 (s, 1H), 7.98 (s, 1H), 7.22 (d, J=8.2 Hz, 2H), 7.00 (d, J=8.2 Hz, 2H), 3.66 (t, J=4.8 Hz, 4H), 3.47 (s, 3H), 3.21 (m, 4H); ¹³C NMR (175 MHz, CDCl₃) δ 166.66, 152.37, 145.14, 143.62, 133.72, 132.32, 131.17, 129.28, 127.99, 66.40, 44.44, 38.50.

### N-(4-chlorophenyl)-N-methyl-6-(piperazin-1-yl)pyrazine-2-carboxamide (APZ-2354)

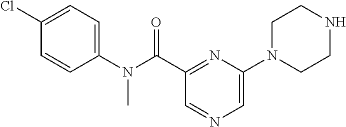 [SMILES: CN(C(=O)c1cncc(N2CCNCC2)n1)c1ccc(Cl)cc1]

N-(4-chlorophenyl)-N-methyl-6-(piperazin-1-yl)pyrazine-2-carboxamide was prepared from tert-butyl 4-(6-((4-chlorophenyl)(methyl)carbamoyl)pyrazin-2-yl)piperazine-1-carboxylate (0.116 mmol, 1 equiv) and trimethylsilyl chloride (0.579 mmol, 5 equiv), which were dissolved in methanol (0.20 M) and stirred at room temperature for 4 hours. Upon complete consumption of starting material, the reaction mixture was concentrated in vacuo and washed with hexanes to afford the title compound as a yellow solid (85%). mp 216-220° C.; R_{f} (10% MeOH/DCM)=0.19; ¹H NMR (500 MHz, CD₃OD) δ 8.40 (s, 1H), 8.27 (s, 1H), 7.38 (s, 2H), 7.25 (s, 2H), 3.69 (s, 4H), 3.48 (s, 3H), 3.22 (s, 4H); ¹³C NMR (175 MHz, CD₃OD) δ 166.01, 154.11, 149.76, 144.20, 133.89, 130.83, 129.94, 128.83, 128.55, 44.43, 42.61, 39.00.

### N-(4-chlorophenyl)-N-ethyl-6-(4-(trifluoromethoxy)phenyl)pyrazine-2-carboxamide (APZ-2355)

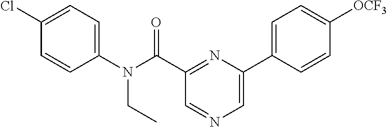 [SMILES: CCN(C(=O)c1cncc(-c2ccc(OC(F)(F)F)cc2)n1)c1ccc(Cl)cc1]

N-(4-chlorophenyl)-N-ethyl-6-(4-(trifluoromethoxy)phenyl)pyrazine-2-carboxamide was prepared via General Procedure A from 6-(4-(trifluoromethoxy)phenyl)pyrazine-2-carboxylic acid. The crude product was purified by flash column chromatography with hexanes/ethyl acetate to afford the title compound as a white pearlescent solid (82%). mp 83-85° C.; R_{f} (30% EA/Hex)=0.31; ¹H NMR (500 MHz, CDCl₃) δ 8.90 (s, 1H), 8.86 (s, 1H), 7.57 (d, J=8.1 Hz, 2H), 7.45-7.19 (m, 4H), 7.08 (d, J=8.1 Hz, 2H), 4.02 (q, J=7.1 Hz, 2H), 1.28 (t, J=7.1 Hz, 3H); ¹³C NMR (175 MHz, CDCl₃) δ 165.34, 150.81, 148.99, 147.90, 144.01, 141.90, 141.52, 134.05, 133.18, 129.57, 129.34, 128.63, 121.26, 120.52 (q, OCF₃, J=257.0 Hz), 45.92, 12.84.

### Example 2: Structures, Properties, and Biological Activity of Selected Compounds

SMILES input strings for selected compounds and compound identifiers are provided in Table 1:

TABLE 1
SMILES Input Strings for Selected Compounds

| Compound | |
|---|---|
| ID | SMILES string |
| APZ-2078 | CN(C(═O)C1═NN(C═N1)C1═CC═C(C)C═C1)C1═CC═C(Cl)C═C1 |
| APZ-2079 | COC1═CC═C(C═C1)N1C═NC(═N1)C(═O)N(C)C1═CC═C(Cl)C═C1 |
| APZ-2080 | CC1═CC═C(C═C1)N1C═NC(═N1)C(═O)NC1═CC═C(Cl)C═C1 |
| APZ-2081 | COC1═CC═C(C═C1)N1C═NC(═N1)C(═O)N(C)C1═CC═C(C)C═C1 |
| APZ-2082 | COC1═CC═C(C═C1)N1C═NC(═N1)C(═O)N(C)C1═CC(C)═C(C)C═C1 |
| APZ-2083 | CN(C(═O)C1═NN(C═N1)C1═CC═C(C)C═C1)C1═CC(C)═C(C)C═C1 |
| APZ-2318 | COC1═CC═C(C═C1)N(C)C(═O)C1═NN(C═N1)C1═CC═C(OC)C═C1 |
| APZ-2319 | COC1═CC═C(C═C1)N(C)C(═O)C1═NN(C═N1)C1═CC═C(C)C═C1 |
| APZ-2320 | CN(C(═O)C1═C(C)N═C(S1)C1═CC═C(C)C═C1)C1═CC═C(Cl)C═C1 |
| APZ-2321 | CN(C(═O)C1═CC(═CN═C1)C1═CC═C(Cl)C═C1)C1═CC═C(Cl)C═C1 |
| APZ-2123 | CN(C(═O)C1═CC(═CC═N1)C1═CC═C(Cl)C═C1)C1═CC═C(Cl)C═C1 |
| APZ-2124 | CN(C(═O)C1═CC(═CC═N1)C1═CC═C(C)C═C1)C1═CC═C(Cl)C═C1 |
| APZ-2125 | O═C(N(C)C1═CC═C(Cl)C═C1)C2═C(C)N═C(C3═CC═C(Cl)C═C3)S2 |
| APZ-2160 | O═C(C1═CC(C2═CC═C(C═C2)C)═CN═C1)N(C3═CC═C(C═C3)Cl)C |
| APZ-2322 | ClC1═CC═C(C2═CC═NC(C(N(C)C3═CC═C(C)C═C3)═O)═C2)C═C1 |
| APZ-2323 | ClC1═CC═CC(C2═CN═CC(C(N(C)C3═CC═C(Cl)C═C3)═O)═C2)═C1 |
| APZ-2324 | O═C1C(S2)═C(N═C2C3═CC═C(Cl)C═C3)CN1C4═CC═C(Cl)C═C4 |
| APZ-2325 | CC1═CC═C(N(C(C2═CC(C3═CC═C(C(F)(F)F)C═C3)═CN═C2)═O)C)C═C1 |
| APZ-2326 | CC1═CC═C(C2═NC═CC(C(N(C)C3═CC═C(Cl)C═C3)═O)═C2)C═C1 |
| APZ-2177 | ClC1═CC═C(C2═NC═CC(C(N(C)C3═CC═C(Cl)C═C3)═O)═C2)C═C1 |
| APZ-2184 | CC1═CC═C(C2═CN═CC(C(N(C)C3═CC═C(Cl)C═C3)═O)═N2)C═C1 |
| APZ-2199 | ClC1═CC═C(C2═CN═CC(C(N(C)C3═CC═C(Cl)C═C3)═O)═N2)C═C1 |
| APZ-2316 | O═C(N(C)C1═CC═C(Cl)C═C1)C2═CN═NC(C3═CC═C(C)C═C3)═C2 |
| APZ-2185 | O═C(N(C)C1═CC═C(Cl)C═C1)C2═CN═NC(C3═CC═C(Cl)C═C3)═C2 |
| APZ-2178 | ClC1═CC═C(N(C(C2═CC(C3═CC═C(C(F)(F)F)C═C3)═CN═C2)═O)C([2H])([2H])[2H])C═C1 |
| APZ-2254 | ClC1═CC═C(N(C(C2═NC(C(C═C3)═CC═C3OC(F)(F)F)═CN═C2)═O)C)C═C1 |
| APZ-2255 | ClC1═CC═C(N(C(C2═NC(C(C═C3)═CC═C3C(F)(F)F)═CN═C2)═O)C═C4)C4═C1 |
| APZ-2256 | ClC1═CC═C(N(C(C2═NC(C(C═C3)═CC═C3C(F)(F)F)═CN═C2)═O)CC4)C4═C1 |
| APZ-2257 | ClC1═CC═C(N(CCC2)C(C3═NC(C(C═C4)═CC═C4C(F)(F)F)═CN═C3)═O)C2═C1 |
| APZ-2275 | O═C(NC1═CC═C(Cl)C═C1C)C2═CN═CC(C3═CC═C(C(F)(F)F)C═C3)═N2 |
| APZ-2276 | ClC(C═C1OC)═CC═C1N(C)C(C2═CN═CC(C3═CC═C(C(F)(F)F)C═C3)═N2)═O |
| APZ-2277 | O═C(N(C)C1═C(C)C═C(Cl)C═C1C)C2═CN═CC(C3═CC═C(C(F)(F)F)C═C3)═N2 |
| APZ-2278 | O═C(N1C2═CC═CC═C2CCC1)C3═CN═CC(C4═CC═C(C(F)(F)F)C═C4)═N3 |
| APZ-2279 | O═C(N(C)C1═CC═C(Cl)C═C1C)C2═CN═CC(C3═CC═C(C(F)(F)F)C═C3)═N2 |
| APZ-2280 | O═C(N(C)C1═CC═C(Cl)C═C1)C2═CN═CC(C3═CC═C(C(F)F)C═C3)═N2 |
| APZ-2281 | O═C(N1[C@@]2([H])CCCC[C@]2([H])CCC1)C3═CN═CC(C4═CC═C(C(F)(F)F)C═C4)═N3 |
| APZ-2282 | ClC1═CC═C2C(NCCN2C(C3═NC(C(C═C4)═CC═C4C(F)(F)F)═CN═C3)═O)═C1 |
| APZ-2283 | ClC1═CC═C(N(C)C(C2═NC(C(C═C3)═CC═C3C(F)(F)F)═CN═C2)═O)C(OC(F)(F)F)═C1 |
| APZ-2284 | ClC1═CC═C(N(C)C(C2═NC(C(C═C3)═CC═C3C(F)(F)F)═CN═C2)═O)C(O)═C1 |
| APZ-2347 | O═C(N1C2═CC═C(Cl)C═C2OCC1)C3═NC(C(C═C4)═CC═C4C(F)(F)F)═CN═C3 |
| APZ-2348 | O═C(N(C(C)(C)C)C1═CC═C(Cl)C═C1)C2═NC(C(C═C3)═CC═C3C(F)(F)F)═CN═C2 |
| APZ-2349 | O═C(N1C2═CC═C(Cl)C═C2COC1)C3═NC(C(C═C4)═CC═C4C(F)(F)F)═CN═C3 |
| APZ-2356 | FC1(F)CCN(C2═NC(C(N(C)C3═CC═C(Cl)C═C3)═O)═CN═C2)CC1 |
| APZ-2357 | O═C(N(C)C1═CC═C(Cl)C═C1)C2═NC(N3CCC(CC3)C(F)(F)F)═CN═C2 |
| APZ-2358 | O═C(N(C)C1═CC═C(Cl)C═C1N)C2═NC(C(C═C3)═CC═C3C(F)(F)F)═CN═C2 |
| APZ-2359 | ClC1═CC(N═C(C2═NC(C(C═C3)═CC═C3C(F)(F)F)═CN═C2)N4C)═C4C═C1 |
| APZ-2029 | CC1═CC═C(NC(═O)C2═NN(C═N2)C2═CC═C(Cl)C═C2)C═C1C |
| APZ-2030 | CN(C(═O)C1═NN(C═N1)C1═CC═C(Cl)C═C1)C1═CC═C(C═C1)C#N |
| APZ-2056 | CN(C(═O)C1═NN(C═N1)C1═CC═CC═C1)C1═CC═C(C)C═C1 |
| APZ-2057 | CN(C(═O)C1═NN(C═N1)C1═CC═CC═C1)C1═CC═CC═C1 |
| APZ-2117 | CN(C(═O)C1═CN═C(O1)C1═CC═C(C)C═C1)C1═CC═C(Cl)C═C1 |
| APZ-2118 | CN(C(═O)C1═CN═C(O1)C1═CC═C(Cl)C═C1)C1═CC═C(Cl)C═C1 |
| APZ-2232 | O═C(C1═NC(C2═CC═C(C═C2)C(F)(F)F)═CN═C1)N3CCN(CC3)C |
| APZ-2250 | O═C(N(C(C)C)C1═CC═C(Cl)C═C1)C2═NC(C3═CC═C(C(F)(F)F)C═C3)═CC═N2 |
| APZ-2251 | O═C(N(CC)C1═CC═C(Cl)C═C1)C2═NC(C3═CC═C(C(F)(F)F)C═C3)═CC═N2 |
| APZ-2266 | O═C(N(C1═CC═C(Cl)C═C1)C(C)C)C2═NC(C3═CC═C(C(F)(F)F)C═C3)═CN═C2 |
| APZ-2267 | O═C(N(C1═CC═C(Cl)C═C1)CC)C2═NC(C3═CC═C(C(F)(F)F)C═C3)═CN═C2 |
| APZ-2268 | [CD3]N(C(C1═NC(C2═CC═C(C═C2)C(F)(F)F)═CN═C1)═O)C3═CC═C(C═C3)Cl |
| APZ-2291 | O═C(N(C)C1═CC═C(Cl)C═C1)C2═NC(C3═CC═C(C4CCC4)C═C3)═CN═C2 |
| APZ-2292 | O═C(N(C)C1═CC═C(Cl)C═C1)C2═NC(C3═CC═C(C4CCCCC4)C═C3)═CN═C2 |
| APZ-2293 | O═C(N(C)C1═CC═C(Cl)C═C1)C2═NC(C3═CC═C(C4CCCC4)C═C3)═CN═C2 |
| APZ-2157 | ClC1═CC═C(N(C)C(C2═CN═CC(C3═CC═C(C(F)(F)F)C═C3)═C2)═O)C═C1 |
| APZ-2158 | ClC1═CC═C(N(C)C(C2═CN═CC(C3═CC═C(CC)C═C3)═C2)═O)C═C1 |
| APZ-2190 | ClC1═C(C2═CN═CC(C(N(C)C3═CC═C(Cl)C═C3)═O)═C2)C═CC═C1 |
| APZ-2252 | O═C(N(C)C1═CN═C(Cl)C═C1)C2═CN═CC(C3═CC═C(C(F)(F)F)C═C3)═N2 |
| APZ-2253 | O═C(N(C1═CC═C(Cl)C═C1)C)C2═CN═CC(C3═CN═C(C(F)(F)F)C═C3)═N2 |
| APZ-2263 | CN(C1═CC═C(C)N═N1)C(C2═NC(C3═CC═C(C(F)(F)F)C═C3)═CN═C2)═O |
| APZ-2264 | CN(C1═CC═C(C)C═N1)C(C2═NC(C3═CC═C(C(F)(F)F)C═C3)═CN═C2)═O |
| APZ-2265 | CN(C1═CN═C(C)C═C1)C(C2═NC(C3═CC═C(C(F)(F)F)C═C3)═CN═C2)═O |
| APZ-2290 | O═C(N(C)C1═CC═C(Cl)C═C1)C2═CC(C3═CC═C(C(C)C)C═C3)═CN═C2 |
| APZ-2302 | O═C(N(C)C1═CC═C(Cl)C═C1)C2═NC(NC3═CC═C(C(F)(F)F)C═C3)═CN═C2 |
| APZ-2301 | O═C(N(C)C1═CC═C(Cl)C═C1)C2═NN(C3═CC═C(C(F)(F)F)C═C3)C═N2 |
| APZ-2300 | O═C(N(C([2H])([2H])[2H])C1═CC═C(Cl)C═C1OC)C2═NC(C3═CC═C(OC(F)(F)F)C═C3)═CN═C2 |
| APZ-2299 | O═C(N(C)C1═CC═C(Cl)C═C1OC)C2═NC(C3═CC═C(OC(F)(F)F)C═C3)═CN═C2 |
| APZ-2298 | O═C(N(C([2H])([2H])[2H])C1═CC═C(Cl)C═C1)C2═NC(C3═CC═C(OC(F)(F)F)C═C3)═CN═C2 |
| APZ-2297 | O═C(N(C([2H])([2H])[2H])C1═CC═C(Cl)C═C1OC)C2═NC(C3═CN═C(C(F)(F)F)C═C3)═CN═C2 |
| APZ-2296 | O═C(N(C)C1═CC═C(Cl)C═C1OC)C2═NC(C3═CN═C(C(F)(F)F)C═C3)═CN═C2 |
| APZ-2295 | O═C(N(C)C1═NC═C(Cl)C═C1C)C2═NC(C3═CN═C(C(F)(F)F)C═C3)═CN═C2 |
| APZ-2294 | O═C(N(C)C1═CC═C(Cl)C═C1C)C2═NC(C3═CN═C(C(F)(F)F)C═C3)═CN═C2 |
| APZ-2274 | O═C(N(C)C1═CC═C(Cl)C═C1)C2═NC(C3═C(C)C═C(C(F)(F)F)C═C3)═CN═C2 |
| APZ-2272 | O═C(N(C)C1═NOC(C)═C1)C2═NC(C3═CC═C(C(F)(F)F)C═C3)═CN═C2 |
| APZ-2271 | O═C(N(C)C1═CN(C)N═C1)C2═NC(C3═CC═C(C(F)(F)F)C═C3)═CN═C2 |
| APZ-2270 | O═C(N1CCN(C2═CC═C(Cl)C═C2)CC1)C3═NC(C4═CC═C(C(F)(F)F)C═C4)═CN═C3 |
| APZ-2269 | O═C(OC1═CC═C(Cl)C═C1)C2═NC(C3═CC═C(C(F)(F)F)C═C3)═CN═C2 |
| APZ-2249 | CN(C1═CN═CS1)C(C2═NC(C3═CC═C(C(F)(F)F)C═C3)═CN═C2)═O |
| APZ-2244 | CN(C1═CC═NC═C1)C(C2═NC(C3═CC═C(C(F)(F)F)C═C3)═CN═C2)═O |
| APZ-2243 | CN(C1═CC═NC═N1)C(C2═NC(C3═CC═C(C(F)(F)F)C═C3)═CN═C2)═O |
| APZ-2242 | CN(C(C1═NC(C2═CC═C(C(F)(F)F)C═C2)═CN═C1)═O)C3CS(CC3)(═O)═O |
| APZ-2241 | CN(C(C1═NC(C2═CC═C(C(F)(F)F)C═C2)═CN═C1)═O)C3═CC═C(Cl)N═N3 |
| APZ-2240 | CN(C(C1═NC(C2═CC═C(C(F)(F)F)C═C2)═CN═C1)═O)C3═CC═C(Cl)C═N3 |
| APZ-2239 | CN(C(C1═NC(C2═CC═C(C(F)(F)F)C═C2)═CN═C1)═O)C3COCC3 |
| APZ-2238 | CN(C(C1═NC(C2═CC═C(C(F)(F)F)C═C2)═CN═C1)═O)C3CN(C)CC3 |
| APZ-2237 | O═C(N1CCCN(C)CC1)C2═NC(C3═CC═C(C(F)(F)F)C═C3)═CN═C2 |
| APZ-2236 | CN(C(C1═NC(C2═CC═C(C(F)(F)F)C═C2)═CN═C1)═O)C3CCCC3 |
| APZ-2235 | O═C(N1CCC(C)CC1)C2═NC(C3═CC═C(C(F)(F)F)C═C3)═CN═C2 |
| APZ-2234 | O═C(N1CCCCC1)C2═NC(C3═CC═C(C(F)(F)F)C═C3)═CN═C2 |
| APZ-2233 | O═C(N1CCOCC1)C2═NC(C3═CC═C(C(F)(F)F)C═C3)═CN═C2 |
| APZ-2218 | O═C(N(C)C1═CC═C(Cl)C═C1)C2═NC═CC(C3═CC═C(C(C)C)C═C3)═N2 |
| APZ-2217 | ClC1═CC═C(N(C(C2═NC(C3═CC═C(C(C)C)C═C3)═CN═C2)═O)C)C═C1 |
| APZ-2216 | O═C(N(C)C1═CC═C(Cl)C═C1)C2═NC═CC(C3═CC═C(C(F)(F)F)C═C3)═N2 |
| APZ-2215 | O═C(N(C)C1═CC═C(Cl)C═C1)C2═NC═CC(C3═CC═C(CC)C═C3)═N2 |
| APZ-2214 | ClC1═CC═C(N(C(C2═NC(C3═CC═C(C(F)(F)F)C═C3)═CN═C2)═O)C)C═C1 |
| APZ-2213 | ClC1═CC═C(N(C(C2═NC(C3═CC═C(CC)C═C3)═CN═C2)═O)C)C═C1 |
| APZ-2212 | O═C(N(C)C1═CC═C(C)C═C1)C2═NC═CC(C3═CC═C(Cl)C═C3)═N2 |
| APZ-2211 | O═C(N(C)C1═CC═C(C)C═C1)C2═NC═CC(C3═CC═C(C)C═C3)═N2 |
| APZ-2198 | O═C(N(C)C1═CC═C(Cl)C═C1)C2═NC═C(C3═CC═C(C(F)(F)F)C═C3)O2 |
| APZ-2197 | O═C(N(C)C1═CC═C(Cl)C═C1)C2═NC═C(C3═CC═C(C(C)C)C═C3)O2 |
| APZ-2196 | O═C(N(C)C1═CC═C(Cl)C═C1)C2═NC═C(C3═CC═C(CC)C═C3)O2 |
| APZ-2195 | O═C(N(C)C1═CC═C(Cl)C═C1)C2═NC═CC(C3═CC═C(C)C═C3)═N2 |
| APZ-2194 | O═C(N(C)C1═CC═C(Cl)C═C1)C2═NC═CC(C3═CC═C(Cl)C═C3)═N2 |
| APZ-2193 | CC1═CC═C(C2═CN═CC(C(N(C)C3═CC═C(C)C═C3)═O)═N2)C═C1 |
| APZ-2192 | ClC1═CC═C(C2═CN═CC(C(N(C)C3═CC═C(C)C═C3)═O)═N2)C═C1 |
| APZ-2183 | ClC1═CC═C(C2═CC═NC(C(N(C([2H])([2H])[2H])C3═CC═C(Cl)C═C3)═O)═C2)C═C1 |
| APZ-2182 | ClC1═CC═C(C2═CC═NC(C(C3═CC═C(Cl)C═C3)═O)═C2)C═C1 |
| APZ-2181 | ClC1═CC═C(C(C2═CC(C3═CC═C(C(F)(F)F)C═C3)═CN═C2)═O)C═C1 |
| APZ-2180 | O═C(C1═CC═C(Cl)C═C1)C2═NC═C(C3═CC═C(Cl)C═C3)O2 |
| APZ-2179 | O═C(N(C([2H])([2H])[2H])C1═CC═C(Cl)C═C1)C2═NC═C(C3═CC═C(Cl)C═C3)O2 |
| APZ-2317 | ClC1═CC═C(C2═CC═CC(C(N(C)C3═CC═C(Cl)C═C3)═O)═N2)C═C1 |
| APZ-2191 | CC1═CC═C(C2═CC═CC(C(N(C)C3═CC═C(Cl)C═C3)═O)═N2)C═C1 |
| APZ-2273 | ClC1═CC═C(C2═NC═NC(C(N(C)C3═CC═C(Cl)C═C3)═O)═C2)C═C1 |
| APZ-2346 | CC1═CC═C(C2═NC═NC(C(N(C)C3═CC═C(Cl)C═C3)═O)═C2)C═C1 |
| APZ-2345 | CN(C1═CC═C(C═C1)Cl)C(C2═NN═C(C3═CC═C(C═C3)Cl)O2)═O |
| APZ-2344 | CN(C1═CC═C(C═C1)Cl)C(C2═NN═C(C3═CC═C(C═C3)C)O2)═O |
| APZ-2343 | ClC1═CC═C(C2═NC═CC(C(N(C)C3═CC═C(Cl)C═C3)═O)═N2)C═C1 |
| APZ-2342 | O═C(N(C)C1═CC═C(C)C═C1)C2═NC═C(C3═CC═C(Cl)C═C3)O2 |
| APZ-2341 | CC1═CC═C(C2═NC═CC(C(N(C)C3═CC═C(Cl)C═C3)═O)═N2)C═C1 |
| APZ-2340 | O═C(N(C)C1═CC═C(C)C═C1)C2═NC═C(C3═CC═C(C)C═C3)O2 |
| APZ-2339 | O═C(N(C1CCC1)C)C2═NN(C3═CC═C(Cl)C═C3)C═N2 |
| APZ-2338 | O═C(N(C1CC1)C)C2═NN(C3═CC═C(Cl)C═C3)C═N2 |
| APZ-2337 | O═C(N(C)C1═CC═CC(C)═C1)C2═NN(C3═CC═C(OC)C═C3)C═N2 |
| APZ-2336 | O═C(N(C)C1═CC═CC(C)═C1)C2═NN(C3═CC═C(C)C═C3)C═N2 |
| APZ-2335 | O═C(N(C)C1═CC═C(C)C(C)═C1)C2═NN(C3═CC═C(CC)C═C3)C═N2 |
| APZ-2334 | CC(C═C1)═CC═C1N2C═NC(N(C)C(C3═CC═C(Cl)C═C3)═O)═N2 |
| APZ-2333 | CC(C═C1)═CC═C1N2C═NC(N(C)C(C3═CC═C(C)C═C3)═O)═N2 |
| APZ-2332 | ClC(C═C1)═CC═C1N2C═NC(N(C)C(C3═CC═C(Cl)C═C3)═O)═N2 |
| APZ-2331 | ClC(C═C1)═CC═C12N2C═NC(N(C)C(C3═CC═C(C)C═C3)═O)═N2 |
| APZ-2165 | ClC(C═C1)═CC═C1N2C═NC(NC(C3═CC═C(C)C═C3)═O)═N2 |
| APZ-2164 | CC(C═C1)═CC═C1N2C═NC(NC(C3═CC═C(C)C═C3)═O)═N2 |
| APZ-2163 | ClC(C═C1)═CC═C1N2C═NC(NC(C3═CC═C(Cl)C═C3)═O)═N2 |
| APZ-2162 | CC(C═C1)═CC═C1N2C═NC(NC(C3═CC═C(Cl)C═C3)═O)═N2 |
| APZ-2122 | ClC1═CC═C(C═C1)N1C═NC(═N1)C(═O)N1CCCCC1 |
| APZ-2121 | CN1CCN(CC1)C(═O)C1═NN(C═N1)C1═CC═C(Cl)C═C1 |
| APZ-2119 | CN(C(═O)C1═NC═C(O1)C1═CC═C(C)C═C1)C1═CC═C(Cl)C═C1 |
| APZ-2161 | O═C(N(C)C1═CC═C(Cl)C═C1)C2═NC═C(C3═CC═C(Cl)C═C3)O2 |
| APZ-2315 | CN(C(═O)C1═C(C)N═C(O1)C1═CC═C(C)C═C1)C1═CC═C(Cl)C═C1 |
| APZ-2314 | CN(C(═O)C1═C(C)N═C(O1)C1═CC═C(Cl)C═C1)C1═CC═C(Cl)C═C1 |
| APZ-2313 | ClC1═CC═C(C═C1)N1C═NC(═N1)C(═O)N1CCCC1 |
| APZ-2312 | CN(C(═O)C1═CN═C(N1)C1═CC═C(Cl)C═C1)C1═CC═C(Cl)C═C1 |
| APZ-2311 | CN(C(═O)C1═CN═C(N1)C1═CC═C(C)C═C1)C1═CC═C(Cl)C═C1 |
| APZ-2310 | CN(C(═O)C1═NC═C(S1)C1═CC═C(Cl)C═C1)C1═CC═C(Cl)C═C1 |
| APZ-2309 | CN(C(═O)C1═NC═C(N1)C1═CC═C(C)C═C1)C1═CC═C(Cl)C═C1 |
| APZ-2308 | CN(C(═O)C1═NC═C(N1)C1═CC═C(Cl)C═C1)C1═CC═C(Cl)C═C1 |
| APZ-2095 | CN(C(═O)C1═CN(N═N1)C1═CC═C(C)C═C1)C1═CC═C(C)C═C1 |
| APZ-2188 | CN(C(═O)C1═CN(N═N1)C1═CC═C(Cl)C═C1)C1═CC═C(C)C═C1 |
| APZ-2094 | CN(C(═O)C1═NC═C(S1)C1═CC═C(C)C═C1)C1═CC═C(Cl)C═C1 |
| APZ-2092 | CN(C(═O)C1═CN═C(S1)C1═CC═C(C)C═C1)C1═CC═C(Cl)C═C1 |
| APZ-2091 | CN(C(═O)C1═CN═C(S1)C1═CC═C(Cl)C═C1)C1═CC═C(Cl)C═C1 |
| APZ-2090 | CN(C(═O)C1═CN(N═N1)C1═CC═C(C)C═C1)C1═CC═C(Cl)C═C1 |
| APZ-2089 | CN(C(═O)C1═CN(N═N1)C1═CC═C(Cl)C═C1)C1═CC═C(Cl)C═C1 |
| APZ-2088 | CN(C(═O)C1═NN(C═N1)C1═CC═C(Cl)C═C1)C1═NC═CS1 |
| APZ-2087 | CN(C(═O)C1═NN(C═N1)C1═CC═C(Cl)C═C1)C1═CC═NC═C1 |
| APZ-2086 | CN(C(═O)C1═NN(C═N1)C1═CC═C(C)C═C1)C1═NC═CS1 |
| APZ-2186 | CN(C1CCCC1)C(═O)C1═NN(C═N1)C1═CC═C(C)C═C1 |
| APZ-2085 | CN(C1CCCCC1)C(═O)C1═NN(C═N1)C1═CC═C(C)C═C1 |
| APZ-2084 | CN(C(═O)C1═NN(C═N1)C1═CC═C(C)C═C1)C1═CC═NC═C1 |
| APZ-2306 | CN(C1CCCC1)C(═O)C1═NN(C═N1)C1═CC═C(Cl)C═C1 |
| APZ-2305 | CN(C1CCCCC1)C(═O)C1═NN(C═N1)C1═CC═C(Cl)C═C1 |
| APZ-2304 | CCC1═CC═C(C═C1)N1C═NC(═N1)C(═O)N(C)C1═CC═C(Cl)C═C1 |
| APZ-2303 | CCC1═CC═C(C═C1)N1C═NC(═N1)C(═O)N(C)C1═CC═C(OC)C═C1 |
| APZ-2072 | CC(C)C1═CC═C(C═C1)N1C═NC(═N1)C(═O)N(C)C1═CC═C(Cl)C═C1 |
| APZ-2071 | COC1═CC═C(C═C1)N(C)C(═O)C1═NN(C═N1)C1═CC═C(C═C1)C(C)C |
| APZ-2070 | CC(C)C1═CC═C(C═C1)N1C═NC(═N1)C(═O)N(C)C1═CC═C(C)C(C)═C1 |
| APZ-2068 | CC(C)C1═CC═C(C═C1)N1C═NC(═N1)C(═O)N(C)C1═CC═C(C)C═C1 |
| APZ-2067 | CCC1═CC═C(C═C1)N1C═NC(═N1)C(═O)N(C)C1═CC═C(C)C═C1 |
| APZ-2066 | CC(C)C1═CC═C(C═C1)N1C═NC(═N1)C(═O)N(C)C1═CC═CC(C)═C1 |
| APZ-2065 | CCC1═CC═C(C═C1)N1C═NC(═N1)C(═O)N(C)C1═CC═CC(C)═C1 |
| APZ-2064 | CN(C(═O)C1═NN(C═N1)C1═CC═C(Cl)C═C1)C1═CC═CC(C)═C1 |
| APZ-2063 | CN(C(═O)C1═NN(C═N1)C1═CC═C(Cl)C═C1)C1═CC═C(C═C1)S(C)(═O)═O |
| APZ-2062 | CN(C(═O)C1═NN(C═N1)C1═CC═C(Cl)C═C1)C1═CC═CC(═C1)C(F)(F)F |
| APZ-2061 | CN(C(═O)C1═NN(C═N1)C1═CC═C(Cl)C═C1)C1═CC═CC═C1C |
| APZ-2055 | CC(═O)C1═CC═C(NC(═O)C2═NN(C═N2)C2═CC═C(Cl)C═C2)C═C1 |
| APZ-2054 | [O−][N+](═O)C1═CC═C(NC(═O)C2═NN(C═N2)C2═CC═C(Cl)C═C2)C═C1 |
| APZ-2053 | CN(C(═O)C1═NN(C═N1)C1═CC═C(Cl)C═C1)C1═CC═C(Cl)C(Cl)═C1 |
| APZ-2052 | CN(C(═O)C1═NN(C═N1)C1═CC═C(Cl)C═C1)C1═CC═C(C═C1)[N+]([O−])═O |
| APZ-2049 | CN(C(═O)C1═NN(C═N1)C1═CC═C(Cl)C═C1)C1═CC═C(C═C1)C(C)═O |
| APZ-2048 | CN(C(═O)C1═NN(C═N1)C1═CC═C(Cl)C═C1)C1═CC═C(C═C1)C(F)(F)F |
| APZ-2046 | ClC1═CC═C(C═C1)N1C═NC(═N1)C(═O)NC1═CC═C(Cl)C(Cl)═C1 |
| APZ-2045 | FC(F)(F)C1═CC═C(NC(═O)C2═NN(C═N2)C2═CC═C(Cl)C═C2)C═C1 |
| APZ-2044 | CN(C(═O)C1═NN(C═N1)C1═CC═C(Cl)C═C1)C1═CC═C(C═C1)C(C)(C)C |
| APZ-2043 | CN(C(═O)C1═NN(C═N1)C1═CC═C(Cl)C═C1)C1═CC═C(F)C═C1 |
| APZ-2042 | ClC1═CC═C(NC(═O)C2═NN(C═N2)C2═CC═C(Cl)C═C2)C═C1 |
| APZ-2041 | FC1═CC═C(NC(═O)C2═NN(C═N2)C2═CC═C(Cl)C═C2)C═C1 |
| APZ-2028 | CN(C(═O)C1═NN(C═N1)C1═CC═C(Cl)C═C1)C1═CC═C(C)C(C)═C1 |
| APZ-2027 | CCC1═CC═C(C═C1)N(C)C(═O)C1═NN(C═N1)C1═CC═C(Cl)C═C1 |
| APZ-2026 | COC1═CC═C(C═C1)N(C)C(═O)C1═NN(C═N1)C1═CC═C(Cl)C═C1 |
| APZ-2025 | CN(C(═O)C1═NN(C═N1)C1═CC═C(Cl)C═C1)C1═CC═C(Cl)C═C1 |
| APZ-2024 | CN(C)C1═CC═C(NC(═O)C2═NN(C═N2)C2═CC═C(Cl)C═C2)C═C1 |
| APZ-2023 | CC(C)(C)C1═CC═C(NC(═O)C2═NN(C═N2)C2═CC═C(Cl)C═C2)C═C1 |
| APZ-2022 | CCC1═CC═C(NC(═O)C2═NN(C═N2)C2═CC═C(Cl)C═C2)C═C1 |
| APZ-2011 | CN(C(═O)C1═NN(C═N1)C1═CC═C(F)C═C1)C1═CC═C(C)C═C1 |
| APZ-2010 | CN(C(═O)C1═NN(C═N1)C1═CC═C(Cl)C═C1)C1═CC═C(C)C═C1 |
| APZ-2330 | CN(C(═O)C1═NN(C═N1)C1═CC═C(Cl)C═C1)C1═CC═CC═C1 |
| APZ-2329 | CN(C(═O)C1═NN(C═N1)C1═CC═C(C)C═C1)C1═CC═CC═C1 |
| APZ-2327 | CN(C1═CC═C(C)C═C1)C(C2═NN(C3═CC═C(C)C═C3)C═N2)═O |
| APZ-2248 | O═C(N(C)C1═CN═C(C)S1)C2═NC(C3═CC═C(C(F)(F)F)C═C3)═CN═C2 |
| APZ-2259 | O═C(N1CCN(C2═CC═C(F)C═C2)CC1)C3═NC(C4═CC═C(C(F)(F)F)C═C4)═CN═C3 |
| APZ-2288 | O═C(N(C)C1═CC═C(Cl)C═C1)C2═CN═CC(C3═CC═C(OC(F)F)C═C3)═N2 |
| APZ-2289 | O═C(N(C)C1═CC═C(C#C)C═C1)C2═CN═CC(C3═CC═C(C(F)(F)F)C═C3)═N2 |
| APZ-2350 | O═C(N(C)C1═CC═C(C2CC2)C═C1)C3═NC(C(C═C4)═CC═C4C(F)(F)F)═CN═C3 |
| APZ-2360 | O═C(N(C)C1═CC═C(Cl)C═C1)C2═CN═CC(N3CCN(C)CC3)═N2 |
| APZ-2285 | O═C(N(C)C1═CC═C(Cl)C═C1)C2═NC(C3═CC(C)═C(C(F)(F)F)C═C3)═CN═C2 |
| APZ-2286 | O═C(C1═NC(C2═CC═C(C(F)(F)F)C═C2)═CN═C1)N(C3═CC═C(C═C3)F)C |
| APZ-2287 | O═C(N1C2═CC═C(F)C═C2CCC1)C3═CN═CC(C4═CC═C(C(F)(F)F)C═C4)═N3 |
| APZ-2353 | ClC1═CC═C(N(C)C(C2═NC(N3CCOCC3)═CN═C2)═O)C═C1 |
| APZ-2354 | ClC1═CC═C(N(C)C(C2═NC(N3CCNCC3)═CN═C2)═O)C═C1 |
| APZ-2355 | O═C(N(CC)C1═CC═C(Cl)C═C1)C2═NC(C(C═C3)═CC═C3OC(F)(F)F)═CN═C2 |
| APZ-2352 | ClC1═CC(C)═C(NC(C2═CN═CC(C3═CC═C(C(F)(F)F)C═C3)═N2)═O)C(C)═C1 |
| APZ-2258 | CN(C1═NC═CS1)C(C2═NC(C3═CC═C(C(F)(F)F)C═C3)═CN═C2)═O |
| APZ-2261 | CN(C1═NC═C(C)S1)C(C2═NC(C3═CC═C(C(F)(F)F)C═C3)═CN═C2)═O |
| APZ-2247 | CN(C1═CC(C)═NO1)C(C2═NC(C3═CC═C(C(F)(F)F)C═C3)═CN═C2)═O |
| APZ-2351 | O═C(N(C([2H])([2H])[2H])C1═CC═C(Cl)C═C1OC)C2═NC(C(C═C3)═CC═C3C(F)(F)F)═CN═C2 |

Synthesized compounds were divided into nine chemotypes having certain common sets of features. Chemotype structures are shown in Scheme 1 below, where variables (R_{1c} and so forth) are defined as described in the Detailed Description above.

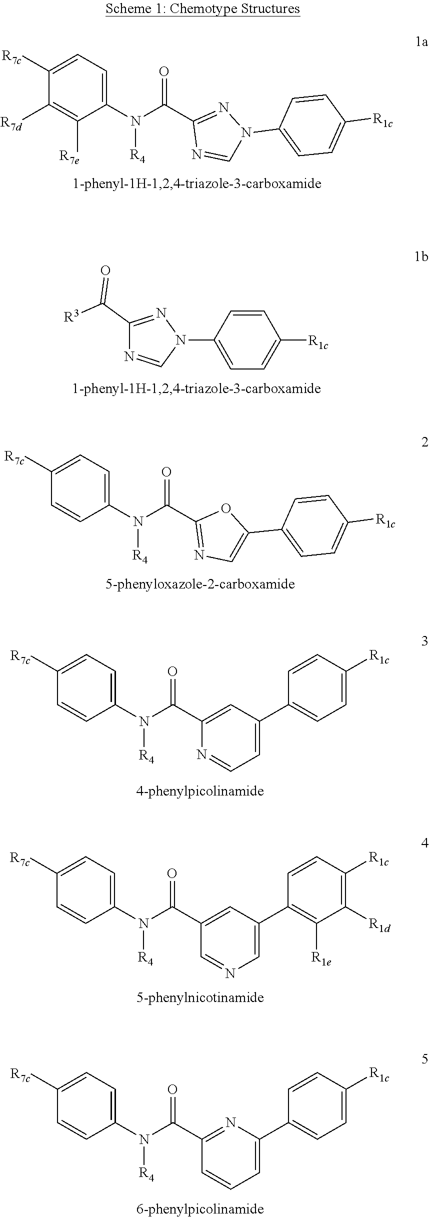

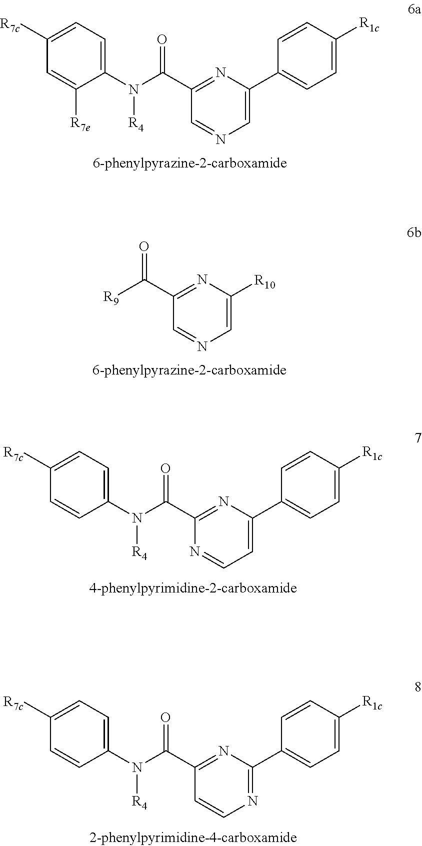

Chemotype 9 (generic structure not pictured) is defined as other chemotypes wherein potency against the intraerythrocytic stages of Plasmodium falciparum is in the micromolar range or greater. Biological data for the chemotypes pictured in Scheme 1 as well as Chemotype 9 is presented in Tables 2-12 below.

Biological assay data was gathered against the asexual blood stage of the W2 (PMID: 3284758) strain of Plasmodium falciparum. All compounds were counter-screened for cytotoxicity using the human liver cell line HepG2 (RRID: CVCL_0027) cultured in galactose media to circumvent the Crabtree effect (PMID: 17361016). Following synthesis, powder of each test compound was diluted to 10 mM in DMSO and put into a 12-point, 1:3 serial dilution in a source 384-well microtiter plate, leading to a dose response ranging from 10 mM to 52 nM. Potency against blood stage parasites was determined by seeding 384-well microtiter plates with 40 μL per well of culture media and human blood infected with ring stage parasites at 2% parasitemia. Plates were then treated using by transferring 40 nL of compound from the source plate into the assay plate using a pin tool. After 72 hrs, plates were fixed, stained, and parasite growth quantified via high-content imaging using the methods previously described (PMID: 33792305). Cytotoxicity was determined by seeding 2,000 HepG2 cells in 40 μL per well of collagen-coated 384-well microtiter plates and, after 24 hrs, treating with a source plate using a pin tool as described above. After 72 hrs, plates were fixed, stained, and HepG2 growth quantified via high-content imaging using the methods previously described (PMID: 33792305). Raw data from all assays were normalized to the positive controls (dihydroartemisinin for P. falciparum blood stage assays and puromycin for HepG2 cytotoxicity assays) and the negative control (DMSO alone) and IC₅₀s were calculated using the curve fit software in CDD Vault.

TABLE 2
Biological Data for Disclosed Substances of Chemotype 1a

| | | | | | | PfABS W2 | HepG2 |
|---|---|---|---|---|---|---|---|
| Compound ID | R_{7c} | R_{7d} | R₇ₑ | R₄ | R_{1c} | (EC₅₀) (μM) | (CC₅₀) (μM) |
| APZ-2078 | Cl | H | H | CH₃ | CH₃ | 1.69 | >10.0 |
| APZ-2079 | Cl | H | H | CH₃ | OCH₃ | 3.86 | >10.0 |
| APZ-2080 | Cl | H | H | CH₃ | CH₃ | >10.0 | >10.0 |
| APZ-2081 | CH₃ | H | H | CH₃ | OCH₃ | 5.78 | >10.0 |
| APZ-2082 | CH₃ | CH₃ | H | CH₃ | OCH₃ | >10.0 | >10.0 |
| APZ-2083 | CH₃ | CH | H | CH₃ | CH₃ | >10.0 | >10.0 |
| APZ-2318 | OCH₃ | H | H | CH₃ | OCH₃ | >10.0 | >10.0 |
| APZ-2078 | OCH₃ | H | H | CH₃ | CH₃ | >4.21 | >10.0 |
| APZ-2029 | CH₃ | CH₃ | H | H | Cl | 1.56 | >10.0 |
| APZ-2030 | CN | H | H | CH₃ | Cl | 3.25 | >10.0 |
| APZ-2056 | CH₃ | H | H | CH₃ | H | 7.14 | >10.0 |
| APZ-2057 | H | H | H | CH₃ | H | >10.0 | >10.0 |
| APZ-2337 | H | CH₃ | H | CH₃ | OCH₃ | 5.77 | >10.0 |
| APZ-2336 | H | CH₃ | H | CH₃ | CH₃ | >2.82 | >10.0 |
| APZ-2335 | CH₃ | CH₃ | H | CH₃ | CH₂CH₃ | 2.01 | >10.0 |
| APZ-2304 | Cl | H | H | CH₃ | CH₂CH₃ | 0.612 | >10.0 |
| APZ-2303 | OCH₃ | H | H | CH₃ | CH₂CH₃ | 0.941 | >10.0 |
| APZ-2072 | Cl | H | H | CH₃ | CH(CH₃)₂ | 0.847 | >10.0 |
| APZ-2071 | OCH₃ | H | H | CH₃ | CH(CH₃)₂ | 1.62 | >10.0 |
| APZ-2070 | CH₃ | CH₃ | H | CH₃ | CH(CH₃)₂ | 4.15 | >10.0 |
| APZ-2068 | CH₃ | H | H | CH₃ | CH(CH₃)₂ | 0.742 | >10.0 |
| APZ-2067 | CH₃ | H | H | CH₃ | CH₂CH₃ | 0.574 | >10.0 |
| APZ-2066 | H | CH₃ | H | CH₃ | CH(CH₃)₂ | >10.0 | >10.0 |
| APZ-2065 | H | CH₃ | H | CH₃ | CH₂CH₃ | >4.26 | >10.0 |
| APZ-2064 | H | CH₃ | H | CH₃ | Cl | >4.03 | >10.0 |
| APZ-2063 | SO₂CH₃ | H | H | CH₃ | Cl | >10.0 | >10.0 |
| APZ-2062 | H | CF₃ | H | CH₃ | Cl | 3.27 | >10.0 |
| APZ-2061 | H | H | CH₃ | CH₃ | Cl | 5.20 | >10.0 |
| APZ-2055 | COCH₃ | H | H | H | Cl | >10.0 | >10.0 |
| APZ-2054 | NO₂ | H | H | H | Cl | >10.0 | >10.0 |
| APZ-2053 | Cl | Cl | H | CH₃ | Cl | 0.927 | >10.0 |
| APZ-2052 | NO₂ | H | H | CH₃ | Cl | 2.28 | >10.0 |
| APZ-2049 | COCH₃ | H | H | CH₃ | Cl | 2.42 | >10.0 |
| APZ-2048 | CF | H | H | CH₃ | Cl | 3.79 | >10.0 |
| APZ-2046 | Cl | Cl | H | H | Cl | >10.0 | >10.0 |
| APZ-2045 | CF₃ | H | H | H | Cl | >10.0 | >10.0 |
| APZ-2044 | C(CH₃)₃ | H | H | CH₃ | Cl | >10.0 | 1.76 |
| APZ-2043 | F | H | H | CH₃ | Cl | 1.78 | >10.0 |
| APZ-2042 | Cl | H | H | H | Cl | >10.0 | >10.0 |
| APZ-2041 | F | H | H | H | Cl | 5.15 | >10.0 |
| APZ-2028 | CH₃ | CH₃ | H | CH₃ | Cl | 2.36 | >10.0 |
| APZ-2027 | CH₂CH₃ | H | H | CH₃ | Cl | 5.24 | >10.0 |
| APZ-2026 | OCH₃ | H | H | H | Cl | 1.91 | >10.0 |
| APZ-2025 | Cl | H | H | CH₃ | Cl | 0.519 | >10.0 |
| APZ-2024 | N(CH₃)₂ | H | H | H | Cl | >10.0 | >10.0 |
| APZ-2023 | C(CH₃)₃ | H | H | H | Cl | >10.0 | >10.0 |
| APZ-2022 | CH₂CH₃ | H | H | H | Cl | >10.0 | >10.0 |
| APZ-2011 | CH₃ | H | H | CH₃ | F | 2.99 | 6.21 |
| APZ-2010 | CH₃ | H | H | CH₃ | Cl | 0.556 | >10.0 |
| APZ-2330 | H | H | H | CH₃ | Cl | >10.0 | >10.0 |
| APZ-2329 | H | H | H | CH₃ | CH₃ | >10.0 | >10.0 |
| APZ-2327 | CH₃ | H | H | CH₃ | CH₃ | 1.73 | >10.0 |
| APZ-2301 | Cl | H | H | CH₃ | CH₃ | 0.185 | >10.0 |

TABLE 3
Biological Data for Disclosed Substances of Chemotype 1b

| | | | | HepG2 |
|---|---|---|---|---|
| | | | PfABS W2 | (CC₅₀) |
| Compound ID | R₃ | R_{1c} | (EC₅₀) (μM) | (μM) |
| APZ-2339 | 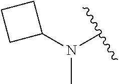 [SMILES: *N(C)C1CCC1] | Cl | >10.0 | >10.0 |
| APZ-2338 | 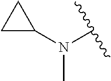 [SMILES: *N(C)C1CC1] | Cl | 5.77 | >10.0 |
| APZ-2122 | 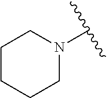 [SMILES: *N1CCCCC1] | Cl | 1.39 | >10.0 |
| APZ-2121 | 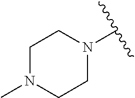 [SMILES: *N1CCN(C)CC1] | Cl | >10.0 | >10.0 |
| APZ-2313 | 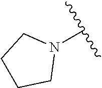 [SMILES: *N1CCCC1] | Cl | 3.50 | >10.0 |
| APZ-2088 | 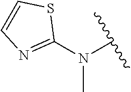 [SMILES: *N(C)c1nccs1] | Cl | >10.0 | >10.0 |
| APZ-2087 | 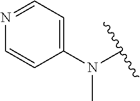 [SMILES: *N(C)c1ccncc1] | Cl | >10.0 | >10.0 |
| APZ-2086 | 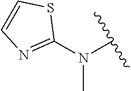 [SMILES: *N(C)c1nccs1] | CH₃ | >10.0 | >10.0 |
| APZ-2186 | 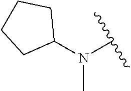 [SMILES: *N(C)C1CCCC1] | CH₃ | >10.0 | >10.0 |
| APZ-2085 | 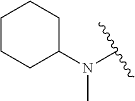 [SMILES: *N(C)C1CCCCC1] | CH₃ | >10.0 | >10.0 |
| APZ-2084 | 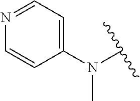 [SMILES: *N(C)c1ccncc1] | CH₃ | >10.0 | >10.0 |
| APZ-2306 | 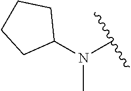 [SMILES: *N(C)C1CCCC1] | Cl | 0.860 | >10.0 |
| APZ-2305 | 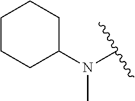 [SMILES: *N(C)C1CCCCC1] | Cl | >10.0 | >10.0 |

TABLE 4
Biological Data for Disclosed Substances of Chemotype 2

| | | | | PfABS W2 | HepG2 (CC₅₀) |
|---|---|---|---|---|---|
| Compound ID | R_{7c} | R₄ | R_{1c} | (EC₅₀) (μM) | (μM) |
| APZ-2198 | Cl | CH₃ | CF₃ | 0.0281 | >10.0 |
| APZ-2197 | Cl | CH₃ | CH(CH₃)₂ | 0.125 | 7.31 |
| APZ-2196 | Cl | CH₃ | CH₂CH₃ | 0.191 | >10.0 |
| APZ-2179 | Cl | CD₃ | Cl | 0.409 | >10.0 |
| APZ-2342 | CH₃ | CH₃ | Cl | 0.185 | >10.0 |
| APZ-2340 | CH₃ | CH₃ | CH₃ | 0.559 | >10.0 |
| APZ-2119 | Cl | CH₃ | CH₃ | 0.672 | >10.0 |
| APZ-2161 | Cl | CH₃ | Cl | 0.098 | >10.0 |
| APZ-2420 | | | | | |

TABLE 5
Biological Data for Disclosed Substances of Chemotype 3

| Compound | | | | PfABS W2 | HepG2 (CC₅₀) |
|---|---|---|---|---|---|
| ID | R_{7c} | R₄ | R_{1c} | (EC₅₀) (μM) | (μM) |
| APZ-2123 | Cl | CH₃ | Cl | 0.169 | >10.0 |
| APZ-2124 | Cl | CH₃ | CH₃ | 0.489 | >10.0 |
| APZ-2322 | CH₃ | CH₃ | Cl | 0.0477 | >9.73 |
| APZ-2183 | Cl | CD₃ | Cl | 0.209 | >10.0 |

TABLE 6
Biological Data for Disclosed Substances of Chemotype 4

| | | | | | | PfABS | |
|---|---|---|---|---|---|---|---|
| | | | | | | W2 | HepG2 |
| Compound | | | | | | (EC₅₀) | (CC₅₀) |
| ID | R_{7c} | R₄ | R₁ₑ | R_{1d} | R_{1c} | (μM) | (μM) |
| APZ-2321 | Cl | CH₃ | H | H | Cl | 0.435 | >10.0 |
| APZ-2160 | Cl | CH₃ | H | H | CH₃ | 0.131 | >10.0 |
| APZ-2323 | CH₃ | CH₃ | H | Cl | H | 1.62 | >10.0 |
| APZ-2325 | CH₃ | CH₃ | H | H | CF₃ | 0.0149 | >10.0 |
| APZ-2178 | Cl | CD₃ | H | H | CF₃ | >3.31 | >10.0 |
| APZ-2157 | Cl | CH₃ | H | H | CF₃ | 0.0158 | >10.0 |
| APZ-2158 | Cl | CH₃ | H | H | CH₂CH₃ | 0.322 | >10.0 |
| APZ-2190 | Cl | CH₃ | Cl | H | H | >3.33 | >10.0 |
| APZ-2290 | Cl | CH₃ | H | H | CH(CH₃)₂ | 0.245 | >10.0 |

TABLE 7
Biological Data for Disclosed Substances of Chemotype 5

| | | | | PfABS W2 | HepG2 (CC₅₀) |
|---|---|---|---|---|---|
| Compound ID | R_{7c} | R₄ | R_{1c} | (EC₅₀) (μM) | (μM) |
| APZ-2317 | Cl | CH₃ | Cl | 0.0411 | >10.0 |
| APZ-2191 | Cl | CH₃ | CH₃ | 0.0347 | >10.0 |

TABLE 8a
Biological Data for Disclosed Substance of Chemotype 6a

| | | | | | PfABS W2 | HepG2 |
|---|---|---|---|---|---|---|
| Compound ID | R_{7c} | R₇ₑ | R₄ | R_{1c} | (EC₅₀) (μM) | (CC₅₀) (μM) |
| APZ-2184 | Cl | H | CH₃ | CH₃ | 0.0512 | >10.0 |
| APZ-2199 | Cl | H | CH₃ | Cl | 0.0231 | >10.0 |
| APZ-2193 | CH₃ | H | CH₃ | CH₃ | 0.0214 | >10.0 |
| APZ-2192 | CH₃ | H | CH₃ | Cl | 0.0104 | >10.0 |
| APZ-2213 | Cl | H | CH₃ | CH₂CH₃ | 0.0174 | >10.0 |
| APZ-2214 | Cl | H | CH₃ | CF₃ | 0.00410 | >10.0 |
| APZ-2217 | Cl | H | CH₃ | CH(CH₃)₂ | 0.0241 | >10.0 |
| APZ-2298 | Cl | H | CD₃ | OCF₃ | 0.0187 | >10.0 |
| APZ-2299 | Cl | OCH₃ | CH₃ | OCF₃ | 0.00970 | >10.0 |
| APZ-2300 | Cl | OCH₃ | CD₃ | OCF₃ | 0.0128 | >10.0 |
| APZ-2289 | CCH | H | CH₃ | CF₃ | 0.0984 | >10.0 |
| APZ-2350 | cyclopropyl | H | CH₃ | CF₃ | 0.101 | >10.0 |
| APZ-2288 | Cl | H | CH₃ | OCF₂H | 0.0405 | >10.0 |
| APZ-2266 | Cl | H | CH(CH₃)₂ | CF₃ | 0.00274 | >10.0 |
| APZ-2267 | Cl | H | CH₂CH₃ | CF₃ | 0.00578 | >10.0 |
| APZ-2268 | Cl | H | CD₃ | CF₃ | 0.00698 | >10.0 |
| APZ-2291 | Cl | H | CH₃ | cyclobutyl | 0.0535 | >10.0 |
| APZ-2292 | Cl | H | CH₃ | cyclohexyl | 0.660 | >10.0 |
| APZ-2293 | Cl | H | CH₃ | cyclopentyl | 0.179 | >9.73 |
| APZ-2287 | F | H | CH₃ | CF₃ | 0.0124 | >10.0 |
| APZ-2355 | Cl | H | CH₂CH₃ | OCF₃ | 0.00971 | >9.97 |
| APZ-2254 | Cl | H | CH₃ | OCF₃ | 0.00825 | >10.0 |
| APZ-2275 | Cl | CH₃ | H | CF₃ | >10.0 | >10.0 |
| APZ-2276 | Cl | OCH₃ | CH₃ | CF₃ | 0.00118 | >10.0 |
| APZ-2279 | Cl | CH₃ | CH₃ | CF₃ | 0.00297 | >10.0 |
| APZ-2280 | Cl | H | CH₃ | CF₂H | 0.0129 | >10.0 |
| APZ-2283 | Cl | OCF₃ | CH₃ | CF₃ | 0.00302 | >10.0 |
| APZ-2284 | Cl | OH | CH₃ | CF₃ | 0.143 | >10.0 |
| APZ-2348 | Cl | H | C(CH₃)₃ | CF₃ | 0.0677 | >3.33 |
| APZ-2358 | Cl | NH₂ | CH₃ | CF₃ | 0.00479 | >10.0 |
| APZ-2351 | Cl | OCH₃ | CD₃ | CF₃ | 0.00645 | >10.0 |

TABLE 8b
Biological Data for Disclosed Substance
of Chemotype 6a, R_{1c} is CF₃

| | | | | PfABS W2 | HepG2 (CC₅₀) |
|---|---|---|---|---|---|
| Compound ID | R_{7c} | R₇ₑ | R₄ | (EC₅₀) (μM) | (μM) |
| APZ-2294 | Cl | CH₃ | CH₃ | 0.0100 | >10.0 |
| APZ-2296 | Cl | OCH₃ | CH₃ | 0.00936 | >10.0 |
| APZ-2297 | Cl | OCH₃ | CD₃ | 0.0123 | >10.0 |
| APZ-2253 | Cl | H | CH₃ | 0.0123 | >10.0 |

TABLE 8c
Biological Data for Disclosed Substance of Chemotype 6b

| | | | PfABS W2 | HepG2 |
|---|---|---|---|---|
| Compound | | | (EC₅₀) | (CC₅₀) |
| ID | R₉ | R₁₀ | (μM) | (μM) |
| APZ-2247 | 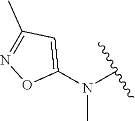 [SMILES: *N(C)c1cc(C)no1] | 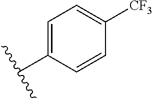 [SMILES: *c1ccc(C(F)(F)F)cc1] | 0.489 | >10.0 |
| APZ-2233 | 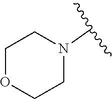 [SMILES: *N1CCOCC1] | 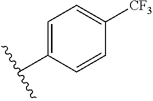 [SMILES: *c1ccc(C(F)(F)F)cc1] | >4.71 | >10.0 |
| APZ-2234 | 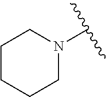 [SMILES: *N1CCCCC1] | 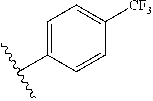 [SMILES: *c1ccc(C(F)(F)F)cc1] | >7.88 | >10.0 |
| APZ-2235 | 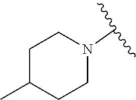 [SMILES: *N1CCC(C)CC1] | 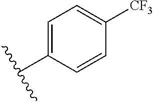 [SMILES: *c1ccc(C(F)(F)F)cc1] | >7.01 | >10.0 |
| APZ-2236 | 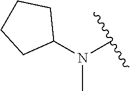 [SMILES: *N(C)C1CCCC1] | 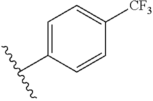 [SMILES: *c1ccc(C(F)(F)F)cc1] | >9.05 | >10.0 |
| APZ-2237 | 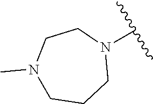 [SMILES: *N1CCCN(C)CC1] | 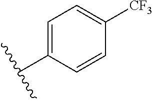 [SMILES: *c1ccc(C(F)(F)F)cc1] | >4.8 | >10.0 |
| APZ-2238 | 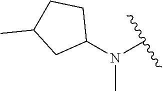 [SMILES: *N(C)C1CCC(C)C1] | 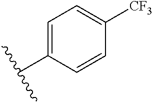 [SMILES: *c1ccc(C(F)(F)F)cc1] | >3.33 | >10.0 |
| APZ-2239 | 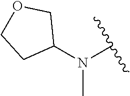 [SMILES: *N(C)C1CCOC1] | 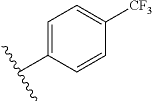 [SMILES: *c1ccc(C(F)(F)F)cc1] | >9.88 | >10.0 |
| APZ-2240 | 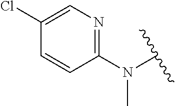 [SMILES: *N(C)c1ccc(Cl)cn1] | 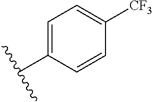 [SMILES: *c1ccc(C(F)(F)F)cc1] | 0.00707 | >10.0 |
| APZ-2241 | 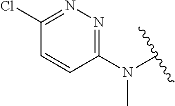 [SMILES: *N(C)c1ccc(Cl)nn1] | 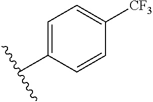 [SMILES: *c1ccc(C(F)(F)F)cc1] | 0.0201 | >10.0 |
| APZ-2242 | 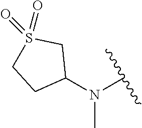 [SMILES: *N(C)C1CCS(=O)(=O)C1] | 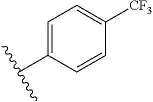 [SMILES: *c1ccc(C(F)(F)F)cc1] | >2.11 | >10.0 |
| APZ-2243 | 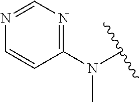 [SMILES: *N(C)c1ccncn1] | 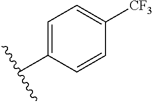 [SMILES: *c1ccc(C(F)(F)F)cc1] | >3.05 | >10.0 |
| APZ-2244 | 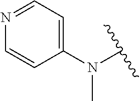 [SMILES: *N(C)c1ccncc1] | 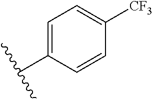 [SMILES: *c1ccc(C(F)(F)F)cc1] | >2.15 | >10.0 |
| APZ-2249 | 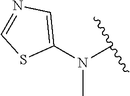 [SMILES: *N(C)c1cncs1] | 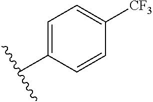 [SMILES: *c1ccc(C(F)(F)F)cc1] | 1.30 | >10.0 |
| APZ-2269 | 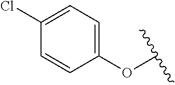 [SMILES: *Oc1ccc(Cl)cc1] | 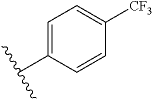 [SMILES: *c1ccc(C(F)(F)F)cc1] | >10.0 | >10.0 |
| APZ-2270 | 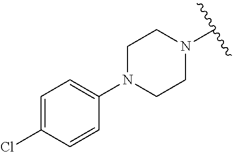 [SMILES: *N1CCN(c2ccc(Cl)cc2)CC1] | 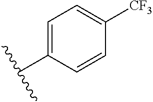 [SMILES: *c1ccc(C(F)(F)F)cc1] | >10.0 | >6.83 |
| APZ-2271 | 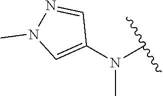 [SMILES: *N(C)c1cnn(C)c1] | 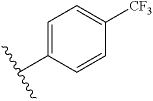 [SMILES: *c1ccc(C(F)(F)F)cc1] | 0.582 | >10.0 |
| APZ-2272 | 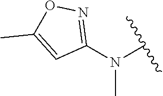 [SMILES: *N(C)c1cc(C)on1] | 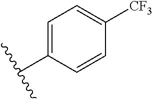 [SMILES: *c1ccc(C(F)(F)F)cc1] | 2.05 | >10.0 |
| APZ-2274 | 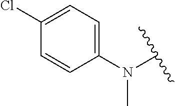 [SMILES: *N(C)c1ccc(Cl)cc1] | 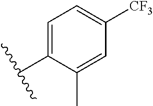 [SMILES: *c1ccc(C(F)(F)F)cc1C] | 0.174 | >10.0 |
| APZ-2295 | 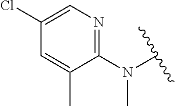 [SMILES: *N(C)c1ncc(Cl)cc1C] | 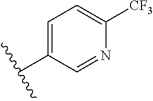 [SMILES: *c1ccc(C(F)(F)F)nc1] | 0.0977 | >10.0 |
| APZ-2302 | 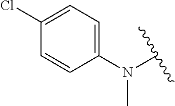 [SMILES: *N(C)c1ccc(Cl)cc1] | 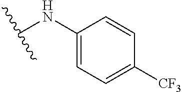 [SMILES: *Nc1ccc(C(F)(F)F)cc1] | 0.689 | 2.84 |
| APZ-2360 | 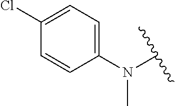 [SMILES: *N(C)c1ccc(Cl)cc1] | 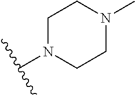 [SMILES: *N1CCN(C)CC1] | 5.77 | >10.0 |
| APZ-2248 | 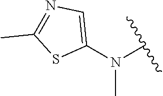 [SMILES: *N(C)c1cnc(C)s1] | 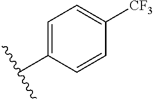 [SMILES: *c1ccc(C(F)(F)F)cc1] | 0.0683 | >10.0 |
| APZ-2259 | 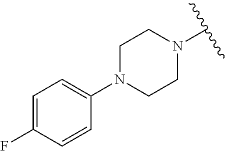 [SMILES: *N1CCN(c2ccc(F)cc2)CC1] | 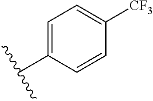 [SMILES: *c1ccc(C(F)(F)F)cc1] | >10.0 | >5.77 |
| APZ-2352 | 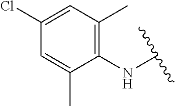 [SMILES: *Nc1c(C)cc(Cl)cc1C] | 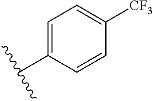 [SMILES: *c1ccc(C(F)(F)F)cc1] | 5.79 | >10.0 |
| APZ-2261 | 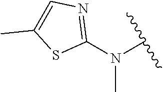 [SMILES: *N(C)c1ncc(C)s1] | 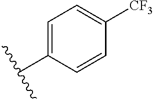 [SMILES: *c1ccc(C(F)(F)F)cc1] | >4.03 | >10.0 |
| APZ-2252 | 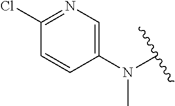 [SMILES: *N(C)c1ccc(Cl)nc1] | 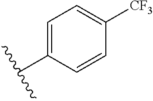 [SMILES: *c1ccc(C(F)(F)F)cc1] | 0.00492 | >10.0 |
| APZ-2263 | 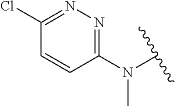 [SMILES: *N(C)c1ccc(Cl)nn1] | 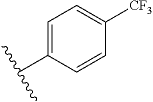 [SMILES: *c1ccc(C(F)(F)F)cc1] | >10.0 | >10.0 |
| APZ-2264 | 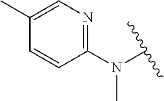 [SMILES: *N(C)c1ccc(C)cn1] | 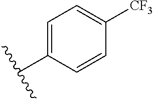 [SMILES: *c1ccc(C(F)(F)F)cc1] | 0.0269 | >10.0 |
| APZ-2265 | 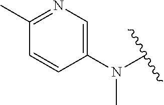 [SMILES: *N(C)c1ccc(C)nc1] | 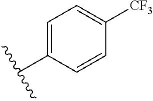 [SMILES: *c1ccc(C(F)(F)F)cc1] | 0.00956 | >6.55 |
| APZ-2258 | 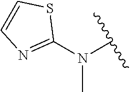 [SMILES: *N(C)c1nccs1] | 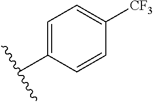 [SMILES: *c1ccc(C(F)(F)F)cc1] | >10.0 | >10.0 |
| APZ-2232 | 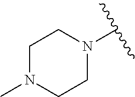 [SMILES: *N1CCN(C)CC1] | 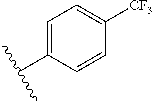 [SMILES: *c1ccc(C(F)(F)F)cc1] | >6.93 | >10.0 |
| APZ-2285 | 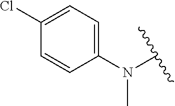 [SMILES: *N(C)c1ccc(Cl)cc1] | 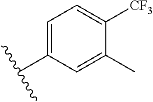 [SMILES: *c1ccc(C(F)(F)F)c(C)c1] | 0.00423 | >10.0 |
| APZ-2353 | 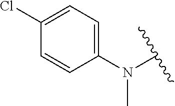 [SMILES: *N(C)c1ccc(Cl)cc1] | 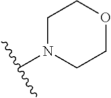 [SMILES: *N1CCOCC1] | >10.0 | >10.0 |
| APZ-2354 | 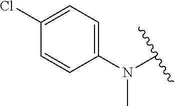 [SMILES: *N(C)c1ccc(Cl)cc1] | 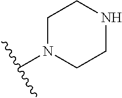 [SMILES: *N1CCNCC1] | >10.0 | >10.0 |
| APZ-2255 | 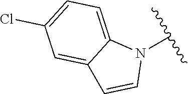 [SMILES: *n1ccc2cc(Cl)ccc21] | 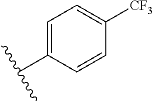 [SMILES: *c1ccc(C(F)(F)F)cc1] | >10.0 | >10.0 |
| APZ-2277 | 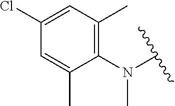 [SMILES: *N(C)c1c(C)cc(Cl)cc1C] | 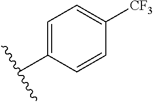 [SMILES: *c1ccc(C(F)(F)F)cc1] | 0.00545 | >10.0 |
| APZ-2281 | 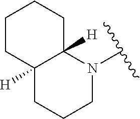 [SMILES: *N1CCC[C@H]2CCCC[C@@H]21] | 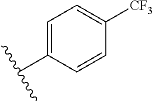 [SMILES: *c1ccc(C(F)(F)F)cc1] | >3.52 | >10.0 |
| APZ-2356 | 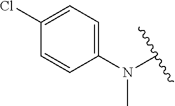 [SMILES: *N(C)c1ccc(Cl)cc1] | 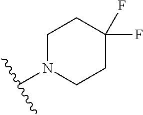 [SMILES: *N1CCC(F)(F)CC1] | 0.474 | >10.0 |
| APZ-2357 | 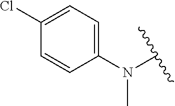 [SMILES: *N(C)c1ccc(Cl)cc1] | 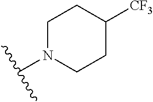 [SMILES: *N1CCC(C(F)(F)F)CC1] | 0.0553 | >3.33 |
| APZ-2286 | 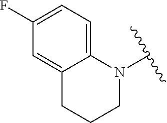 [SMILES: *N1CCCc2cc(F)ccc21] | 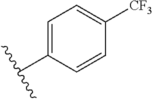 [SMILES: *c1ccc(C(F)(F)F)cc1] | 0.107 | >10.0 |
| APZ-2256 | 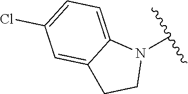 [SMILES: *N1CCc2cc(Cl)ccc21] | 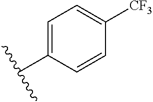 [SMILES: *c1ccc(C(F)(F)F)cc1] | >10.0 | >10.0 |
| APZ-2257 | 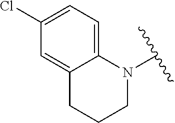 [SMILES: *N1CCCc2cc(Cl)ccc21] | 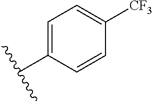 [SMILES: *c1ccc(C(F)(F)F)cc1] | 0.0260 | >10.0 |
| APZ-2728 | 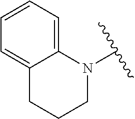 [SMILES: *N1CCCc2ccccc21] | 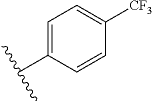 [SMILES: *c1ccc(C(F)(F)F)cc1] | 0.433 | 4.07 |
| APZ-2282 | 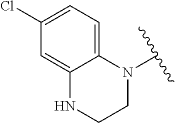 [SMILES: *N1CCNc2cc(Cl)ccc21] | 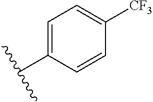 [SMILES: *c1ccc(C(F)(F)F)cc1] | 1.24 | >10.0 |
| APZ-2347 | 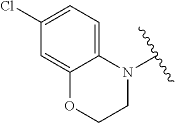 [SMILES: *N1CCOc2cc(Cl)ccc21] | 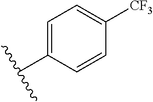 [SMILES: *c1ccc(C(F)(F)F)cc1] | 0.295 | >10.0 |
| APZ-2349 | 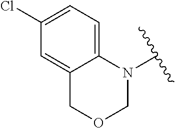 [SMILES: *N1COCc2cc(Cl)ccc21] | 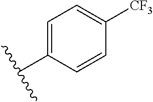 [SMILES: *c1ccc(C(F)(F)F)cc1] | 0.808 | >5.77 |

TABLE 9
Biological Data for Disclosed Substances of Chemotype 7

| | | | | PfABS | |
|---|---|---|---|---|---|
| | | | | W2 | HepG2 |
| Compound | | | | (EC₅₀) | (CC₅₀) |
| ID | R_{7c} | R₄ | R_{1c} | (μM) | (μM) |
| APZ-2195 | Cl | CH₃ | Cl | 0.0129 | >10.0 |
| APZ-2194 | Cl | CH₃ | CH₃ | 0.0382 | >10.0 |
| APZ-2211 | CH₃ | CH₃ | CH₃ | 0.0417 | >10.0 |
| APZ-2212 | CH₃ | CH₃ | Cl | 0.0246 | >10.0 |
| APZ-2215 | Cl | CH₃ | CH₂CH₃ | 0.0256 | >10.0 |
| APZ-2216 | Cl | CH₃ | CF₃ | 0.00456 | >10.0 |
| APZ-2250 | Cl | CH(CH₃)₂ | CF₃ | 0.00142 | >6.44 |
| APZ-2251 | Cl | CH₂CH₃ | CF₃ | 0.00282 | >6.44 |
| APZ-2218 | Cl | CH₃ | CH(CH₃)₂ | 0.0418 | >10.0 |

TABLE 11
Biological Data for Disclosed Substances of Chemotype 9

| | | PfABS W2 | HepG2 (CC₅₀) |
|---|---|---|---|
| Compound ID | Structure | (EC₅₀) (μM) | (μM) |
| APZ-2320 | 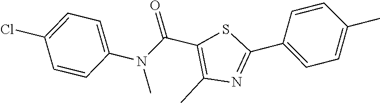 [SMILES: Cc1ccc(-c2nc(C)c(C(=O)N(C)c3ccc(Cl)cc3)s2)cc1] | >10.0 | >10.0 |
| APZ-2125 | 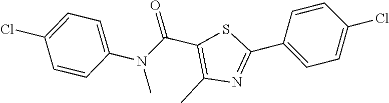 [SMILES: Cc1nc(-c2ccc(Cl)cc2)sc1C(=O)N(C)c1ccc(Cl)cc1] | >10.0 | >10.0 |
| APZ-2324 | 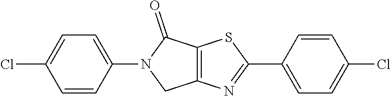 [SMILES: O=C1c2sc(-c3ccc(Cl)cc3)nc2CN1c1ccc(Cl)cc1] | >10.0 | >10.0 |
| APZ-2326 | 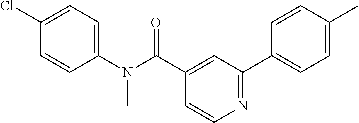 [SMILES: Cc1ccc(-c2cc(C(=O)N(C)c3ccc(Cl)cc3)ccn2)cc1] | >7.98 | >10.0 |
| APZ-2177 | 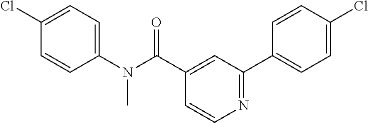 [SMILES: CN(C(=O)c1ccnc(-c2ccc(Cl)cc2)c1)c1ccc(Cl)cc1] | 3.69 | >10.0 |
| APZ-2316 | 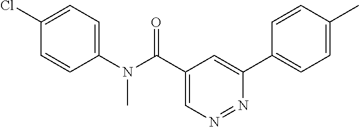 [SMILES: Cc1ccc(-c2cc(C(=O)N(C)c3ccc(Cl)cc3)cnn2)cc1] | >10.0 | >10.0 |
| APZ-2185 | 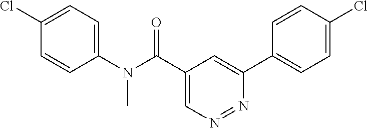 [SMILES: CN(C(=O)c1cnnc(-c2ccc(Cl)cc2)c1)c1ccc(Cl)cc1] | >8.62 | >10.0 |
| APZ-2117 | 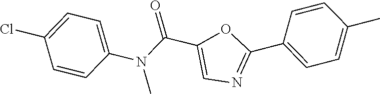 [SMILES: Cc1ccc(-c2ncc(C(=O)N(C)c3ccc(Cl)cc3)o2)cc1] | >10.0 | >10.0 |
| APZ-2118 | 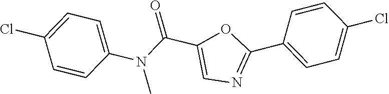 [SMILES: CN(C(=O)c1cnc(-c2ccc(Cl)cc2)o1)c1ccc(Cl)cc1] | >10.0 | >10.0 |
| APZ-2182 | 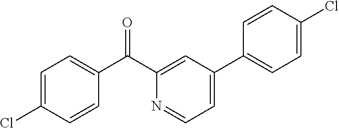 [SMILES: O=C(c1ccc(Cl)cc1)c1cc(-c2ccc(Cl)cc2)ccn1] | >10.0 | >10.0 |
| APZ-2180 | 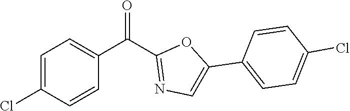 [SMILES: O=C(c1ccc(Cl)cc1)c1ncc(-c2ccc(Cl)cc2)o1] | >10.0 | >10.0 |
| APZ-2273 | 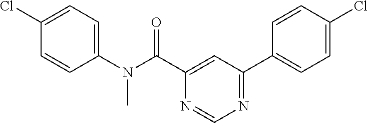 [SMILES: CN(C(=O)c1cc(-c2ccc(Cl)cc2)ncn1)c1ccc(Cl)cc1] | >10.0 | >10.0 |
| APZ-2346 | 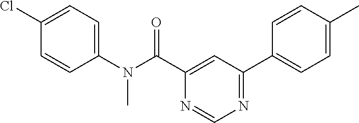 [SMILES: Cc1ccc(-c2cc(C(=O)N(C)c3ccc(Cl)cc3)ncn2)cc1] | >10.0 | >10.0 |
| APZ-2345 | 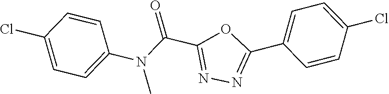 [SMILES: CN(C(=O)c1nnc(-c2ccc(Cl)cc2)o1)c1ccc(Cl)cc1] | >10.0 | >10.0 |
| APZ-2344 | 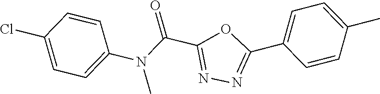 [SMILES: Cc1ccc(-c2nnc(C(=O)N(C)c3ccc(Cl)cc3)o2)cc1] | >10.0 | >10.0 |
| APZ-2334 | 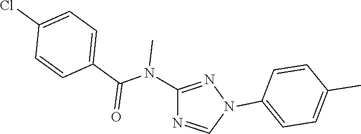 [SMILES: Cc1ccc(-n2cnc(N(C)C(=O)c3ccc(Cl)cc3)n2)cc1] | 1.80 | >10.0 |
| APZ-2333 | 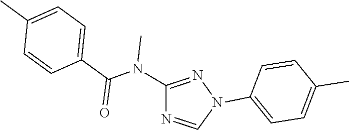 | >3.38 | >10.0 |
| APZ-2332 | 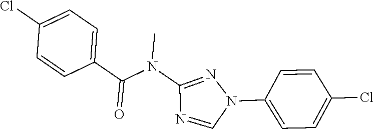 [SMILES: CN(C(=O)c1ccc(Cl)cc1)c1ncn(-c2ccc(Cl)cc2)n1] | >1.52 | >10.0 |
| APZ-2331 | 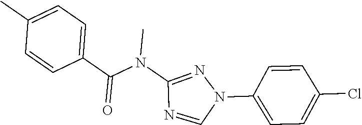 [SMILES: Cc1ccc(C(=O)N(C)c2ncn(-c3ccc(Cl)cc3)n2)cc1] | 4.24 | >10.0 |
| APZ-2165 | 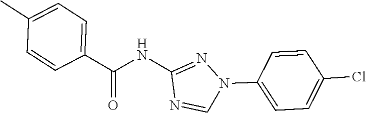 [SMILES: Cc1ccc(C(=O)Nc2ncn(-c3ccc(Cl)cc3)n2)cc1] | 3.87 | >10.0 |
| APZ-2164 | 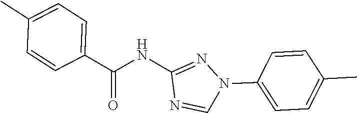 | 2.49 | >10.0 |
| APZ-2163 | 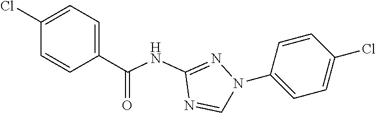 [SMILES: O=C(Nc1ncn(-c2ccc(Cl)cc2)n1)c1ccc(Cl)cc1] | 3.08 | >10.0 |
| APZ-2162 | 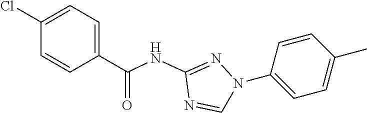 [SMILES: Cc1ccc(-n2cnc(NC(=O)c3ccc(Cl)cc3)n2)cc1] | 2.99 | >10.0 |
| APZ-2315 | 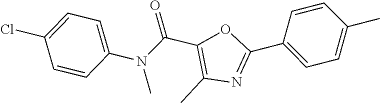 [SMILES: Cc1ccc(-c2nc(C)c(C(=O)N(C)c3ccc(Cl)cc3)o2)cc1] | >10.0 | >10.0 |
| APZ-2314 | 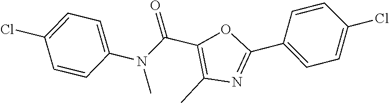 [SMILES: Cc1nc(-c2ccc(Cl)cc2)oc1C(=O)N(C)c1ccc(Cl)cc1] | >4.73 | >10.0 |
| APZ-2312 | 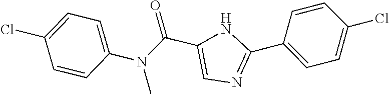 [SMILES: CN(C(=O)c1cnc(-c2ccc(Cl)cc2)[nH]1)c1ccc(Cl)cc1] | >10.0 | >10.0 |
| APZ-2311 | 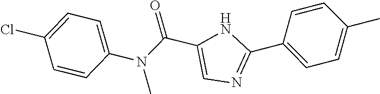 [SMILES: Cc1ccc(-c2ncc(C(=O)N(C)c3ccc(Cl)cc3)[nH]2)cc1] | 3.42 | >10.0 |
| APZ-2310 | 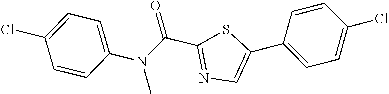 [SMILES: CN(C(=O)c1ncc(-c2ccc(Cl)cc2)s1)c1ccc(Cl)cc1] | >6.75 | >10.0 |
| APZ-2309 | 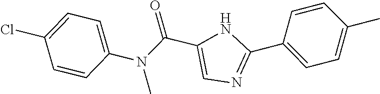 [SMILES: Cc1ccc(-c2ncc(C(=O)N(C)c3ccc(Cl)cc3)[nH]2)cc1] | >10.0 | >10.0 |
| APZ-2308 | 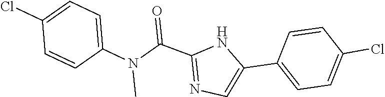 [SMILES: CN(C(=O)c1ncc(-c2ccc(Cl)cc2)[nH]1)c1ccc(Cl)cc1] | >7.09 | >10.0 |
| APZ-2095 | 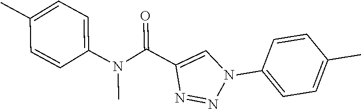 | >10.0 | >10.0 |
| APZ-2188 | 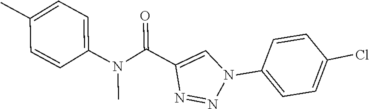 [SMILES: Cc1ccc(N(C)C(=O)c2cn(-c3ccc(Cl)cc3)nn2)cc1] | >10.0 | >10.0 |
| APZ-2094 | 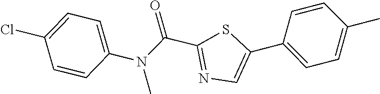 [SMILES: Cc1ccc(-c2cnc(C(=O)N(C)c3ccc(Cl)cc3)s2)cc1] | >10.0 | >10.0 |
| APZ-2092 | 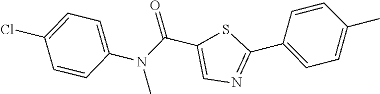 [SMILES: Cc1ccc(-c2ncc(C(=O)N(C)c3ccc(Cl)cc3)s2)cc1] | >10.0 | >10.0 |
| APZ-2091 | 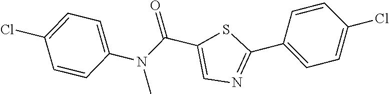 [SMILES: CN(C(=O)c1cnc(-c2ccc(Cl)cc2)s1)c1ccc(Cl)cc1] | >10.0 | >10.0 |
| APZ-2090 | 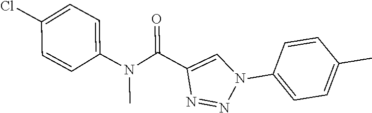 [SMILES: Cc1ccc(-n2cc(C(=O)N(C)c3ccc(Cl)cc3)nn2)cc1] | >10.0 | >10.0 |
| APZ-2089 | 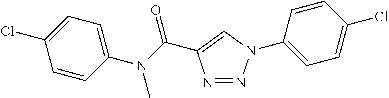 [SMILES: CN(C(=O)c1cn(-c2ccc(Cl)cc2)nn1)c1ccc(Cl)cc1] | >10.0 | >10.0 |
| APZ-2359 | 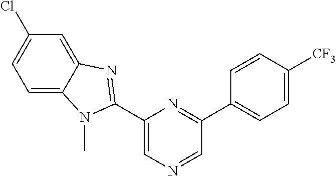 [SMILES: Cn1c(-c2cncc(-c3ccc(C(F)(F)F)cc3)n2)nc2cc(Cl)ccc21] | >10.0 | >5.77 |

TABLE 12
Biological Data for Additional Disclosed Substance

| | | PfABS W2 | HepG2 (CC₅₀) |
|---|---|---|---|
| Compound ID | Structure | (EC₅₀) (μM) | (μM) |
| APZ-2368 | 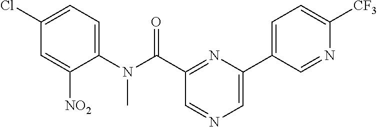 [SMILES: CN(C(=O)c1cncc(-c2ccc(C(F)(F)F)nc2)n1)c1ccc(Cl)cc1[N+](=O)[O-]] | 0.0861 | >3.33 |
| APZ-2367 | 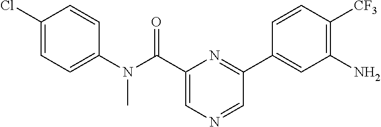 [SMILES: CN(C(=O)c1cncc(-c2ccc(C(F)(F)F)c(N)c2)n1)c1ccc(Cl)cc1] | 0.0848 | >3.33 |
| APZ-2366 | 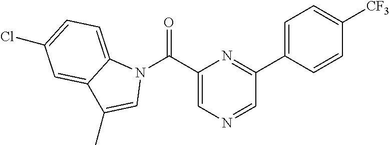 [SMILES: Cc1cn(C(=O)c2cncc(-c3ccc(C(F)(F)F)cc3)n2)c2ccc(Cl)cc12] | >5.77 | >10.0 |
| APZ-2365 | 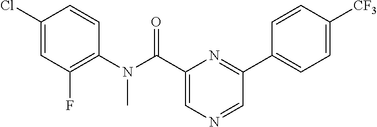 [SMILES: CN(C(=O)c1cncc(-c2ccc(C(F)(F)F)cc2)n1)c1ccc(Cl)cc1F] | 0.00488 | >5.77 |
| APZ-2372 | 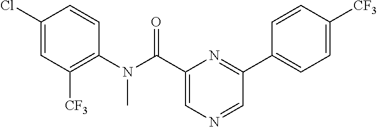 [SMILES: CN(C(=O)c1cncc(-c2ccc(C(F)(F)F)cc2)n1)c1ccc(Cl)cc1C(F)(F)F] | 0.0109 | >5.77 |
| APZ-2370 | 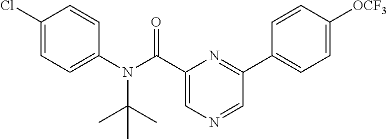 [SMILES: CC(C)(C)N(C(=O)c1cncc(-c2ccc(OC(F)(F)F)cc2)n1)c1ccc(Cl)cc1] | 0.221 | >10.0 |
| APZ-2369 | 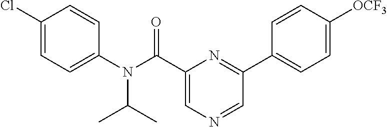 [SMILES: CC(C)N(C(=O)c1cncc(-c2ccc(OC(F)(F)F)cc2)n1)c1ccc(Cl)cc1] | 0.00490 | >10.0 |
| APZ-2364 | 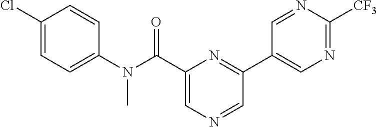 [SMILES: CN(C(=O)c1cncc(-c2cnc(C(F)(F)F)nc2)n1)c1ccc(Cl)cc1] | 0.0169 | >10.0 |
| APZ-2363 | 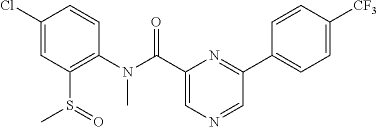 [SMILES: CN(C(=O)c1cncc(-c2ccc(C(F)(F)F)cc2)n1)c1ccc(Cl)cc1S(C)=O] | 0.0842 | >5.77 |
| APZ-2362 | 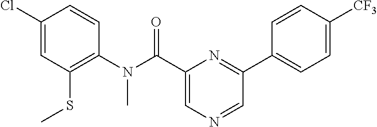 [SMILES: CSc1cc(Cl)ccc1N(C)C(=O)c1cncc(-c2ccc(C(F)(F)F)cc2)n1] | 0.00274 | >10.0 |
| APZ-2361 | 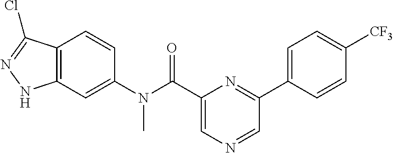 [SMILES: CN(C(=O)c1cncc(-c2ccc(C(F)(F)F)cc2)n1)c1ccc2c(Cl)n[nH]c2c1] | 0.135 | >5.77 |
| APZ-2375 | 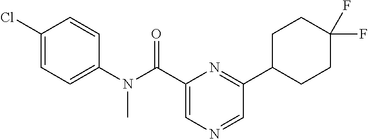 [SMILES: CN(C(=O)c1cncc(C2CCC(F)(F)CC2)n1)c1ccc(Cl)cc1] | 0.320 | >10.0 |
| APZ-2373 | 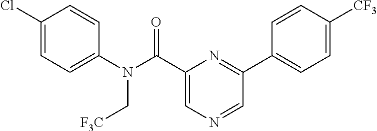 [SMILES: O=C(c1cncc(-c2ccc(C(F)(F)F)cc2)n1)N(CC(F)(F)F)c1ccc(Cl)cc1] | 0.00476 | >5.77 |
| APZ-2371 | 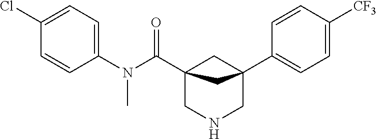 [SMILES: CN(c1ccc(Cl)cc1)C(=O)[C@]12CNC[C@](c3ccc(C(F)(F)F)cc3)(C1)C2] | >4.75 | >3.33 |
| APZ-2376 | 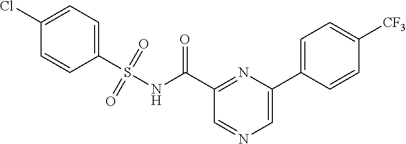 [SMILES: O=C(NS(=O)(=O)c1ccc(Cl)cc1)c1cncc(-c2ccc(C(F)(F)F)cc2)n1] | >10.0 | >10.0 |
| APZ-2377 | 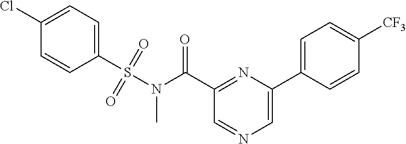 [SMILES: CN(C(=O)c1cncc(-c2ccc(C(F)(F)F)cc2)n1)S(=O)(=O)c1ccc(Cl)cc1] | >10.0 | >10.0 |
| APZ-2381 | 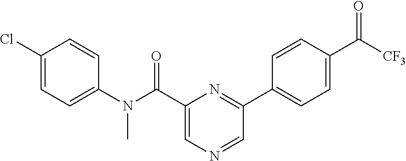 [SMILES: CN(C(=O)c1cncc(-c2ccc(C(=O)C(F)(F)F)cc2)n1)c1ccc(Cl)cc1] | 1.18 | >10.0 |
| APZ-2382 | 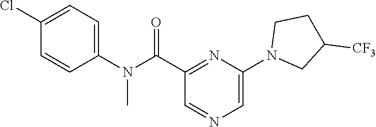 [SMILES: CN(C(=O)c1cncc(N2CCC(C(F)(F)F)C2)n1)c1ccc(Cl)cc1] | 0.195 | 8.02 |
| APZ-2392 | 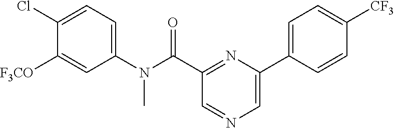 [SMILES: CN(C(=O)c1cncc(-c2ccc(C(F)(F)F)cc2)n1)c1ccc(Cl)c(OC(F)(F)F)c1] | 0.0275 | >10.0 |
| APZ-2393 | 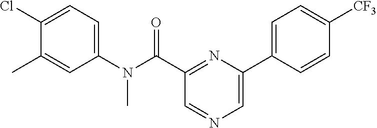 [SMILES: Cc1cc(N(C)C(=O)c2cncc(-c3ccc(C(F)(F)F)cc3)n2)ccc1Cl] | 0.0234 | >10.0 |
| APZ-2394 | 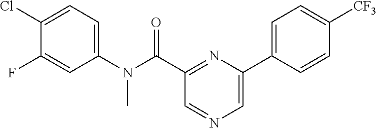 [SMILES: CN(C(=O)c1cncc(-c2ccc(C(F)(F)F)cc2)n1)c1ccc(Cl)c(F)c1] | 0.00421 | >10.0 |
| APZ-2400 | 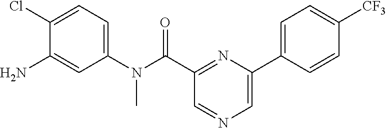 [SMILES: CN(C(=O)c1cncc(-c2ccc(C(F)(F)F)cc2)n1)c1ccc(Cl)c(N)c1] | 0.00961 | 5.88 |
| APZ-2401 | 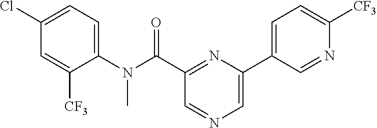 [SMILES: CN(C(=O)c1cncc(-c2ccc(C(F)(F)F)nc2)n1)c1ccc(Cl)cc1C(F)(F)F] | 0.0393 | >10.0 |
| APZ-2402 | 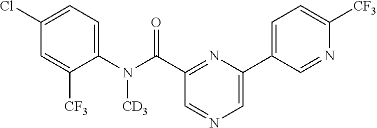 [SMILES: [2H]C([2H])([2H])N(C(=O)c1cncc(-c2ccc(C(F)(F)F)nc2)n1)c1ccc(Cl)cc1C(F)(F)F] | 0.0314 | >7.80 |
| APZ-2403 | 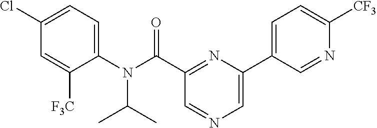 [SMILES: CC(C)N(C(=O)c1cncc(-c2ccc(C(F)(F)F)nc2)n1)c1ccc(Cl)cc1C(F)(F)F] | 5.94 | >5.41 |
| APZ-2404 | 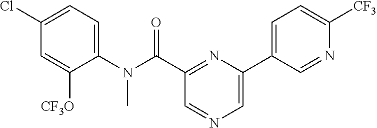 | 0.00861 | >8.75 |
| APZ-2405 | 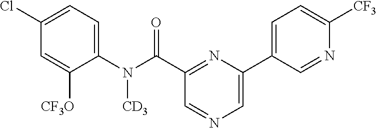 | 0.0119 | >3.66 |
| APZ-2406 | 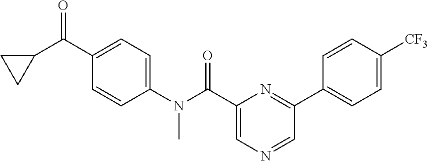 [SMILES: CN(C(=O)c1cncc(-c2ccc(C(F)(F)F)cc2)n1)c1ccc(C(=O)C2CC2)cc1] | 6.33 | 6.94 |
| APZ-2407 | 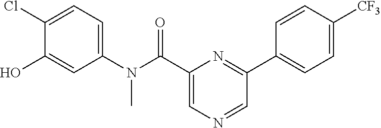 [SMILES: CN(C(=O)c1cncc(-c2ccc(C(F)(F)F)cc2)n1)c1ccc(Cl)c(O)c1] | 0.0455 | >10.0 |
| APZ-2410 | 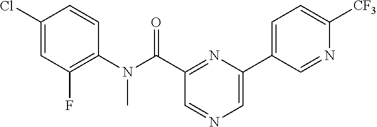 [SMILES: CN(C(=O)c1cncc(-c2ccc(C(F)(F)F)nc2)n1)c1ccc(Cl)cc1F] | 0.0352 | >8.73 |
| APZ-2411 | 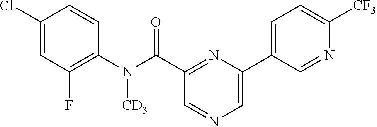 [SMILES: [2H]C([2H])([2H])N(C(=O)c1cncc(-c2ccc(C(F)(F)F)nc2)n1)c1ccc(Cl)cc1F] | 0.0346 | >10.0 |
| APZ-2412 | 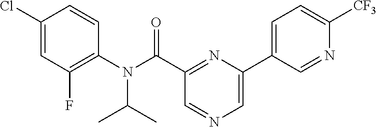 [SMILES: CC(C)N(C(=O)c1cncc(-c2ccc(C(F)(F)F)nc2)n1)c1ccc(Cl)cc1F] | >5.74 | 1.87 |
| APZ-2413 | 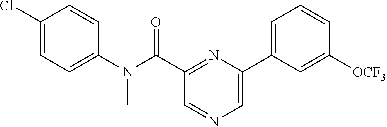 [SMILES: CN(C(=O)c1cncc(-c2cccc(OC(F)(F)F)c2)n1)c1ccc(Cl)cc1] | 0.0880 | 6.93 |
| APZ-2414 | 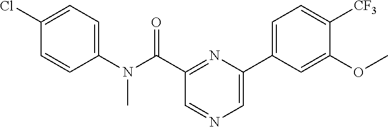 [SMILES: COc1cc(-c2cncc(C(=O)N(C)c3ccc(Cl)cc3)n2)ccc1C(F)(F)F] | 0.0160 | >10.0 |
| APZ-2415 | 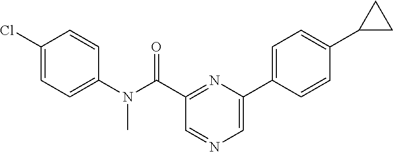 [SMILES: CN(C(=O)c1cncc(-c2ccc(C3CC3)cc2)n1)c1ccc(Cl)cc1] | 0.108 | >10.0 |
| APZ-2420 | 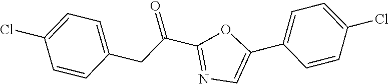 [SMILES: O=C(Cc1ccc(Cl)cc1)c1ncc(-c2ccc(Cl)cc2)o1] | >4.83 | >10.0 |
| APZ-2421 | 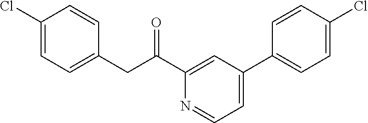 [SMILES: O=C(Cc1ccc(Cl)cc1)c1cc(-c2ccc(Cl)cc2)ccn1] | >4.89 | >10.0 |
| APZ-2422 | 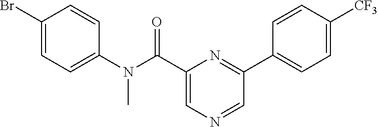 [SMILES: CN(C(=O)c1cncc(-c2ccc(C(F)(F)F)cc2)n1)c1ccc(Br)cc1] | 0.0313 | >4.01 |
| APZ-2423 | 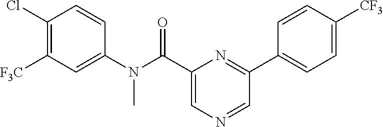 [SMILES: CN(C(=O)c1cncc(-c2ccc(C(F)(F)F)cc2)n1)c1ccc(Cl)c(C(F)(F)F)c1] | 0.143 | 6.55 |
| APZ-2424 | 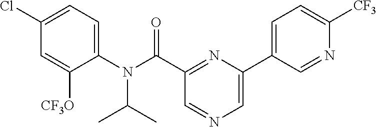 | 1.76 | 6.12 |
| APZ-2438 | 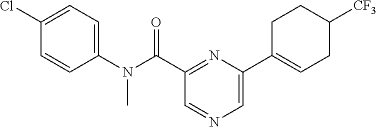 [SMILES: CN(C(=O)c1cncc(C2=CCC(C(F)(F)F)CC2)n1)c1ccc(Cl)cc1] | 0.0163 | >10.0 |
| APZ-2439 | 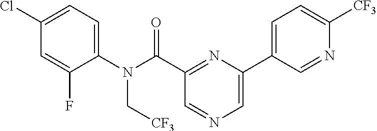 [SMILES: O=C(c1cncc(-c2ccc(C(F)(F)F)nc2)n1)N(CC(F)(F)F)c1ccc(Cl)cc1F] | 0.0799 | >4.44 |
| APZ-2440 | 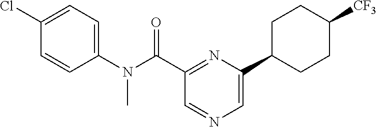 [SMILES: CN(C(=O)c1cncc([C@H]2CC[C@@H](C(F)(F)F)CC2)n1)c1ccc(Cl)cc1] | 0.826 | >10.0 |
| APZ-2441 | 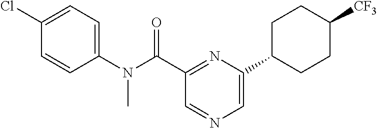 [SMILES: CN(C(=O)c1cncc([C@H]2CC[C@H](C(F)(F)F)CC2)n1)c1ccc(Cl)cc1] | 0.0169 | >10.0 |
| APZ-2442 | 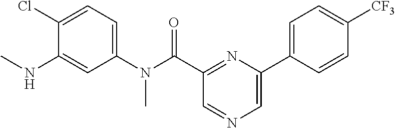 [SMILES: CNc1cc(N(C)C(=O)c2cncc(-c3ccc(C(F)(F)F)cc3)n2)ccc1Cl] | 0.0255 | >8.86 |
| APZ-2443 | 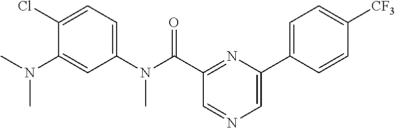 [SMILES: CN(C)c1cc(N(C)C(=O)c2cncc(-c3ccc(C(F)(F)F)cc3)n2)ccc1Cl] | 0.269 | 5.51 |
| APZ-2444 | 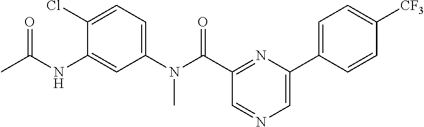 [SMILES: CC(=O)Nc1cc(N(C)C(=O)c2cncc(-c3ccc(C(F)(F)F)cc3)n2)ccc1Cl] | 0.737 | 7.12 |
| APZ-2445 | 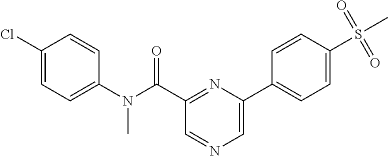 [SMILES: CN(C(=O)c1cncc(-c2ccc(S(C)(=O)=O)cc2)n1)c1ccc(Cl)cc1] | 1.57 | >10.0 |
| APZ-2446 | 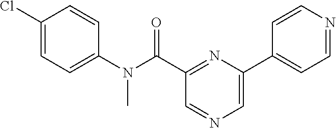 [SMILES: CN(C(=O)c1cncc(-c2ccncc2)n1)c1ccc(Cl)cc1] | >4.68 | 4.44 |
| APZ-2447 | 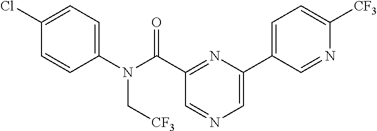 [SMILES: O=C(c1cncc(-c2ccc(C(F)(F)F)nc2)n1)N(CC(F)(F)F)c1ccc(Cl)cc1] | 0.00597 | >9.61 |
| APZ-2448 | 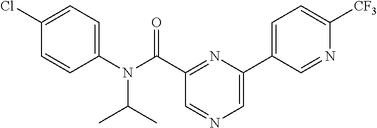 [SMILES: CC(C)N(C(=O)c1cncc(-c2ccc(C(F)(F)F)nc2)n1)c1ccc(Cl)cc1] | 0.000818 | >9.63 |
| APZ-2449 | 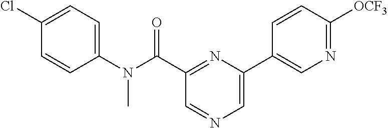 [SMILES: CN(C(=O)c1cncc(-c2ccc(OC(F)(F)F)nc2)n1)c1ccc(Cl)cc1] | 0.0120 | >10.0 |
| APZ-2450 | 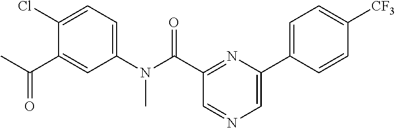 [SMILES: CC(=O)c1cc(N(C)C(=O)c2cncc(-c3ccc(C(F)(F)F)cc3)n2)ccc1Cl] | 0.0394 | >7.86 |
| APZ-2462 | 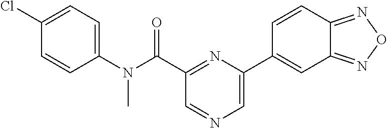 [SMILES: CN(C(=O)c1cncc(-c2ccc3nonc3c2)n1)c1ccc(Cl)cc1] | 0.0621 | >10.0 |
| APZ-2463 | 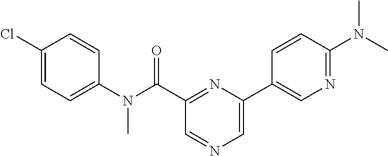 [SMILES: CN(C)c1ccc(-c2cncc(C(=O)N(C)c3ccc(Cl)cc3)n2)cn1] | 0.922 | 6.20 |
| APZ-2464 | 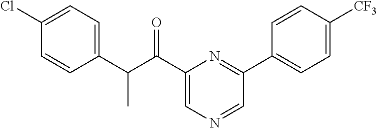 [SMILES: CC(C(=O)c1cncc(-c2ccc(C(F)(F)F)cc2)n1)c1ccc(Cl)cc1] | 1.08 | >10.0 |
| APZ-2465 | 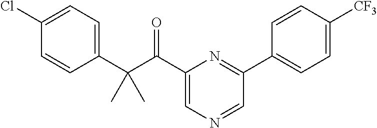 [SMILES: CC(C)(C(=O)c1cncc(-c2ccc(C(F)(F)F)cc2)n1)c1ccc(Cl)cc1] | 0.0872 | >10.0 |
| APZ-2469 | 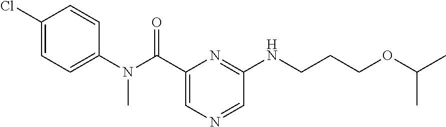 [SMILES: CC(C)OCCCNc1cncc(C(=O)N(C)c2ccc(Cl)cc2)n1] | >3.83 | >3.33 |
| APZ-2561 | 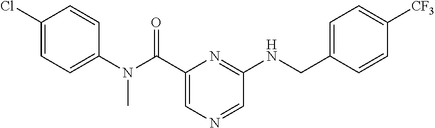 [SMILES: CN(C(=O)c1cncc(NCc2ccc(C(F)(F)F)cc2)n1)c1ccc(Cl)cc1] | >3.37 | 2.32 |
| APZ-2562 | 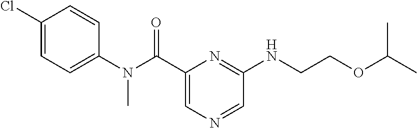 [SMILES: CC(C)OCCNc1cncc(C(=O)N(C)c2ccc(Cl)cc2)n1] | 4.46 | >10.0 |

Solubility was assessed for selected compounds using an HPLC assay. Solubilities of selected compounds are presented in Table 13:

TABLE 13
Solubilities of Selected Compounds

| Compound ID | Solubility (μM) at pH 7.4 |
|---|---|
| APZ-2161 | 4.22 |
| APZ-2216 | 9.91 |
| APZ-2198 | <2.50 |
| APZ-2214 | 10.3 |
| APZ-2240 | 11.2 |
| APZ-2327 | 19.1 |
| APZ-2213 | <2.50 |
| APZ-2217 | <2.50 |
| APZ-2241 | 10.9 |
| APZ-2317 | <2.50 |
| APZ-2010 | 4.35 |
| APZ-2025 | 3.23 |
| APZ-2306 | 184 |
| APZ-2160 | 12.8 |
| APZ-2322 | 9.02 |
| APZ-2199 | 7.92 |
| APZ-2123 | <2.50 |
| APZ-2257 | <2.50 |
| APZ-2157 | 9.66 |
| APZ-2248 | 15.2 |

Solubility tended to decrease with an increase in the number of lipophilic groups and a decrease in the number of nitrogens.

### Example 3: Generation of Blood Stage Mutants to Support Mechanism of Action Studies

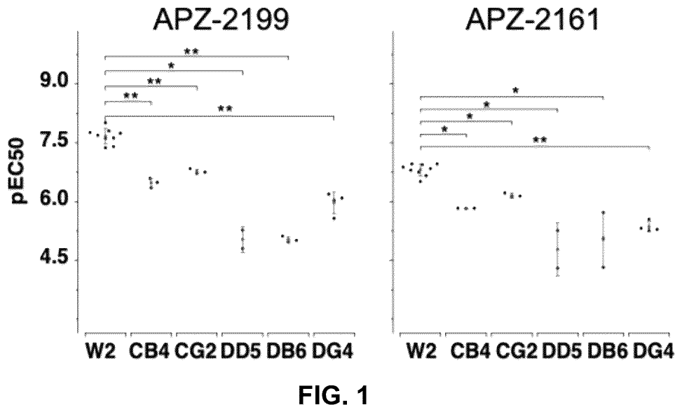

In vitro evolution and whole genome analysis (IVIEWGA) is one method used extensively in malaria parasites for target discovery. In this method, parasites are exposed to a sublethal concentration of a compound with antiparasitic activity. Once resistance is observed, the genomes of the resistant clones are analyzed using whole genome sequencing (WGS) and are compared to the sensitive parent clone to identify the genetic basis of resistance. We used this strategy to identify APZ-induced genetic changes. To this end, we generated APZ-resistant parasites to both frontrunners by treating cultures of 108 P. falciparum W2 strain parasites with 10×EC₅₀ constantly over two weeks. Parasites appeared in culture after 18 days and were subcloned, resulting in 2 mutants raised against APZ-2161 and 3 mutants raised against APZ-2199. Mutants were found to have EC₅₀ shifts compared to the sensitive parent ranging from 4.5-9.7-fold for those generated using APZ-2161 and 50-430-fold for those generated using APZ-2199. Testing APZ-2161-generated mutants against APZ-2199 and APZ-2199-generated mutants against APZ-2161 revealed cross-resistance, suggesting both cores are hitting the same target (FIG. 1). We next processed DNA from APZ-resistant and -sensitive P. falciparum parasites for WGS analyses. Copy number analysis did not reveal duplications or deletions in APZ-resistant parasites when compared to APZ-sensitive parasites and thus indicated that APZ-induced resistance does not induce genomic structural changes in novel or known loci that confer resistance across a variety of antimalarial chemotypes. We also compared base pair composition of APZ-resistant and -sensitive parasites using an optimized workflow for P. falciparum. We considered loci with single nucleotide polymorphisms (SNPs) or small insertions and deletions (INDELs) detected in 5 of 5 APZ-mutant clones sequenced and 0 of 3 APZ-sensitive forms to be of high relevance. Using this stringent criterion, we identified changes in 4 loci (Table 14). Interestingly, none of the modifications occur in genes representing targets of approved antimalarials or antimalarials in clinical trials. We detected changes encoding for modifications in the 3′ untranslated region (3 prime UTR variant) of three genes. These modifications could alter gene expression dynamics in APZ-resistant parasites. We also observed a modification encoding for missense mutation in the protein coding region of a conserved Plasmodium membrane protein (Table 14). This modification in APZ-resistant parasites results in an amino acid change at the protein level (Lys→Ser) which could support reduced APZ binding to this putative target. Together, these results suggest the mechanism of action of APZs is different than currently available antimalarials. Furthermore, these data combined with the high selectivity of our compounds over human cells further suggests that the biochemical target(s) are both unique and species-specific.

TABLE 14
Genes with SNPs or INDELs in APZ-Resistant Parasites (Mutants)
Compared to APZ-Sensitive Parents (Controls)

| | | | Modification |
|---|---|---|---|
| Gene Description | Mutants | Controls | Type |
| PF3D&_0801800 (mannose-6-phosphate, isomerase, putative) | 5/5 | 0/3 | 3′ UTR variant |
| 3PF3D7_1015800 (ribonucleoside-diphosphate reductase small | 5/5 | 0/3 | 3′ UTR variant |
| chain, putative) | | | |
| PF3D7_1467400 (ribosomal protein L22, apicoplast, putative) | 5/5 | 0/3 | 3′ UTR variant |
| PF3D7_0804500 (conserved Plasmodium membrane protein, | 5/5 | 0/3 | Missense |
| unknown function) | | | variant |

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is:

1. A compound comprising Formula I: 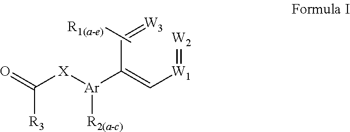
wherein each of R₁ₐ—R₁ₑ is independently selected from hydrogen, Cl, CH₃, OCH₃, CH₂CH₃, CF₃, CH(CH₃)₂, OCF₃, OCF₂H, CF₂H, NH₂, C(═O)CF₃, S(═O)₂CH₃, cyclobutyl, cyclopentyl, cyclohexyl, or any combination thereof;
wherein W₁, W₂, and W₃ are independently N or substituted or unsubstituted C;
wherein Ar is a 5- or 6-membered aromatic or heteroaromatic ring or a bridged cycloalkyl or heterocycloalkyl ring;
wherein each of R₂ₐ—R_{2c}, if present, is hydrogen, C1-C4 linear or branched alkyl, or when R₄ is present, one of R₂ₐ—R_{2c} and R₄ together form a C1-C4 alkyl bridge;
wherein X is NR₈ or is absent;
wherein R₈ is C1-C4 linear or branched alkyl, CH₂CF₃, or hydrogen;
wherein R₃ is selected from 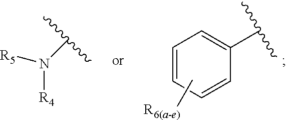
wherein R₄ is selected from deuterated or undeuterated C1-C4 linear or branched alkyl; hydrogen; or together with R₅ forms a substituted or unsubstituted cycloalkyl, heterocycloalkyl, aryl, or heteroaryl group containing from 3 to 9 ring atoms, or R₄ and one of R₂ₐ—R_{2c} together form a C1-C4 alkyl bridge;
wherein R₅ is selected from substituted or unsubstituted C3-C9 cycloalkyl, heterocycloalkyl, aryl, or heteroaryl, or together with R₄ forms a substituted or unsubstituted cycloalkyl, heterocycloalkyl, aryl, or heteroaryl group containing from 3 to 9 ring atoms; and
wherein each of R₆ₐ—R₆ₑ is independently selected from hydrogen, halogen, hydroxyl, cyano, nitro, substituted or unsubstituted C1-C4 linear or branched alkyl, alkoxy, alkylsulfonyl, substituted or unsubstituted amino, substituted or unsubstituted amide, substituted or unsubstituted ketone, or any combination thereof,
provided the compound of Formula I is not

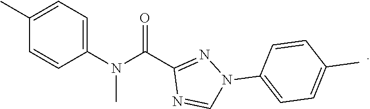

2. The compound of claim 1, having the structure 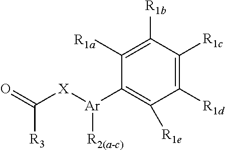
wherein R₁ₐ, R_{1b}, R_{1d}, and R₁ₑ are hydrogen and R_{1c} is selected from hydrogen, Cl, CH₃, OCH₃, CH₂CH₃, CF₃, CH(CH₃)₂OCF₃, OCF₂H, CF₂H, cyclobutyl, cyclopentyl, or cyclohexyl.

3. The compound of claim 1, having the structure 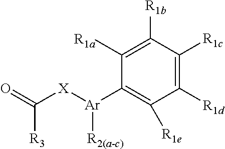
wherein R₁ₐ and R_{1b} are hydrogen, R_{1c} is selected from hydrogen, Cl, CH₃, CH₂CH₃, CF₃, OCH₃, CH(CH₃)₂, OCF₃, OCF₂H, CF₂H, cyclobutyl, cyclopentyl, or cyclohexyl, and R_{1d} and R₁ₑ are independently selected from hydrogen, Cl, and CH₃.

4. The compound of claim 1, wherein each of R₂ₐ—R_{2c}, if present, is hydrogen or methyl, or when R₄ is present, one of R₂ₐ—R_{2c} and R₄ together form a C1 alkyl bridge.

5. The compound of claim 1, wherein R₃ is 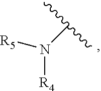
wherein R₄ is hydrogen, methyl, ethyl, isopropyl, tert-butyl, CD₃, CH₂CF₃, or together with R₅ forms a substituted or unsubstituted cycloalkyl or heterocycloalkyl, group containing from 3 to 9 ring atoms, or R₄ and one of R₂ₐ—R_{2c}, if present, together form a C1 alkyl bridge; and
wherein R₅ is selected from substituted or unsubstituted C3-C9 cycloalkyl, heterocycloalkyl, aryl, or heteroaryl, or together with R₄ forms a substituted or unsubstituted cycloalkyl or heterocycloalkyl group containing from 3 to 9 ring atoms.

6. The compound of claim 5, wherein R₃ is selected from

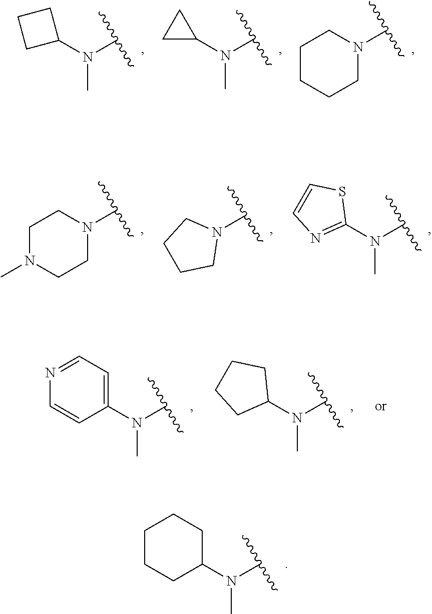

7. The compound of claim 5, wherein R₄ is H, methyl, ethyl, isopropyl, tert-butyl, or CD₃, and wherein R₅ is 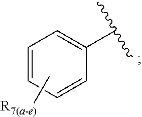
wherein each of R₇ₐ—R₇ₑ is independently selected from hydrogen, halogen, hydroxyl, cyano, nitro, trifluoromethoxy, substituted or unsubstituted C1-C4 linear or branched alkyl, alkoxy, alkylsulfonyl, substituted or unsubstituted amino, substituted or unsubstituted amide, or any combination thereof.

8. The compound of claim 7, wherein R₅ is 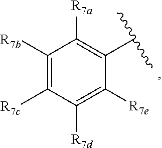
wherein R₇ₐ and R_{7b} are hydrogen;
wherein R_{7c} is selected from Cl, CH₃, OCH₃, CN, H, SO₂CH₃, COCH₃, NO₂, NHCOCH₃, CF₃, OH, F, N(CH₃)₂, CH₂CH₃, CH(CH₃)₂, or C(CH₃)₃;
wherein R_{7d} is selected from Cl, H, CH₃, or CF₃; and
wherein R₇ₑ is selected from H or CH₃.

9. The compound of claim 7, wherein R₇ₐ, R_{7b}, R_{7d}, and R₇ₑ are hydrogen and wherein R_{7c} is selected from Cl or CH₃.

10. The compound of claim 1, wherein R₃ is 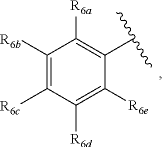
and wherein R₆ₐ—R₆ₑ are independently selected from Cl, CH₃, OCH₃, CN, H, SO₂CH₃, COCH₃, NO₂, NHCOCH₃, CF₃, OH, F, N(CH₃)₂, CH₂CH₃, CH(CH₃)₂, C(CH₃)₃, or any combination thereof.

11. The compound of claim 1, wherein R₆ₐ, R_{6b}, R_{6d}, and R₆ₑ are hydrogen, and wherein R_{6c} is selected from Cl or methyl.

12. The compound of claim 1, wherein R₈ is H or methyl.

13. The compound of claim 1, wherein Ar is selected from

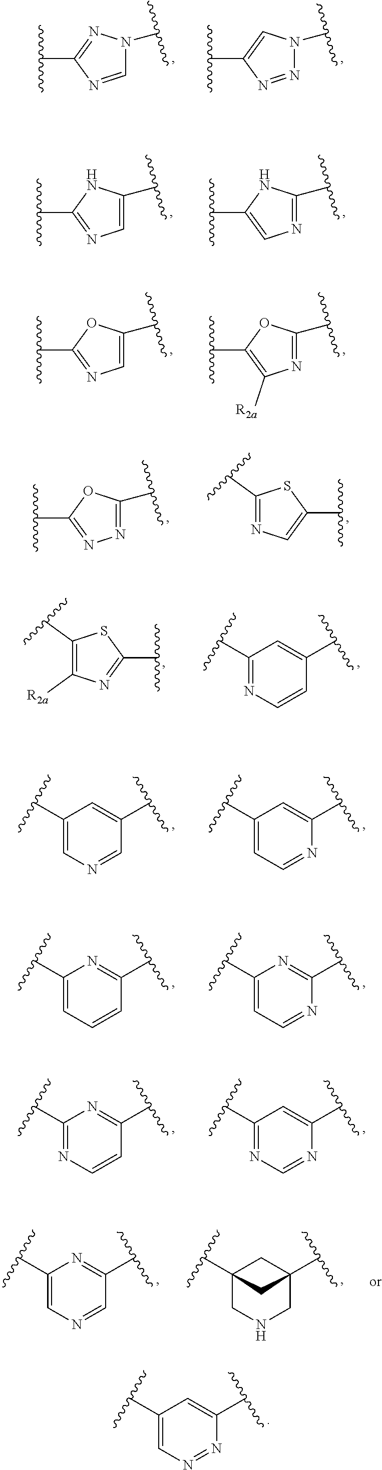

14. The compound of claim 1, wherein the compound is selected from 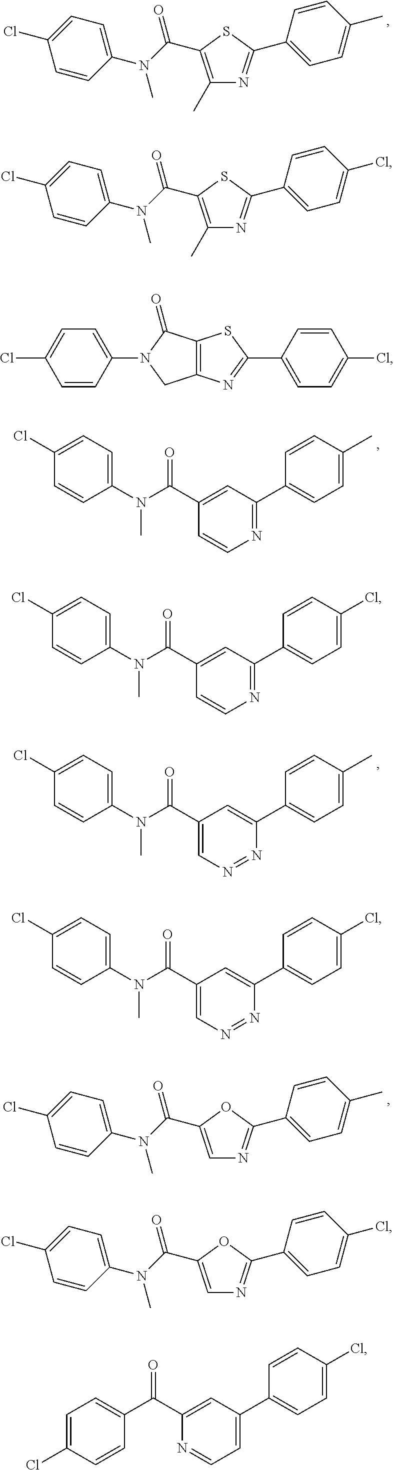 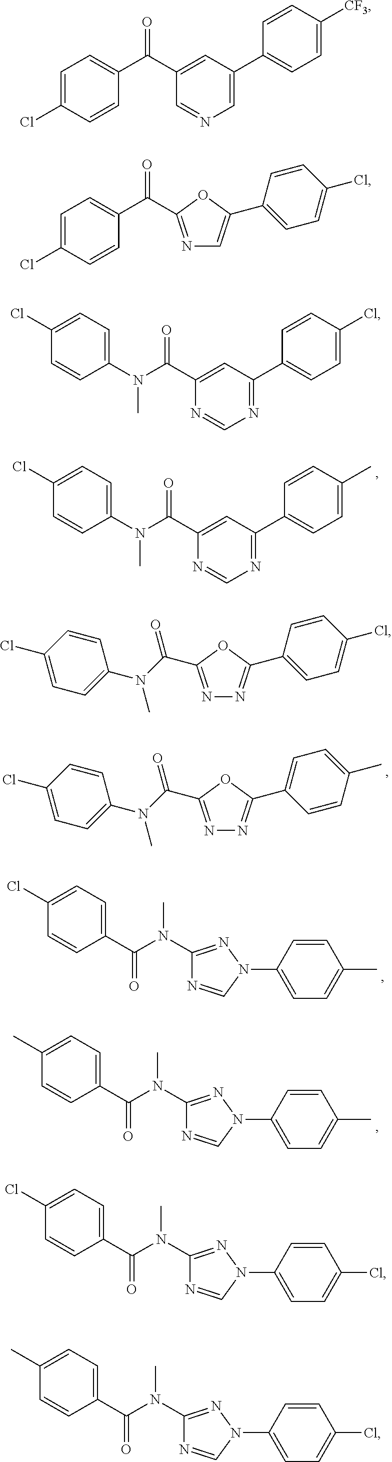 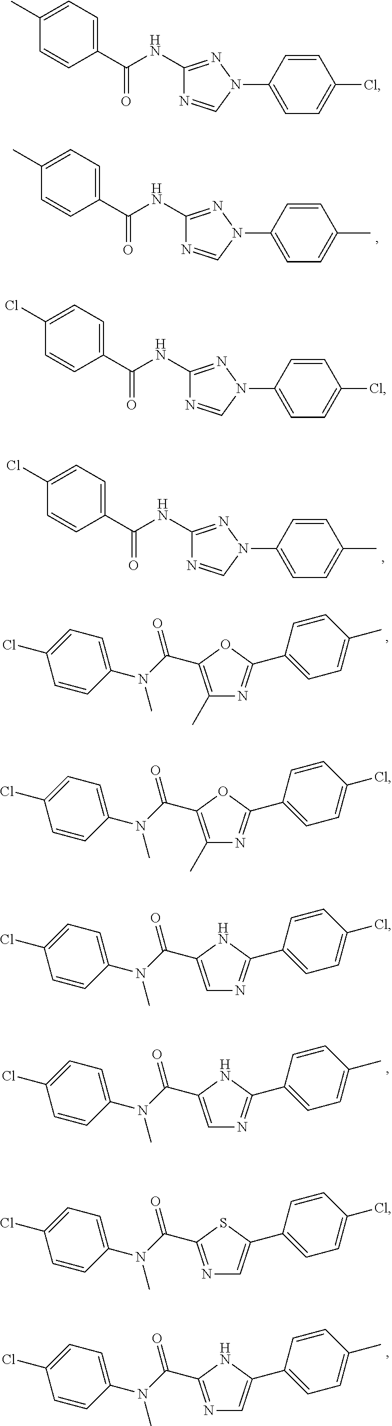 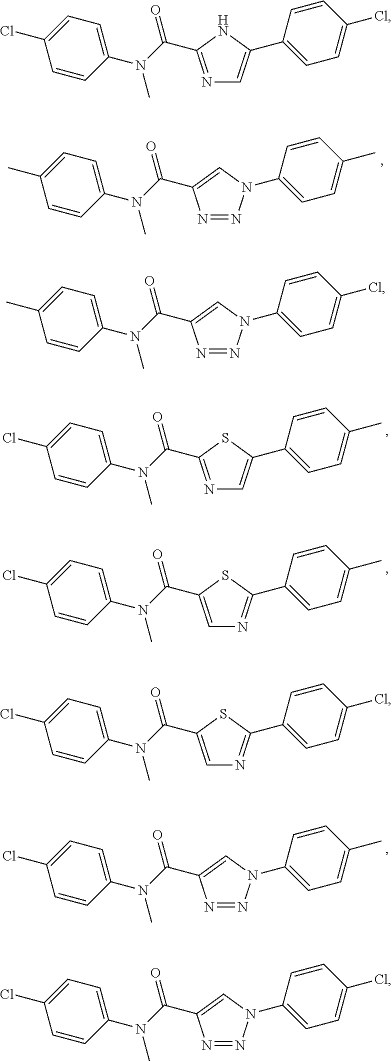
or any combination thereof.

15. The compound of claim 1, wherein the compound is selected from 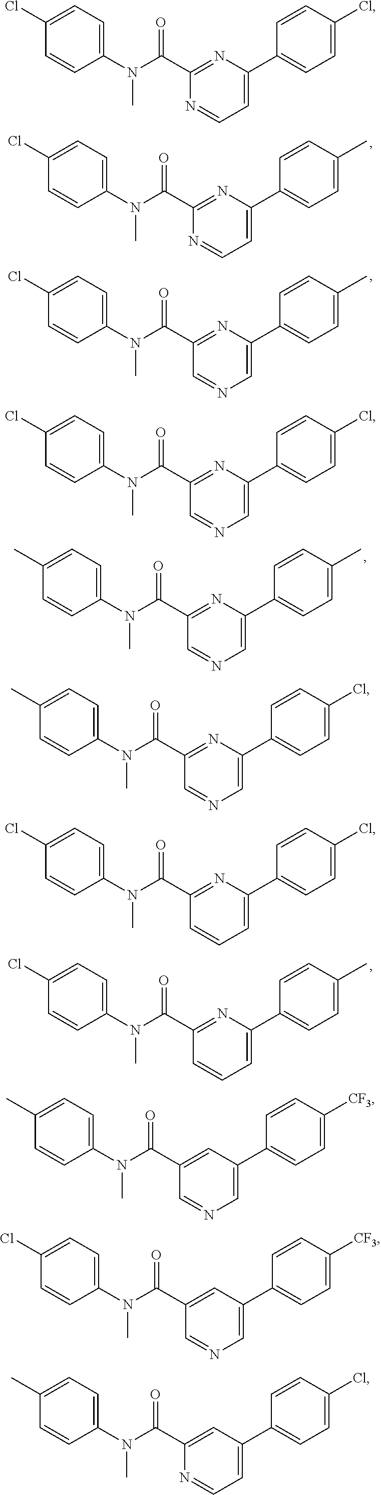 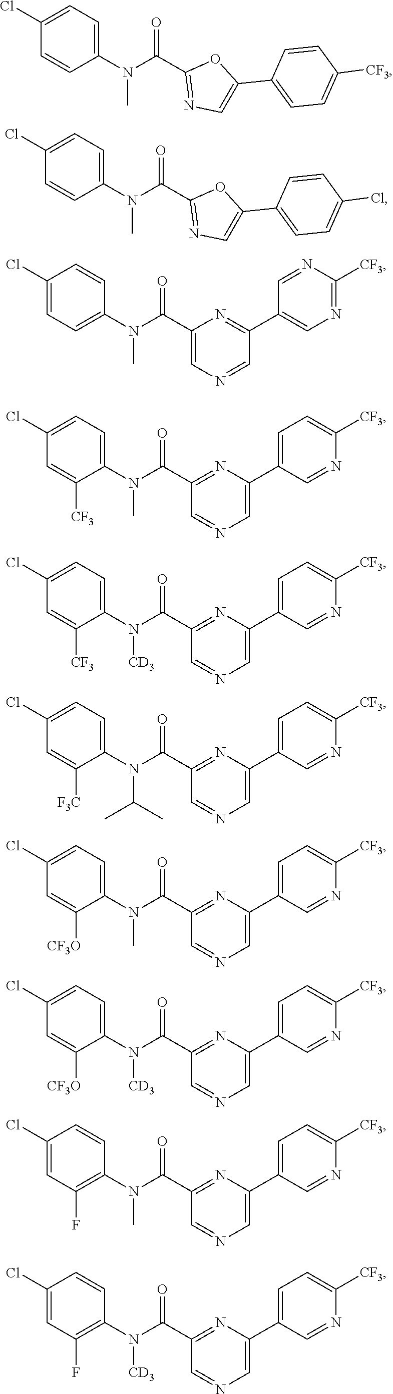 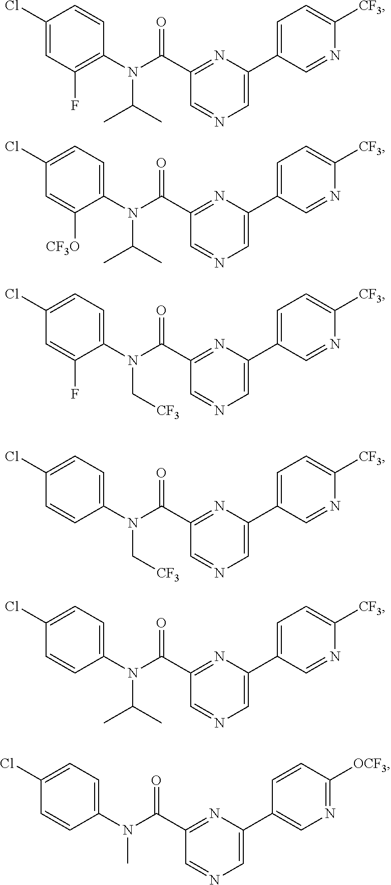
or any combination thereof.

16. A compound comprising Formula II: 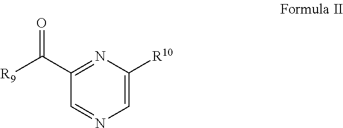
wherein R₁₀ is selected from substituted or unsubstituted C5-C6 cycloalkyl, substituted or unsubstituted C5-C6 heterocycloalkyl, substituted or unsubstituted C5-C6 cycloalkenyl, substituted or unsubstituted C5-C6 heterocycloalkenyl, 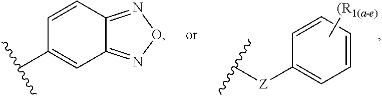
wherein Z is NH or is absent,
wherein each of R₁ₐ—R₁ₑ is independently selected from hydrogen, Cl, CH₃, OCH₃, CH₂CH₃, CF₃, CH(CH₃)₂, OCF₃, OCF₂H, CF₂H, cyclobutyl, cyclopentyl, cyclohexyl, or any combination thereof,
wherein R₉ is —Y—R₁₃;
wherein Y is selected from OR₁₂, NR₁₁R₁₂, CHR₁₁, or C(R₁₁)₂;
wherein R₁₁ is C1-C4 alkyl or deuterated alkyl;
wherein R₁₂ is a substituted or unsubstituted C5-C7 cycloalkyl, heterocycloalkyl, aryl, or heteroaryl group; or
wherein R₁₁ and R₁₂ together form a substituted or unsubstituted C5-C10 cycloalkyl, heterocycloalkyl, aryl, or heteroaryl group.

17. The compound of claim 16, having the structure

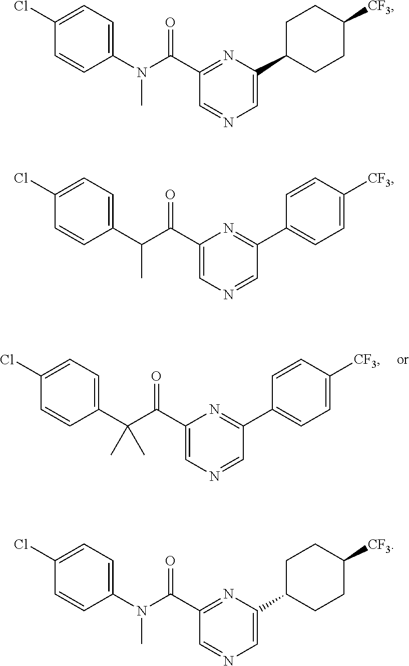 [SMILES: CN(C(=O)c1cncc([C@H]2CC[C@@H](C(F)(F)F)CC2)n1)c1ccc(Cl)cc1 ; CC(C(=O)c1cncc(-c2ccc(C(F)(F)F)cc2)n1)c1ccc(Cl)cc1 ; CC(C)(C(=O)c1cncc(-c2ccc(C(F)(F)F)cc2)n1)c1ccc(Cl)cc1 ; CN(C(=O)c1cncc([C@H]2CC[C@H](C(F)(F)F)CC2)n1)c1ccc(Cl)cc1]

18. A pharmaceutical composition comprising the compound claim 1 or a pharmaceutically acceptable salt thereof.

19. The pharmaceutical composition of claim 18, wherein the pharmaceutical composition has an EC₅₀ against Plasmodium falciparum asexual blood stages (W2) of less than or equal to 1 μM.

20. A method for treating or preventing an infection caused by a Plasmodium species in a subject, the method comprising administering a therapeutically effective amount of the pharmaceutical composition of claim 18 to the subject.
